(19) United States
(12) Patent Application Publication
(10) Pub. No.: US 2024/0083891 A1
(43) Pub. Date: Mar. 14, 2024

(54) MATERIALS FOR ORGANIC ELECTROLUMINESCENT DEVICES

(71) Applicant: Merck Patent GmbH, Darmstadt (DE)

(72) Inventors: Amir Hossain PARHAM, Darmstadt (DE); Philipp STOESSEL, Darmstadt (DE); Christian EHRENREICH, Darmstadt (DE); Jonas Valentin KROEBER, Darmstadt (DE); Christian EICKHOFF, Darmstadt (DE)

(21) Appl. No.: 18/270,734

(22) PCT Filed: Jan. 3, 2022
(86) PCT No.: PCT/EP2022/050004

§ 371 Date: Jul. 3, 2023

(30) Foreign Application Priority Data
Jan. 5, 2021 (EP) 21150212.5

(51) Int. Cl. C07D 413/10; C07D 413/14; C07D 417/10; C07D 417/14; C07D 419/14; C07D 498/04; C07D 513/04; H10K 85/60

(52) U.S. Cl. CPC C07D 413/10; C07D 413/14; C07D 417/10; C07D 417/14; C07D 419/14; C07D 498/04; C07D 513/04; H10K 85/622; H10K 85/636; H10K 85/654; H10K 85/6572; H10K 85/6574; H10K 85/6576; H10K 2101/20

(57) ABSTRACT

The present invention relates to compounds which are suitable for use in electronic devices, and to electronic devices, in particular organic electroluminescent devices, containing these compounds.

The present invention relates to electronic devices, especially organic electroluminescent devices, comprising triphenylene derivatives.

Emitting materials used in organic electroluminescent devices (OLEDs) are frequently phosphorescent organometallic complexes. In general terms, there is still a need for improvement in OLEDs, especially also in OLEDs which exhibit triplet emission (phosphorescence), for example with regard to efficiency, operating voltage and lifetime. The properties of phosphorescent OLEDs are not just determined by the triplet emitters used. More particularly, the other materials used, such as matrix materials or charge transport materials, are also of particular significance here. Improvements to these materials can thus also lead to improvements in the OLED properties. Suitable matrix materials for OLEDs are, for example, triphenylene derivatives as disclosed, for example, in WO 2011/137157 or WO 2012/048781.

The problem addressed by the present invention is that of providing compounds which are suitable for use in an OLED, especially as matrix material for phosphorescent emitters or as electron transport material, and which lead to improved properties therein. It is a further object of the present invention to provide further organic semiconductors for organic electroluminescent devices, in order thus to enable the person skilled in the art to have a greater possible choice of materials for the production of OLEDs.

It has been found that, surprisingly, this object is achieved by particular triphenylene derivatives substituted by electron-deficient heteroaryl groups having at least two nitrogen atoms that are described below, and these are of good suitability for use in OLEDs. These OLEDs especially have a relatively long lifetime, but also improved efficiency and relatively low operating voltage. The present invention therefore provides these compounds and electronic devices, especially organic electroluminescent devices, comprising these compounds.

The present invention provides a compound of formula (1)

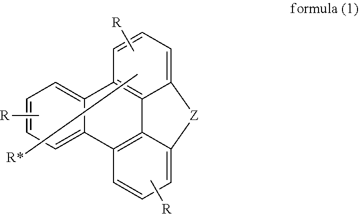

where the R radicals may also occur more than once and the symbols used are:
Z is O or S;
R* is a group of the following formula (2), where the dotted bond represents the bond to the base skeleton in the formula (1),

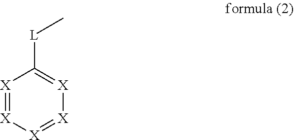

X is the same or different at each instance and is CR or N; or two adjacent X groups are a group of the following formula (3), (4) or (5), with the proviso that at least two and at most three X groups are N,

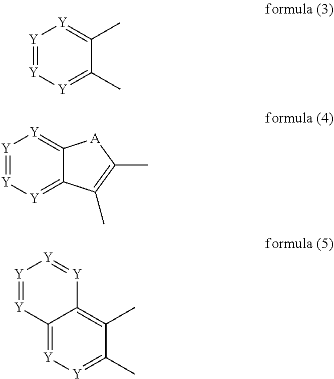

where the dotted bonds indicate the linkage of this group in the formula (2);
Y is the same or different at each instance and is CR or N;
A is NR, O, S or CR₂;
L is a single bond or an aromatic or heteroaromatic ring system which has 5 to 24 aromatic ring atoms and may be substituted by one or more R radicals;
R is the same or different at each instance and is H, D, F, Cl, Br, I, N(R¹)₂, CN, NO₂, OR¹, SR¹, COOR¹, C(═O)N(R¹)₂, Si(R¹)₃, B(OR¹)₂, C(═O)R¹, P(═O)(R¹)₂, S(═O)R¹, S(═O)₂R¹, OSO₂R¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals, where one or more nonadjacent CH₂ groups may be replaced by Si(R¹)₂, C═O, NR¹, O, S or CONR¹, or an aromatic or heteroaromatic ring system which has 5 to 60 aromatic ring atoms, preferably 5 to 40 aromatic ring atoms, and may be substituted in each case by one or more R¹ radicals; at the same time, two R radicals together may also form an aliphatic or heteroaliphatic ring system;
R¹ is the same or different at each instance and is H, D, F, Cl, Br, I, CN, NO₂, OR², SR², Si(R²)₃, B(OR²)₂, C(═O)R², P(═O)(R²)₂, S(═O)R², S(═O)₂R², OSO₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may each be substituted by one or more R² radicals, where one or more nonadjacent CH₂ groups may be replaced by Si(R²)₂, C═O, NR², O, S or CONR² and where one or more hydrogen atoms in the alkyl, alkenyl or alkynyl group may be replaced by D, F, Cl, Br, I or CN, or an aromatic or heteroaromatic ring system which has 5 to 40 aromatic ring atoms and may be substituted in each case by one or more R² radicals; at the same time, two or more R¹ radicals together may form an aliphatic ring system;
R² is the same or different at each instance and is H, D, F, CN or an aliphatic, aromatic or heteroaromatic organic radical, especially a hydrocarbyl radical, having 1 to 20 carbon atoms, in which one or more hydrogen atoms may also be replaced by F.

If two adjacent X groups are a group of the formula (3), (4) or (5), the remaining X groups in formula (2) are the same or different and are CR or N, with proviso that at least two X are N.

An aryl group in the context of this invention contains 6 to 40 carbon atoms and contains no heteroatoms in the ring system. A heteroaryl group in the context of this invention contains 2 to 40 carbon atoms and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused (annelated) aryl or heteroaryl group, for example naphthalene, anthracene, phenanthrene, quinoline, isoquinoline, etc. Aromatics joined to one another by a single bond, for example biphenyl, by contrast, are not referred to as an aryl or heteroaryl group but as an aromatic ring system.

An aromatic ring system in the context of this invention contains 6 to 60 carbon atoms, preferably 6 to 40 carbon atoms, in the ring system and does not contain any heteroatoms in the ring system. A heteroaromatic ring system in the context of this invention contains 2 to 60 carbon atoms, preferably 2 to 40 carbon atoms, and at least one heteroatom in the ring system, with the proviso that the sum total of carbon atoms and heteroatoms is at least 5. The heteroatoms are preferably selected from N, O and/or S. These shall likewise be understood to mean systems in which two or more aryl or heteroaryl groups are joined directly to one another, for example biphenyl, terphenyl, bipyridine or phenylpyridine. For example, systems such as fluorene or 9,9′-spirobifluorene shall also be regarded as aromatic ring systems in the context of this invention.

In the context of the present invention, the term “alkyl group” is used as an umbrella term for linear, branched and cyclic alkyl groups. Analogously, the terms “alkenyl group” and “alkynyl group” are used as umbrella terms for linear, branched and cyclic alkenyl and alkynyl groups respectively.

In the context of the present invention, an aliphatic hydrocarbyl radical or an alkyl group or an alkenyl or alkynyl group which may contain 1 to 40 carbon atoms and in which individual hydrogen atoms or CH₂ groups may also be substituted by the abovementioned groups is preferably understood to mean the methyl, ethyl, n-propyl, i-propyl, n-butyl, i-butyl, s-butyl, t-butyl, 2-methylbutyl, n-pentyl, s-pentyl, neopentyl, cyclopentyl, n-hexyl, neohexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl radicals. An alkoxy group OR¹ having 1 to 40 carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group SR¹ having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, Cl, Br, I, CN or NO₂, preferably F, Cl or CN, more preferably F or CN.

An aromatic or heteroaromatic ring system which has 5-60 aromatic ring atoms and may also be substituted in each case by the abovementioned radicals and which may be joined to the aromatic or heteroaromatic system via any desired positions is understood to mean especially groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived from combinations of these systems.

The wording that two or more radicals together may form a ring, in the context of the present description, should be understood to mean, inter alia, that the two radicals are joined to one another by a chemical bond with formal elimination of two hydrogen atoms. This is illustrated by the following scheme:

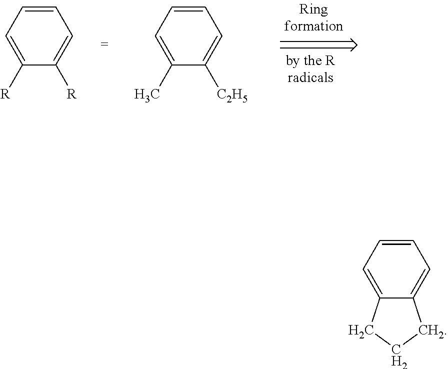

In addition, however, the abovementioned wording shall also be understood to mean that, if one of the two radicals is hydrogen, the second radical binds to the position to which the hydrogen atom was bonded, forming a ring. This will be illustrated by the following scheme:

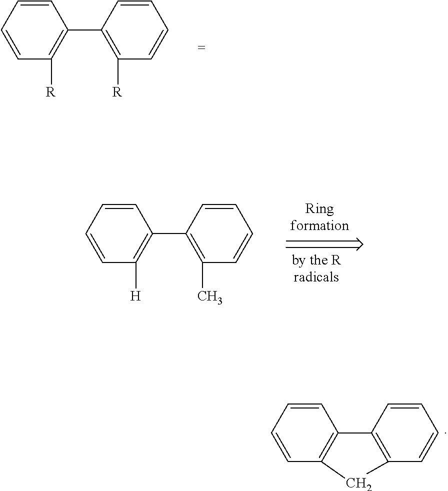

The R* group, i.e. the group of the formula (2), may either be bonded to the ring to which no Z group is bonded, so as to result in the compounds of the following formula (6), or maybe bonded to the same ring as the Z group, so as to result in the compounds of the following formula (7):

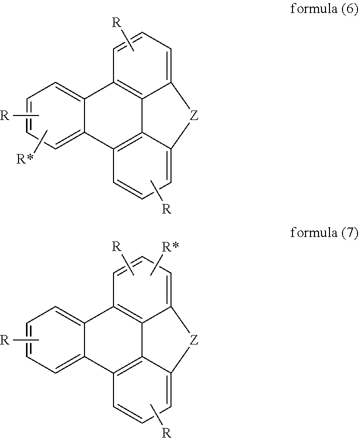

where the R radicals may also occur more than once, and the symbols used have the definitions given above.

Preference is given to the compounds of the following formulae (6a), (6b), (7a) and (7b):

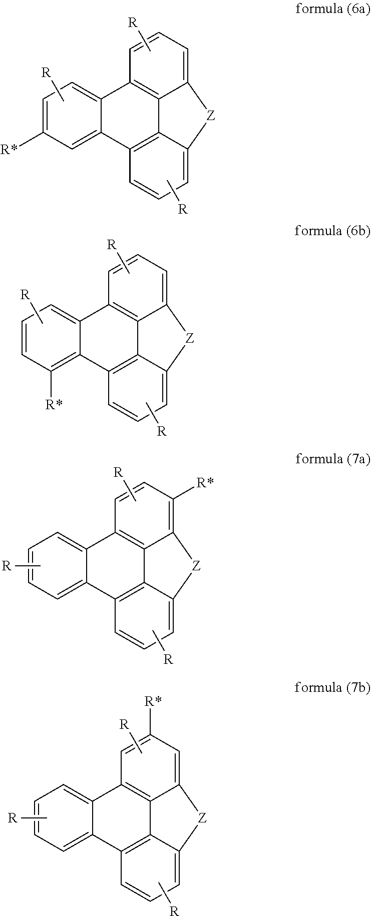

where the R radicals may also occur more than once, and the symbols used have the definitions given above.

Particular preference is given to the compounds of the formulae (6a), (7a) and (7b), especially the compounds of the formula (6a): In a preferred embodiment of the compounds set out above and hereinafter, Z is O.

In a further preferred embodiment of the invention, the compound of the formula (1) all of the preferred embodiments contains not more than two substituents R that are a group other than H and D, and more preferably not more than one substituents R is a group other than H and D. The substituents R other than H and D are preferably bonded here to a different ring than the R* group. Particular preference is given to the compounds of the following formulae (6a-1) to (7b-4):

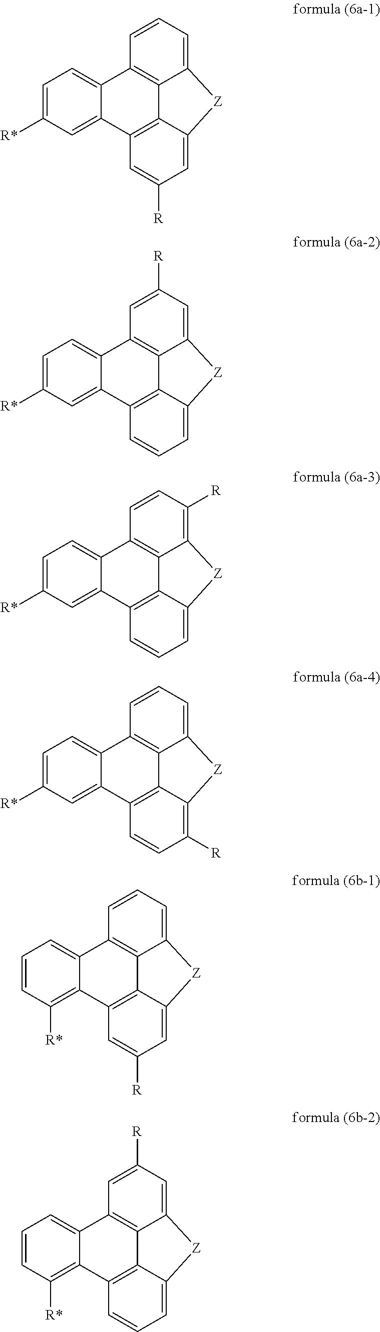

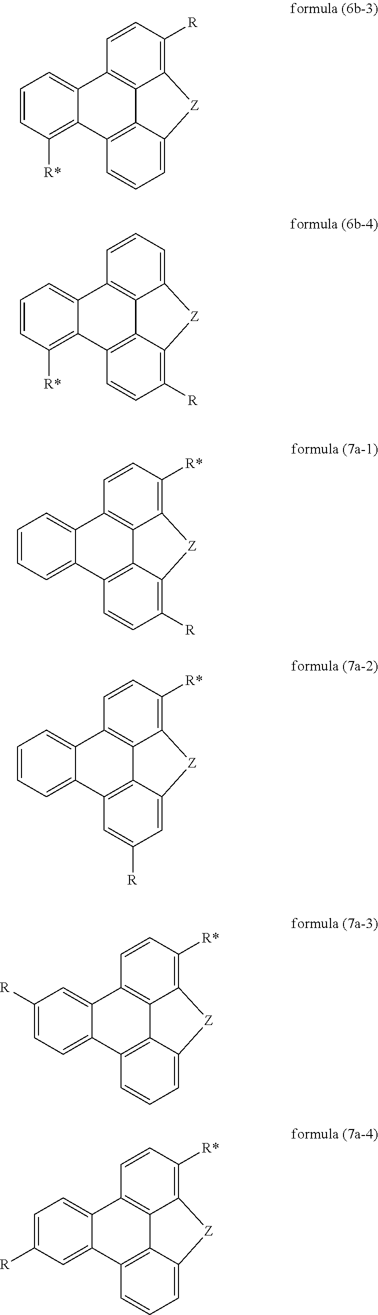

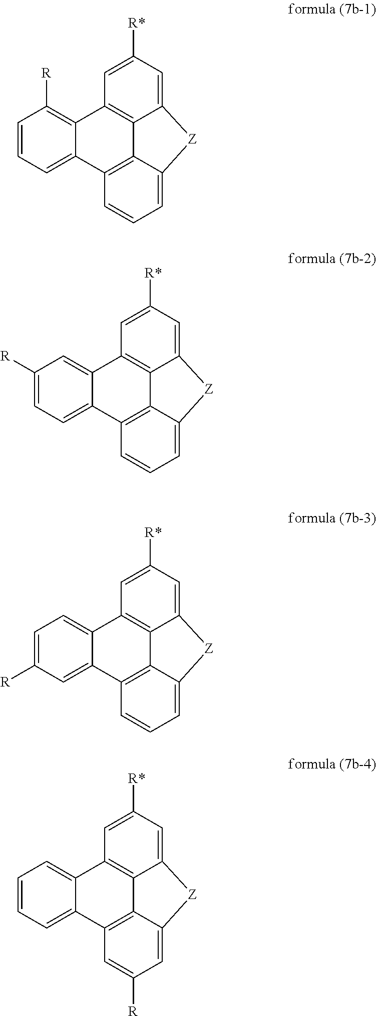

where the symbols used have the definitions given above.

There follows a description of preferred embodiments of R*, i.e. preferred groups of the formula (2).

In a preferred embodiment of the invention, the L group is a single bond or a divalent aromatic or heteroaromatic ring system which has 6 to 18 aromatic ring atoms and may be substituted in each case by one or more R radicals. More preferably, L is a single bond or an aromatic ring system which has 6 to 12 aromatic ring atoms and may be substituted by one or more R radicals, or a dibenzofuran or dibenzothiophene group that may be substituted by one or more R radicals. Most preferably, L is a single bond, a meta- or para-bonded phenylene group, or a dibenzofuran group. The dibenzofuran or dibenzothiophene group is preferably bonded in the 1,3, 1,6, 1,7, 1,8, 3,6, 3,8 or 3,9 position. The preference that L may be a dibenzofuran or dibenzothiophene group is applicable especially when L is a triazine group.

When L is an aromatic or heteroaromatic ring system, this is preferably selected from the structures of the following formulae (L-1) to (L-26):

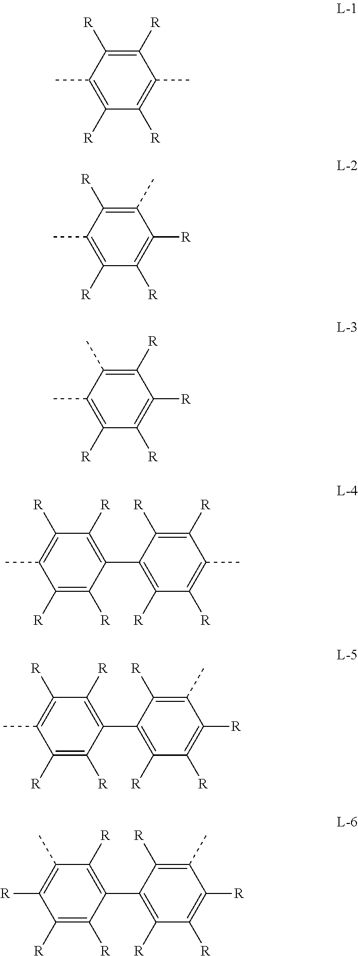

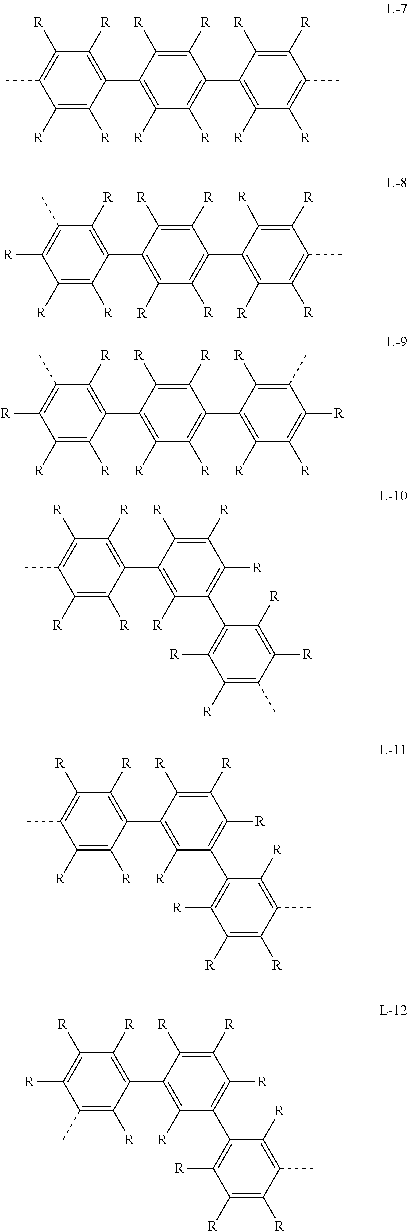

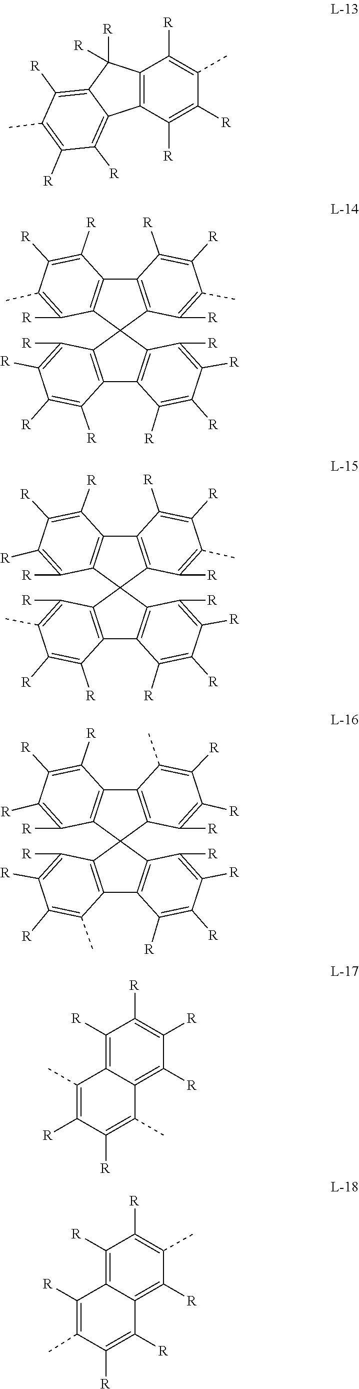

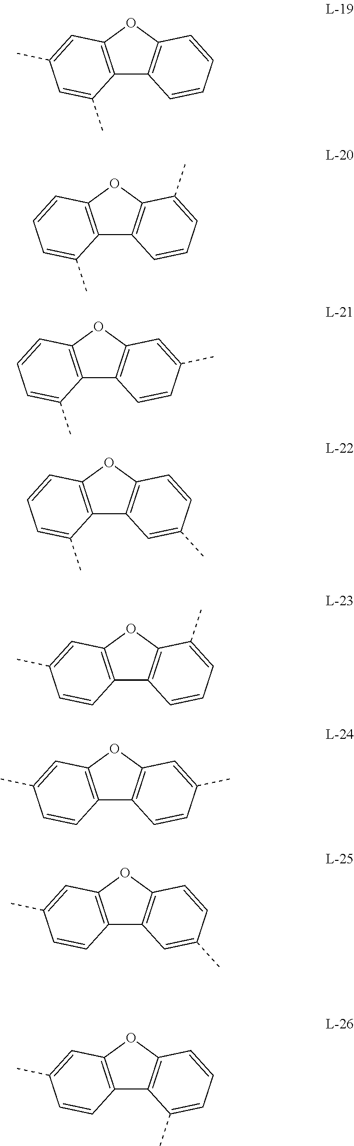

where the symbols used have the meanings given above and the dotted bonds represent the bonds to the heteroaryl group in formula (2) and to the base skeleton of the compound of the formula (1).

More preferably, L is a single bond or optionally substituted phenylene or biphenyl group, i.e. a group of the formula (L-1) to (L-6), especially (L-1), (L-2) or (L-6).

In a preferred embodiment of the formula (2), all X are the same or different and are CR or N, with the proviso that at least two X are N. These are preferably structures of the following formula (8):

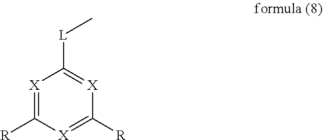

where the symbols used have the definitions given above, two or three X are N, and R is the same or different at each instance and is an aromatic or heteroaromatic ring system which has 5 to 40 aromatic ring atoms and may be substituted by one or more R¹ radicals.

Preferred embodiments of the formula (8) are the groups of the following formulae (8a), (8b) and (8c), particular preference being given to the groups of the formula (8a),

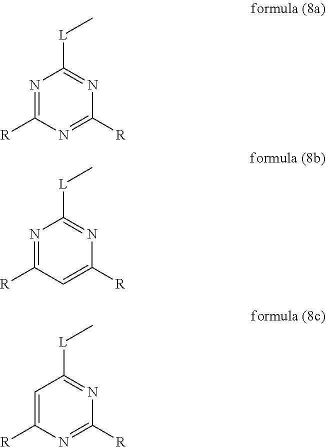

where the symbols used have the definitions given above and R is the same or different at each instance and is an aromatic or heteroaromatic ring system which has 5 to 40 aromatic ring atoms and may be substituted by one or more R¹ radicals.

In a further preferred embodiment of the formula (2), two adjacent X are a group of the formula (3), (4) or (5), where Y is the same or different and is CR, and, of the remaining X, exactly two X are N and the third X is CR, such that the structure is one according to one of the following formulae (9) to (18):

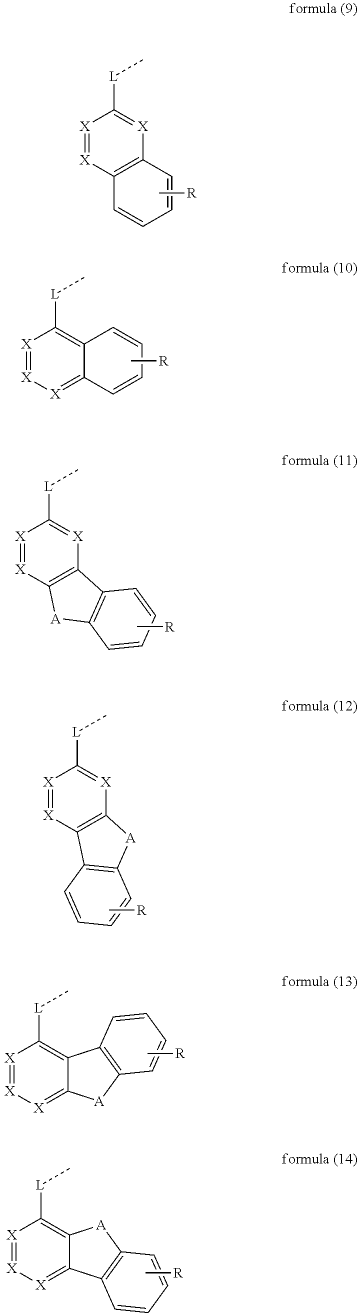

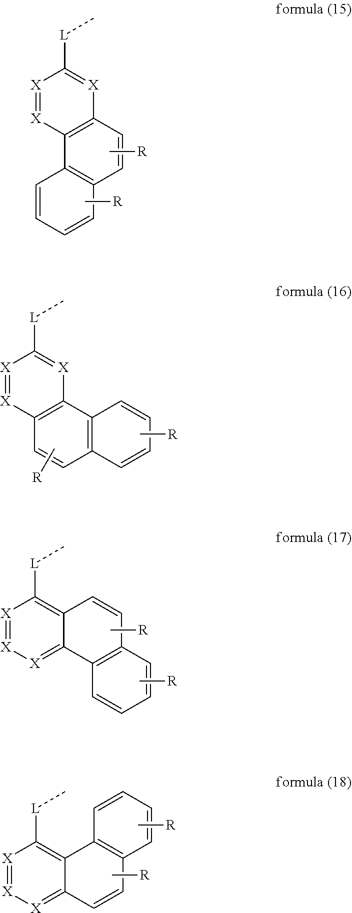

where the symbols have the definitions given above, R may also occur repeatedly, and exactly two X are N.

Preferred embodiments of the formulae (9) to (18) are the structures of the following formulae (9a) to (18a):

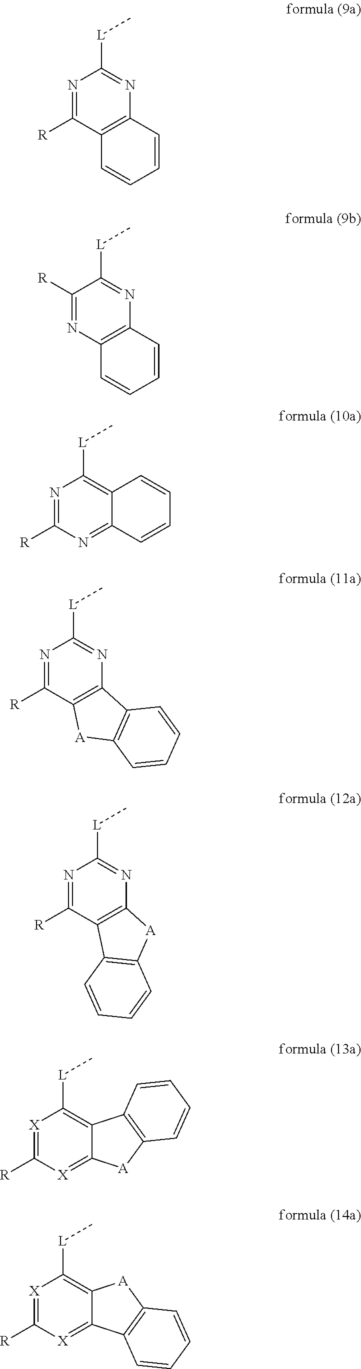

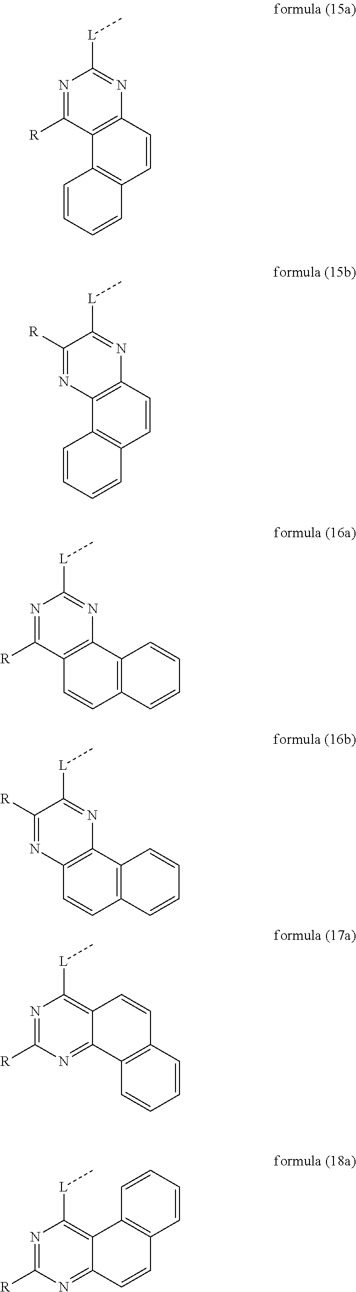

where the symbols used have the definitions given above and R is the same or different at each instance and is an aromatic or heteroaromatic ring system which has 5 to 40 aromatic ring atoms and may be substituted by one or more R¹ radicals.

In a further preferred embodiment of the invention, A is O or NR.

In a preferred embodiment of the formulae (8) to (18) or (8a) to (18a), R is the same or different at each instance and is an aromatic or heteroaromatic ring system which has 6 to 30 aromatic ring atoms and may be substituted by one or more R¹ radicals. More preferably, R is the same or different at each instance and is an aromatic or heteroaromatic ring system which has 6 to 24 aromatic ring atoms, especially 6 to 13 aromatic ring atoms, and may be substituted by one or more, preferably nonaromatic, R¹ radicals. In the formulae (9a) to (18a), R is most preferably selected from phenyl, d₅-phenyl, meta- or para-biphenyl, dibenzofuran or carbazole, where these groups may each be substituted by one or more R¹ radicals, but are preferably unsubstituted.

There follows a description of preferred substituents R, R¹ and R² in the compounds of the invention. In a particularly preferred embodiment of the invention, the preferences specified hereinafter for R, R¹ and R² occur simultaneously and are applicable to the structures of the formula (1) and to all preferred embodiments.

In a preferred embodiment of the invention, R is the same or different at each instance and is selected from the group consisting of H, D, F, CN, OR¹, a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may each be substituted by one or more R¹ radicals, but is preferably unsubstituted, and where one or more nonadjacent CH₂ groups may be replaced by O, or an aromatic or heteroaromatic ring system which has 6 to 30 aromatic ring atoms and may be substituted in each case by one or more R¹ radicals; at the same time, two R radicals together may also form an aliphatic, aromatic or heteroaromatic ring system. More preferably, R is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group in each case may be substituted by one or more R¹ radicals, but is preferably unsubstituted, or an aromatic or heteroaromatic ring system which has 6 to 24 aromatic ring atoms and may be substituted in each case by one or more R¹ radicals, preferably nonaromatic R¹ radicals. Most preferably, R is the same or different at each instance and is selected from the group consisting of H, D or an aromatic or heteroaromatic ring system which has 6 to 24 aromatic ring atoms and may be substituted in each case by one or more R¹ radicals, preferably nonaromatic R¹ radicals.

The substituents R bonded here to the triphenylene base skeleton are preferably the same or different at each instance and are selected from the group consisting of H, D and an aromatic ring system which has 6 to 24 aromatic ring atoms, more preferably 6 to 12 aromatic ring atoms, and may be substituted by one or more aromatic R¹ radicals, but is preferably unsubstituted. More preferably, the substituents R bonded to the triphenylene base skeleton are H or D, especially H.

Suitable aromatic or heteroaromatic ring systems R are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene which may be joined via the 1 or 2 position, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, quinazoline, benzimidazole, phenanthrene, triphenylene or a combination of two or three of these groups, each of which may be substituted by one or more R¹ radicals. When R is a heteroaryl group, especially triazine, pyrimidine, quinazoline or carbazole, preference may also be given to aromatic or heteroaromatic R¹ radicals on this heteroaryl group.

The R groups here on the base skeleton of the compound of the formula (1), when they are an aromatic or heteroaromatic ring system, and the R groups in the formulae (8) to (18a), are preferably selected from the groups of the following formulae R-1 to R-83:

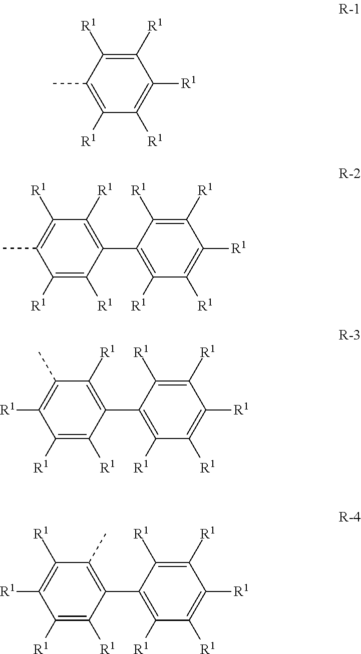

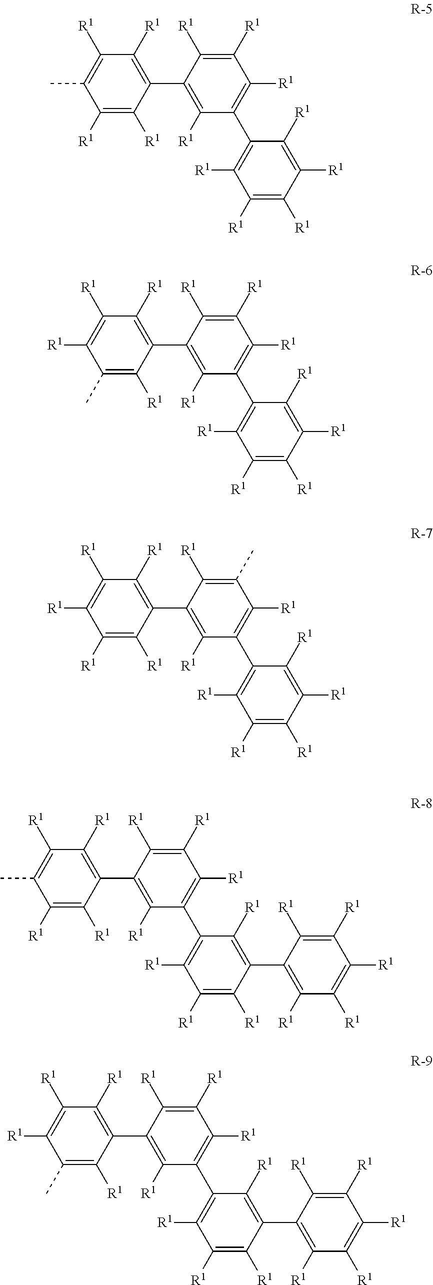

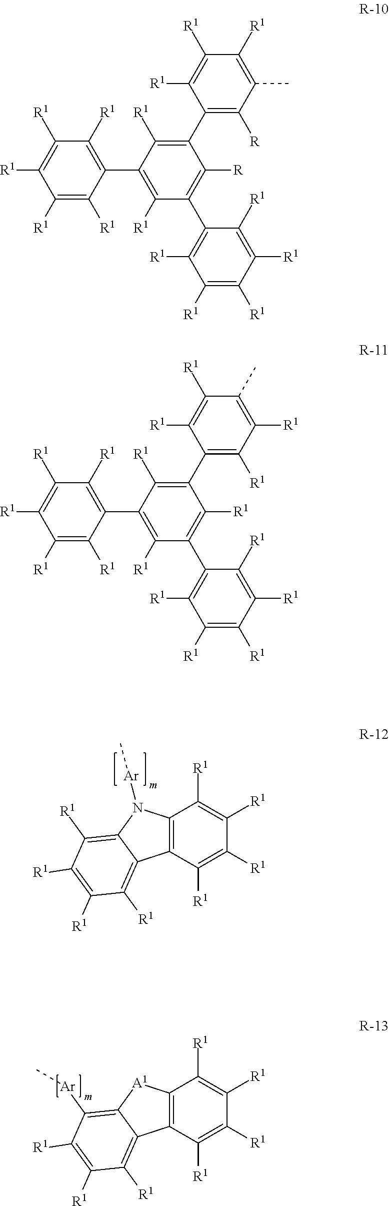

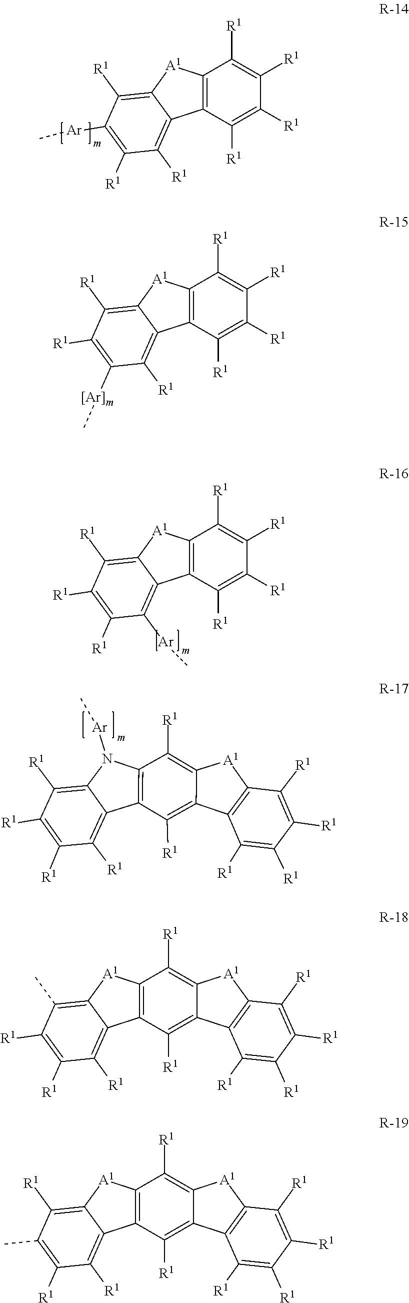

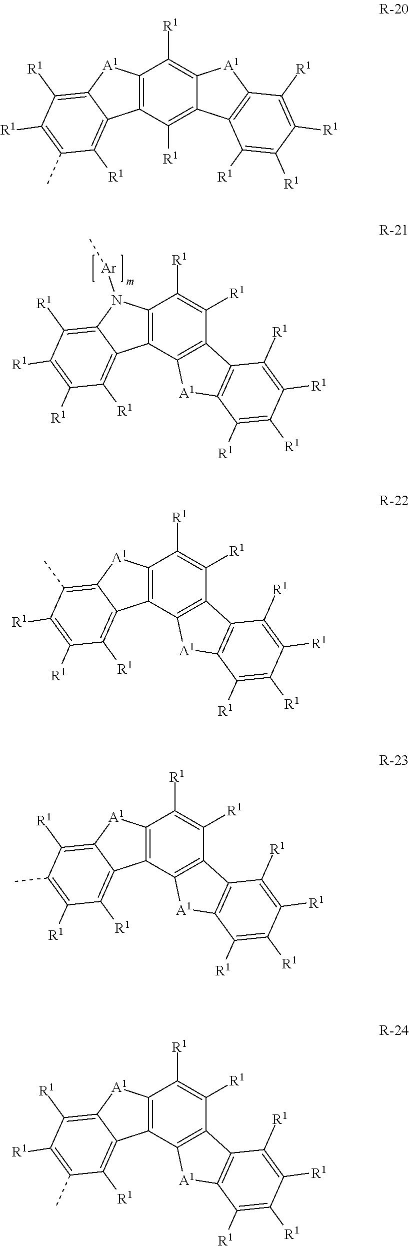

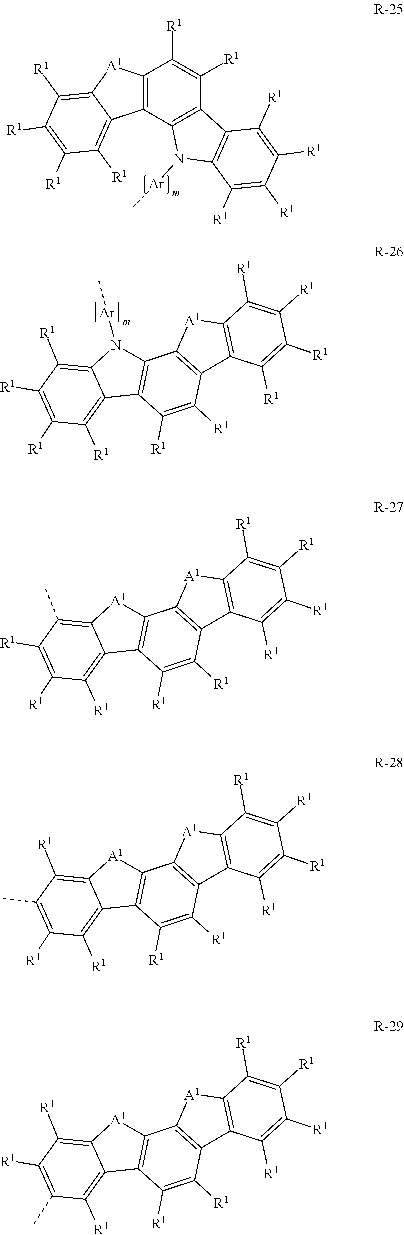

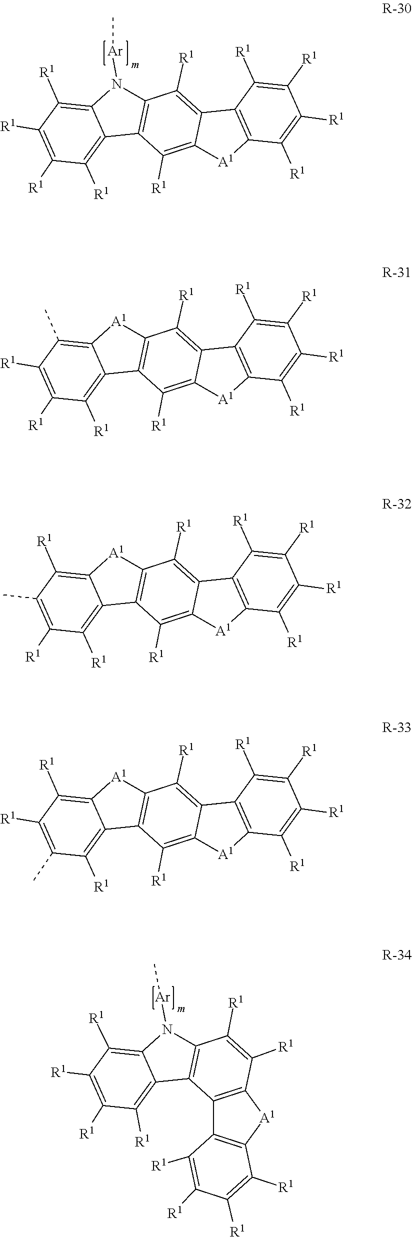

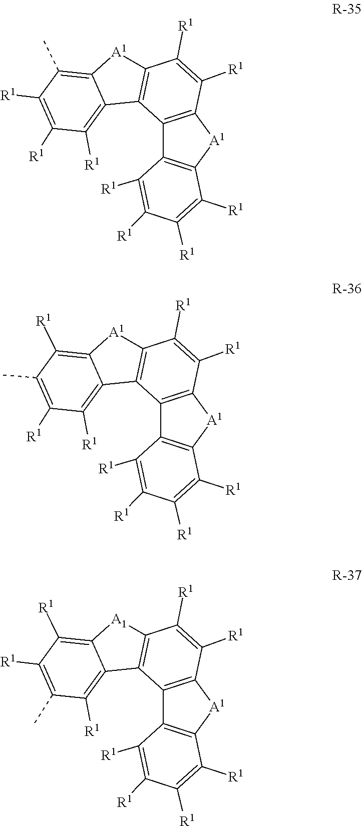

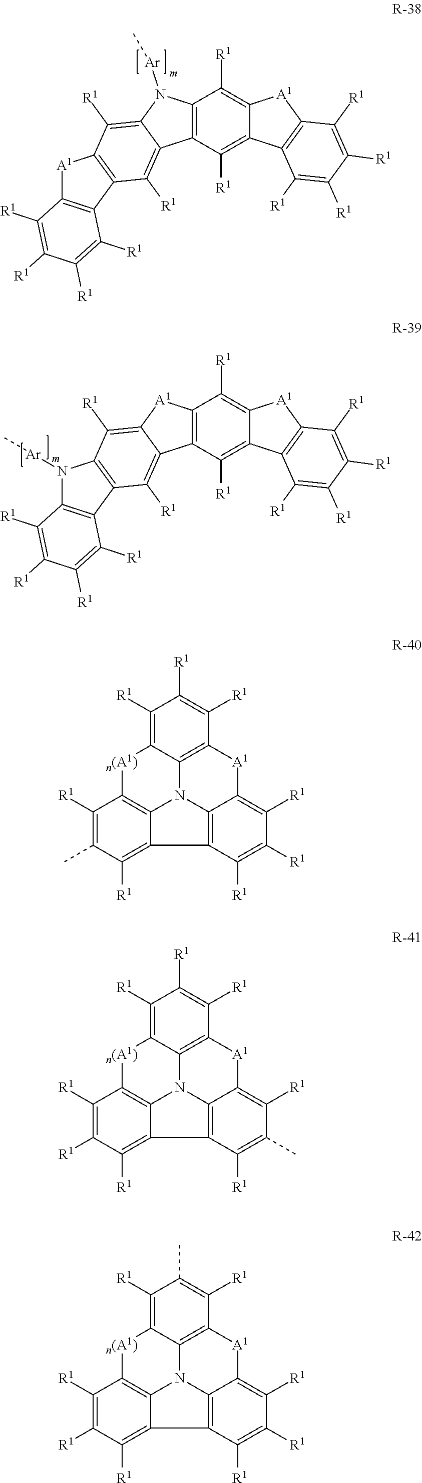

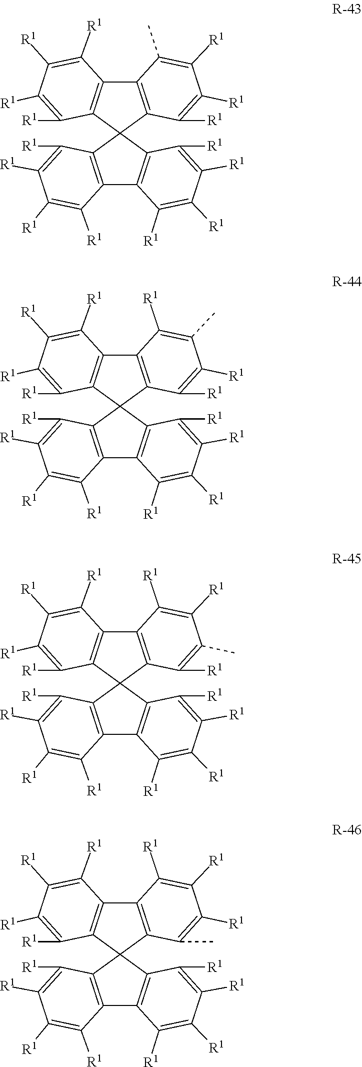

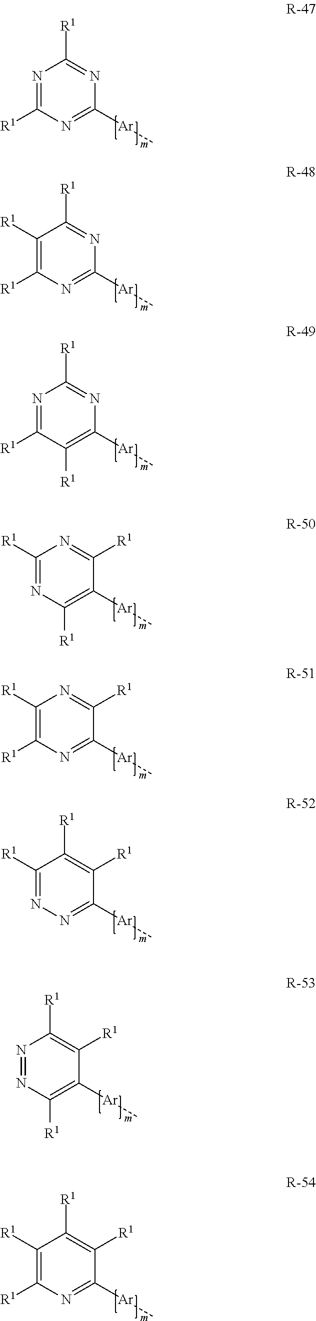

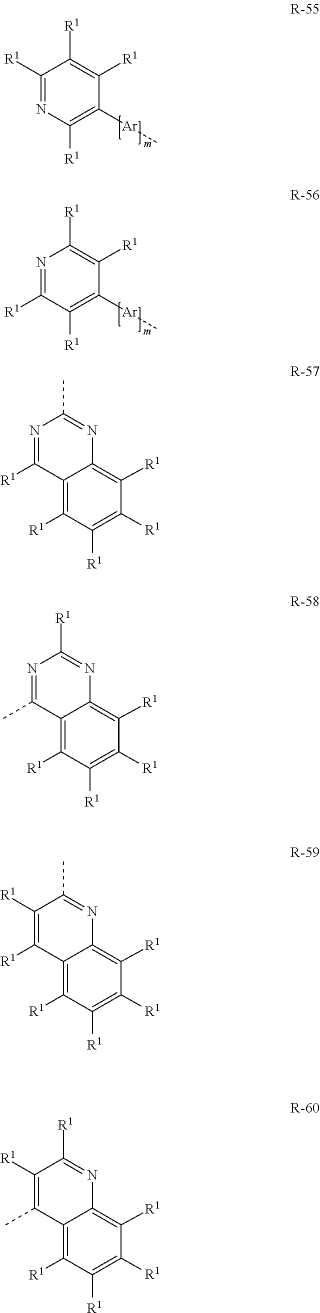

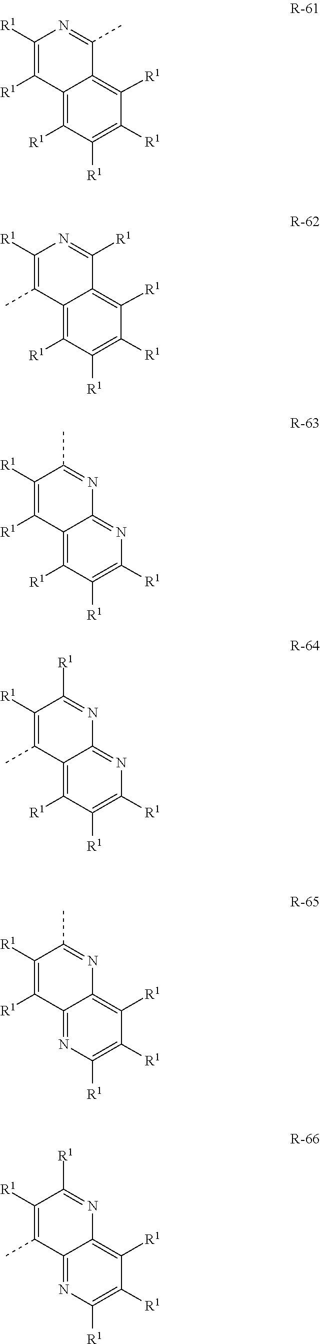

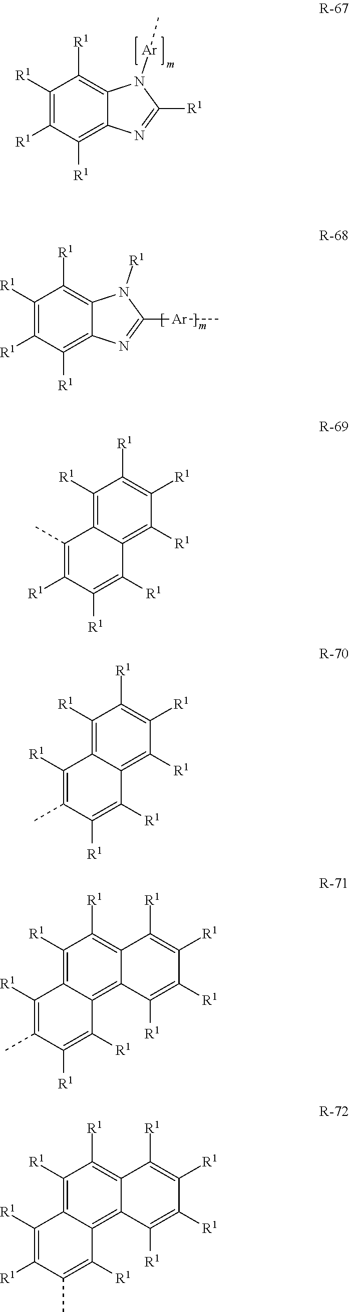

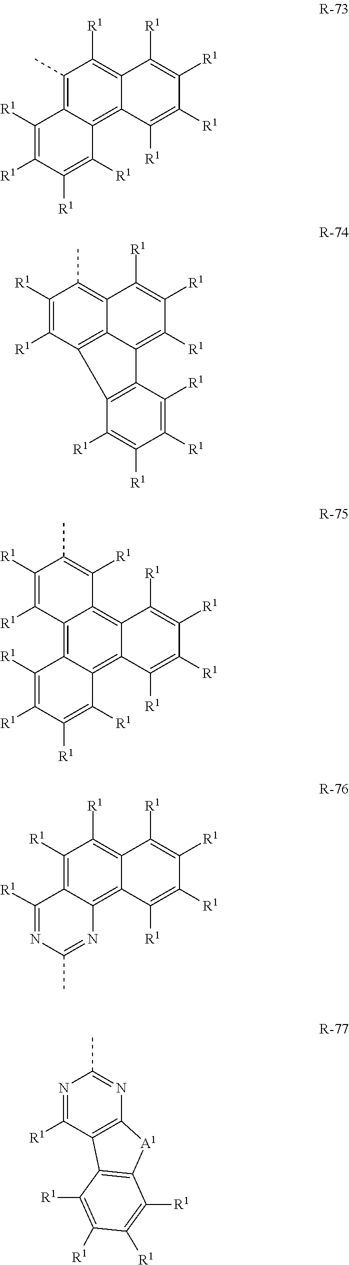

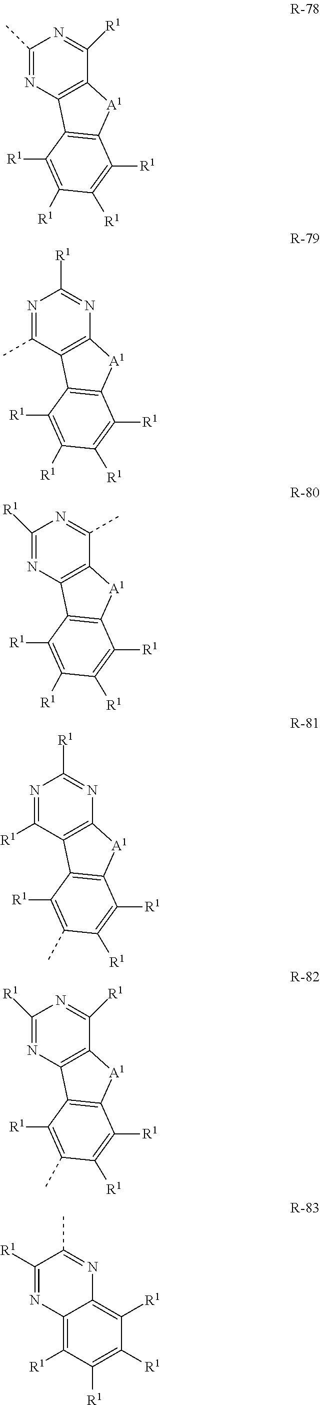

where R¹ has the definitions given above, the dotted bond represents the position of the bond of the group and in addition:
Ar is the same or different at each instance and is a bivalent aromatic or heteroaromatic ring system which has 6 to 18 aromatic ring atoms and may be substituted in each case by one or more R¹ radicals;
A¹ is the same or different at each instance and is C(R¹)₂, NR¹, O or S;
n is 0 or 1, where n=0 means that no A¹ group is bonded at this position and R¹ radicals are bonded to the corresponding carbon atoms instead;
m is 0 or 1, where m=0 means that the Ar group is absent is bonded:

When the abovementioned R-1 to R-83 groups have two or more A¹ groups, possible options for these include all combinations from the definition of A¹. Preferred embodiments in that case are those in which one A¹ group is NR¹ and the other A¹ group is C(R¹)₂ or in which both A¹ groups are NR¹ or in which both A¹ groups are O. In a particularly preferred embodiment of the invention, in R groups having two or more A¹ groups, at least one A¹ group is C(R¹)₂ or is NR¹.

When A¹ is NR¹, the substituent R¹ bonded to the nitrogen atom is preferably an aromatic or heteroaromatic ring system which has 5 to 24 aromatic ring atoms and may also be substituted by one or more R² radicals. In a particularly preferred embodiment, this R¹ substituent is the same or different at each instance and is an aromatic or heteroaromatic ring system which has 6 to 24 aromatic ring atoms, preferably 6 to 12 aromatic ring atoms, and which does not have any fused aryl groups or heteroaryl groups in which two or more aromatic or heteroaromatic 6-membered ring groups are fused directly to one another, and which may also be substituted in each case by one or more R² radicals. Particular preference is given to phenyl, biphenyl, terphenyl and quaterphenyl having bonding patterns as listed above for R-1 to R-11, where these structures may be substituted by one or more R² radicals, but are preferably unsubstituted.

When A¹ is C(R¹)₂, the substituents R¹ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R² radicals. Most preferably, R¹ is a methyl group or a phenyl group. In this case, the R¹ radicals together may also form a ring system, which leads to a spiro system.

In a preferred embodiment of the invention, the R radicals on the triphenylene base skeleton are the same or different at each instance and are H or an aromatic heteroaromatic ring system which has 6 to 24 aromatic ring atoms, preferably 6 to 13 aromatic ring atoms, and may be substituted in each case by one or more R¹ radicals. More preferably, R is the same or different at each instance and is H or phenyl, especially H.

In a further preferred embodiment, the compound of the invention, apart from the R* group, does not have any electron-deficient heteroaryl groups as substituents R, R¹ or R². The electron-deficient heteroaralkyl is either a six-membered heteroaryl group having at least one nitrogen atom or a five-membered heteroaralkyl having at least two heteroatoms, at least one of which is a nitrogen atom, where other aryl or heteroaryl groups may be fused to these groups.

In a further preferred embodiment of the invention, R¹ is the same or different at each instance and is selected from the group consisting of H, D, F, CN, OR², a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may in each case be substituted by one or more R² radicals, and where one or more nonadjacent CH₂ groups may be replaced by O, or an aromatic or heteroaromatic ring system which has 6 to 30 aromatic ring atoms and may be substituted in each case by one or more R² radicals; at the same time, two or more R¹ radicals together may form an aliphatic ring system. In a particularly preferred embodiment of the invention, R¹ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R² radicals, but is preferably unsubstituted, or an aromatic or heteroaromatic ring system which has 6 to 24 aromatic ring atoms, preferably 6 to 13 aromatic ring atoms, and may be substituted in each case by one or more R² radicals, but is preferably unsubstituted.

In a further preferred embodiment of the invention, R² is the same or different at each instance and is H, F, an alkyl group having 1 to 4 carbon atoms or an aryl group having 6 to 10 carbon atoms, which may be substituted by an alkyl group having 1 to 4 carbon atoms, but is preferably unsubstituted.

At the same time, the alkyl groups in compounds of the invention which are processed by vacuum evaporation preferably have not more than five carbon atoms, more preferably not more than 4 carbon atoms, most preferably not more than 1 carbon atom. For compounds that are processed from solution, suitable compounds are also those substituted by alkyl groups, especially branched alkyl groups, having up to 10 carbon atoms or those substituted by oligoarylene groups, for example ortho-, meta- or para-terphenyl or branched terphenyl or quaterphenyl groups.

When the compounds of the formula (1) or the preferred embodiments are used as matrix material for a phosphorescent emitter or in a layer directly adjoining a phosphorescent layer, it is further preferable when the compound does not contain any fused aryl or heteroaryl groups in which more than two six-membered rings are fused directly to one another. It is especially preferable that the R, R¹ and R² groups do not contain any fused aryl or heteroaryl groups in which two or more six-membered rings are fused directly to one another. An exception to this is formed by phenanthrene, triphenylene, quinazoline and quinoxaline, which, because of their higher triplet energy, may be preferable in spite of the presence of fused aromatic six-membered rings.

The abovementioned preferred embodiments may be combined with one another as desired within the restrictions defined in claim 1. In a particularly preferred embodiment of the invention, the abovementioned preferences occur simultaneously.

Examples of suitable compounds according to the above-detailed embodiments are the compounds detailed in the following table:

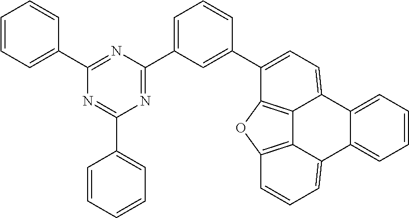 [SMILES: c1ccc(-c2nc(-c3ccccc3)nc(-c3cccc(-c4ccc5c6ccccc6c6cccc7oc4c5c76)c3)n2)cc1]

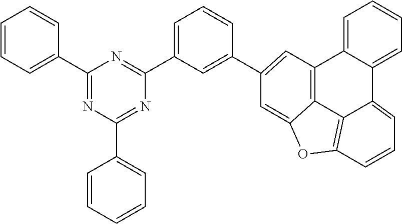 [SMILES: c1ccc(-c2nc(-c3ccccc3)nc(-c3cccc(-c4cc5oc6cccc7c8ccccc8c(c4)c5c67)c3)n2)cc1]

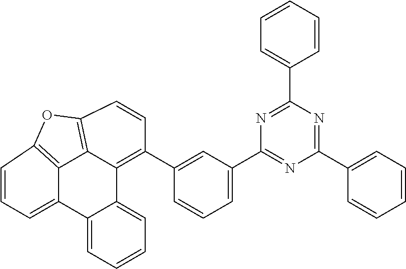 [SMILES: c1ccc(-c2nc(-c3ccccc3)nc(-c3cccc(-c4ccc5oc6cccc7c8ccccc8c4c5c67)c3)n2)cc1]

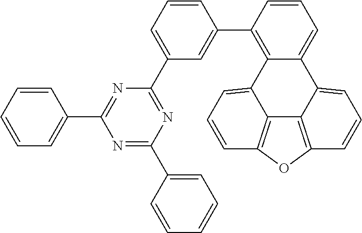 [SMILES: c1ccc(-c2nc(-c3ccccc3)nc(-c3cccc(-c4cccc5c6cccc7oc8cccc(c45)c8c76)c3)n2)cc1]

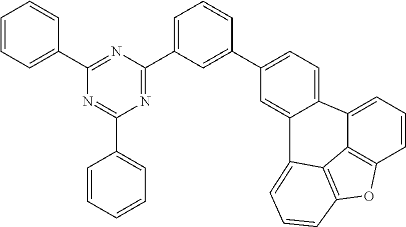 [SMILES: c1ccc(-c2nc(-c3ccccc3)nc(-c3cccc(-c4ccc5c(c4)c4cccc6oc7cccc5c7c64)c3)n2)cc1]

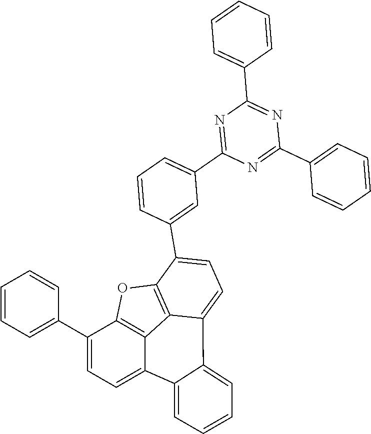 [SMILES: c1ccc(-c2nc(-c3ccccc3)nc(-c3cccc(-c4ccc5c6ccccc6c6ccc(-c7ccccc7)c7oc4c5c76)c3)n2)cc1]

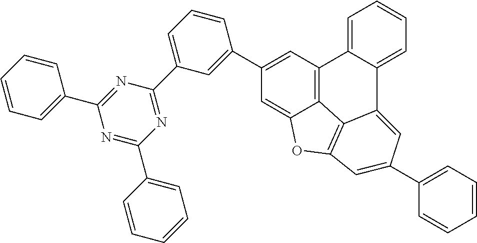 [SMILES: c1ccc(-c2cc3oc4cc(-c5cccc(-c6nc(-c7ccccc7)nc(-c7ccccc7)n6)c5)cc5c6ccccc6c(c2)c3c45)cc1]

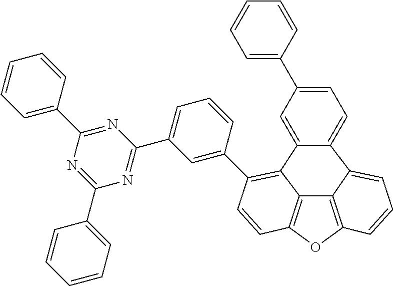 [SMILES: c1ccc(-c2ccc3c(c2)c2c(-c4cccc(-c5nc(-c6ccccc6)nc(-c6ccccc6)n5)c4)ccc4oc5cccc3c5c42)cc1]

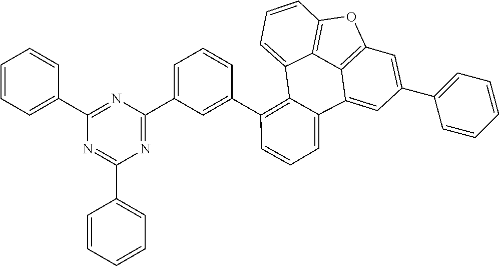 [SMILES: c1ccc(-c2cc3oc4cccc5c6c(-c7cccc(-c8nc(-c9ccccc9)nc(-c9ccccc9)n8)c7)cccc6c(c2)c3c45)cc1]

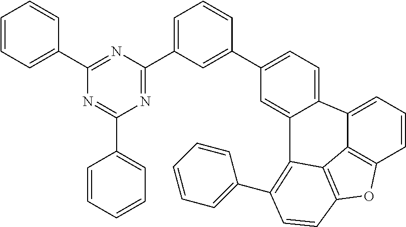 [SMILES: c1ccc(-c2nc(-c3ccccc3)nc(-c3cccc(-c4ccc5c(c4)c4c(-c6ccccc6)ccc6oc7cccc5c7c64)c3)n2)cc1]

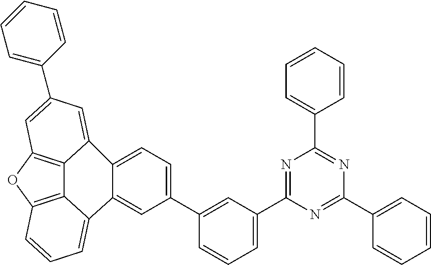 [SMILES: c1ccc(-c2cc3oc4cccc5c6cc(-c7cccc(-c8nc(-c9ccccc9)nc(-c9ccccc9)n8)c7)ccc6c(c2)c3c45)cc1]

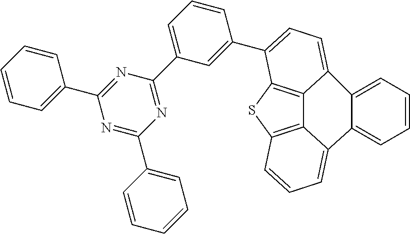 [SMILES: c1ccc(-c2nc(-c3ccccc3)nc(-c3cccc(-c4ccc5c6ccccc6c6cccc7sc4c5c76)c3)n2)cc1]

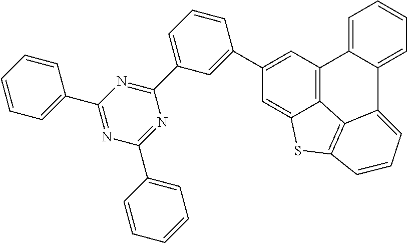 [SMILES: c1ccc(-c2nc(-c3ccccc3)nc(-c3cccc(-c4cc5sc6cccc7c8ccccc8c(c4)c5c67)c3)n2)cc1]

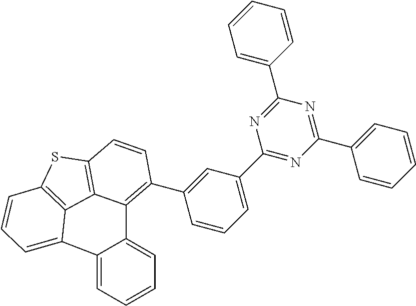 [SMILES: c1ccc(-c2nc(-c3ccccc3)nc(-c3cccc(-c4ccc5sc6cccc7c8ccccc8c4c5c67)c3)n2)cc1]

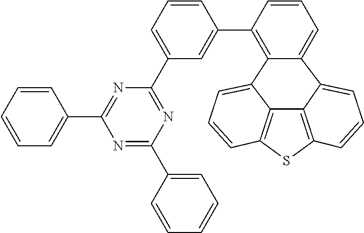 [SMILES: c1ccc(-c2nc(-c3ccccc3)nc(-c3cccc(-c4cccc5c6cccc7sc8cccc(c45)c8c76)c3)n2)cc1]

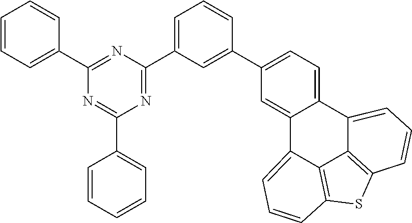 [SMILES: c1ccc(-c2nc(-c3ccccc3)nc(-c3cccc(-c4ccc5c(c4)c4cccc6sc7cccc5c7c64)c3)n2)cc1]

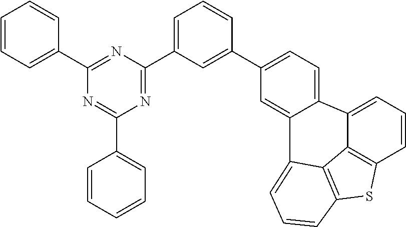 [SMILES: c1ccc(-c2nc(-c3ccccc3)nc(-c3cccc(-c4ccc5c(c4)c4cccc6sc7cccc5c7c64)c3)n2)cc1]

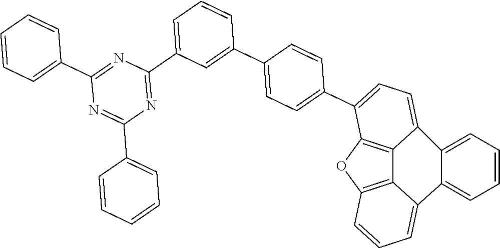 [SMILES: c1ccc(-c2nc(-c3ccccc3)nc(-c3cccc(-c4ccc(-c5ccc6c7ccccc7c7cccc8oc5c6c87)cc4)c3)n2)cc1]

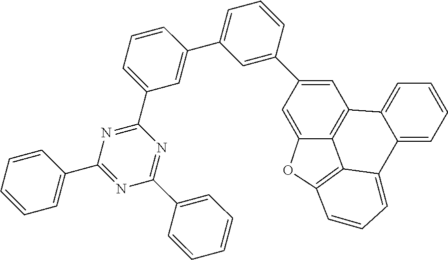 [SMILES: c1ccc(-c2nc(-c3ccccc3)nc(-c3cccc(-c4cccc(-c5cc6oc7cccc8c9ccccc9c(c5)c6c78)c4)c3)n2)cc1]

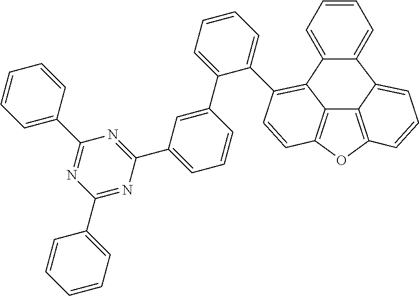 [SMILES: c1ccc(-c2nc(-c3ccccc3)nc(-c3cccc(-c4ccccc4-c4ccc5oc6cccc7c8ccccc8c4c5c67)c3)n2)cc1]

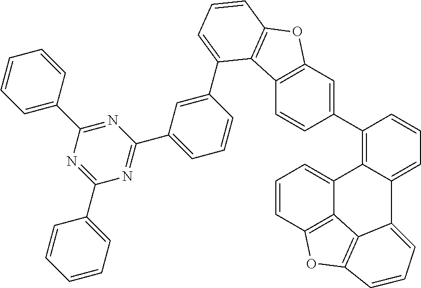 [SMILES: c1ccc(-c2nc(-c3ccccc3)nc(-c3cccc(-c4cccc5oc6cc(-c7cccc8c9cccc%10oc%11cccc(c78)c%11c%109)ccc6c45)c3)n2)cc1]

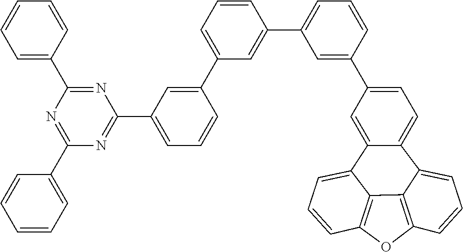 [SMILES: c1ccc(-c2nc(-c3ccccc3)nc(-c3cccc(-c4cccc(-c5cccc(-c6ccc7c(c6)c6cccc8oc9cccc7c9c86)c5)c4)c3)n2)cc1]

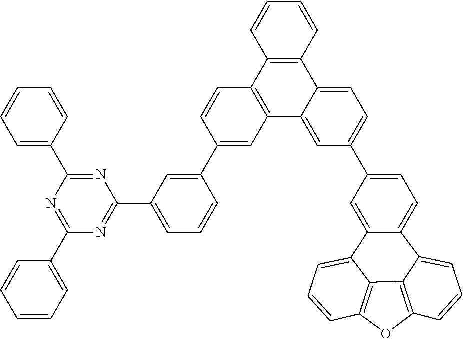 [SMILES: c1ccc(-c2nc(-c3ccccc3)nc(-c3cccc(-c4ccc5c6ccccc6c6ccc(-c7ccc8c(c7)c7cccc9oc%10cccc8c%10c97)cc6c5c4)c3)n2)cc1]

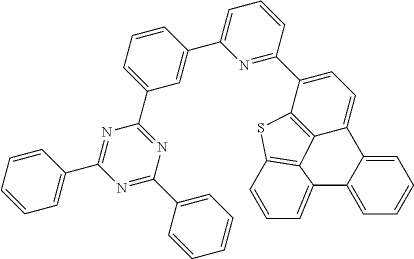 [SMILES: c1ccc(-c2nc(-c3ccccc3)nc(-c3cccc(-c4cccc(-c5ccc6c7ccccc7c7cccc8sc5c6c87)n4)c3)n2)cc1]

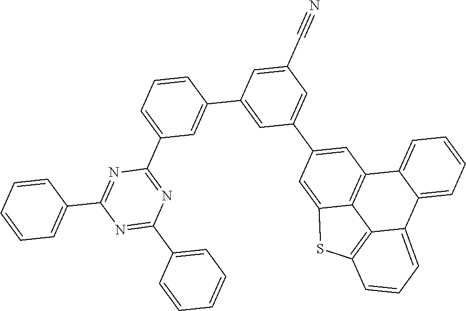 [SMILES: N#Cc1cc(-c2cccc(-c3nc(-c4ccccc4)nc(-c4ccccc4)n3)c2)cc(-c2cc3sc4cccc5c6ccccc6c(c2)c3c45)c1]

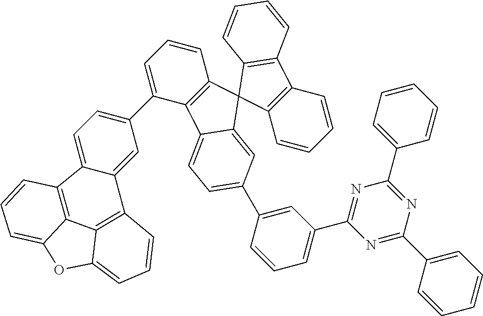 [SMILES: c1ccc(-c2nc(-c3ccccc3)nc(-c3cccc(-c4ccc5c(c4)C4(c6ccccc6-c6ccccc64)c4cccc(-c6ccc7c(c6)c6cccc8oc9cccc7c9c86)c4-5)c3)n2)cc1]

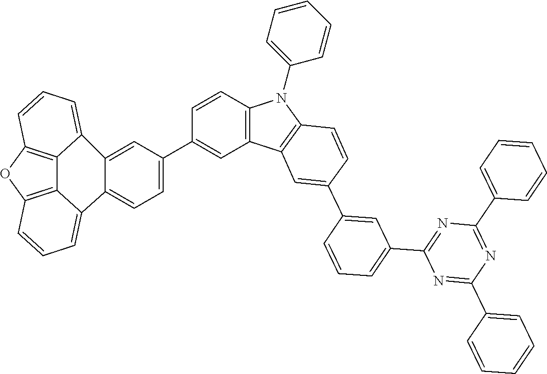 [SMILES: c1ccc(-c2nc(-c3ccccc3)nc(-c3cccc(-c4ccc5c(c4)c4cc(-c6ccc7c(c6)c6cccc8oc9cccc7c9c86)ccc4n5-c4ccccc4)c3)n2)cc1]

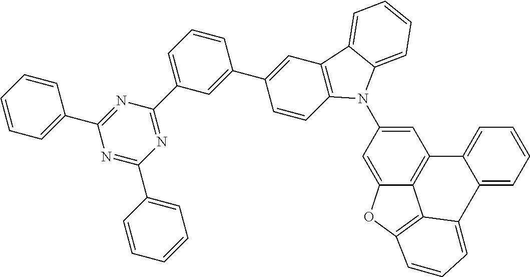 [SMILES: c1ccc(-c2nc(-c3ccccc3)nc(-c3cccc(-c4ccc5c(c4)c4ccccc4n5-c4cc5oc6cccc7c8ccccc8c(c4)c5c67)c3)n2)cc1]

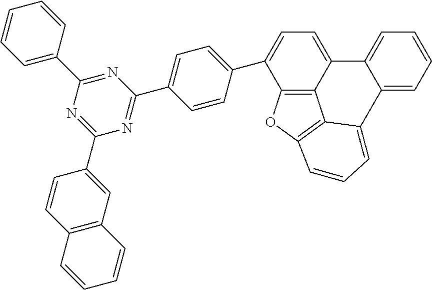 [SMILES: c1ccc(-c2nc(-c3ccc(-c4ccc5c6ccccc6c6cccc7oc4c5c76)cc3)nc(-c3ccc4ccccc4c3)n2)cc1]

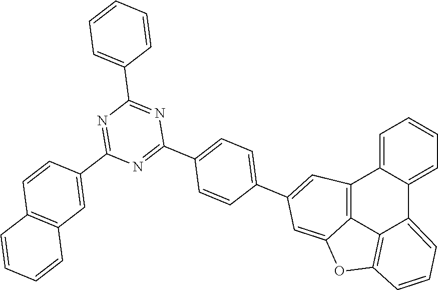 [SMILES: c1ccc(-c2nc(-c3ccc(-c4cc5oc6cccc7c8ccccc8c(c4)c5c67)cc3)nc(-c3ccc4ccccc4c3)n2)cc1]

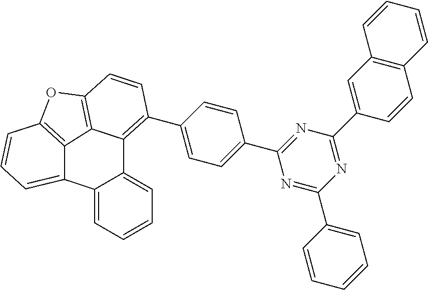 [SMILES: c1ccc(-c2nc(-c3ccc(-c4ccc5oc6cccc7c8ccccc8c4c5c67)cc3)nc(-c3ccc4ccccc4c3)n2)cc1]

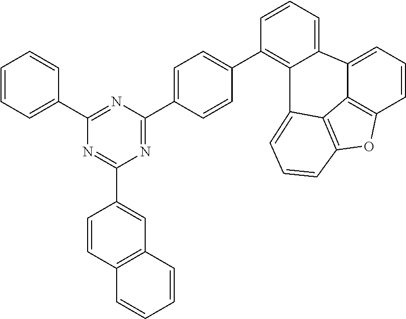 [SMILES: c1ccc(-c2nc(-c3ccc(-c4cccc5c6cccc7oc8cccc(c45)c8c76)cc3)nc(-c3ccc4ccccc4c3)n2)cc1]

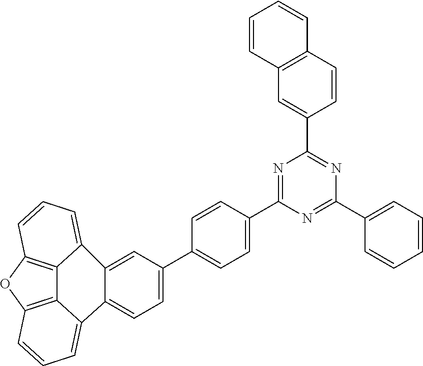 [SMILES: c1ccc(-c2nc(-c3ccc(-c4ccc5c(c4)c4cccc6oc7cccc5c7c64)cc3)nc(-c3ccc4ccccc4c3)n2)cc1]

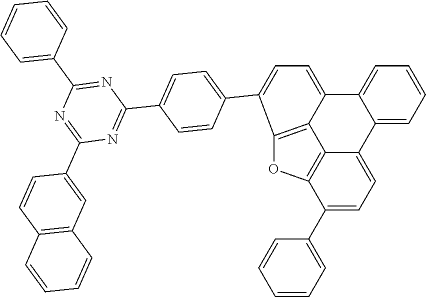 [SMILES: c1ccc(-c2nc(-c3ccc(-c4ccc5c6ccccc6c6ccc(-c7ccccc7)c7oc4c5c76)cc3)nc(-c3ccc4ccccc4c3)n2)cc1]

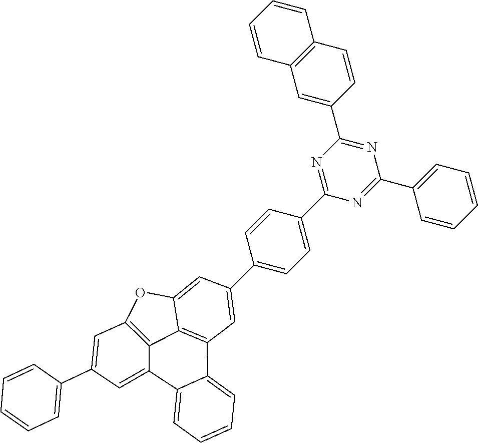 [SMILES: c1ccc(-c2cc3oc4cc(-c5ccc(-c6nc(-c7ccccc7)nc(-c7ccc8ccccc8c7)n6)cc5)cc5c6ccccc6c(c2)c3c45)cc1]

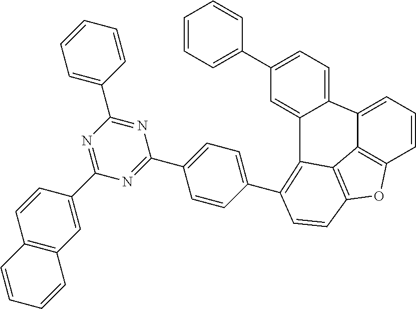 [SMILES: c1ccc(-c2ccc3c(c2)c2c(-c4ccc(-c5nc(-c6ccccc6)nc(-c6ccc7ccccc7c6)n5)cc4)ccc4oc5cccc3c5c42)cc1]

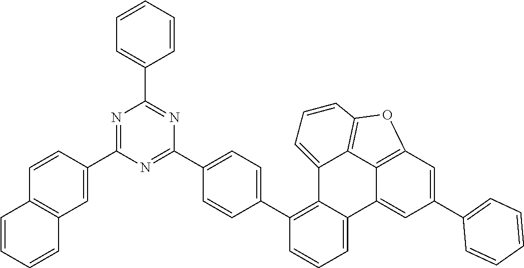 [SMILES: c1ccc(-c2cc3oc4cccc5c6c(-c7ccc(-c8nc(-c9ccccc9)nc(-c9ccc%10ccccc%10c9)n8)cc7)cccc6c(c2)c3c45)cc1]

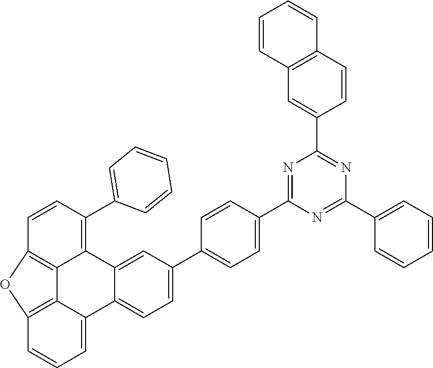 [SMILES: c1ccc(-c2nc(-c3ccc(-c4ccc5c(c4)c4c(-c6ccccc6)ccc6oc7cccc5c7c64)cc3)nc(-c3ccc4ccccc4c3)n2)cc1]

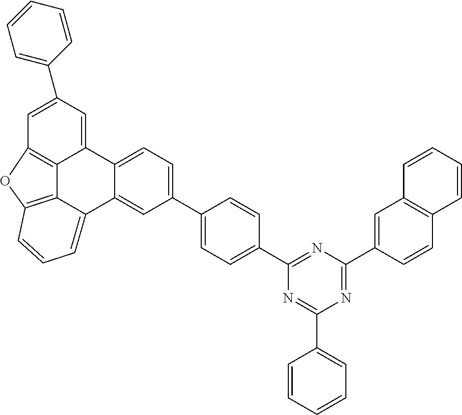 [SMILES: c1ccc(-c2cc3oc4cccc5c6cc(-c7ccc(-c8nc(-c9ccccc9)nc(-c9ccc%10ccccc%10c9)n8)cc7)ccc6c(c2)c3c45)cc1]

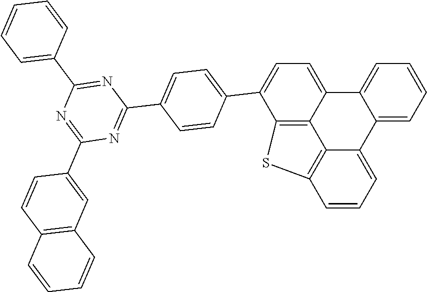 [SMILES: c1ccc(-c2nc(-c3ccc(-c4ccc5c6ccccc6c6cccc7sc4c5c76)cc3)nc(-c3ccc4ccccc4c3)n2)cc1]

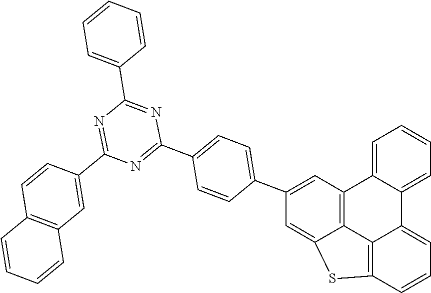 [SMILES: c1ccc(-c2nc(-c3ccc(-c4cc5sc6cccc7c8ccccc8c(c4)c5c67)cc3)nc(-c3ccc4ccccc4c3)n2)cc1]

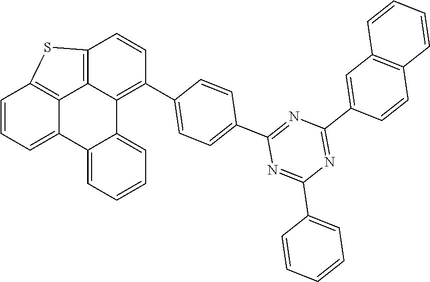 [SMILES: c1ccc(-c2nc(-c3ccc(-c4ccc5sc6cccc7c8ccccc8c4c5c67)cc3)nc(-c3ccc4ccccc4c3)n2)cc1]

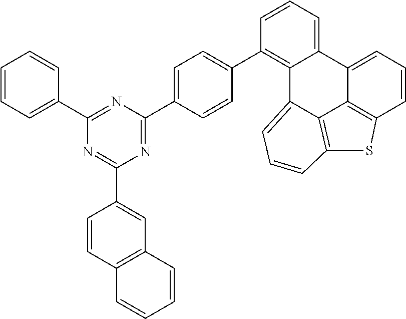 [SMILES: c1ccc(-c2nc(-c3ccc(-c4cccc5c6cccc7sc8cccc(c45)c8c76)cc3)nc(-c3ccc4ccccc4c3)n2)cc1]

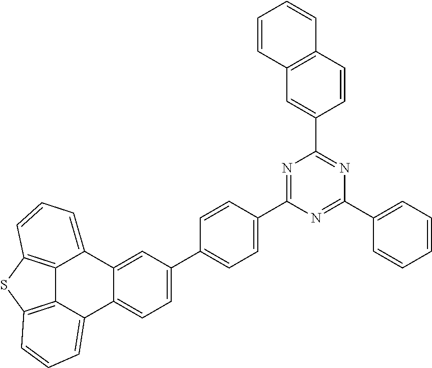 [SMILES: c1ccc(-c2nc(-c3ccc(-c4ccc5c(c4)c4cccc6sc7cccc5c7c64)cc3)nc(-c3ccc4ccccc4c3)n2)cc1]

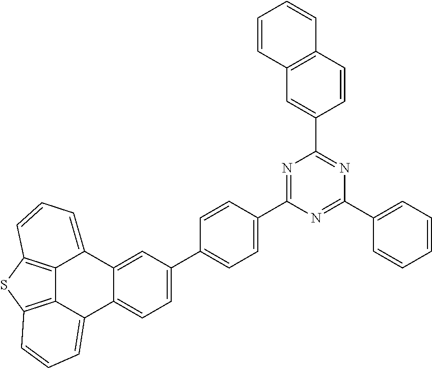 [SMILES: c1ccc(-c2nc(-c3ccc(-c4ccc5c(c4)c4cccc6sc7cccc5c7c64)cc3)nc(-c3ccc4ccccc4c3)n2)cc1]

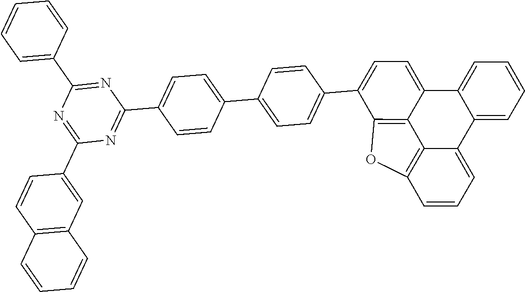 [SMILES: c1ccc(-c2nc(-c3ccc(-c4ccc(-c5ccc6c7ccccc7c7cccc8oc5c6c87)cc4)cc3)nc(-c3ccc4ccccc4c3)n2)cc1]

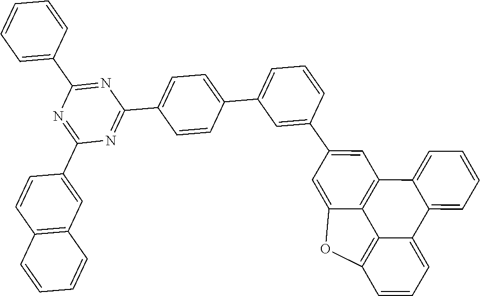 [SMILES: c1ccc(-c2nc(-c3ccc(-c4cccc(-c5cc6oc7cccc8c9ccccc9c(c5)c6c78)c4)cc3)nc(-c3ccc4ccccc4c3)n2)cc1]

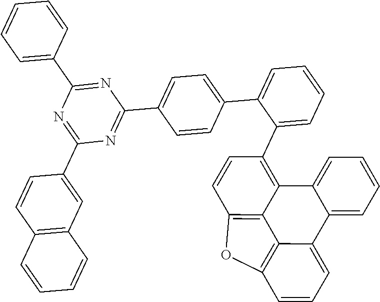 [SMILES: c1ccc(-c2nc(-c3ccc(-c4ccccc4-c4ccc5oc6cccc7c8ccccc8c4c5c67)cc3)nc(-c3ccc4ccccc4c3)n2)cc1]

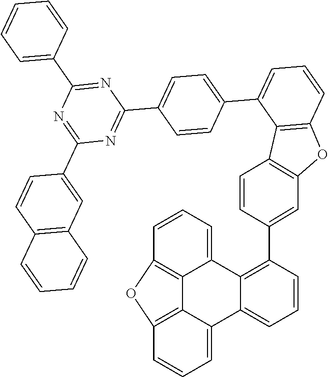 [SMILES: c1ccc(-c2nc(-c3ccc(-c4cccc5oc6cc(-c7cccc8c9cccc%10oc%11cccc(c78)c%11c%109)ccc6c45)cc3)nc(-c3ccc4ccccc4c3)n2)cc1]

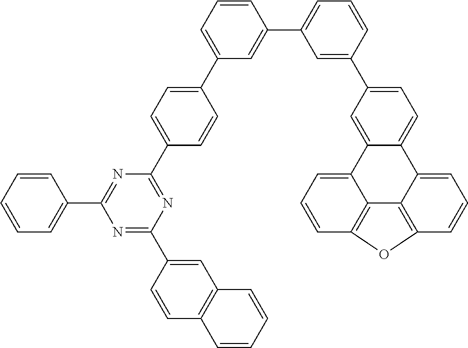 [SMILES: c1ccc(-c2nc(-c3ccc(-c4cccc(-c5cccc(-c6ccc7c(c6)c6cccc8oc9cccc7c9c86)c5)c4)cc3)nc(-c3ccc4ccccc4c3)n2)cc1]

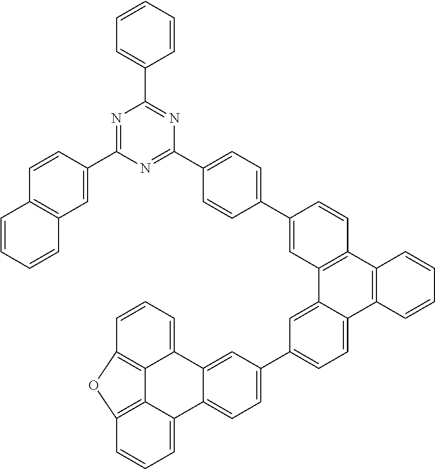 [SMILES: c1ccc(-c2nc(-c3ccc(-c4ccc5c6ccccc6c6ccc(-c7ccc8c(c7)c7cccc9oc%10cccc8c%10c97)cc6c5c4)cc3)nc(-c3ccc4ccccc4c3)n2)cc1]

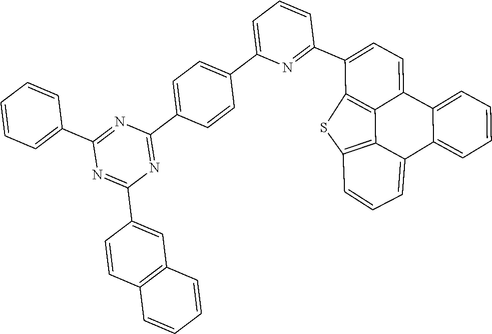 [SMILES: c1ccc(-c2nc(-c3ccc(-c4cccc(-c5ccc6c7ccccc7c7cccc8sc5c6c87)n4)cc3)nc(-c3ccc4ccccc4c3)n2)cc1]

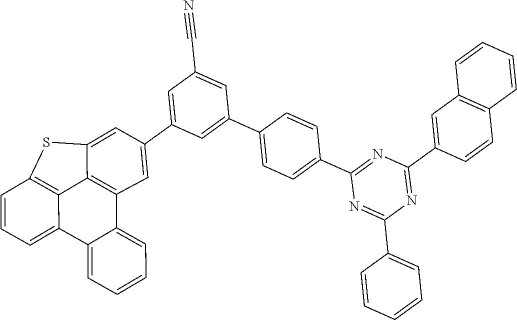 [SMILES: N#Cc1cc(-c2ccc(-c3nc(-c4ccccc4)nc(-c4ccc5ccccc5c4)n3)cc2)cc(-c2cc3sc4cccc5c6ccccc6c(c2)c3c45)c1]

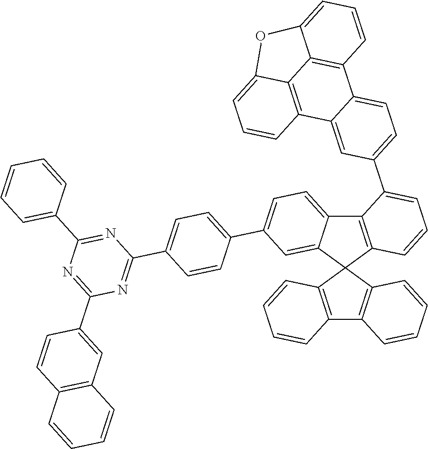 [SMILES: c1ccc(-c2nc(-c3ccc(-c4ccc5c(c4)C4(c6ccccc6-c6ccccc64)c4cccc(-c6ccc7c(c6)c6cccc8oc9cccc7c9c86)c4-5)cc3)nc(-c3ccc4ccccc4c3)n2)cc1]

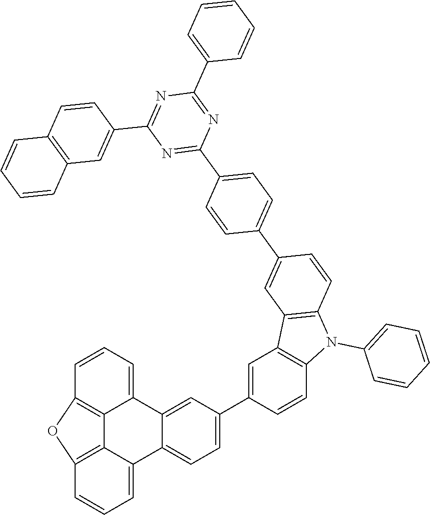 [SMILES: c1ccc(-c2nc(-c3ccc(-c4ccc5c(c4)c4cc(-c6ccc7c(c6)c6cccc8oc9cccc7c9c86)ccc4n5-c4ccccc4)cc3)nc(-c3ccc4ccccc4c3)n2)cc1]

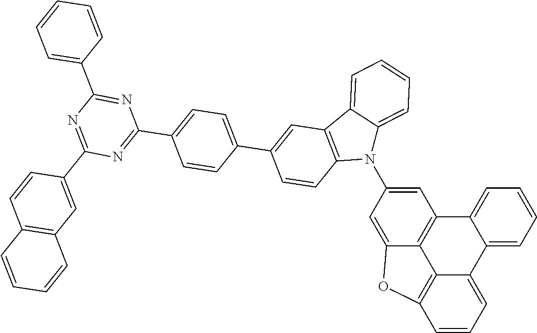 [SMILES: c1ccc(-c2nc(-c3ccc(-c4ccc5c(c4)c4ccccc4n5-c4cc5oc6cccc7c8ccccc8c(c4)c5c67)cc3)nc(-c3ccc4ccccc4c3)n2)cc1]

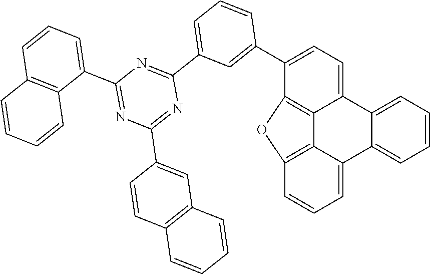 [SMILES: c1cc(-c2nc(-c3ccc4ccccc4c3)nc(-c3cccc4ccccc34)n2)cc(-c2ccc3c4ccccc4c4cccc5oc2c3c54)c1]

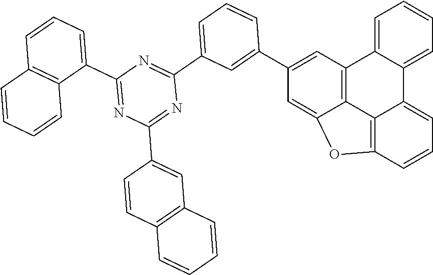 [SMILES: c1cc(-c2cc3oc4cccc5c6ccccc6c(c2)c3c45)cc(-c2nc(-c3ccc4ccccc4c3)nc(-c3cccc4ccccc34)n2)c1]

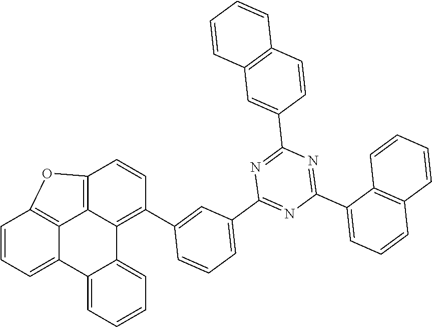 [SMILES: c1cc(-c2nc(-c3ccc4ccccc4c3)nc(-c3cccc4ccccc34)n2)cc(-c2ccc3oc4cccc5c6ccccc6c2c3c45)c1]

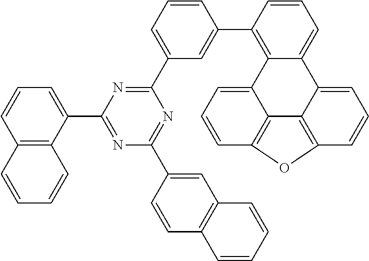 [SMILES: c1cc(-c2nc(-c3ccc4ccccc4c3)nc(-c3cccc4ccccc34)n2)cc(-c2cccc3c4cccc5oc6cccc(c23)c6c54)c1]

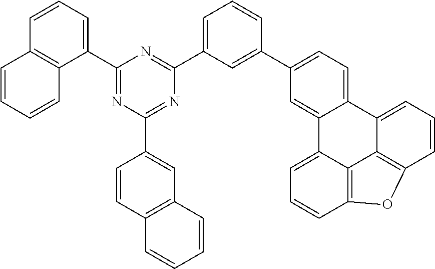 [SMILES: c1cc(-c2ccc3c(c2)c2cccc4oc5cccc3c5c42)cc(-c2nc(-c3ccc4ccccc4c3)nc(-c3cccc4ccccc34)n2)c1]

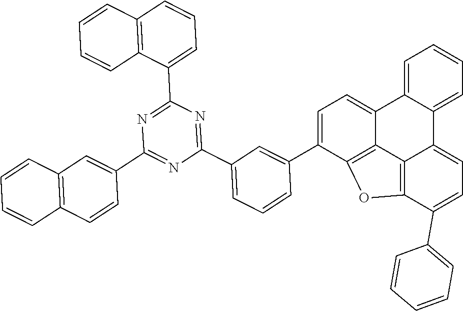 [SMILES: c1ccc(-c2ccc3c4ccccc4c4ccc(-c5cccc(-c6nc(-c7ccc8ccccc8c7)nc(-c7cccc8ccccc78)n6)c5)c5oc2c3c54)cc1]

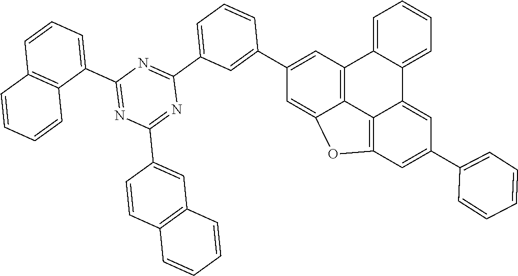 [SMILES: c1ccc(-c2cc3oc4cc(-c5cccc(-c6nc(-c7ccc8ccccc8c7)nc(-c7cccc8ccccc78)n6)c5)cc5c6ccccc6c(c2)c3c45)cc1]

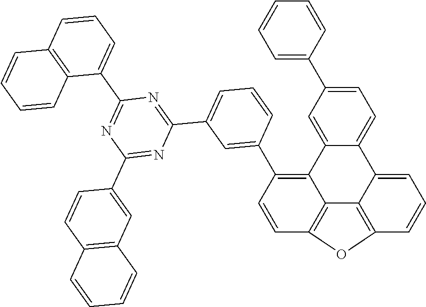 [SMILES: c1ccc(-c2ccc3c(c2)c2c(-c4cccc(-c5nc(-c6ccc7ccccc7c6)nc(-c6cccc7ccccc67)n5)c4)ccc4oc5cccc3c5c42)cc1]

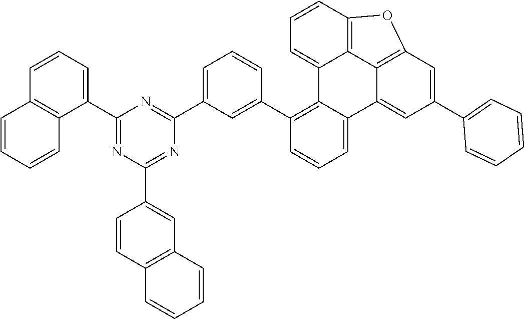 [SMILES: c1ccc(-c2cc3oc4cccc5c6c(-c7cccc(-c8nc(-c9ccc%10ccccc%10c9)nc(-c9cccc%10ccccc9%10)n8)c7)cccc6c(c2)c3c45)cc1]

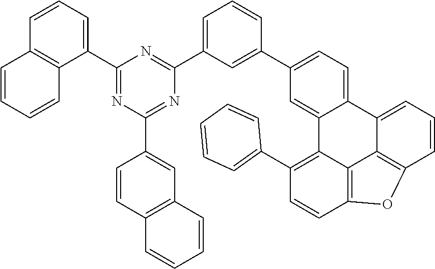 [SMILES: c1ccc(-c2ccc3oc4cccc5c6ccc(-c7cccc(-c8nc(-c9ccc%10ccccc%10c9)nc(-c9cccc%10ccccc9%10)n8)c7)cc6c2c3c45)cc1]

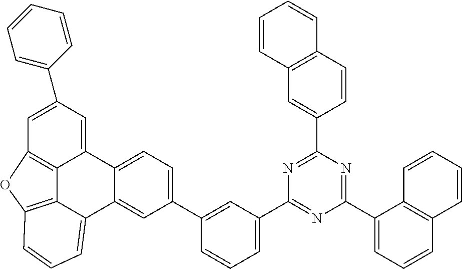 [SMILES: c1ccc(-c2cc3oc4cccc5c6cc(-c7cccc(-c8nc(-c9ccc%10ccccc%10c9)nc(-c9cccc%10ccccc9%10)n8)c7)ccc6c(c2)c3c45)cc1]

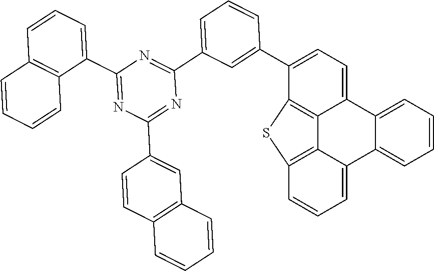 [SMILES: c1cc(-c2nc(-c3ccc4ccccc4c3)nc(-c3cccc4ccccc34)n2)cc(-c2ccc3c4ccccc4c4cccc5sc2c3c54)c1]

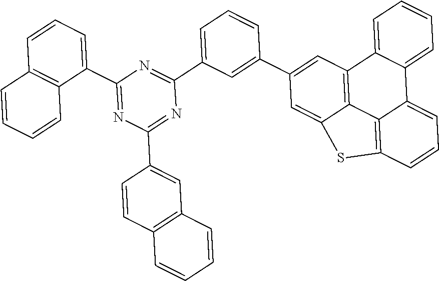 [SMILES: c1cc(-c2cc3sc4cccc5c6ccccc6c(c2)c3c45)cc(-c2nc(-c3ccc4ccccc4c3)nc(-c3cccc4ccccc34)n2)c1]

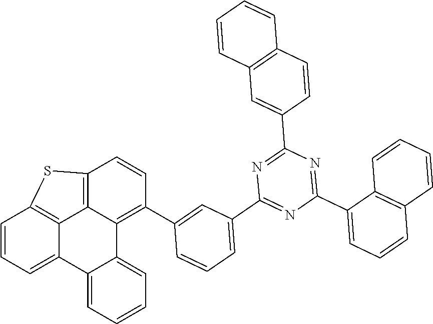 [SMILES: c1cc(-c2nc(-c3ccc4ccccc4c3)nc(-c3cccc4ccccc34)n2)cc(-c2ccc3sc4cccc5c6ccccc6c2c3c45)c1]

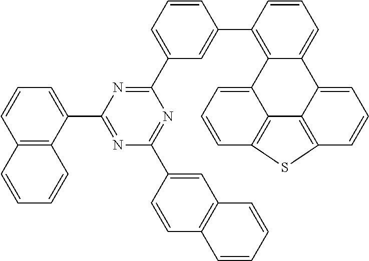 [SMILES: c1cc(-c2nc(-c3ccc4ccccc4c3)nc(-c3cccc4ccccc34)n2)cc(-c2cccc3c4cccc5sc6cccc(c23)c6c54)c1]

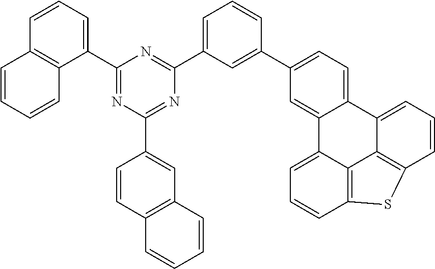 [SMILES: c1cc(-c2ccc3c(c2)c2cccc4sc5cccc3c5c42)cc(-c2nc(-c3ccc4ccccc4c3)nc(-c3cccc4ccccc34)n2)c1]

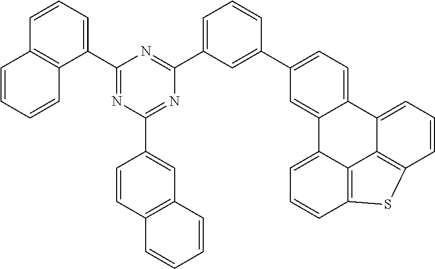 [SMILES: c1cc(-c2ccc3c(c2)c2cccc4sc5cccc3c5c42)cc(-c2nc(-c3ccc4ccccc4c3)nc(-c3cccc4ccccc34)n2)c1]

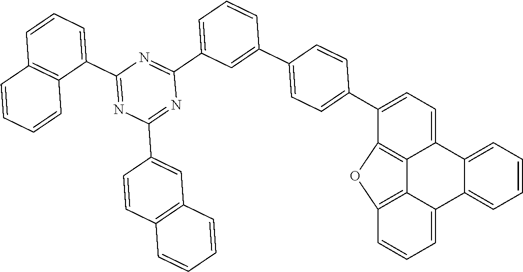 [SMILES: c1cc(-c2ccc(-c3ccc4c5ccccc5c5cccc6oc3c4c65)cc2)cc(-c2nc(-c3ccc4ccccc4c3)nc(-c3cccc4ccccc34)n2)c1]

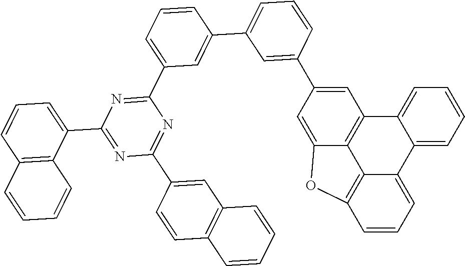 [SMILES: c1cc(-c2cccc(-c3nc(-c4ccc5ccccc5c4)nc(-c4cccc5ccccc45)n3)c2)cc(-c2cc3oc4cccc5c6ccccc6c(c2)c3c45)c1]

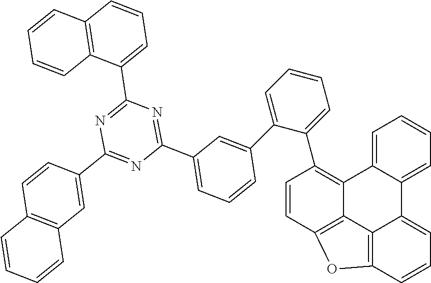 [SMILES: c1cc(-c2nc(-c3ccc4ccccc4c3)nc(-c3cccc4ccccc34)n2)cc(-c2ccccc2-c2ccc3oc4cccc5c6ccccc6c2c3c45)c1]

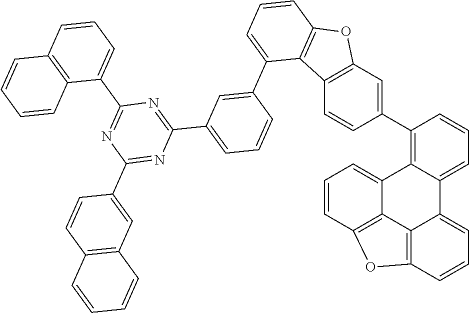 [SMILES: c1cc(-c2nc(-c3ccc4ccccc4c3)nc(-c3cccc4ccccc34)n2)cc(-c2cccc3oc4cc(-c5cccc6c7cccc8oc9cccc(c56)c9c87)ccc4c23)c1]

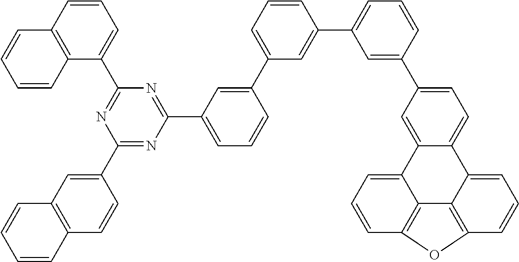 [SMILES: c1cc(-c2cccc(-c3ccc4c(c3)c3cccc5oc6cccc4c6c53)c2)cc(-c2cccc(-c3nc(-c4ccc5ccccc5c4)nc(-c4cccc5ccccc45)n3)c2)c1]

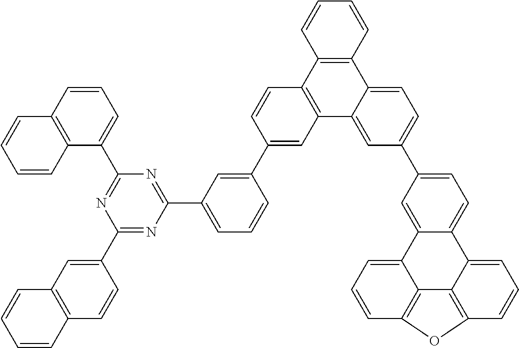 [SMILES: c1cc(-c2ccc3c4ccccc4c4ccc(-c5ccc6c(c5)c5cccc7oc8cccc6c8c75)cc4c3c2)cc(-c2nc(-c3ccc4ccccc4c3)nc(-c3cccc4ccccc34)n2)c1]

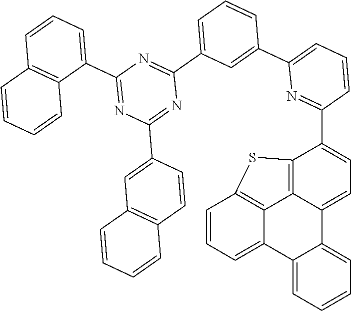 [SMILES: c1cc(-c2cccc(-c3ccc4c5ccccc5c5cccc6sc3c4c65)n2)cc(-c2nc(-c3ccc4ccccc4c3)nc(-c3cccc4ccccc34)n2)c1]

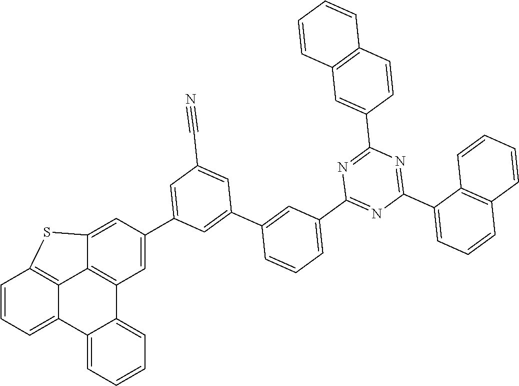 [SMILES: N#Cc1cc(-c2cccc(-c3nc(-c4ccc5ccccc5c4)nc(-c4cccc5ccccc45)n3)c2)cc(-c2cc3sc4cccc5c6ccccc6c(c2)c3c45)c1]

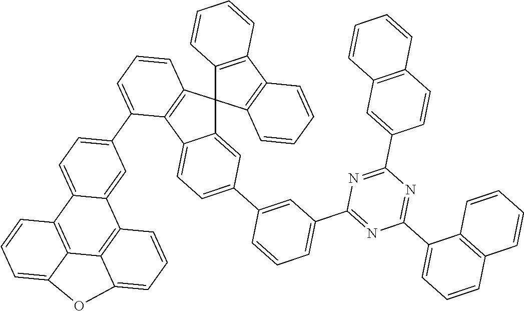 [SMILES: c1cc(-c2ccc3c(c2)C2(c4ccccc4-c4ccccc42)c2cccc(-c4ccc5c(c4)c4cccc6oc7cccc5c7c64)c2-3)cc(-c2nc(-c3ccc4ccccc4c3)nc(-c3cccc4ccccc34)n2)c1]

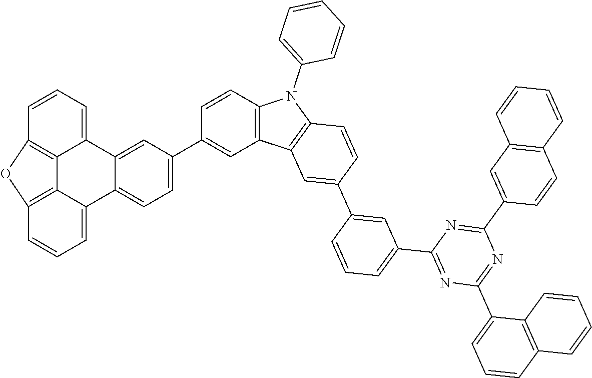 [SMILES: c1ccc(-n2c3ccc(-c4cccc(-c5nc(-c6ccc7ccccc7c6)nc(-c6cccc7ccccc67)n5)c4)cc3c3cc(-c4ccc5c(c4)c4cccc6oc7cccc5c7c64)ccc32)cc1]

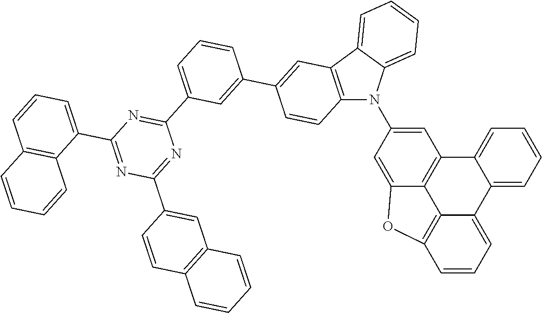 [SMILES: c1cc(-c2ccc3c(c2)c2ccccc2n3-c2cc3oc4cccc5c6ccccc6c(c2)c3c45)cc(-c2nc(-c3ccc4ccccc4c3)nc(-c3cccc4ccccc34)n2)c1]

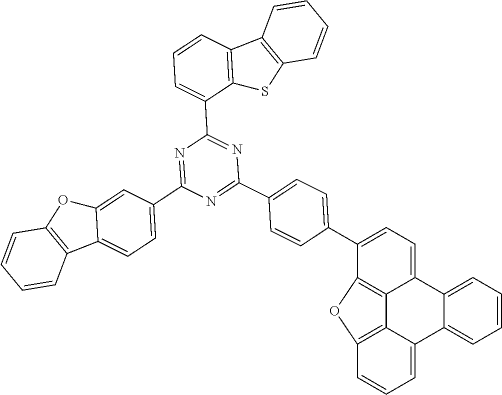 [SMILES: c1ccc2c(c1)oc1cc(-c3nc(-c4ccc(-c5ccc6c7ccccc7c7cccc8oc5c6c87)cc4)nc(-c4cccc5c4sc4ccccc45)n3)ccc12]

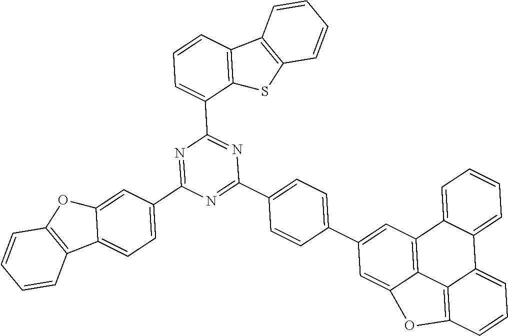 [SMILES: c1ccc2c(c1)oc1cc(-c3nc(-c4ccc(-c5cc6oc7cccc8c9ccccc9c(c5)c6c78)cc4)nc(-c4cccc5c4sc4ccccc45)n3)ccc12]

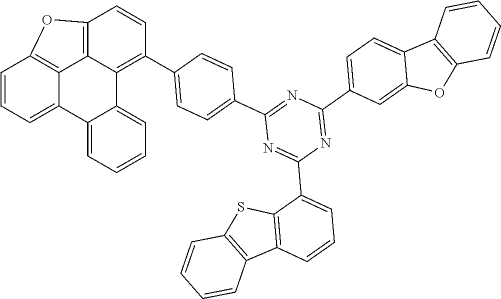 [SMILES: c1ccc2c(c1)oc1cc(-c3nc(-c4ccc(-c5ccc6oc7cccc8c9ccccc9c5c6c78)cc4)nc(-c4cccc5c4sc4ccccc45)n3)ccc12]

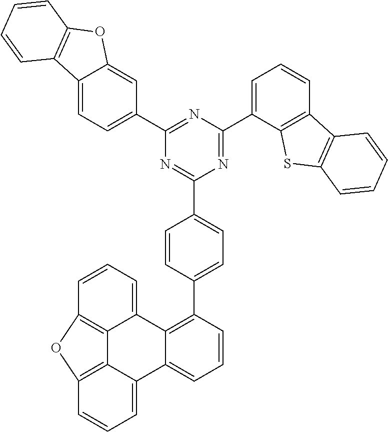 [SMILES: c1ccc2c(c1)oc1cc(-c3nc(-c4ccc(-c5cccc6c7cccc8oc9cccc(c56)c9c87)cc4)nc(-c4cccc5c4sc4ccccc45)n3)ccc12]

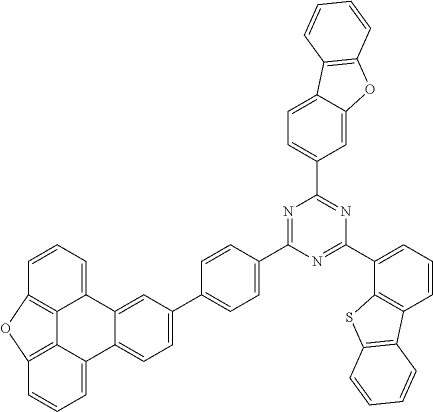 [SMILES: c1ccc2c(c1)oc1cc(-c3nc(-c4ccc(-c5ccc6c(c5)c5cccc7oc8cccc6c8c75)cc4)nc(-c4cccc5c4sc4ccccc45)n3)ccc12]

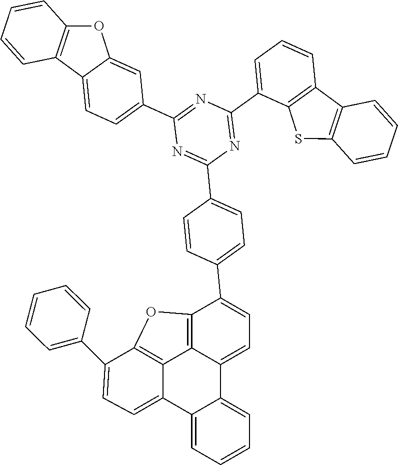 [SMILES: c1ccc(-c2ccc3c4ccccc4c4ccc(-c5ccc(-c6nc(-c7ccc8c(c7)oc7ccccc78)nc(-c7cccc8c7sc7ccccc78)n6)cc5)c5oc2c3c54)cc1]

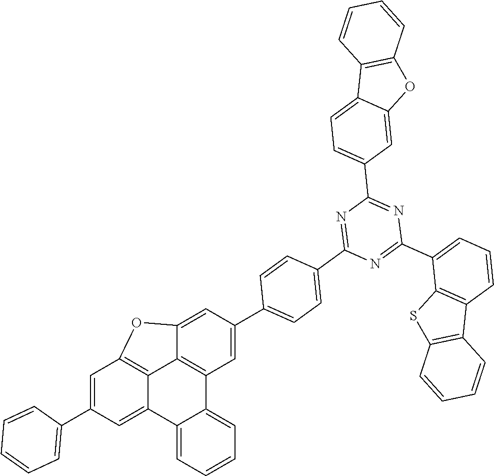 [SMILES: c1ccc(-c2cc3oc4cc(-c5ccc(-c6nc(-c7ccc8c(c7)oc7ccccc78)nc(-c7cccc8c7sc7ccccc78)n6)cc5)cc5c6ccccc6c(c2)c3c45)cc1]

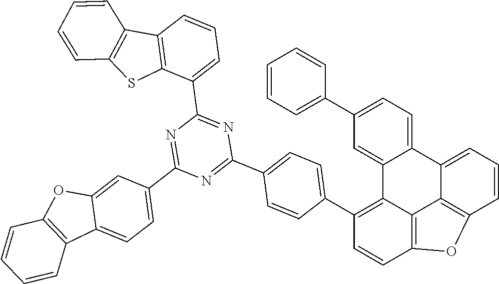 [SMILES: c1ccc(-c2ccc3c(c2)c2c(-c4ccc(-c5nc(-c6ccc7c(c6)oc6ccccc67)nc(-c6cccc7c6sc6ccccc67)n5)cc4)ccc4oc5cccc3c5c42)cc1]

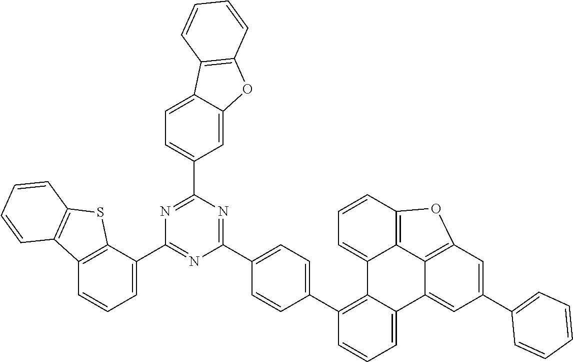 [SMILES: c1ccc(-c2cc3oc4cccc5c6c(-c7ccc(-c8nc(-c9ccc%10c(c9)oc9ccccc9%10)nc(-c9cccc%10c9sc9ccccc9%10)n8)cc7)cccc6c(c2)c3c45)cc1]

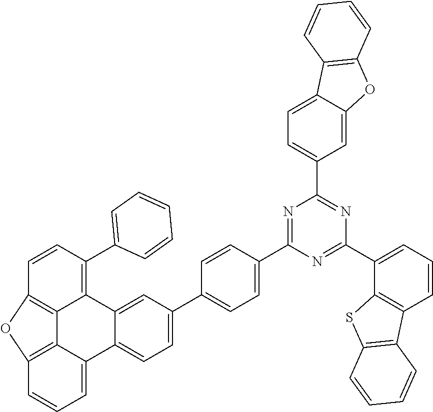 [SMILES: c1ccc(-c2ccc3oc4cccc5c6ccc(-c7ccc(-c8nc(-c9ccc%10c(c9)oc9ccccc9%10)nc(-c9cccc%10c9sc9ccccc9%10)n8)cc7)cc6c2c3c45)cc1]

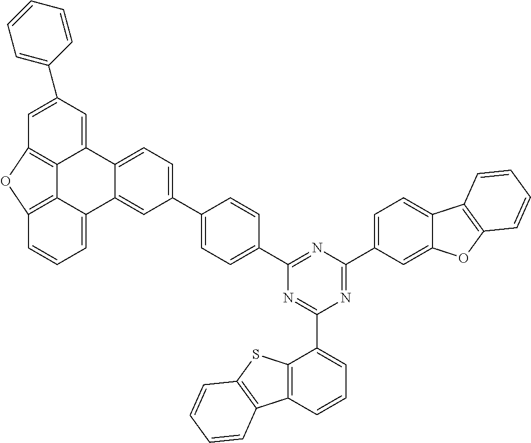 [SMILES: c1ccc(-c2cc3oc4cccc5c6cc(-c7ccc(-c8nc(-c9ccc%10c(c9)oc9ccccc9%10)nc(-c9cccc%10c9sc9ccccc9%10)n8)cc7)ccc6c(c2)c3c45)cc1]

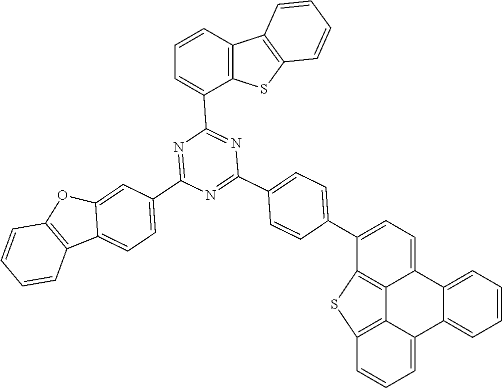 [SMILES: c1ccc2c(c1)oc1cc(-c3nc(-c4ccc(-c5ccc6c7ccccc7c7cccc8sc5c6c87)cc4)nc(-c4cccc5c4sc4ccccc45)n3)ccc12]

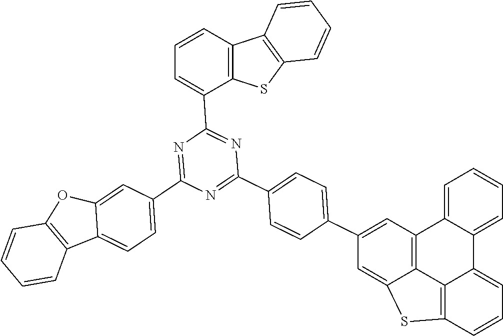 [SMILES: c1ccc2c(c1)oc1cc(-c3nc(-c4ccc(-c5cc6sc7cccc8c9ccccc9c(c5)c6c78)cc4)nc(-c4cccc5c4sc4ccccc45)n3)ccc12]

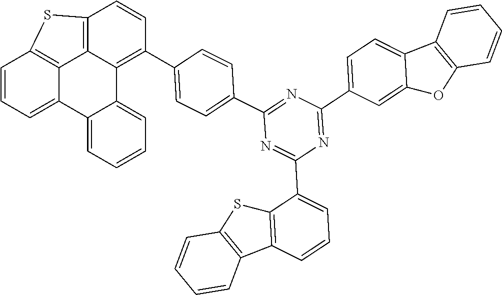 [SMILES: c1ccc2c(c1)oc1cc(-c3nc(-c4ccc(-c5ccc6sc7cccc8c9ccccc9c5c6c78)cc4)nc(-c4cccc5c4sc4ccccc45)n3)ccc12]

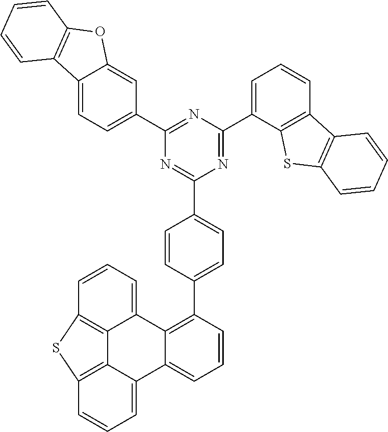 [SMILES: c1ccc2c(c1)oc1cc(-c3nc(-c4ccc(-c5cccc6c7cccc8sc9cccc(c56)c9c87)cc4)nc(-c4cccc5c4sc4ccccc45)n3)ccc12]

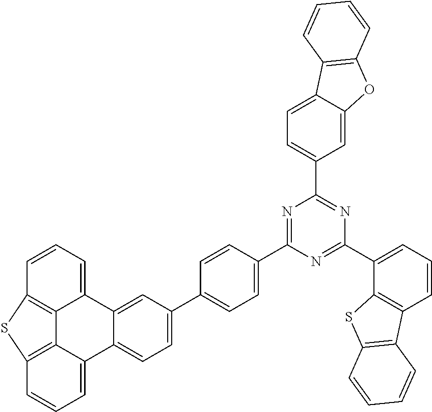 [SMILES: c1ccc2c(c1)oc1cc(-c3nc(-c4ccc(-c5ccc6c(c5)c5cccc7sc8cccc6c8c75)cc4)nc(-c4cccc5c4sc4ccccc45)n3)ccc12]

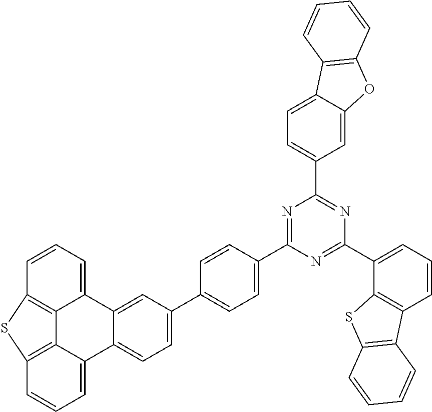 [SMILES: c1ccc2c(c1)oc1cc(-c3nc(-c4ccc(-c5ccc6c(c5)c5cccc7sc8cccc6c8c75)cc4)nc(-c4cccc5c4sc4ccccc45)n3)ccc12]

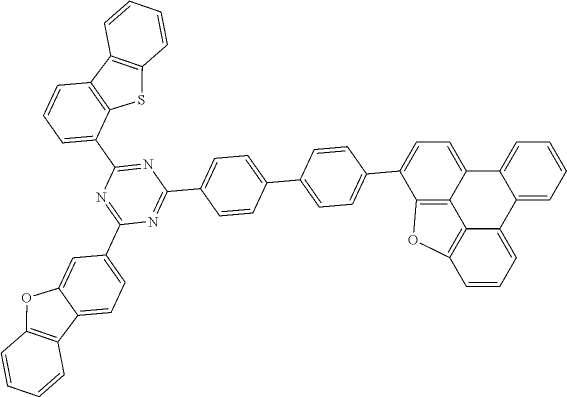 [SMILES: c1ccc2c(c1)oc1cc(-c3nc(-c4ccc(-c5ccc(-c6ccc7c8ccccc8c8cccc9oc6c7c98)cc5)cc4)nc(-c4cccc5c4sc4ccccc45)n3)ccc12]

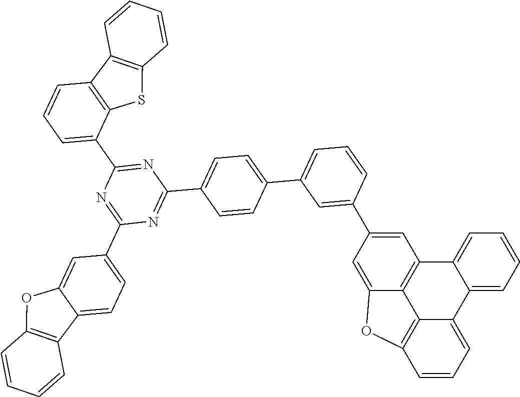 [SMILES: c1cc(-c2ccc(-c3nc(-c4ccc5c(c4)oc4ccccc45)nc(-c4cccc5c4sc4ccccc45)n3)cc2)cc(-c2cc3oc4cccc5c6ccccc6c(c2)c3c45)c1]

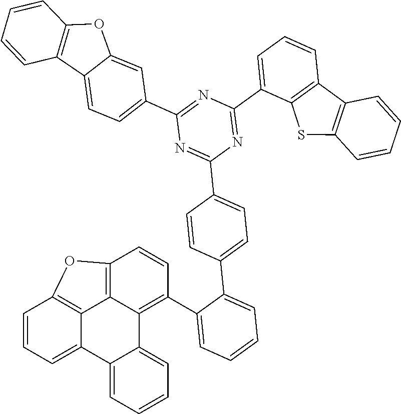 [SMILES: c1ccc(-c2ccc3oc4cccc5c6ccccc6c2c3c45)c(-c2ccc(-c3nc(-c4ccc5c(c4)oc4ccccc45)nc(-c4cccc5c4sc4ccccc45)n3)cc2)c1]

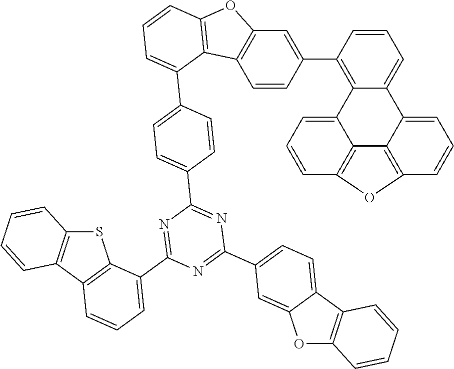 [SMILES: c1ccc2c(c1)oc1cc(-c3nc(-c4ccc(-c5cccc6oc7cc(-c8cccc9c%10cccc%11oc%12cccc(c89)c%12c%11%10)ccc7c56)cc4)nc(-c4cccc5c4sc4ccccc45)n3)ccc12]

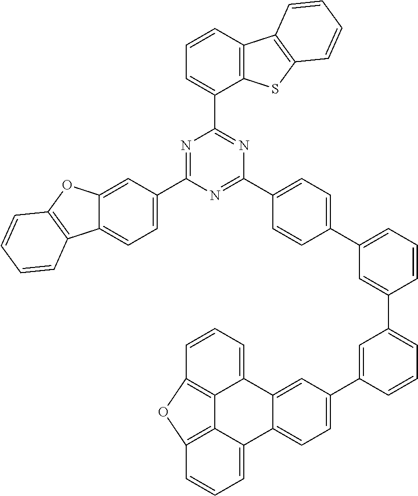 [SMILES: c1cc(-c2ccc(-c3nc(-c4ccc5c(c4)oc4ccccc45)nc(-c4cccc5c4sc4ccccc45)n3)cc2)cc(-c2cccc(-c3ccc4c(c3)c3cccc5oc6cccc4c6c53)c2)c1]

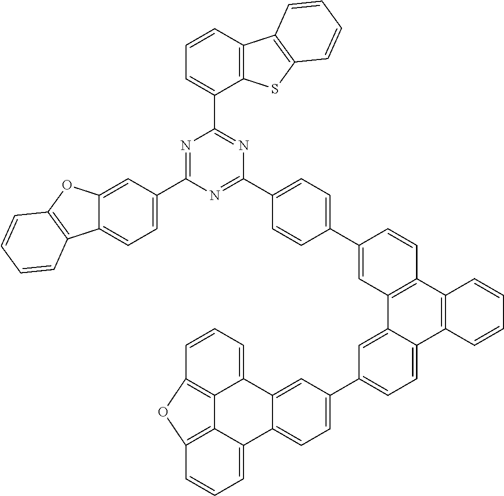 [SMILES: c1ccc2c(c1)oc1cc(-c3nc(-c4ccc(-c5ccc6c7ccccc7c7ccc(-c8ccc9c(c8)c8cccc%10oc%11cccc9c%11c%108)cc7c6c5)cc4)nc(-c4cccc5c4sc4ccccc45)n3)ccc12]

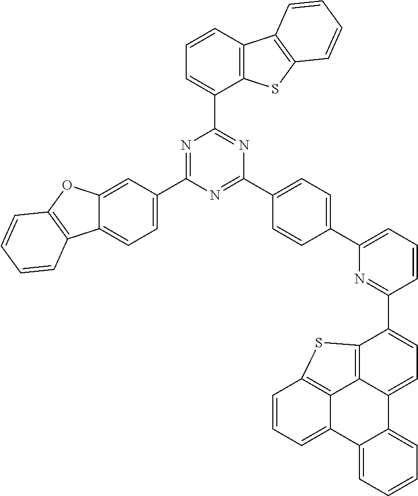 [SMILES: c1cc(-c2ccc(-c3nc(-c4ccc5c(c4)oc4ccccc45)nc(-c4cccc5c4sc4ccccc45)n3)cc2)nc(-c2ccc3c4ccccc4c4cccc5sc2c3c54)c1]

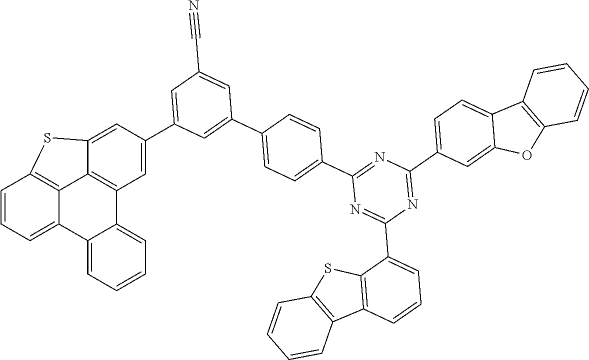 [SMILES: N#Cc1cc(-c2ccc(-c3nc(-c4ccc5c(c4)oc4ccccc45)nc(-c4cccc5c4sc4ccccc45)n3)cc2)cc(-c2cc3sc4cccc5c6ccccc6c(c2)c3c45)c1]

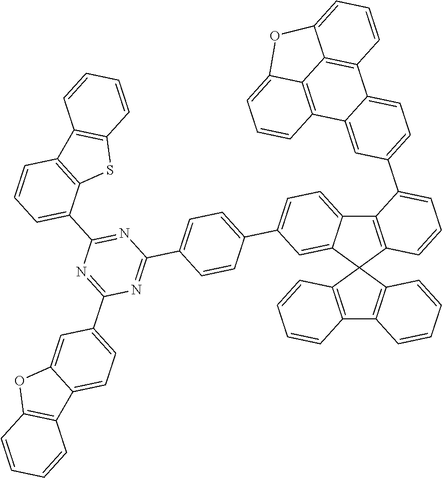 [SMILES: c1ccc2c(c1)-c1ccccc1C21c2cc(-c3ccc(-c4nc(-c5ccc6c(c5)oc5ccccc56)nc(-c5cccc6c5sc5ccccc56)n4)cc3)ccc2-c2c(-c3ccc4c(c3)c3cccc5oc6cccc4c6c53)cccc21]

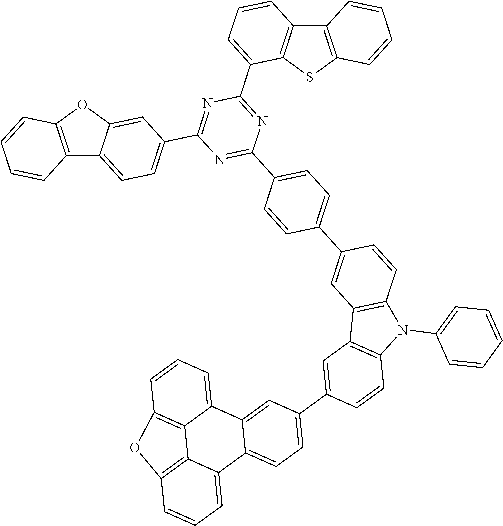 [SMILES: c1ccc(-n2c3ccc(-c4ccc(-c5nc(-c6ccc7c(c6)oc6ccccc67)nc(-c6cccc7c6sc6ccccc67)n5)cc4)cc3c3cc(-c4ccc5c(c4)c4cccc6oc7cccc5c7c64)ccc32)cc1]

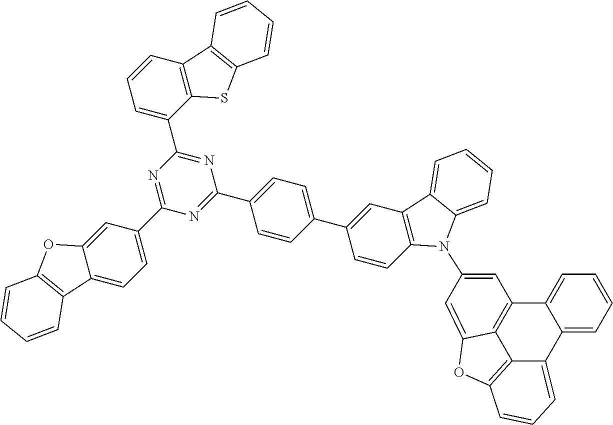 [SMILES: c1ccc2c(c1)oc1cc(-c3nc(-c4ccc(-c5ccc6c(c5)c5ccccc5n6-c5cc6oc7cccc8c9ccccc9c(c5)c6c78)cc4)nc(-c4cccc5c4sc4ccccc45)n3)ccc12]

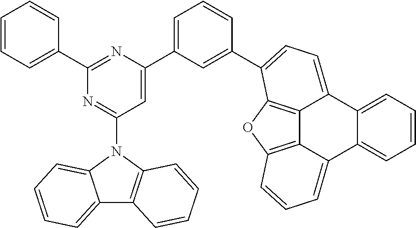 [SMILES: c1ccc(-c2nc(-c3cccc(-c4ccc5c6ccccc6c6cccc7oc4c5c76)c3)cc(-n3c4ccccc4c4ccccc43)n2)cc1]

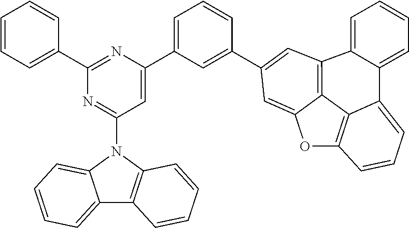 [SMILES: c1ccc(-c2nc(-c3cccc(-c4cc5oc6cccc7c8ccccc8c(c4)c5c67)c3)cc(-n3c4ccccc4c4ccccc43)n2)cc1]

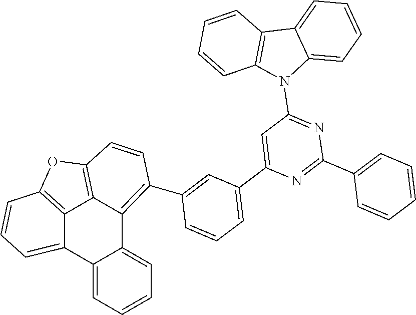 [SMILES: c1ccc(-c2nc(-c3cccc(-c4ccc5oc6cccc7c8ccccc8c4c5c67)c3)cc(-n3c4ccccc4c4ccccc43)n2)cc1]

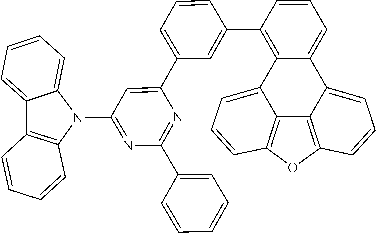 [SMILES: c1ccc(-c2nc(-c3cccc(-c4cccc5c6cccc7oc8cccc(c45)c8c76)c3)cc(-n3c4ccccc4c4ccccc43)n2)cc1]

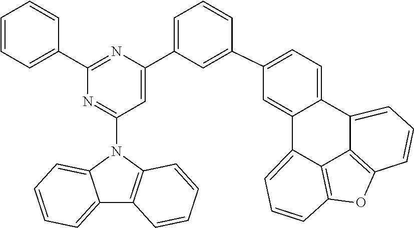 [SMILES: c1ccc(-c2nc(-c3cccc(-c4ccc5c(c4)c4cccc6oc7cccc5c7c64)c3)cc(-n3c4ccccc4c4ccccc43)n2)cc1]

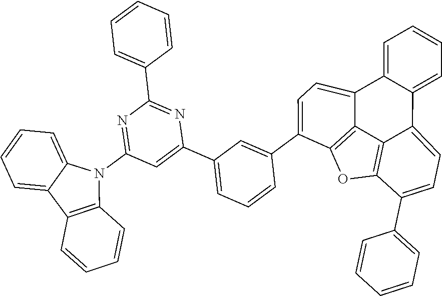 [SMILES: c1ccc(-c2nc(-c3cccc(-c4ccc5c6ccccc6c6ccc(-c7ccccc7)c7oc4c5c76)c3)cc(-n3c4ccccc4c4ccccc43)n2)cc1]

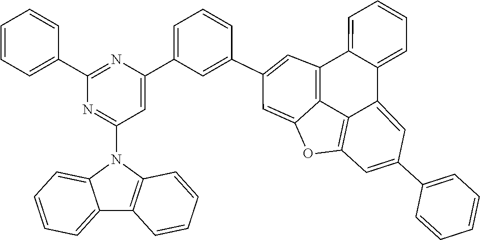 [SMILES: c1ccc(-c2cc3oc4cc(-c5cccc(-c6cc(-n7c8ccccc8c8ccccc87)nc(-c7ccccc7)n6)c5)cc5c6ccccc6c(c2)c3c45)cc1]

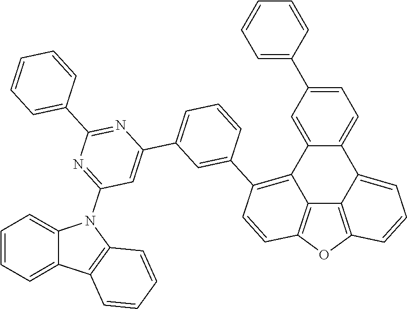 [SMILES: c1ccc(-c2ccc3c(c2)c2c(-c4cccc(-c5cc(-n6c7ccccc7c7ccccc76)nc(-c6ccccc6)n5)c4)ccc4oc5cccc3c5c42)cc1]

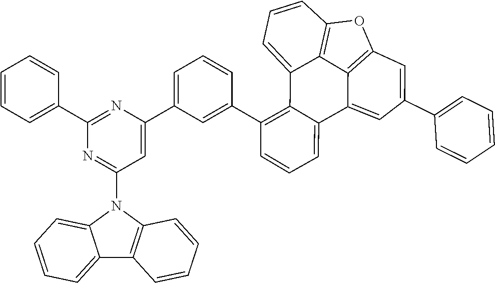 [SMILES: c1ccc(-c2cc3oc4cccc5c6c(-c7cccc(-c8cc(-n9c%10ccccc%10c%10ccccc%109)nc(-c9ccccc9)n8)c7)cccc6c(c2)c3c45)cc1]

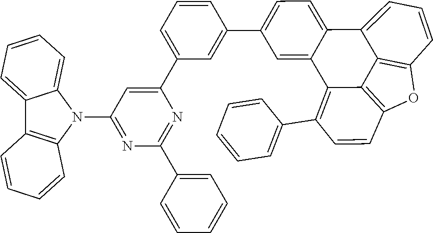 [SMILES: c1ccc(-c2nc(-c3cccc(-c4ccc5c(c4)c4c(-c6ccccc6)ccc6oc7cccc5c7c64)c3)cc(-n3c4ccccc4c4ccccc43)n2)cc1]

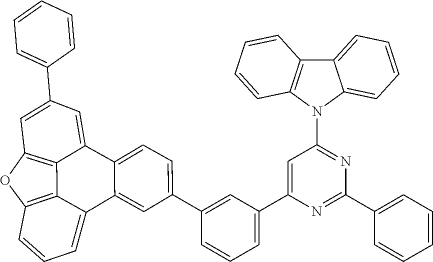 [SMILES: c1ccc(-c2cc3oc4cccc5c6cc(-c7cccc(-c8cc(-n9c%10ccccc%10c%10ccccc%109)nc(-c9ccccc9)n8)c7)ccc6c(c2)c3c45)cc1]

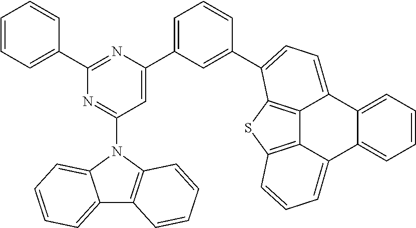 [SMILES: c1ccc(-c2nc(-c3cccc(-c4ccc5c6ccccc6c6cccc7sc4c5c76)c3)cc(-n3c4ccccc4c4ccccc43)n2)cc1]

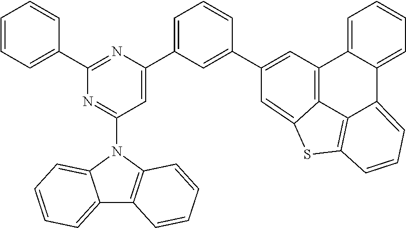 [SMILES: c1ccc(-c2nc(-c3cccc(-c4cc5sc6cccc7c8ccccc8c(c4)c5c67)c3)cc(-n3c4ccccc4c4ccccc43)n2)cc1]

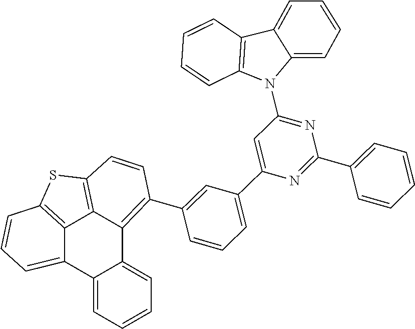 [SMILES: c1ccc(-c2nc(-c3cccc(-c4ccc5sc6cccc7c8ccccc8c4c5c67)c3)cc(-n3c4ccccc4c4ccccc43)n2)cc1]

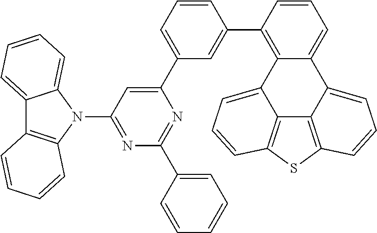 [SMILES: c1ccc(-c2nc(-c3cccc(-c4cccc5c6cccc7sc8cccc(c45)c8c76)c3)cc(-n3c4ccccc4c4ccccc43)n2)cc1]

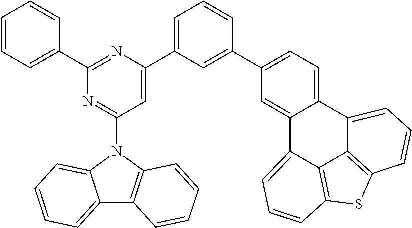 [SMILES: c1ccc(-c2nc(-c3cccc(-c4ccc5c(c4)c4cccc6sc7cccc5c7c64)c3)cc(-n3c4ccccc4c4ccccc43)n2)cc1]

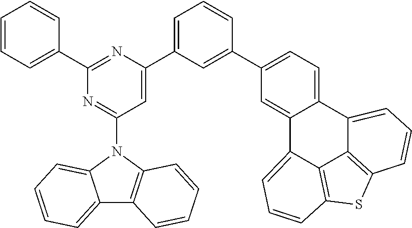 [SMILES: c1ccc(-c2nc(-c3cccc(-c4ccc5c(c4)c4cccc6sc7cccc5c7c64)c3)cc(-n3c4ccccc4c4ccccc43)n2)cc1]

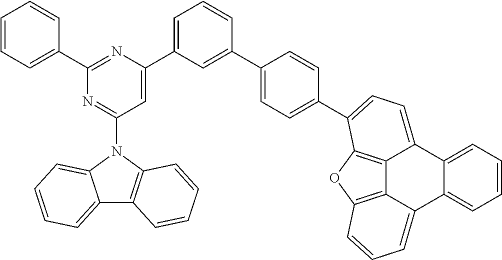 [SMILES: c1ccc(-c2nc(-c3cccc(-c4ccc(-c5ccc6c7ccccc7c7cccc8oc5c6c87)cc4)c3)cc(-n3c4ccccc4c4ccccc43)n2)cc1]

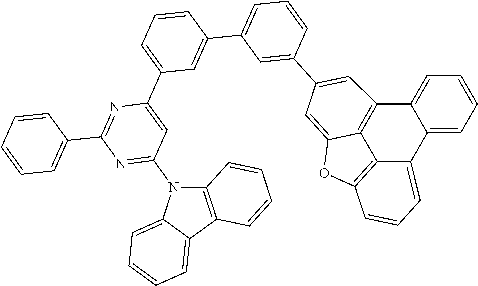 [SMILES: c1ccc(-c2nc(-c3cccc(-c4cccc(-c5cc6oc7cccc8c9ccccc9c(c5)c6c78)c4)c3)cc(-n3c4ccccc4c4ccccc43)n2)cc1]

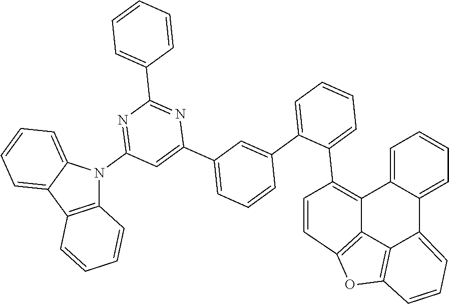 [SMILES: c1ccc(-c2nc(-c3cccc(-c4ccccc4-c4ccc5oc6cccc7c8ccccc8c4c5c67)c3)cc(-n3c4ccccc4c4ccccc43)n2)cc1]

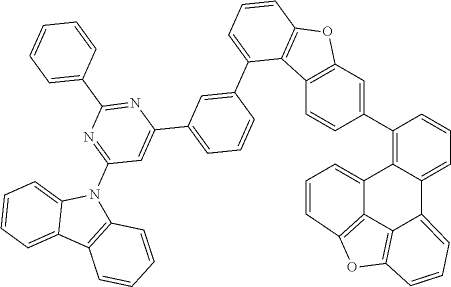 [SMILES: c1ccc(-c2nc(-c3cccc(-c4cccc5oc6cc(-c7cccc8c9cccc%10oc%11cccc(c78)c%11c%109)ccc6c45)c3)cc(-n3c4ccccc4c4ccccc43)n2)cc1]

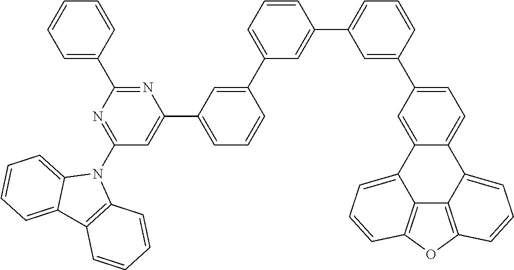 [SMILES: c1ccc(-c2nc(-c3cccc(-c4cccc(-c5cccc(-c6ccc7c(c6)c6cccc8oc9cccc7c9c86)c5)c4)c3)cc(-n3c4ccccc4c4ccccc43)n2)cc1]

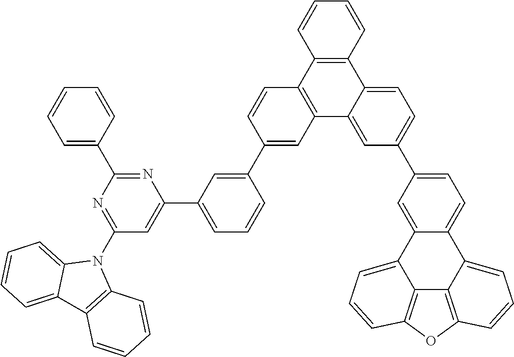 [SMILES: c1ccc(-c2nc(-c3cccc(-c4ccc5c6ccccc6c6ccc(-c7ccc8c(c7)c7cccc9oc%10cccc8c%10c97)cc6c5c4)c3)cc(-n3c4ccccc4c4ccccc43)n2)cc1]

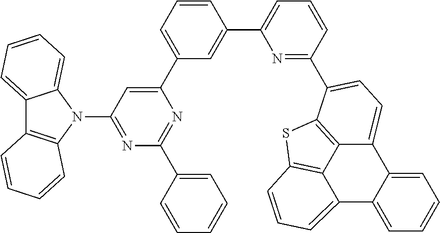 [SMILES: c1ccc(-c2nc(-c3cccc(-c4cccc(-c5ccc6c7ccccc7c7cccc8sc5c6c87)n4)c3)cc(-n3c4ccccc4c4ccccc43)n2)cc1]

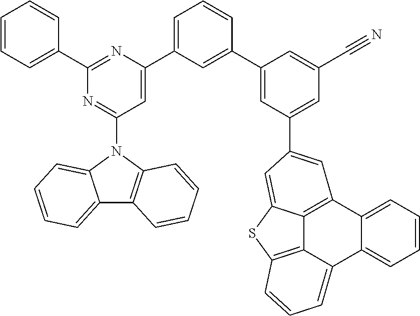 [SMILES: N#Cc1cc(-c2cccc(-c3cc(-n4c5ccccc5c5ccccc54)nc(-c4ccccc4)n3)c2)cc(-c2cc3sc4cccc5c6ccccc6c(c2)c3c45)c1]

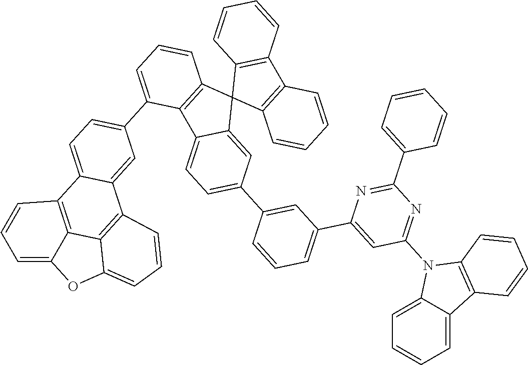 [SMILES: c1ccc(-c2nc(-c3cccc(-c4ccc5c(c4)C4(c6ccccc6-c6ccccc64)c4cccc(-c6ccc7c(c6)c6cccc8oc9cccc7c9c86)c4-5)c3)cc(-n3c4ccccc4c4ccccc43)n2)cc1]

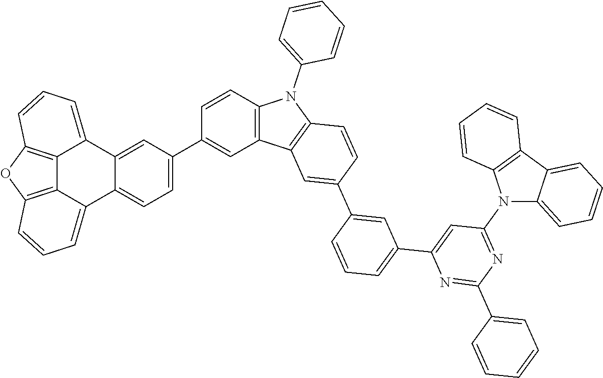 [SMILES: c1ccc(-c2nc(-c3cccc(-c4ccc5c(c4)c4cc(-c6ccc7c(c6)c6cccc8oc9cccc7c9c86)ccc4n5-c4ccccc4)c3)cc(-n3c4ccccc4c4ccccc43)n2)cc1]

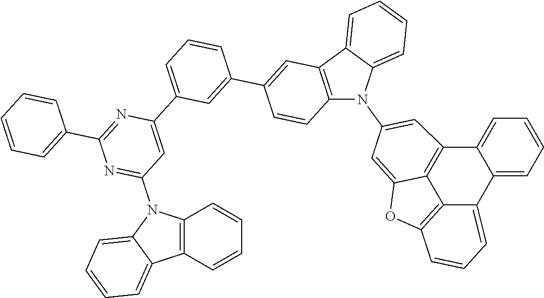 [SMILES: c1ccc(-c2nc(-c3cccc(-c4ccc5c(c4)c4ccccc4n5-c4cc5oc6cccc7c8ccccc8c(c4)c5c67)c3)cc(-n3c4ccccc4c4ccccc43)n2)cc1]

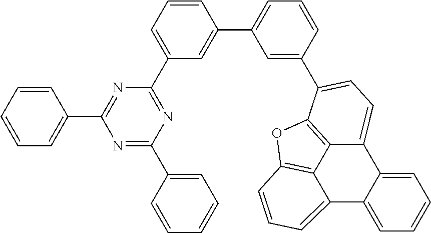 [SMILES: c1ccc(-c2nc(-c3ccccc3)nc(-c3cccc(-c4cccc(-c5ccc6c7ccccc7c7cccc8oc5c6c87)c4)c3)n2)cc1]

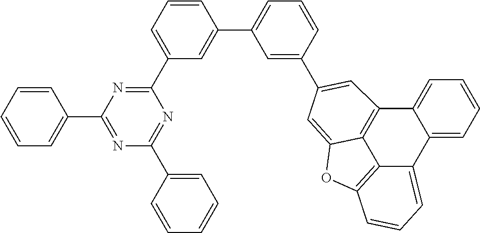 [SMILES: c1ccc(-c2nc(-c3ccccc3)nc(-c3cccc(-c4cccc(-c5cc6oc7cccc8c9ccccc9c(c5)c6c78)c4)c3)n2)cc1]

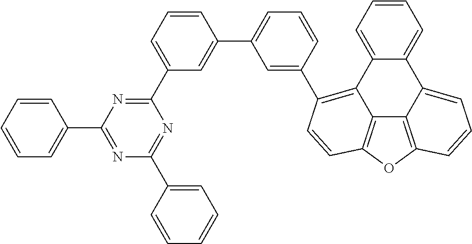 [SMILES: c1ccc(-c2nc(-c3ccccc3)nc(-c3cccc(-c4cccc(-c5ccc6oc7cccc8c9ccccc9c5c6c78)c4)c3)n2)cc1]

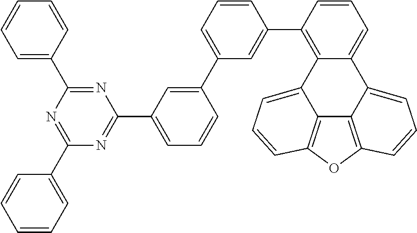 [SMILES: c1ccc(-c2nc(-c3ccccc3)nc(-c3cccc(-c4cccc(-c5cccc6c7cccc8oc9cccc(c56)c9c87)c4)c3)n2)cc1]

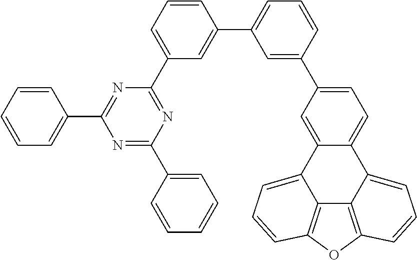 [SMILES: c1ccc(-c2nc(-c3ccccc3)nc(-c3cccc(-c4cccc(-c5ccc6c(c5)c5cccc7oc8cccc6c8c75)c4)c3)n2)cc1]

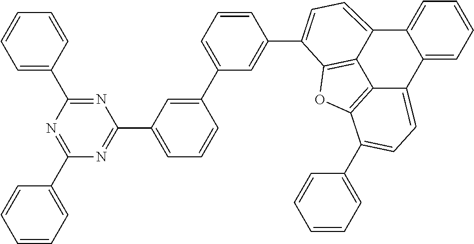 [SMILES: c1ccc(-c2nc(-c3ccccc3)nc(-c3cccc(-c4cccc(-c5ccc6c7ccccc7c7ccc(-c8ccccc8)c8oc5c6c87)c4)c3)n2)cc1]

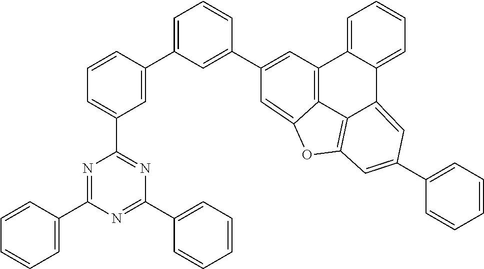 [SMILES: c1ccc(-c2cc3oc4cc(-c5cccc(-c6cccc(-c7nc(-c8ccccc8)nc(-c8ccccc8)n7)c6)c5)cc5c6ccccc6c(c2)c3c45)cc1]

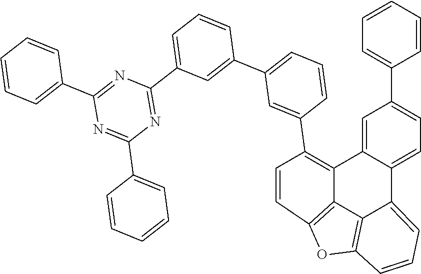 [SMILES: c1ccc(-c2ccc3c(c2)c2c(-c4cccc(-c5cccc(-c6nc(-c7ccccc7)nc(-c7ccccc7)n6)c5)c4)ccc4oc5cccc3c5c42)cc1]

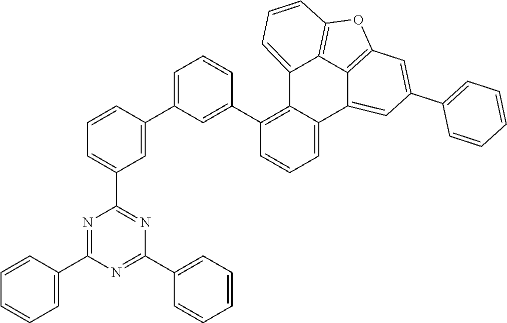 [SMILES: c1ccc(-c2cc3oc4cccc5c6c(-c7cccc(-c8cccc(-c9nc(-c%10ccccc%10)nc(-c%10ccccc%10)n9)c8)c7)cccc6c(c2)c3c45)cc1]

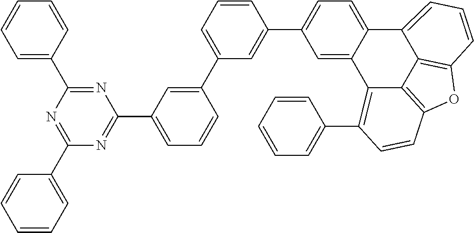 [SMILES: c1ccc(-c2nc(-c3ccccc3)nc(-c3cccc(-c4cccc(-c5ccc6c(c5)c5c(-c7ccccc7)ccc7oc8cccc6c8c75)c4)c3)n2)cc1]

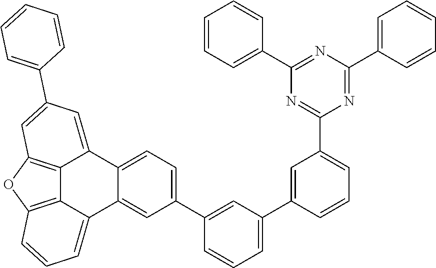 [SMILES: c1ccc(-c2cc3oc4cccc5c6cc(-c7cccc(-c8cccc(-c9nc(-c%10ccccc%10)nc(-c%10ccccc%10)n9)c8)c7)ccc6c(c2)c3c45)cc1]

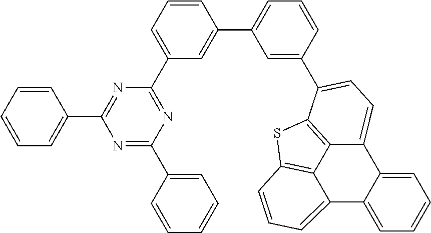 [SMILES: c1ccc(-c2nc(-c3ccccc3)nc(-c3cccc(-c4cccc(-c5ccc6c7ccccc7c7cccc8sc5c6c87)c4)c3)n2)cc1]

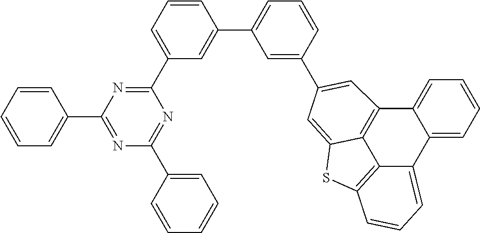 [SMILES: c1ccc(-c2nc(-c3ccccc3)nc(-c3cccc(-c4cccc(-c5cc6sc7cccc8c9ccccc9c(c5)c6c78)c4)c3)n2)cc1]

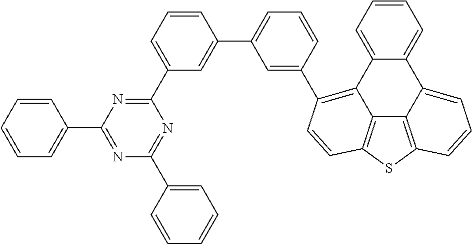 [SMILES: c1ccc(-c2nc(-c3ccccc3)nc(-c3cccc(-c4cccc(-c5ccc6sc7cccc8c9ccccc9c5c6c78)c4)c3)n2)cc1]

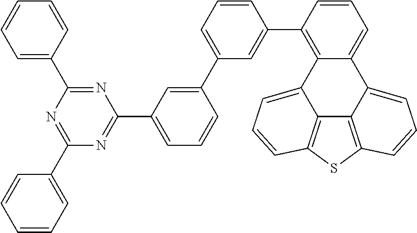 [SMILES: c1ccc(-c2nc(-c3ccccc3)nc(-c3cccc(-c4cccc(-c5cccc6c7cccc8sc9cccc(c56)c9c87)c4)c3)n2)cc1]

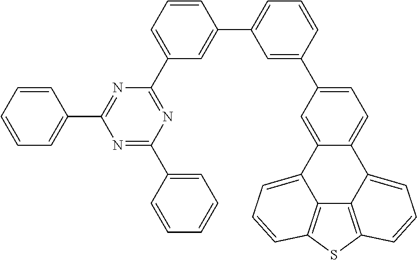 [SMILES: c1ccc(-c2nc(-c3ccccc3)nc(-c3cccc(-c4cccc(-c5ccc6c(c5)c5cccc7sc8cccc6c8c75)c4)c3)n2)cc1]

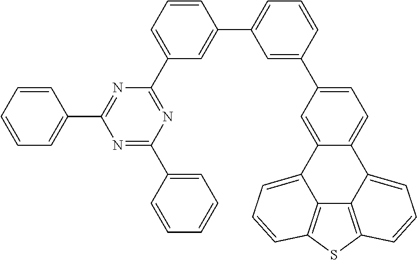 [SMILES: c1ccc(-c2nc(-c3ccccc3)nc(-c3cccc(-c4cccc(-c5ccc6c(c5)c5cccc7sc8cccc6c8c75)c4)c3)n2)cc1]

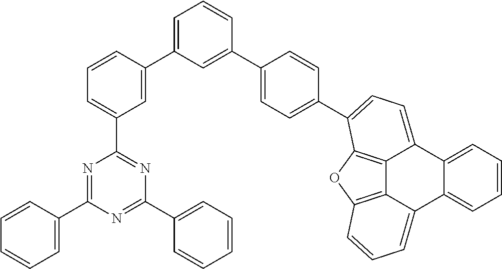 [SMILES: c1ccc(-c2nc(-c3ccccc3)nc(-c3cccc(-c4cccc(-c5ccc(-c6ccc7c8ccccc8c8cccc9oc6c7c98)cc5)c4)c3)n2)cc1]

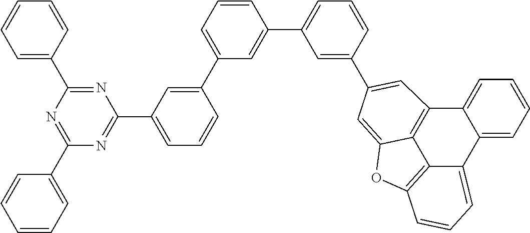 [SMILES: c1ccc(-c2nc(-c3ccccc3)nc(-c3cccc(-c4cccc(-c5cccc(-c6cc7oc8cccc9c%10ccccc%10c(c6)c7c89)c5)c4)c3)n2)cc1]

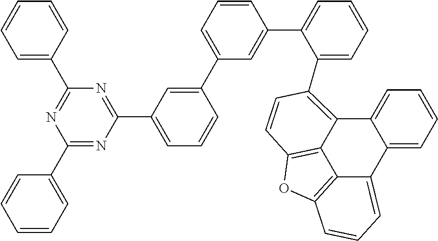 [SMILES: c1ccc(-c2nc(-c3ccccc3)nc(-c3cccc(-c4cccc(-c5ccccc5-c5ccc6oc7cccc8c9ccccc9c5c6c78)c4)c3)n2)cc1]

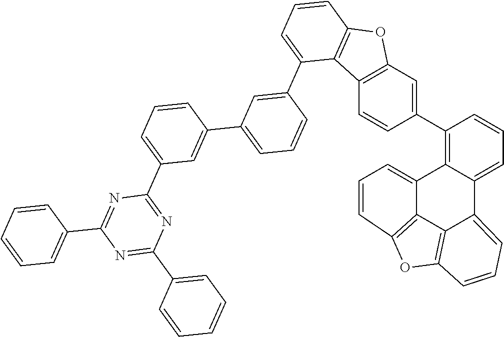 [SMILES: c1ccc(-c2nc(-c3ccccc3)nc(-c3cccc(-c4cccc(-c5cccc6oc7cc(-c8cccc9c%10cccc%11oc%12cccc(c89)c%12c%11%10)ccc7c56)c4)c3)n2)cc1]

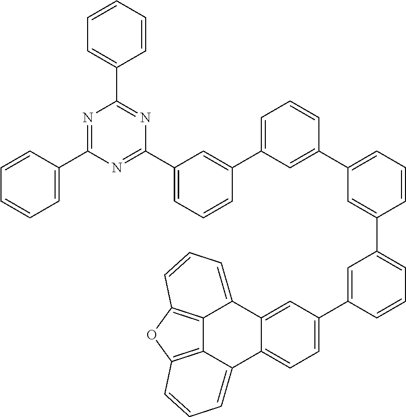 [SMILES: c1ccc(-c2nc(-c3ccccc3)nc(-c3cccc(-c4cccc(-c5cccc(-c6cccc(-c7ccc8c(c7)c7cccc9oc%10cccc8c%10c97)c6)c5)c4)c3)n2)cc1]

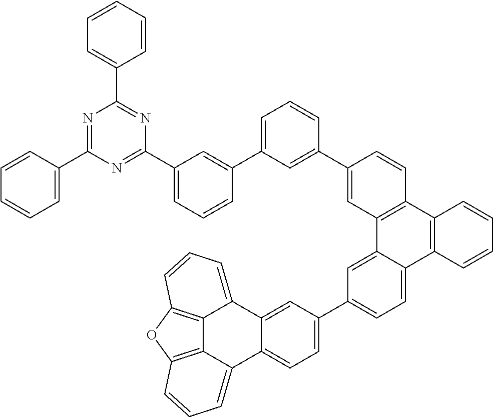 [SMILES: c1ccc(-c2nc(-c3ccccc3)nc(-c3cccc(-c4cccc(-c5ccc6c7ccccc7c7ccc(-c8ccc9c(c8)c8cccc%10oc%11cccc9c%11c%108)cc7c6c5)c4)c3)n2)cc1]

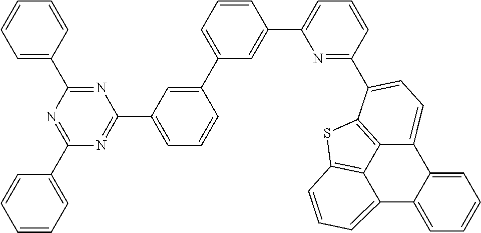 [SMILES: c1ccc(-c2nc(-c3ccccc3)nc(-c3cccc(-c4cccc(-c5cccc(-c6ccc7c8ccccc8c8cccc9sc6c7c98)n5)c4)c3)n2)cc1]

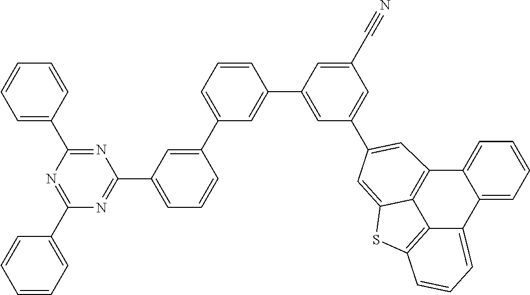 [SMILES: N#Cc1cc(-c2cccc(-c3cccc(-c4nc(-c5ccccc5)nc(-c5ccccc5)n4)c3)c2)cc(-c2cc3sc4cccc5c6ccccc6c(c2)c3c45)c1]

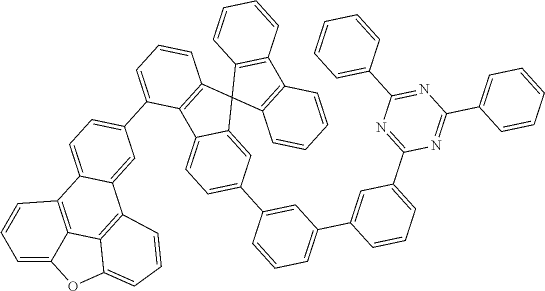 [SMILES: c1ccc(-c2nc(-c3ccccc3)nc(-c3cccc(-c4cccc(-c5ccc6c(c5)C5(c7ccccc7-c7ccccc75)c5cccc(-c7ccc8c(c7)c7cccc9oc%10cccc8c%10c97)c5-6)c4)c3)n2)cc1]

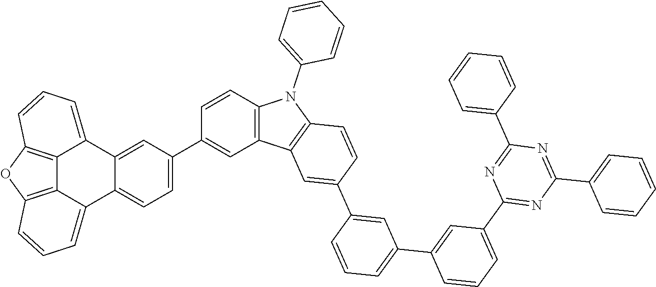 [SMILES: c1ccc(-c2nc(-c3ccccc3)nc(-c3cccc(-c4cccc(-c5ccc6c(c5)c5cc(-c7ccc8c(c7)c7cccc9oc%10cccc8c%10c97)ccc5n6-c5ccccc5)c4)c3)n2)cc1]

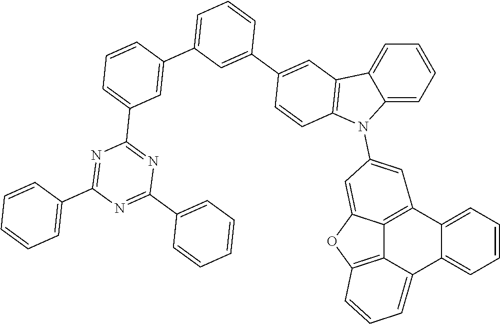 [SMILES: c1ccc(-c2nc(-c3ccccc3)nc(-c3cccc(-c4cccc(-c5ccc6c(c5)c5ccccc5n6-c5cc6oc7cccc8c9ccccc9c(c5)c6c78)c4)c3)n2)cc1]

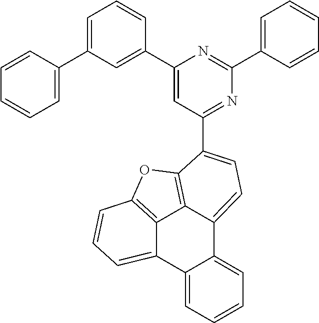 [SMILES: c1ccc(-c2cccc(-c3cc(-c4ccc5c6ccccc6c6cccc7oc4c5c76)nc(-c4ccccc4)n3)c2)cc1]

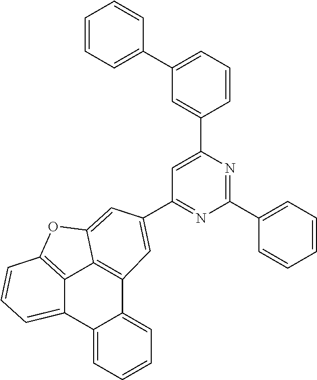 [SMILES: c1ccc(-c2cccc(-c3cc(-c4cc5oc6cccc7c8ccccc8c(c4)c5c67)nc(-c4ccccc4)n3)c2)cc1]

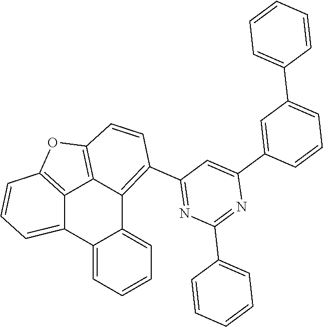 [SMILES: c1ccc(-c2cccc(-c3cc(-c4ccc5oc6cccc7c8ccccc8c4c5c67)nc(-c4ccccc4)n3)c2)cc1]

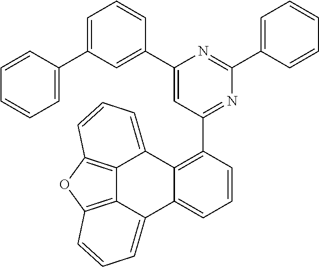 [SMILES: c1ccc(-c2cccc(-c3cc(-c4cccc5c6cccc7oc8cccc(c45)c8c76)nc(-c4ccccc4)n3)c2)cc1]

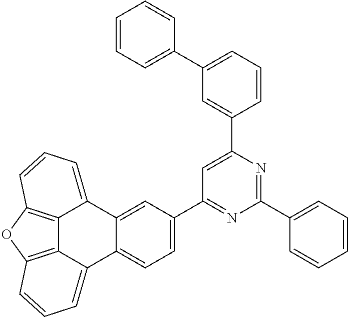

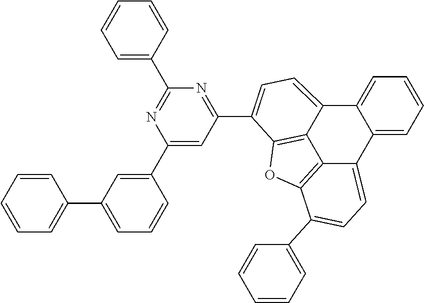 [SMILES: c1ccc(-c2cccc(-c3cc(-c4ccc5c6ccccc6c6ccc(-c7ccccc7)c7oc4c5c76)nc(-c4ccccc4)n3)c2)cc1]

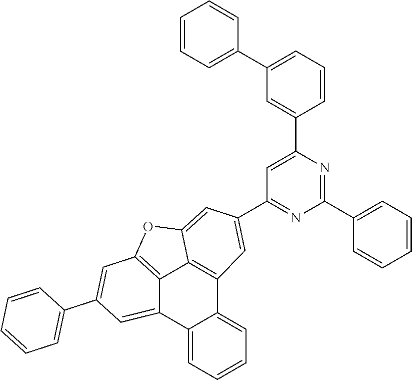 [SMILES: c1ccc(-c2cccc(-c3cc(-c4cc5oc6cc(-c7ccccc7)cc7c8ccccc8c(c4)c5c67)nc(-c4ccccc4)n3)c2)cc1]

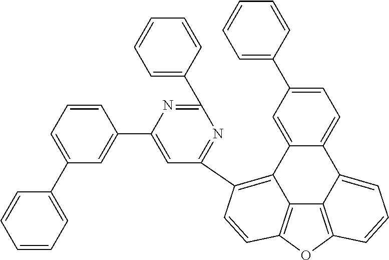 [SMILES: c1ccc(-c2cccc(-c3cc(-c4ccc5oc6cccc7c8ccc(-c9ccccc9)cc8c4c5c67)nc(-c4ccccc4)n3)c2)cc1]

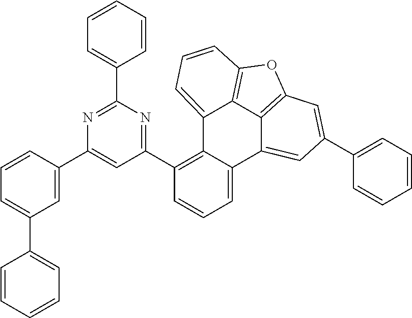 [SMILES: c1ccc(-c2cccc(-c3cc(-c4cccc5c6cc(-c7ccccc7)cc7oc8cccc(c45)c8c76)nc(-c4ccccc4)n3)c2)cc1]

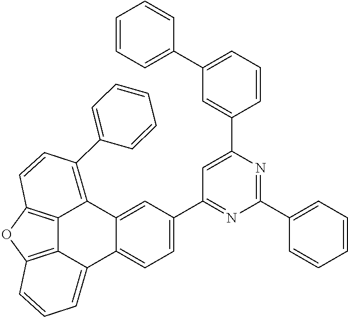

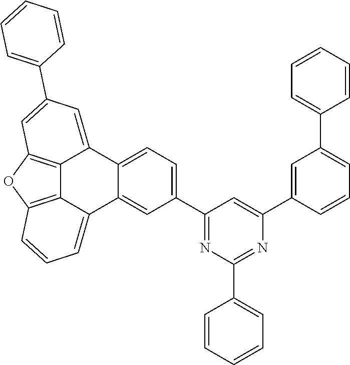

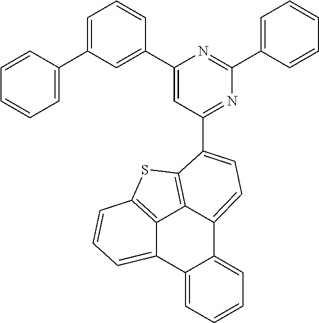 [SMILES: c1ccc(-c2cccc(-c3cc(-c4ccc5c6ccccc6c6cccc7sc4c5c76)nc(-c4ccccc4)n3)c2)cc1]

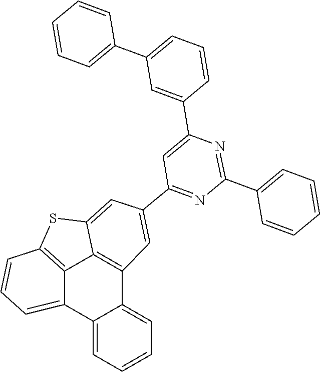 [SMILES: c1ccc(-c2cccc(-c3cc(-c4cc5sc6cccc7c8ccccc8c(c4)c5c67)nc(-c4ccccc4)n3)c2)cc1]

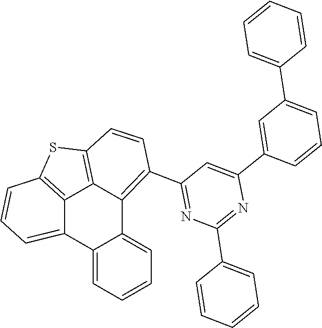 [SMILES: c1ccc(-c2cccc(-c3cc(-c4ccc5sc6cccc7c8ccccc8c4c5c67)nc(-c4ccccc4)n3)c2)cc1]

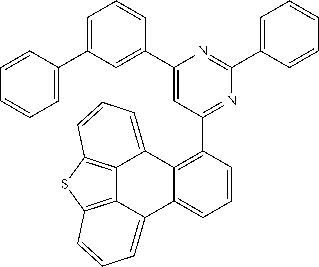 [SMILES: c1ccc(-c2cccc(-c3cc(-c4cccc5c6cccc7sc8cccc(c45)c8c76)nc(-c4ccccc4)n3)c2)cc1]

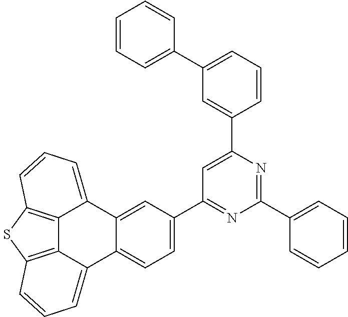

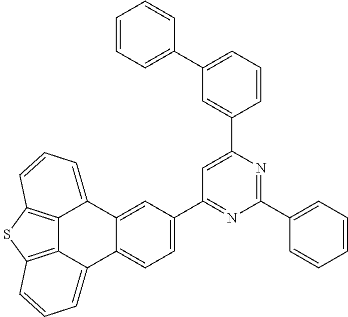

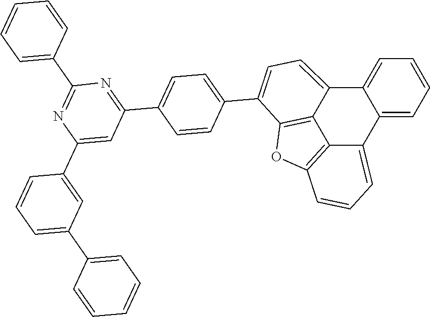 [SMILES: c1ccc(-c2cccc(-c3cc(-c4ccc(-c5ccc6c7ccccc7c7cccc8oc5c6c87)cc4)nc(-c4ccccc4)n3)c2)cc1]

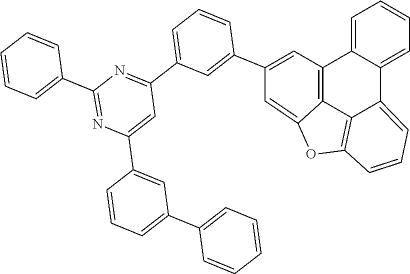 [SMILES: c1ccc(-c2cccc(-c3cc(-c4cccc(-c5cc6oc7cccc8c9ccccc9c(c5)c6c78)c4)nc(-c4ccccc4)n3)c2)cc1]

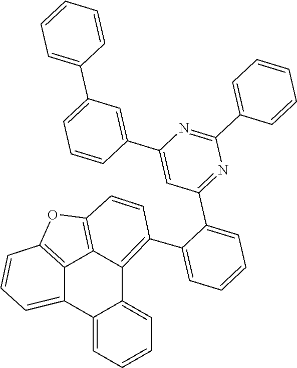 [SMILES: c1ccc(-c2cccc(-c3cc(-c4ccccc4-c4ccc5oc6cccc7c8ccccc8c4c5c67)nc(-c4ccccc4)n3)c2)cc1]

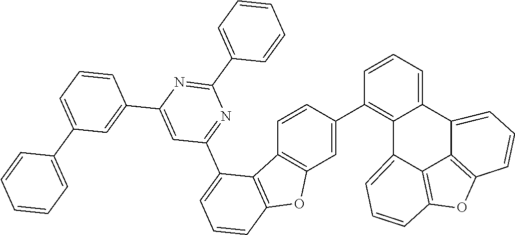 [SMILES: c1ccc(-c2cccc(-c3cc(-c4cccc5oc6cc(-c7cccc8c9cccc%10oc%11cccc(c78)c%11c%109)ccc6c45)nc(-c4ccccc4)n3)c2)cc1]

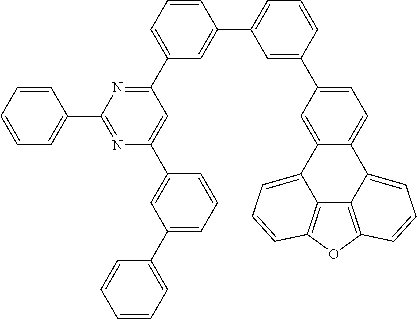 [SMILES: c1ccc(-c2cccc(-c3cc(-c4cccc(-c5cccc(-c6ccc7c(c6)c6cccc8oc9cccc7c9c86)c5)c4)nc(-c4ccccc4)n3)c2)cc1]

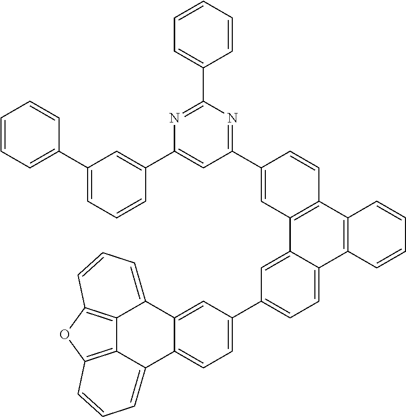 [SMILES: c1ccc(-c2cccc(-c3cc(-c4ccc5c6ccccc6c6ccc(-c7ccc8c(c7)c7cccc9oc%10cccc8c%10c97)cc6c5c4)nc(-c4ccccc4)n3)c2)cc1]

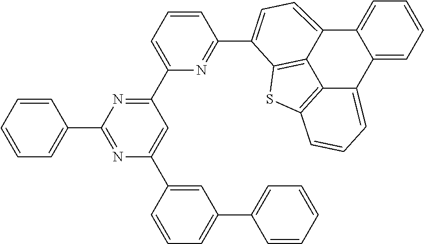 [SMILES: c1ccc(-c2cccc(-c3cc(-c4cccc(-c5ccc6c7ccccc7c7cccc8sc5c6c87)n4)nc(-c4ccccc4)n3)c2)cc1]

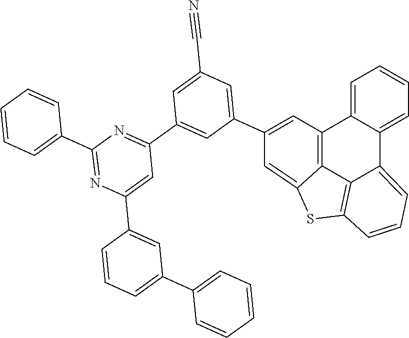 [SMILES: N#Cc1cc(-c2cc3sc4cccc5c6ccccc6c(c2)c3c45)cc(-c2cc(-c3cccc(-c4ccccc4)c3)nc(-c3ccccc3)n2)c1]

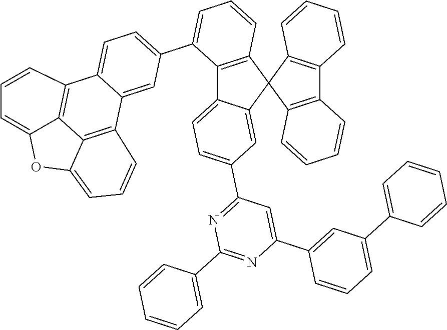 [SMILES: c1ccc(-c2cccc(-c3cc(-c4ccc5c(c4)C4(c6ccccc6-c6ccccc64)c4cccc(-c6ccc7c(c6)c6cccc8oc9cccc7c9c86)c4-5)nc(-c4ccccc4)n3)c2)cc1]

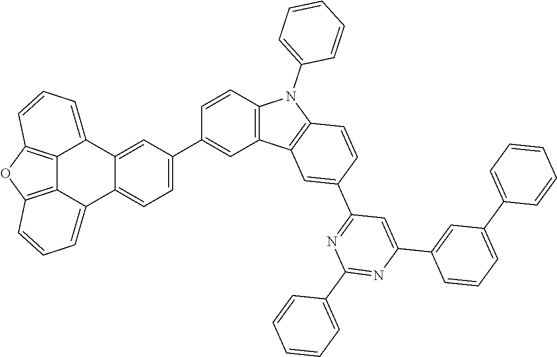 [SMILES: c1ccc(-c2cccc(-c3cc(-c4ccc5c(c4)c4cc(-c6ccc7c(c6)c6cccc8oc9cccc7c9c86)ccc4n5-c4ccccc4)nc(-c4ccccc4)n3)c2)cc1]

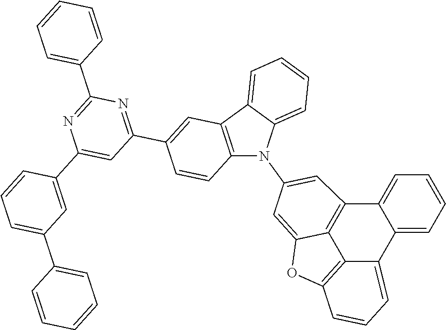 [SMILES: c1ccc(-c2cccc(-c3cc(-c4ccc5c(c4)c4ccccc4n5-c4cc5oc6cccc7c8ccccc8c(c4)c5c67)nc(-c4ccccc4)n3)c2)cc1]

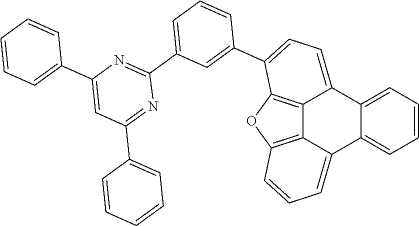 [SMILES: c1ccc(-c2cc(-c3ccccc3)nc(-c3cccc(-c4ccc5c6ccccc6c6cccc7oc4c5c76)c3)n2)cc1]

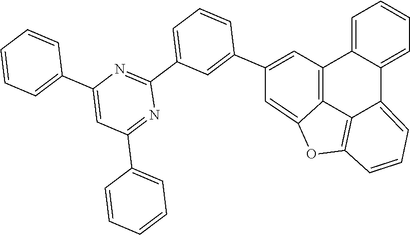 [SMILES: c1ccc(-c2cc(-c3ccccc3)nc(-c3cccc(-c4cc5oc6cccc7c8ccccc8c(c4)c5c67)c3)n2)cc1]

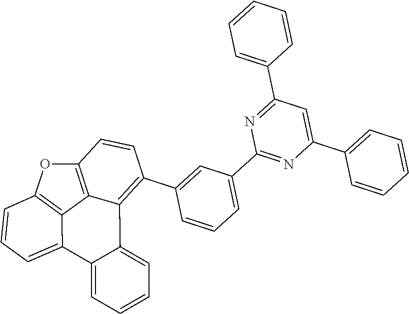 [SMILES: c1ccc(-c2cc(-c3ccccc3)nc(-c3cccc(-c4ccc5oc6cccc7c8ccccc8c4c5c67)c3)n2)cc1]

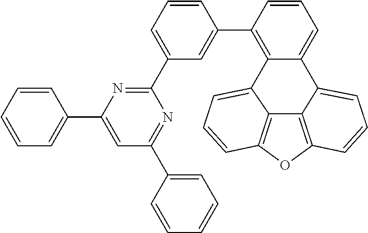 [SMILES: c1ccc(-c2cc(-c3ccccc3)nc(-c3cccc(-c4cccc5c6cccc7oc8cccc(c45)c8c76)c3)n2)cc1]

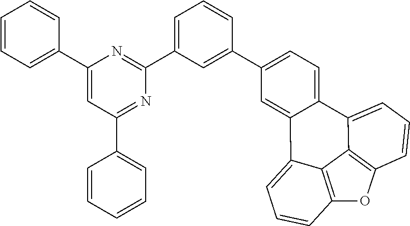 [SMILES: c1ccc(-c2cc(-c3ccccc3)nc(-c3cccc(-c4ccc5c(c4)c4cccc6oc7cccc5c7c64)c3)n2)cc1]

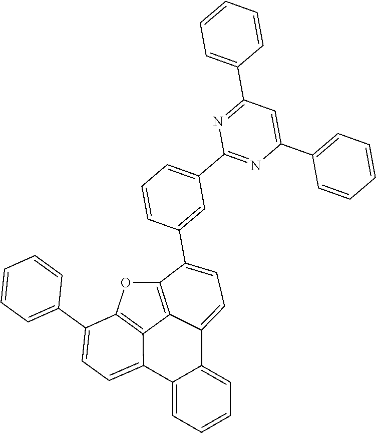 [SMILES: c1ccc(-c2cc(-c3ccccc3)nc(-c3cccc(-c4ccc5c6ccccc6c6ccc(-c7ccccc7)c7oc4c5c76)c3)n2)cc1]

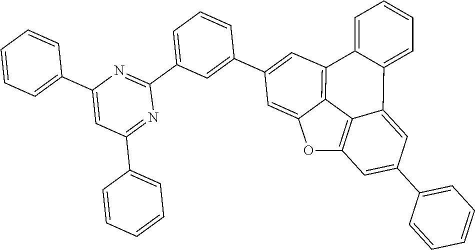 [SMILES: c1ccc(-c2cc3oc4cc(-c5cccc(-c6nc(-c7ccccc7)cc(-c7ccccc7)n6)c5)cc5c6ccccc6c(c2)c3c45)cc1]

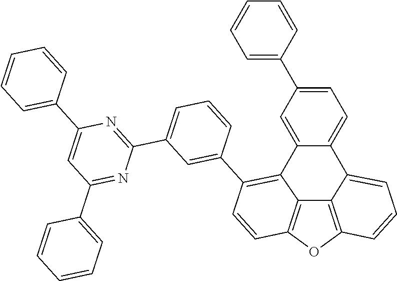 [SMILES: c1ccc(-c2ccc3c(c2)c2c(-c4cccc(-c5nc(-c6ccccc6)cc(-c6ccccc6)n5)c4)ccc4oc5cccc3c5c42)cc1]

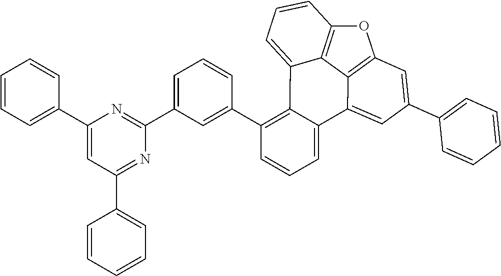 [SMILES: c1ccc(-c2cc3oc4cccc5c6c(-c7cccc(-c8nc(-c9ccccc9)cc(-c9ccccc9)n8)c7)cccc6c(c2)c3c45)cc1]

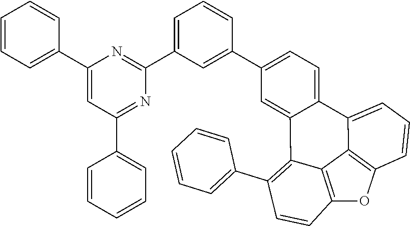 [SMILES: c1ccc(-c2cc(-c3ccccc3)nc(-c3cccc(-c4ccc5c(c4)c4c(-c6ccccc6)ccc6oc7cccc5c7c64)c3)n2)cc1]

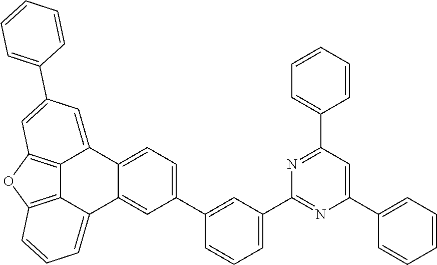 [SMILES: c1ccc(-c2cc3oc4cccc5c6cc(-c7cccc(-c8nc(-c9ccccc9)cc(-c9ccccc9)n8)c7)ccc6c(c2)c3c45)cc1]

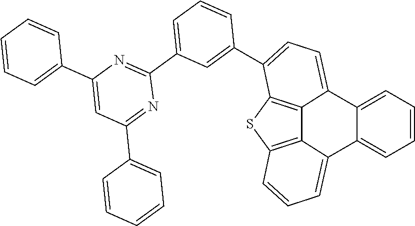 [SMILES: c1ccc(-c2cc(-c3ccccc3)nc(-c3cccc(-c4ccc5c6ccccc6c6cccc7sc4c5c76)c3)n2)cc1]

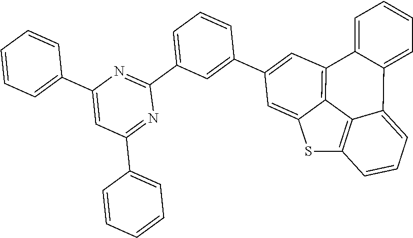 [SMILES: c1ccc(-c2cc(-c3ccccc3)nc(-c3cccc(-c4cc5sc6cccc7c8ccccc8c(c4)c5c67)c3)n2)cc1]

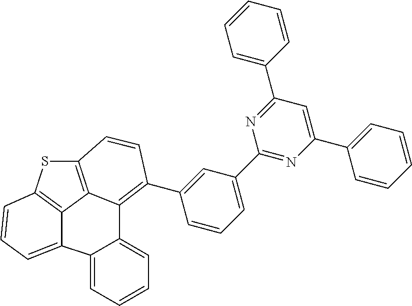 [SMILES: c1ccc(-c2cc(-c3ccccc3)nc(-c3cccc(-c4ccc5sc6cccc7c8ccccc8c4c5c67)c3)n2)cc1]

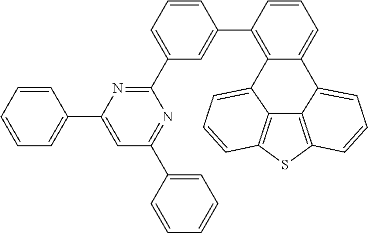 [SMILES: c1ccc(-c2cc(-c3ccccc3)nc(-c3cccc(-c4cccc5c6cccc7sc8cccc(c45)c8c76)c3)n2)cc1]

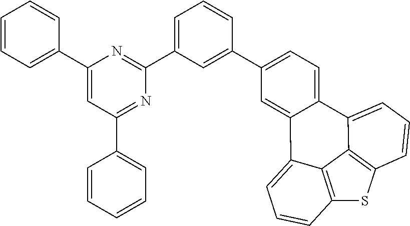 [SMILES: c1ccc(-c2cc(-c3ccccc3)nc(-c3cccc(-c4ccc5c(c4)c4cccc6sc7cccc5c7c64)c3)n2)cc1]

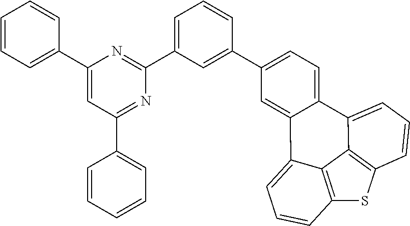 [SMILES: c1ccc(-c2cc(-c3ccccc3)nc(-c3cccc(-c4ccc5c(c4)c4cccc6sc7cccc5c7c64)c3)n2)cc1]

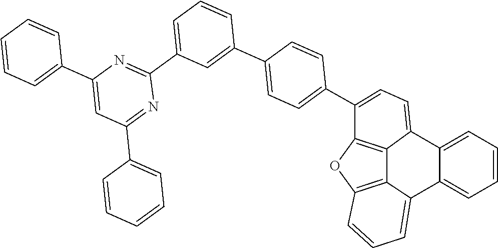 [SMILES: c1ccc(-c2cc(-c3ccccc3)nc(-c3cccc(-c4ccc(-c5ccc6c7ccccc7c7cccc8oc5c6c87)cc4)c3)n2)cc1]

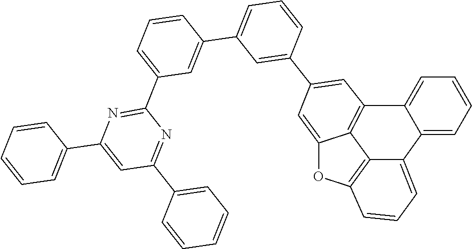 [SMILES: c1ccc(-c2cc(-c3ccccc3)nc(-c3cccc(-c4cccc(-c5cc6oc7cccc8c9ccccc9c(c5)c6c78)c4)c3)n2)cc1]

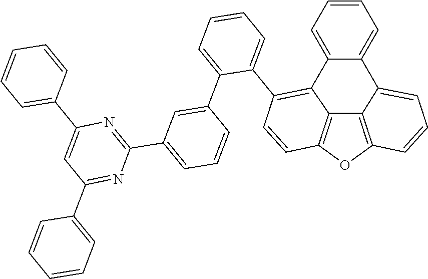 [SMILES: c1ccc(-c2cc(-c3ccccc3)nc(-c3cccc(-c4ccccc4-c4ccc5oc6cccc7c8ccccc8c4c5c67)c3)n2)cc1]

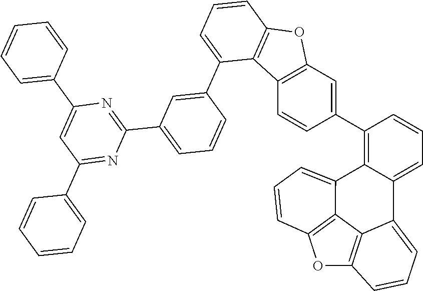 [SMILES: c1ccc(-c2cc(-c3ccccc3)nc(-c3cccc(-c4cccc5oc6cc(-c7cccc8c9cccc%10oc%11cccc(c78)c%11c%109)ccc6c45)c3)n2)cc1]

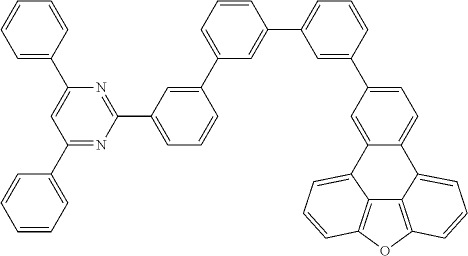 [SMILES: c1ccc(-c2cc(-c3ccccc3)nc(-c3cccc(-c4cccc(-c5cccc(-c6ccc7c(c6)c6cccc8oc9cccc7c9c86)c5)c4)c3)n2)cc1]

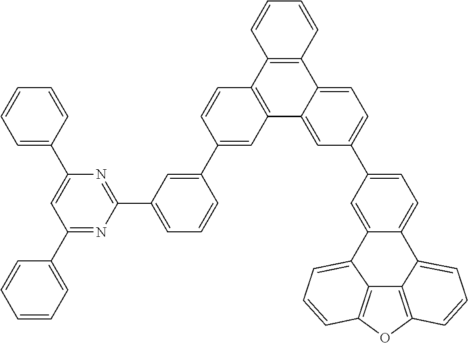 [SMILES: c1ccc(-c2cc(-c3ccccc3)nc(-c3cccc(-c4ccc5c6ccccc6c6ccc(-c7ccc8c(c7)c7cccc9oc%10cccc8c%10c97)cc6c5c4)c3)n2)cc1]

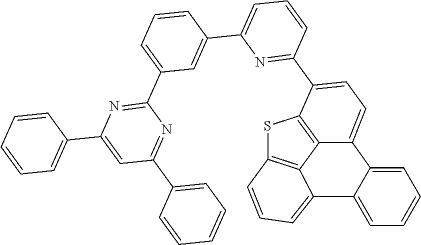 [SMILES: c1ccc(-c2cc(-c3ccccc3)nc(-c3cccc(-c4cccc(-c5ccc6c7ccccc7c7cccc8sc5c6c87)n4)c3)n2)cc1]

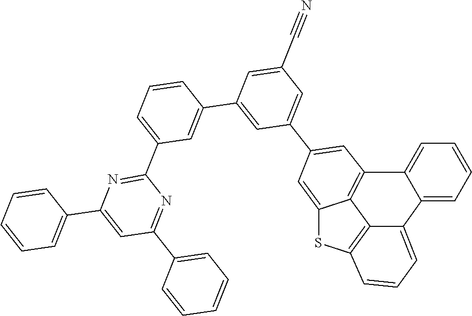 [SMILES: N#Cc1cc(-c2cccc(-c3nc(-c4ccccc4)cc(-c4ccccc4)n3)c2)cc(-c2cc3sc4cccc5c6ccccc6c(c2)c3c45)c1]

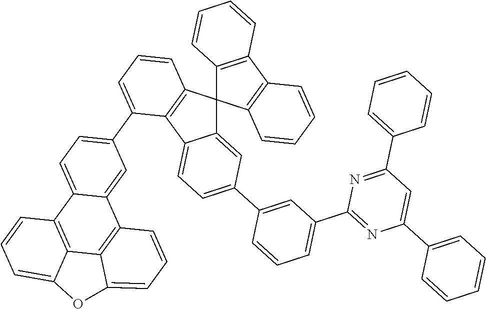 [SMILES: c1ccc(-c2cc(-c3ccccc3)nc(-c3cccc(-c4ccc5c(c4)C4(c6ccccc6-c6ccccc64)c4cccc(-c6ccc7c(c6)c6cccc8oc9cccc7c9c86)c4-5)c3)n2)cc1]

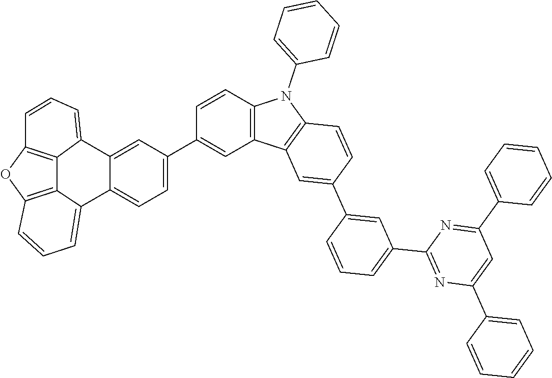 [SMILES: c1ccc(-c2cc(-c3ccccc3)nc(-c3cccc(-c4ccc5c(c4)c4cc(-c6ccc7c(c6)c6cccc8oc9cccc7c9c86)ccc4n5-c4ccccc4)c3)n2)cc1]

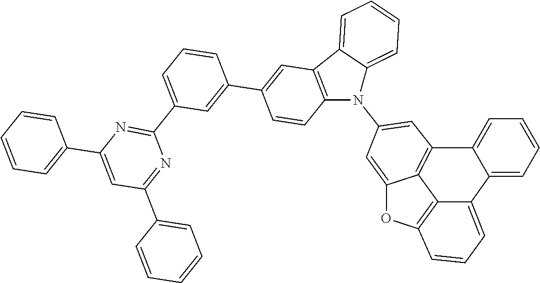 [SMILES: c1ccc(-c2cc(-c3ccccc3)nc(-c3cccc(-c4ccc5c(c4)c4ccccc4n5-c4cc5oc6cccc7c8ccccc8c(c4)c5c67)c3)n2)cc1]

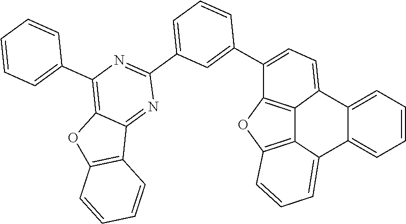 [SMILES: c1ccc(-c2nc(-c3cccc(-c4ccc5c6ccccc6c6cccc7oc4c5c76)c3)nc3c2oc2ccccc23)cc1]

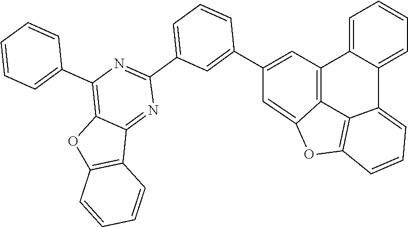 [SMILES: c1ccc(-c2nc(-c3cccc(-c4cc5oc6cccc7c8ccccc8c(c4)c5c67)c3)nc3c2oc2ccccc23)cc1]

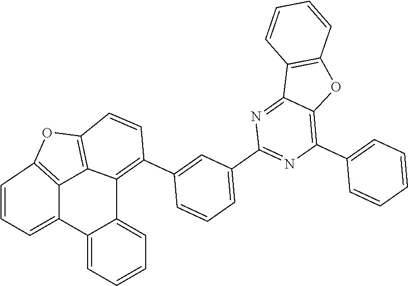 [SMILES: c1ccc(-c2nc(-c3cccc(-c4ccc5oc6cccc7c8ccccc8c4c5c67)c3)nc3c2oc2ccccc23)cc1]

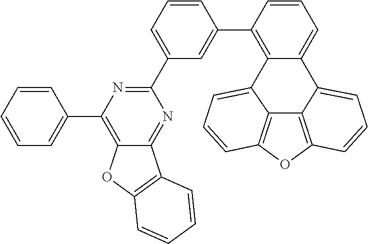 [SMILES: c1ccc(-c2nc(-c3cccc(-c4cccc5c6cccc7oc8cccc(c45)c8c76)c3)nc3c2oc2ccccc23)cc1]

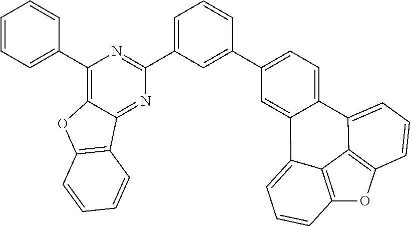 [SMILES: c1ccc(-c2nc(-c3cccc(-c4ccc5c(c4)c4cccc6oc7cccc5c7c64)c3)nc3c2oc2ccccc23)cc1]

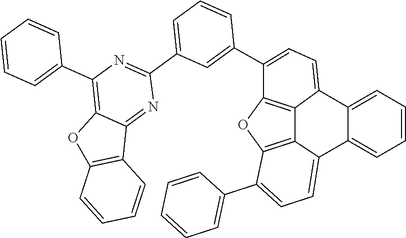 [SMILES: c1ccc(-c2nc(-c3cccc(-c4ccc5c6ccccc6c6ccc(-c7ccccc7)c7oc4c5c76)c3)nc3c2oc2ccccc23)cc1]

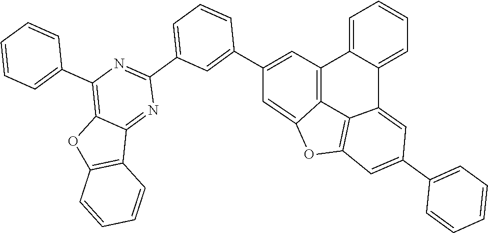 [SMILES: c1ccc(-c2cc3oc4cc(-c5cccc(-c6nc(-c7ccccc7)c7oc8ccccc8c7n6)c5)cc5c6ccccc6c(c2)c3c45)cc1]

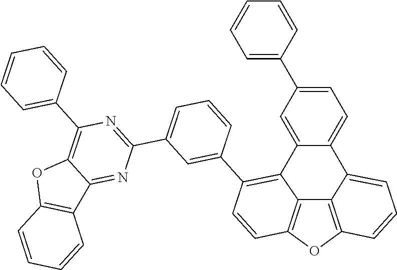 [SMILES: c1ccc(-c2ccc3c(c2)c2c(-c4cccc(-c5nc(-c6ccccc6)c6oc7ccccc7c6n5)c4)ccc4oc5cccc3c5c42)cc1]

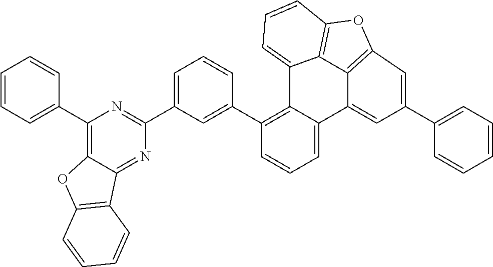 [SMILES: c1ccc(-c2cc3oc4cccc5c6c(-c7cccc(-c8nc(-c9ccccc9)c9oc%10ccccc%10c9n8)c7)cccc6c(c2)c3c45)cc1]

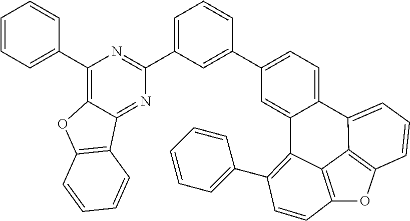 [SMILES: c1ccc(-c2nc(-c3cccc(-c4ccc5c(c4)c4c(-c6ccccc6)ccc6oc7cccc5c7c64)c3)nc3c2oc2ccccc23)cc1]

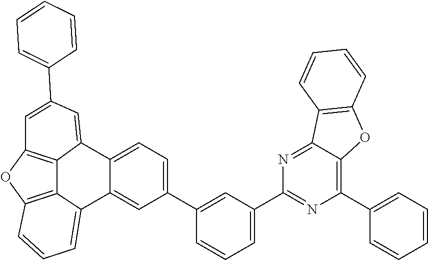 [SMILES: c1ccc(-c2cc3oc4cccc5c6cc(-c7cccc(-c8nc(-c9ccccc9)c9oc%10ccccc%10c9n8)c7)ccc6c(c2)c3c45)cc1]

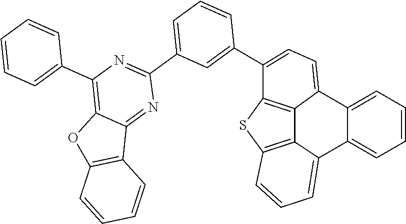 [SMILES: c1ccc(-c2nc(-c3cccc(-c4ccc5c6ccccc6c6cccc7sc4c5c76)c3)nc3c2oc2ccccc23)cc1]

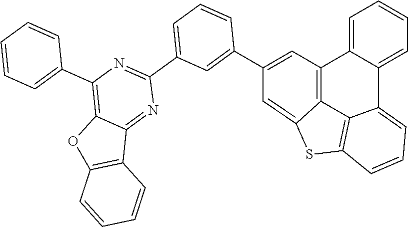 [SMILES: c1ccc(-c2nc(-c3cccc(-c4cc5sc6cccc7c8ccccc8c(c4)c5c67)c3)nc3c2oc2ccccc23)cc1]

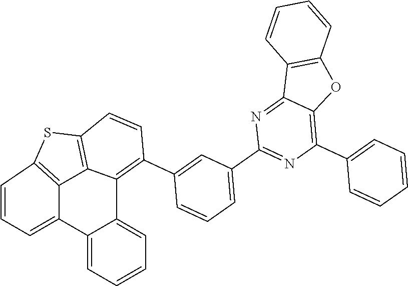 [SMILES: c1ccc(-c2nc(-c3cccc(-c4ccc5sc6cccc7c8ccccc8c4c5c67)c3)nc3c2oc2ccccc23)cc1]

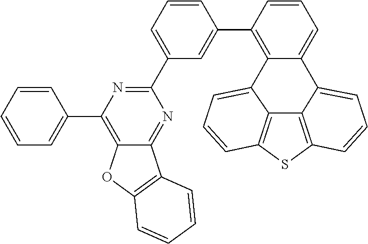 [SMILES: c1ccc(-c2nc(-c3cccc(-c4cccc5c6cccc7sc8cccc(c45)c8c76)c3)nc3c2oc2ccccc23)cc1]

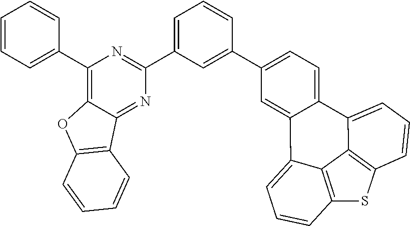 [SMILES: c1ccc(-c2nc(-c3cccc(-c4ccc5c(c4)c4cccc6sc7cccc5c7c64)c3)nc3c2oc2ccccc23)cc1]

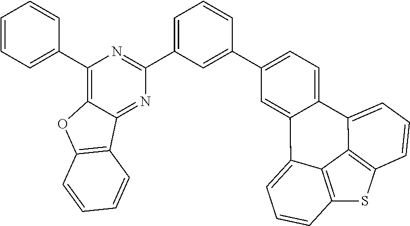 [SMILES: c1ccc(-c2nc(-c3cccc(-c4ccc5c(c4)c4cccc6sc7cccc5c7c64)c3)nc3c2oc2ccccc23)cc1]

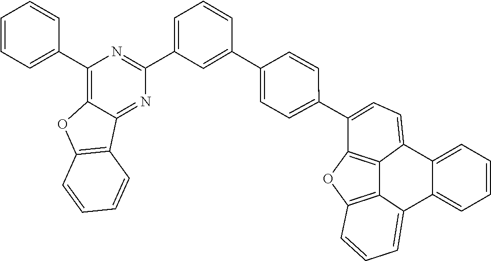 [SMILES: c1ccc(-c2nc(-c3cccc(-c4ccc(-c5ccc6c7ccccc7c7cccc8oc5c6c87)cc4)c3)nc3c2oc2ccccc23)cc1]

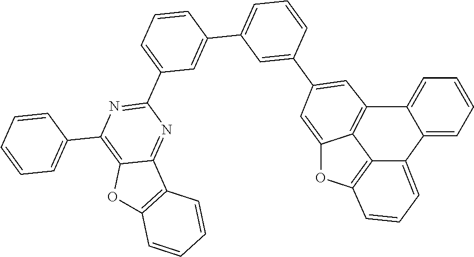 [SMILES: c1ccc(-c2nc(-c3cccc(-c4cccc(-c5cc6oc7cccc8c9ccccc9c(c5)c6c78)c4)c3)nc3c2oc2ccccc23)cc1]

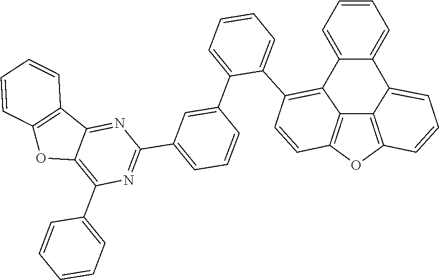 [SMILES: c1ccc(-c2nc(-c3cccc(-c4ccccc4-c4ccc5oc6cccc7c8ccccc8c4c5c67)c3)nc3c2oc2ccccc23)cc1]

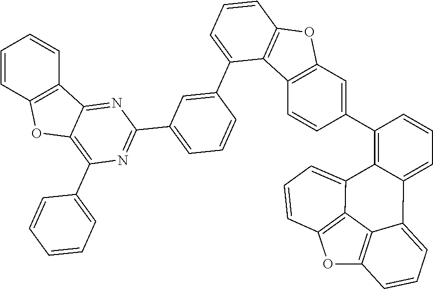 [SMILES: c1ccc(-c2nc(-c3cccc(-c4cccc5oc6cc(-c7cccc8c9cccc%10oc%11cccc(c78)c%11c%109)ccc6c45)c3)nc3c2oc2ccccc23)cc1]

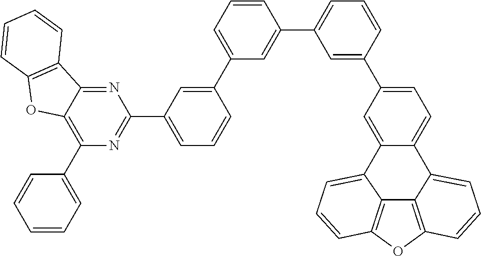 [SMILES: c1ccc(-c2nc(-c3cccc(-c4cccc(-c5cccc(-c6ccc7c(c6)c6cccc8oc9cccc7c9c86)c5)c4)c3)nc3c2oc2ccccc23)cc1]

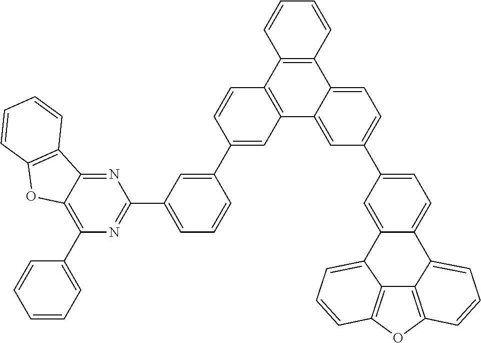 [SMILES: c1ccc(-c2nc(-c3cccc(-c4ccc5c6ccccc6c6ccc(-c7ccc8c(c7)c7cccc9oc%10cccc8c%10c97)cc6c5c4)c3)nc3c2oc2ccccc23)cc1]

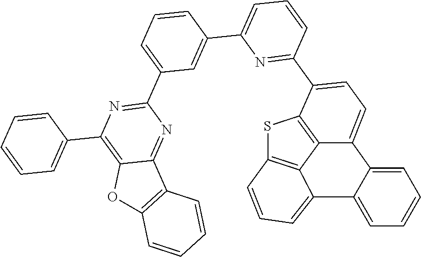 [SMILES: c1ccc(-c2nc(-c3cccc(-c4cccc(-c5ccc6c7ccccc7c7cccc8sc5c6c87)n4)c3)nc3c2oc2ccccc23)cc1]

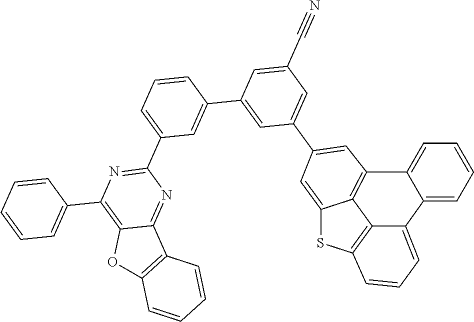 [SMILES: N#Cc1cc(-c2cccc(-c3nc(-c4ccccc4)c4oc5ccccc5c4n3)c2)cc(-c2cc3sc4cccc5c6ccccc6c(c2)c3c45)c1]

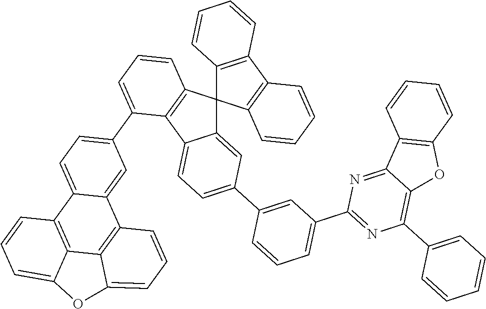 [SMILES: c1ccc(-c2nc(-c3cccc(-c4ccc5c(c4)C4(c6ccccc6-c6ccccc64)c4cccc(-c6ccc7c(c6)c6cccc8oc9cccc7c9c86)c4-5)c3)nc3c2oc2ccccc23)cc1]

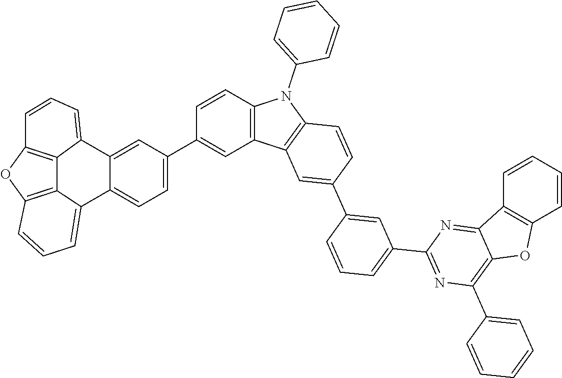 [SMILES: c1ccc(-c2nc(-c3cccc(-c4ccc5c(c4)c4cc(-c6ccc7c(c6)c6cccc8oc9cccc7c9c86)ccc4n5-c4ccccc4)c3)nc3c2oc2ccccc23)cc1]

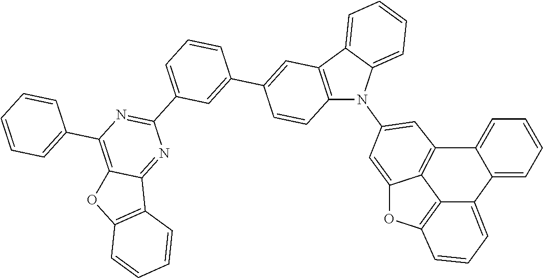 [SMILES: c1ccc(-c2nc(-c3cccc(-c4ccc5c(c4)c4ccccc4n5-c4cc5oc6cccc7c8ccccc8c(c4)c5c67)c3)nc3c2oc2ccccc23)cc1]

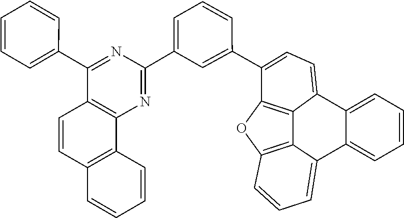 [SMILES: c1ccc(-c2nc(-c3cccc(-c4ccc5c6ccccc6c6cccc7oc4c5c76)c3)nc3c2ccc2ccccc23)cc1]

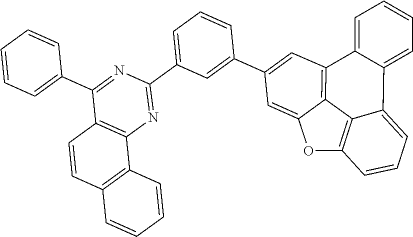 [SMILES: c1ccc(-c2nc(-c3cccc(-c4cc5oc6cccc7c8ccccc8c(c4)c5c67)c3)nc3c2ccc2ccccc23)cc1]

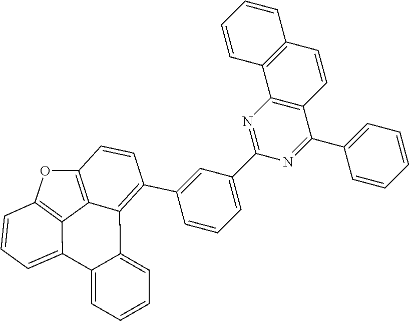 [SMILES: c1ccc(-c2nc(-c3cccc(-c4ccc5oc6cccc7c8ccccc8c4c5c67)c3)nc3c2ccc2ccccc23)cc1]

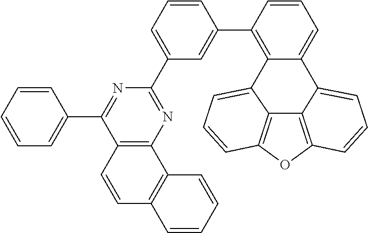 [SMILES: c1ccc(-c2nc(-c3cccc(-c4cccc5c6cccc7oc8cccc(c45)c8c76)c3)nc3c2ccc2ccccc23)cc1]

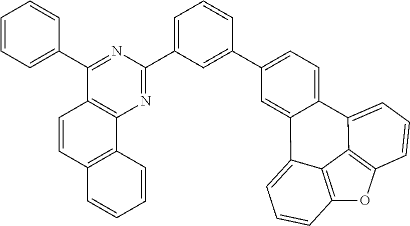 [SMILES: c1ccc(-c2nc(-c3cccc(-c4ccc5c(c4)c4cccc6oc7cccc5c7c64)c3)nc3c2ccc2ccccc23)cc1]

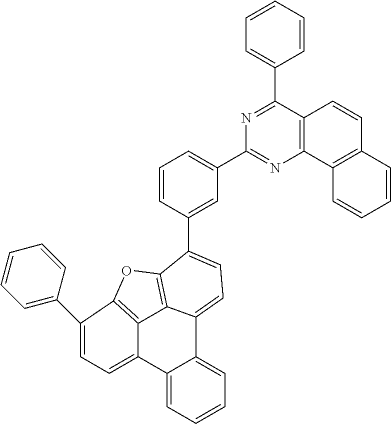 [SMILES: c1ccc(-c2nc(-c3cccc(-c4ccc5c6ccccc6c6ccc(-c7ccccc7)c7oc4c5c76)c3)nc3c2ccc2ccccc23)cc1]

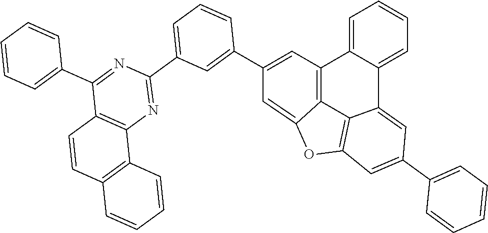 [SMILES: c1ccc(-c2cc3oc4cc(-c5cccc(-c6nc(-c7ccccc7)c7ccc8ccccc8c7n6)c5)cc5c6ccccc6c(c2)c3c45)cc1]

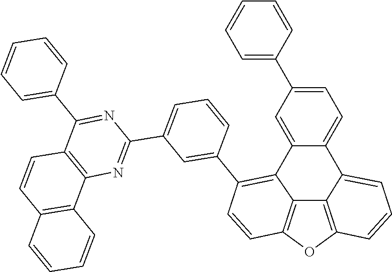 [SMILES: c1ccc(-c2ccc3c(c2)c2c(-c4cccc(-c5nc(-c6ccccc6)c6ccc7ccccc7c6n5)c4)ccc4oc5cccc3c5c42)cc1]

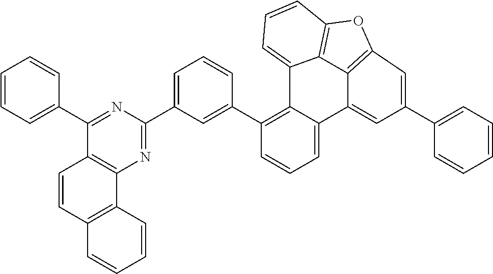 [SMILES: c1ccc(-c2cc3oc4cccc5c6c(-c7cccc(-c8nc(-c9ccccc9)c9ccc%10ccccc%10c9n8)c7)cccc6c(c2)c3c45)cc1]

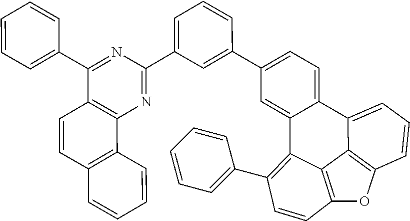 [SMILES: c1ccc(-c2nc(-c3cccc(-c4ccc5c(c4)c4c(-c6ccccc6)ccc6oc7cccc5c7c64)c3)nc3c2ccc2ccccc23)cc1]

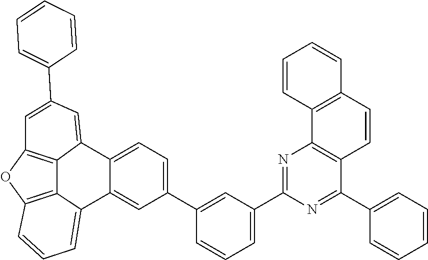 [SMILES: c1ccc(-c2cc3oc4cccc5c6cc(-c7cccc(-c8nc(-c9ccccc9)c9ccc%10ccccc%10c9n8)c7)ccc6c(c2)c3c45)cc1]

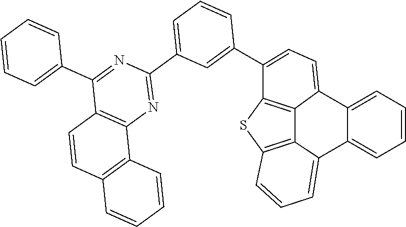 [SMILES: c1ccc(-c2nc(-c3cccc(-c4ccc5c6ccccc6c6cccc7sc4c5c76)c3)nc3c2ccc2ccccc23)cc1]

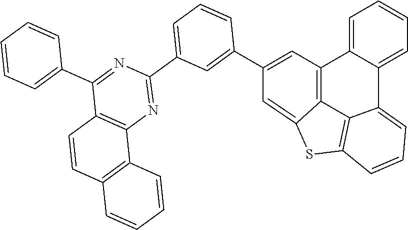 [SMILES: c1ccc(-c2nc(-c3cccc(-c4cc5sc6cccc7c8ccccc8c(c4)c5c67)c3)nc3c2ccc2ccccc23)cc1]

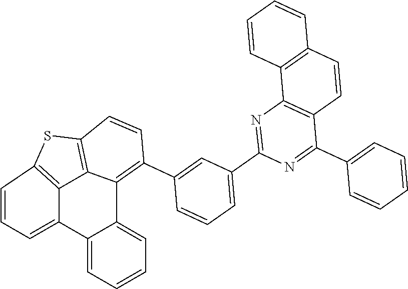 [SMILES: c1ccc(-c2nc(-c3cccc(-c4ccc5sc6cccc7c8ccccc8c4c5c67)c3)nc3c2ccc2ccccc23)cc1]

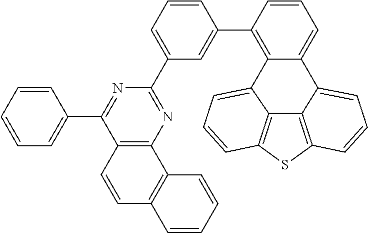 [SMILES: c1ccc(-c2nc(-c3cccc(-c4cccc5c6cccc7sc8cccc(c45)c8c76)c3)nc3c2ccc2ccccc23)cc1]

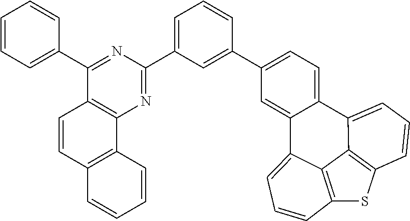 [SMILES: c1ccc(-c2nc(-c3cccc(-c4ccc5c(c4)c4cccc6sc7cccc5c7c64)c3)nc3c2ccc2ccccc23)cc1]

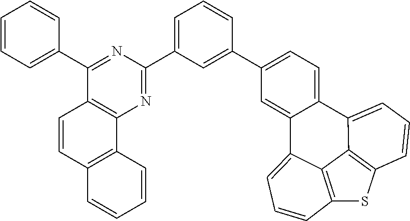 [SMILES: c1ccc(-c2nc(-c3cccc(-c4ccc5c(c4)c4cccc6sc7cccc5c7c64)c3)nc3c2ccc2ccccc23)cc1]

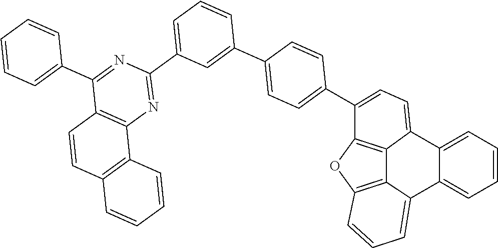 [SMILES: c1ccc(-c2nc(-c3cccc(-c4ccc(-c5ccc6c7ccccc7c7cccc8oc5c6c87)cc4)c3)nc3c2ccc2ccccc23)cc1]

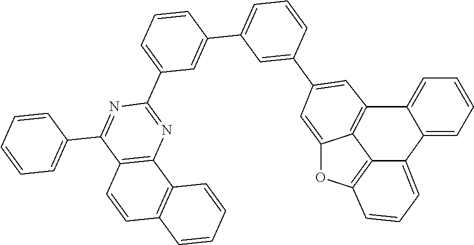 [SMILES: c1ccc(-c2nc(-c3cccc(-c4cccc(-c5cc6oc7cccc8c9ccccc9c(c5)c6c78)c4)c3)nc3c2ccc2ccccc23)cc1]

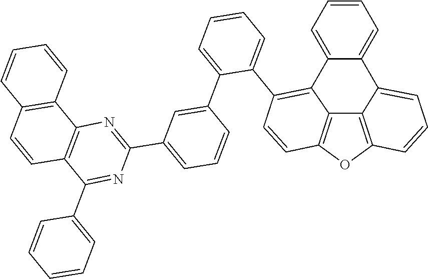 [SMILES: c1ccc(-c2nc(-c3cccc(-c4ccccc4-c4ccc5oc6cccc7c8ccccc8c4c5c67)c3)nc3c2ccc2ccccc23)cc1]

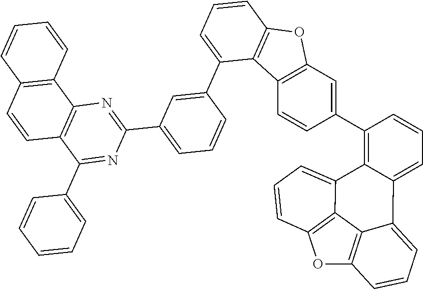 [SMILES: c1ccc(-c2nc(-c3cccc(-c4cccc5oc6cc(-c7cccc8c9cccc%10oc%11cccc(c78)c%11c%109)ccc6c45)c3)nc3c2ccc2ccccc23)cc1]

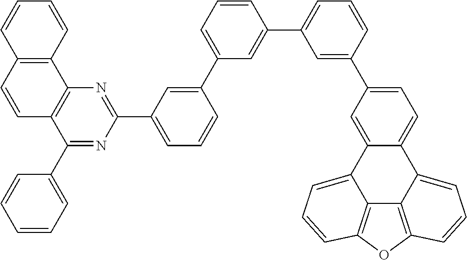 [SMILES: c1ccc(-c2nc(-c3cccc(-c4cccc(-c5cccc(-c6ccc7c(c6)c6cccc8oc9cccc7c9c86)c5)c4)c3)nc3c2ccc2ccccc23)cc1]

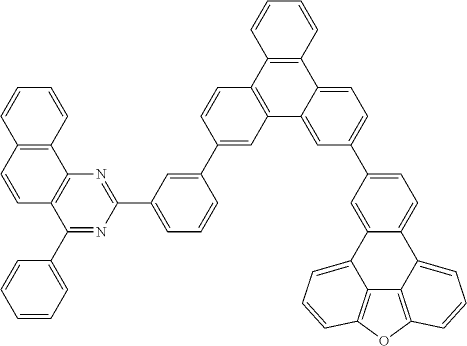 [SMILES: c1ccc(-c2nc(-c3cccc(-c4ccc5c6ccccc6c6ccc(-c7ccc8c(c7)c7cccc9oc%10cccc8c%10c97)cc6c5c4)c3)nc3c2ccc2ccccc23)cc1]

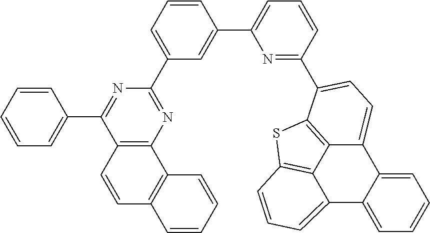 [SMILES: c1ccc(-c2nc(-c3cccc(-c4cccc(-c5ccc6c7ccccc7c7cccc8sc5c6c87)n4)c3)nc3c2ccc2ccccc23)cc1]

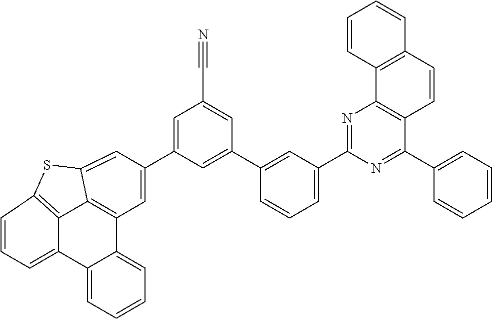 [SMILES: N#Cc1cc(-c2cccc(-c3nc(-c4ccccc4)c4ccc5ccccc5c4n3)c2)cc(-c2cc3sc4cccc5c6ccccc6c(c2)c3c45)c1]

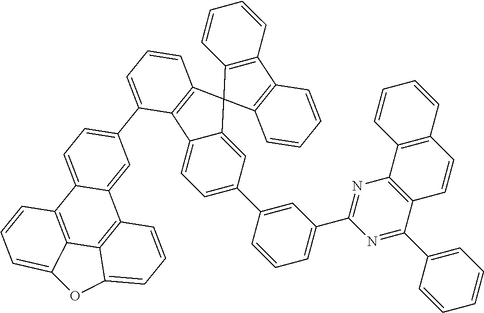 [SMILES: c1ccc(-c2nc(-c3cccc(-c4ccc5c(c4)C4(c6ccccc6-c6ccccc64)c4cccc(-c6ccc7c(c6)c6cccc8oc9cccc7c9c86)c4-5)c3)nc3c2ccc2ccccc23)cc1]

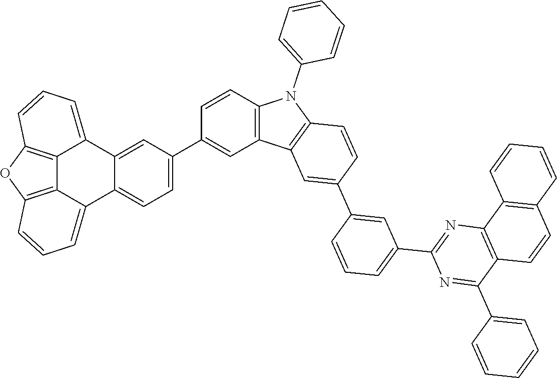 [SMILES: c1ccc(-c2nc(-c3cccc(-c4ccc5c(c4)c4cc(-c6ccc7c(c6)c6cccc8oc9cccc7c9c86)ccc4n5-c4ccccc4)c3)nc3c2ccc2ccccc23)cc1]

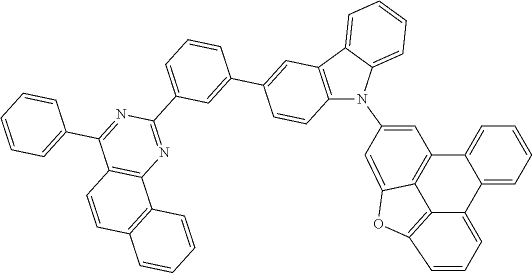 [SMILES: c1ccc(-c2nc(-c3cccc(-c4ccc5c(c4)c4ccccc4n5-c4cc5oc6cccc7c8ccccc8c(c4)c5c67)c3)nc3c2ccc2ccccc23)cc1]

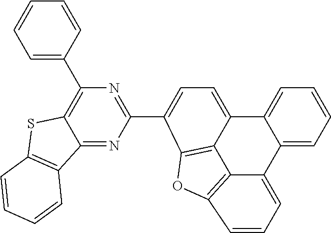 [SMILES: c1ccc(-c2nc(-c3ccc4c5ccccc5c5cccc6oc3c4c65)nc3c2sc2ccccc23)cc1]

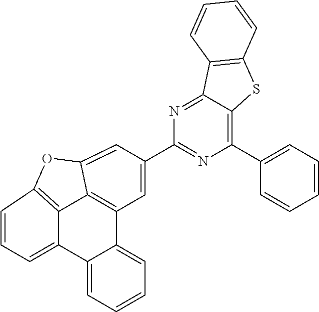 [SMILES: c1ccc(-c2nc(-c3cc4oc5cccc6c7ccccc7c(c3)c4c56)nc3c2sc2ccccc23)cc1]

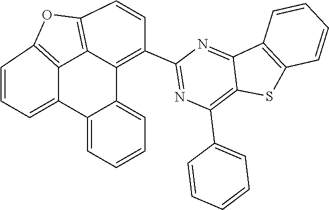 [SMILES: c1ccc(-c2nc(-c3ccc4oc5cccc6c7ccccc7c3c4c56)nc3c2sc2ccccc23)cc1]

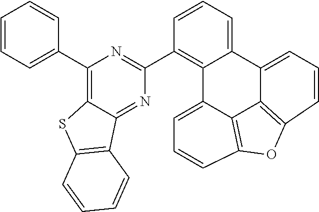 [SMILES: c1ccc(-c2nc(-c3cccc4c5cccc6oc7cccc(c34)c7c65)nc3c2sc2ccccc23)cc1]

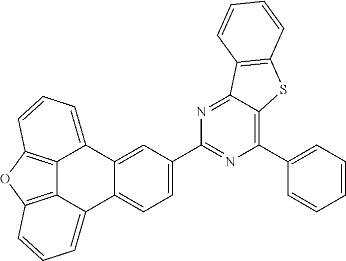 [SMILES: c1ccc(-c2nc(-c3ccc4c(c3)c3cccc5oc6cccc4c6c53)nc3c2sc2ccccc23)cc1]

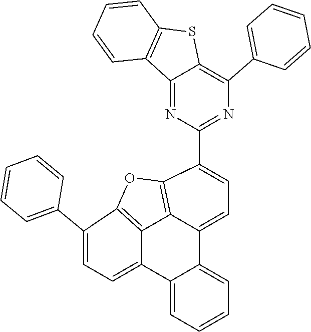 [SMILES: c1ccc(-c2ccc3c4ccccc4c4ccc(-c5nc(-c6ccccc6)c6sc7ccccc7c6n5)c5oc2c3c54)cc1]

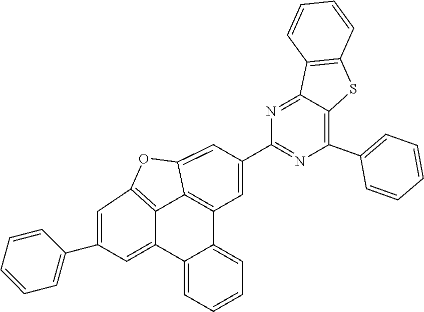 [SMILES: c1ccc(-c2cc3oc4cc(-c5nc(-c6ccccc6)c6sc7ccccc7c6n5)cc5c6ccccc6c(c2)c3c45)cc1]

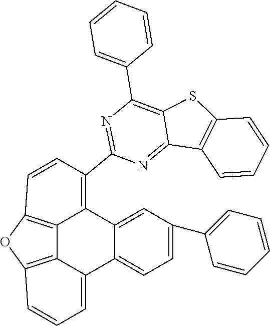 [SMILES: c1ccc(-c2ccc3c(c2)c2c(-c4nc(-c5ccccc5)c5sc6ccccc6c5n4)ccc4oc5cccc3c5c42)cc1]

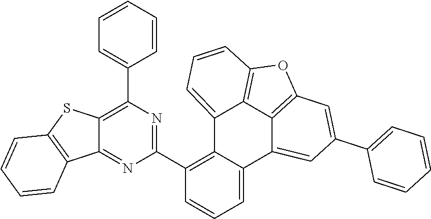 [SMILES: c1ccc(-c2cc3oc4cccc5c6c(-c7nc(-c8ccccc8)c8sc9ccccc9c8n7)cccc6c(c2)c3c45)cc1]

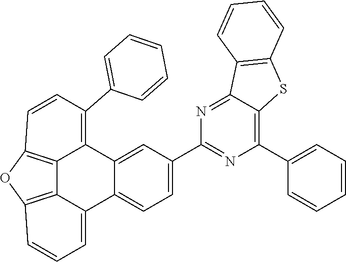 [SMILES: c1ccc(-c2nc(-c3ccc4c(c3)c3c(-c5ccccc5)ccc5oc6cccc4c6c53)nc3c2sc2ccccc23)cc1]

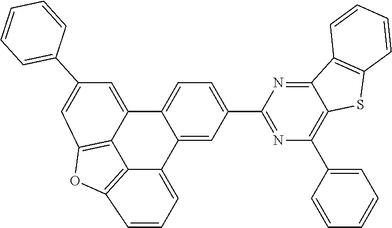 [SMILES: c1ccc(-c2cc3oc4cccc5c6cc(-c7nc(-c8ccccc8)c8sc9ccccc9c8n7)ccc6c(c2)c3c45)cc1]

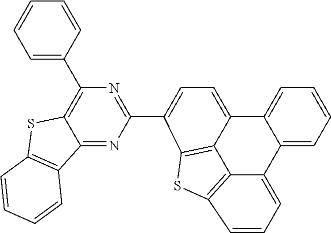 [SMILES: c1ccc(-c2nc(-c3ccc4c5ccccc5c5cccc6sc3c4c65)nc3c2sc2ccccc23)cc1]

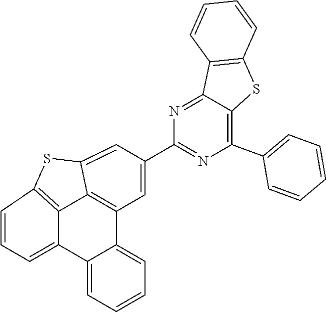 [SMILES: c1ccc(-c2nc(-c3cc4sc5cccc6c7ccccc7c(c3)c4c56)nc3c2sc2ccccc23)cc1]

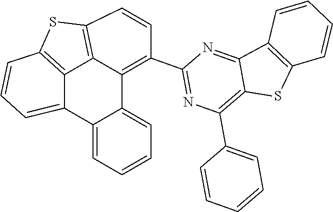 [SMILES: c1ccc(-c2nc(-c3ccc4sc5cccc6c7ccccc7c3c4c56)nc3c2sc2ccccc23)cc1]

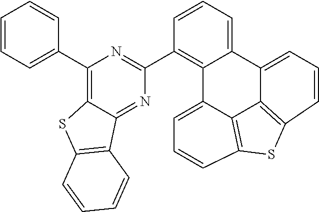 [SMILES: c1ccc(-c2nc(-c3cccc4c5cccc6sc7cccc(c34)c7c65)nc3c2sc2ccccc23)cc1]

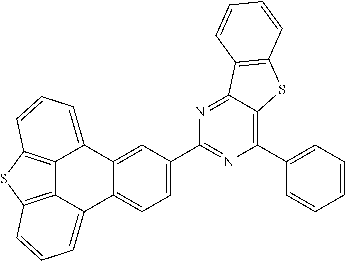 [SMILES: c1ccc(-c2nc(-c3ccc4c(c3)c3cccc5sc6cccc4c6c53)nc3c2sc2ccccc23)cc1]

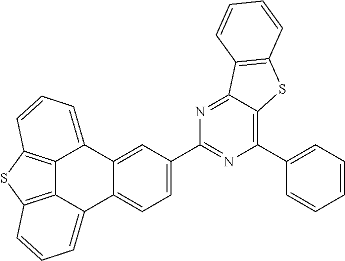 [SMILES: c1ccc(-c2nc(-c3ccc4c(c3)c3cccc5sc6cccc4c6c53)nc3c2sc2ccccc23)cc1]

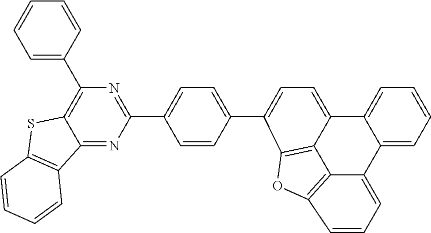 [SMILES: c1ccc(-c2nc(-c3ccc(-c4ccc5c6ccccc6c6cccc7oc4c5c76)cc3)nc3c2sc2ccccc23)cc1]

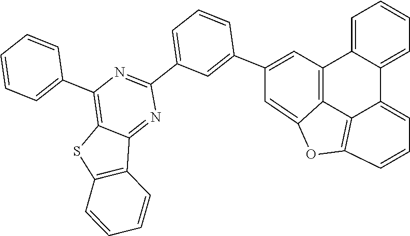 [SMILES: c1ccc(-c2nc(-c3cccc(-c4cc5oc6cccc7c8ccccc8c(c4)c5c67)c3)nc3c2sc2ccccc23)cc1]

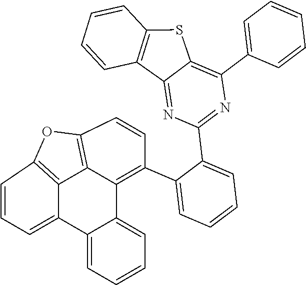 [SMILES: c1ccc(-c2nc(-c3ccccc3-c3ccc4oc5cccc6c7ccccc7c3c4c56)nc3c2sc2ccccc23)cc1]

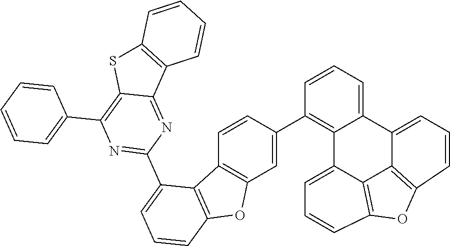 [SMILES: c1ccc(-c2nc(-c3cccc4oc5cc(-c6cccc7c8cccc9oc%10cccc(c67)c%10c98)ccc5c34)nc3c2sc2ccccc23)cc1]

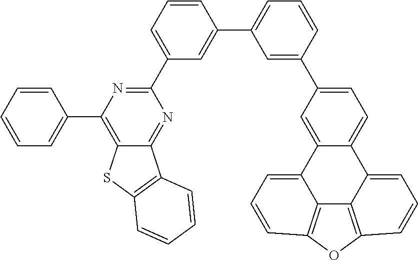 [SMILES: c1ccc(-c2nc(-c3cccc(-c4cccc(-c5ccc6c(c5)c5cccc7oc8cccc6c8c75)c4)c3)nc3c2sc2ccccc23)cc1]

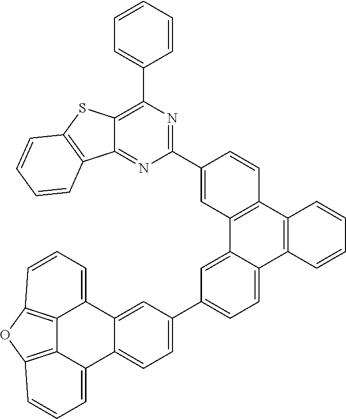 [SMILES: c1ccc(-c2nc(-c3ccc4c5ccccc5c5ccc(-c6ccc7c(c6)c6cccc8oc9cccc7c9c86)cc5c4c3)nc3c2sc2ccccc23)cc1]

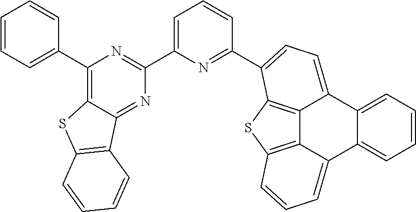 [SMILES: c1ccc(-c2nc(-c3cccc(-c4ccc5c6ccccc6c6cccc7sc4c5c76)n3)nc3c2sc2ccccc23)cc1]

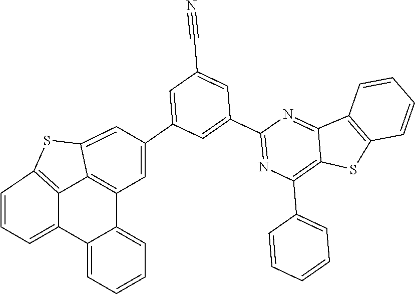 [SMILES: N#Cc1cc(-c2cc3sc4cccc5c6ccccc6c(c2)c3c45)cc(-c2nc(-c3ccccc3)c3sc4ccccc4c3n2)c1]

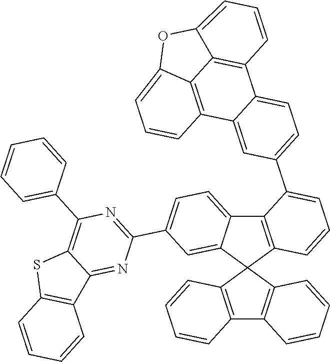 [SMILES: c1ccc(-c2nc(-c3ccc4c(c3)C3(c5ccccc5-c5ccccc53)c3cccc(-c5ccc6c(c5)c5cccc7oc8cccc6c8c75)c3-4)nc3c2sc2ccccc23)cc1]

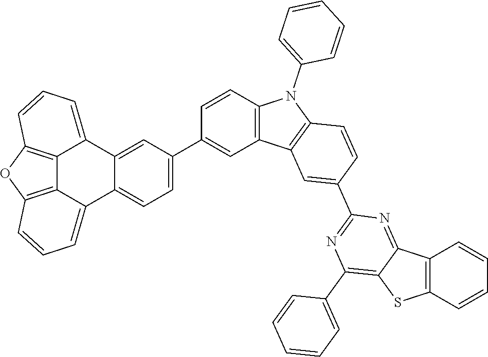 [SMILES: c1ccc(-c2nc(-c3ccc4c(c3)c3cc(-c5ccc6c(c5)c5cccc7oc8cccc6c8c75)ccc3n4-c3ccccc3)nc3c2sc2ccccc23)cc1]

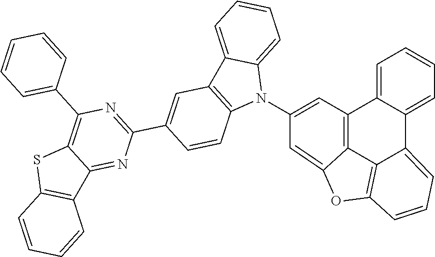 [SMILES: c1ccc(-c2nc(-c3ccc4c(c3)c3ccccc3n4-c3cc4oc5cccc6c7ccccc7c(c3)c4c56)nc3c2sc2ccccc23)cc1]

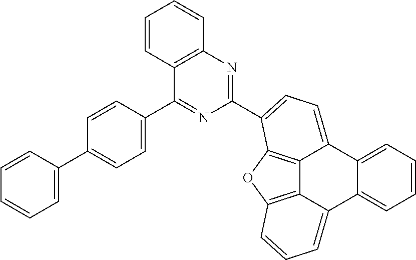 [SMILES: c1ccc(-c2ccc(-c3nc(-c4ccc5c6ccccc6c6cccc7oc4c5c76)nc4ccccc34)cc2)cc1]

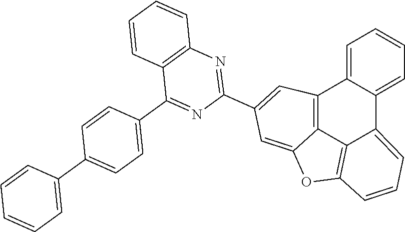 [SMILES: c1ccc(-c2ccc(-c3nc(-c4cc5oc6cccc7c8ccccc8c(c4)c5c67)nc4ccccc34)cc2)cc1]

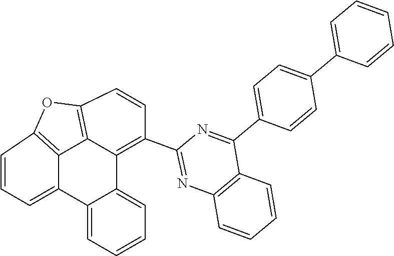 [SMILES: c1ccc(-c2ccc(-c3nc(-c4ccc5oc6cccc7c8ccccc8c4c5c67)nc4ccccc34)cc2)cc1]

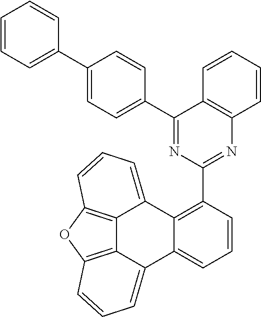 [SMILES: c1ccc(-c2ccc(-c3nc(-c4cccc5c6cccc7oc8cccc(c45)c8c76)nc4ccccc34)cc2)cc1]

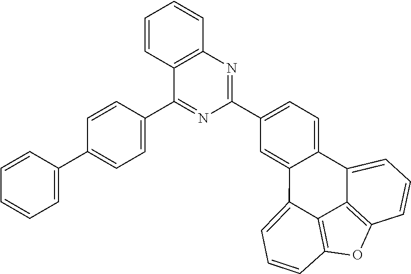 [SMILES: c1ccc(-c2ccc(-c3nc(-c4ccc5c(c4)c4cccc6oc7cccc5c7c64)nc4ccccc34)cc2)cc1]

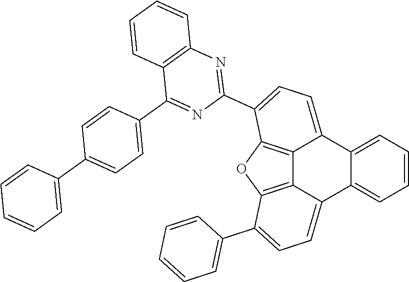 [SMILES: c1ccc(-c2ccc(-c3nc(-c4ccc5c6ccccc6c6ccc(-c7ccccc7)c7oc4c5c76)nc4ccccc34)cc2)cc1]

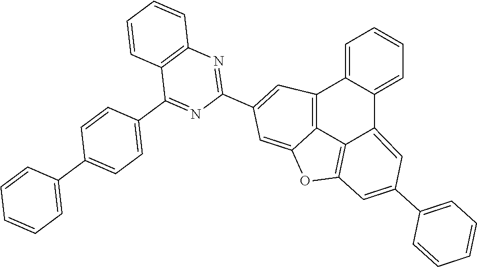 [SMILES: c1ccc(-c2ccc(-c3nc(-c4cc5oc6cc(-c7ccccc7)cc7c8ccccc8c(c4)c5c67)nc4ccccc34)cc2)cc1]

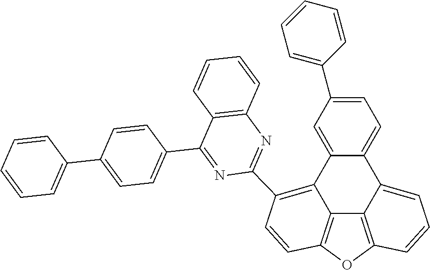 [SMILES: c1ccc(-c2ccc(-c3nc(-c4ccc5oc6cccc7c8ccc(-c9ccccc9)cc8c4c5c67)nc4ccccc34)cc2)cc1]

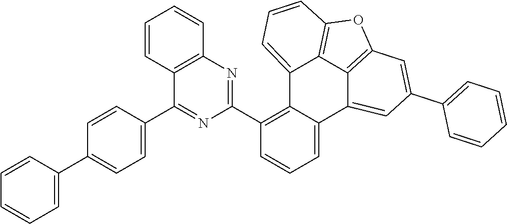 [SMILES: c1ccc(-c2ccc(-c3nc(-c4cccc5c6cc(-c7ccccc7)cc7oc8cccc(c45)c8c76)nc4ccccc34)cc2)cc1]

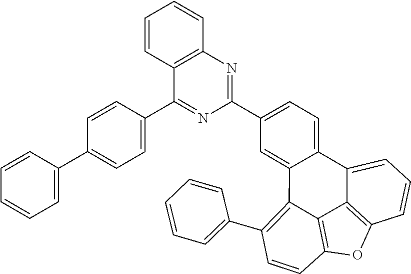 [SMILES: c1ccc(-c2ccc(-c3nc(-c4ccc5c(c4)c4c(-c6ccccc6)ccc6oc7cccc5c7c64)nc4ccccc34)cc2)cc1]

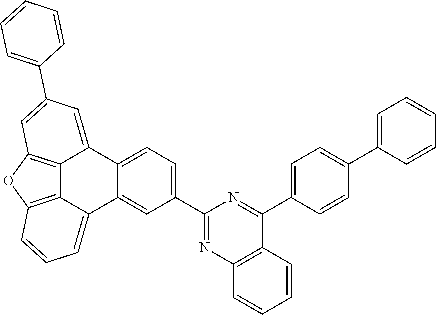 [SMILES: c1ccc(-c2ccc(-c3nc(-c4ccc5c(c4)c4cccc6oc7cc(-c8ccccc8)cc5c7c64)nc4ccccc34)cc2)cc1]

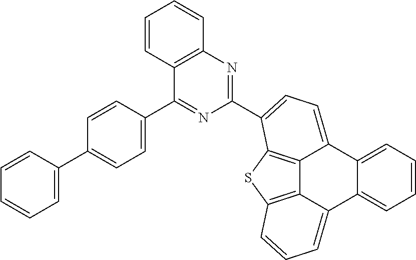 [SMILES: c1ccc(-c2ccc(-c3nc(-c4ccc5c6ccccc6c6cccc7sc4c5c76)nc4ccccc34)cc2)cc1]

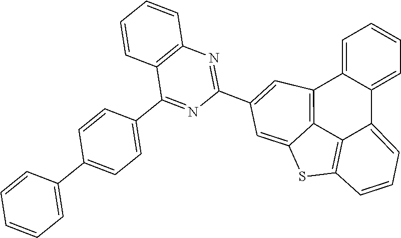 [SMILES: c1ccc(-c2ccc(-c3nc(-c4cc5sc6cccc7c8ccccc8c(c4)c5c67)nc4ccccc34)cc2)cc1]

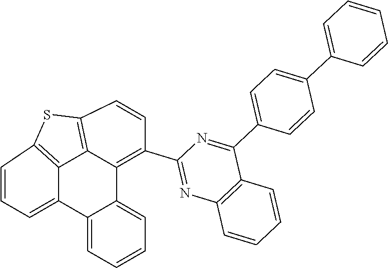 [SMILES: c1ccc(-c2ccc(-c3nc(-c4ccc5sc6cccc7c8ccccc8c4c5c67)nc4ccccc34)cc2)cc1]

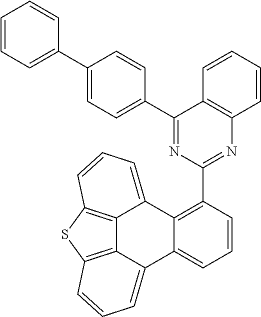 [SMILES: c1ccc(-c2ccc(-c3nc(-c4cccc5c6cccc7sc8cccc(c45)c8c76)nc4ccccc34)cc2)cc1]

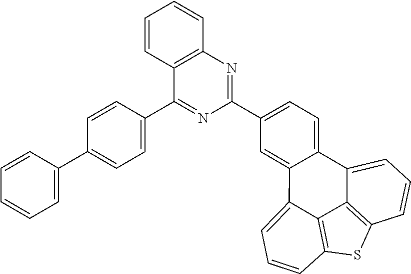 [SMILES: c1ccc(-c2ccc(-c3nc(-c4ccc5c(c4)c4cccc6sc7cccc5c7c64)nc4ccccc34)cc2)cc1]

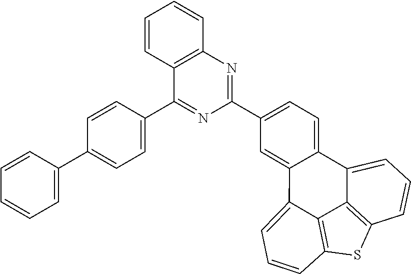 [SMILES: c1ccc(-c2ccc(-c3nc(-c4ccc5c(c4)c4cccc6sc7cccc5c7c64)nc4ccccc34)cc2)cc1]

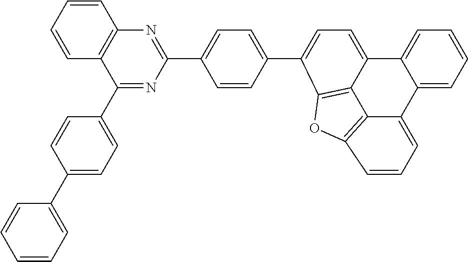 [SMILES: c1ccc(-c2ccc(-c3nc(-c4ccc(-c5ccc6c7ccccc7c7cccc8oc5c6c87)cc4)nc4ccccc34)cc2)cc1]

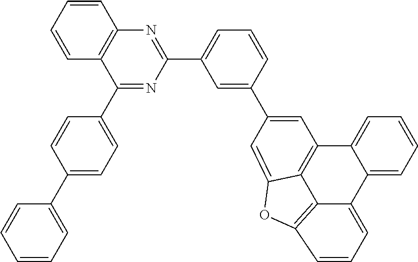 [SMILES: c1ccc(-c2ccc(-c3nc(-c4cccc(-c5cc6oc7cccc8c9ccccc9c(c5)c6c78)c4)nc4ccccc34)cc2)cc1]

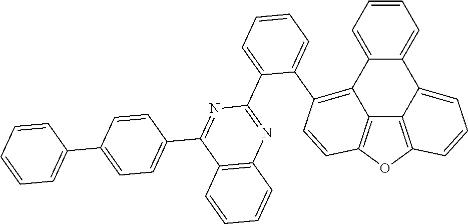 [SMILES: c1ccc(-c2ccc(-c3nc(-c4ccccc4-c4ccc5oc6cccc7c8ccccc8c4c5c67)nc4ccccc34)cc2)cc1]

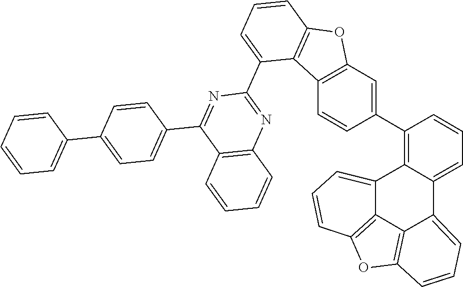 [SMILES: c1ccc(-c2ccc(-c3nc(-c4cccc5oc6cc(-c7cccc8c9cccc%10oc%11cccc(c78)c%11c%109)ccc6c45)nc4ccccc34)cc2)cc1]

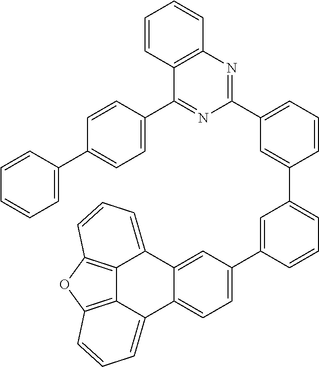 [SMILES: c1ccc(-c2ccc(-c3nc(-c4cccc(-c5cccc(-c6ccc7c(c6)c6cccc8oc9cccc7c9c86)c5)c4)nc4ccccc34)cc2)cc1]

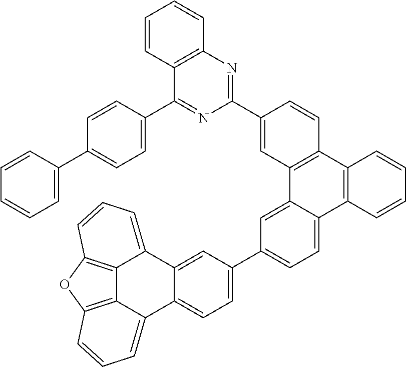 [SMILES: c1ccc(-c2ccc(-c3nc(-c4ccc5c6ccccc6c6ccc(-c7ccc8c(c7)c7cccc9oc%10cccc8c%10c97)cc6c5c4)nc4ccccc34)cc2)cc1]

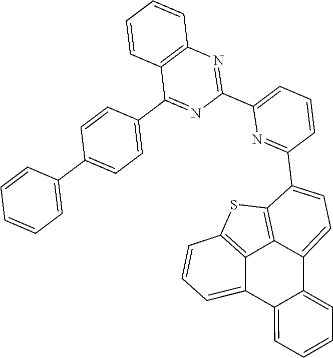 [SMILES: c1ccc(-c2ccc(-c3nc(-c4cccc(-c5ccc6c7ccccc7c7cccc8sc5c6c87)n4)nc4ccccc34)cc2)cc1]

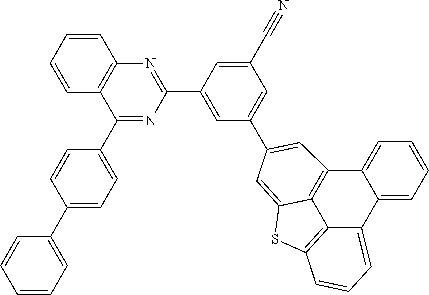 [SMILES: N#Cc1cc(-c2cc3sc4cccc5c6ccccc6c(c2)c3c45)cc(-c2nc(-c3ccc(-c4ccccc4)cc3)c3ccccc3n2)c1]

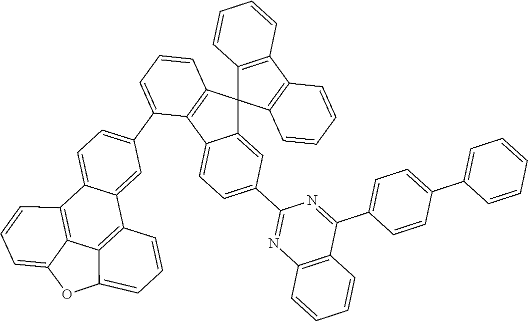 [SMILES: c1ccc(-c2ccc(-c3nc(-c4ccc5c(c4)C4(c6ccccc6-c6ccccc64)c4cccc(-c6ccc7c(c6)c6cccc8oc9cccc7c9c86)c4-5)nc4ccccc34)cc2)cc1]

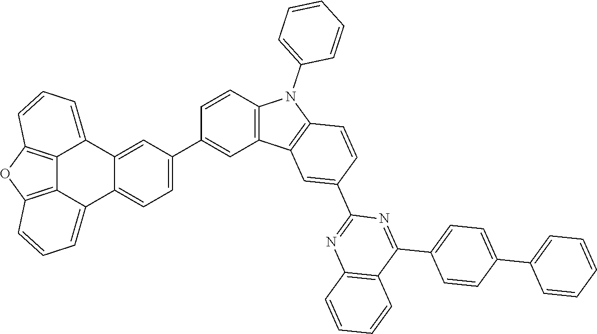 [SMILES: c1ccc(-c2ccc(-c3nc(-c4ccc5c(c4)c4cc(-c6ccc7c(c6)c6cccc8oc9cccc7c9c86)ccc4n5-c4ccccc4)nc4ccccc34)cc2)cc1]

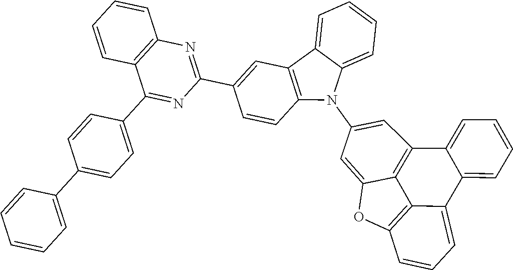 [SMILES: c1ccc(-c2ccc(-c3nc(-c4ccc5c(c4)c4ccccc4n5-c4cc5oc6cccc7c8ccccc8c(c4)c5c67)nc4ccccc34)cc2)cc1]

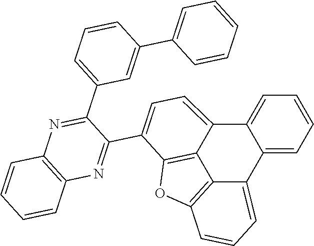 [SMILES: c1ccc(-c2cccc(-c3nc4ccccc4nc3-c3ccc4c5ccccc5c5cccc6oc3c4c65)c2)cc1]

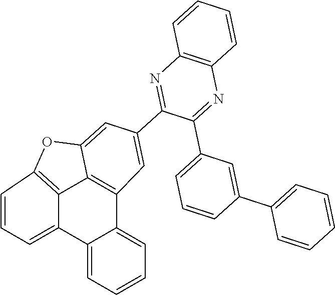 [SMILES: c1ccc(-c2cccc(-c3nc4ccccc4nc3-c3cc4oc5cccc6c7ccccc7c(c3)c4c56)c2)cc1]

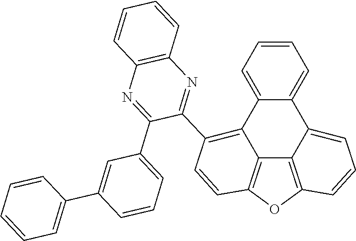 [SMILES: c1ccc(-c2cccc(-c3nc4ccccc4nc3-c3ccc4oc5cccc6c7ccccc7c3c4c56)c2)cc1]

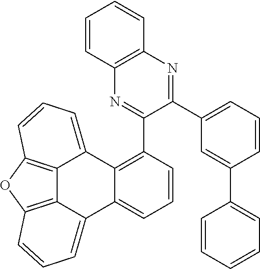 [SMILES: c1ccc(-c2cccc(-c3nc4ccccc4nc3-c3cccc4c5cccc6oc7cccc(c34)c7c65)c2)cc1]

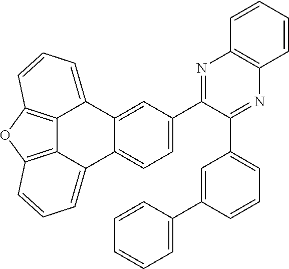 [SMILES: c1ccc(-c2cccc(-c3nc4ccccc4nc3-c3ccc4c(c3)c3cccc5oc6cccc4c6c53)c2)cc1]

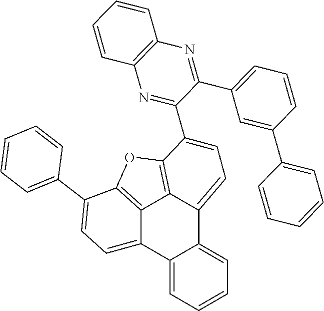 [SMILES: c1ccc(-c2cccc(-c3nc4ccccc4nc3-c3ccc4c5ccccc5c5ccc(-c6ccccc6)c6oc3c4c65)c2)cc1]

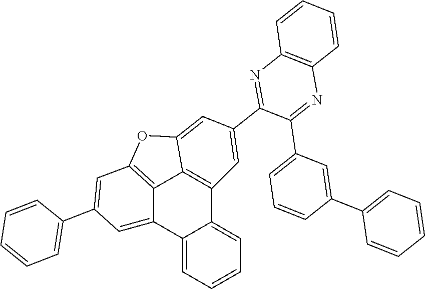 [SMILES: c1ccc(-c2cccc(-c3nc4ccccc4nc3-c3cc4oc5cc(-c6ccccc6)cc6c7ccccc7c(c3)c4c56)c2)cc1]

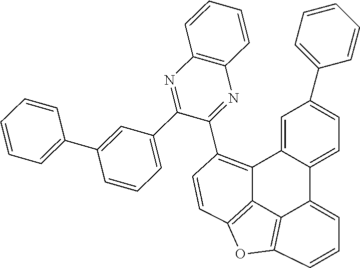 [SMILES: c1ccc(-c2cccc(-c3nc4ccccc4nc3-c3ccc4oc5cccc6c7ccc(-c8ccccc8)cc7c3c4c56)c2)cc1]

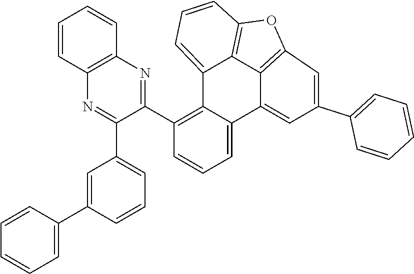 [SMILES: c1ccc(-c2cccc(-c3nc4ccccc4nc3-c3cccc4c5cc(-c6ccccc6)cc6oc7cccc(c34)c7c65)c2)cc1]

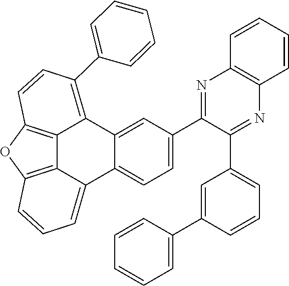 [SMILES: c1ccc(-c2cccc(-c3nc4ccccc4nc3-c3ccc4c(c3)c3c(-c5ccccc5)ccc5oc6cccc4c6c53)c2)cc1]

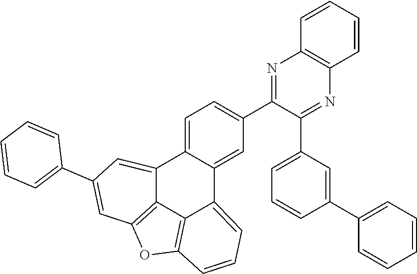 [SMILES: c1ccc(-c2cccc(-c3nc4ccccc4nc3-c3ccc4c(c3)c3cccc5oc6cc(-c7ccccc7)cc4c6c53)c2)cc1]

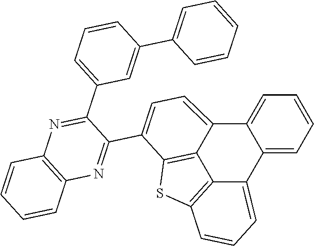 [SMILES: c1ccc(-c2cccc(-c3nc4ccccc4nc3-c3ccc4c5ccccc5c5cccc6sc3c4c65)c2)cc1]

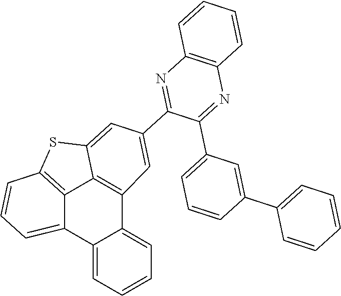 [SMILES: c1ccc(-c2cccc(-c3nc4ccccc4nc3-c3cc4sc5cccc6c7ccccc7c(c3)c4c56)c2)cc1]

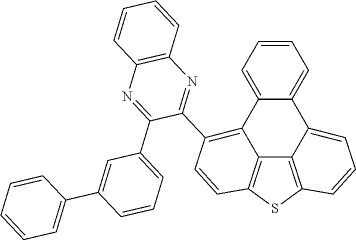 [SMILES: c1ccc(-c2cccc(-c3nc4ccccc4nc3-c3ccc4sc5cccc6c7ccccc7c3c4c56)c2)cc1]

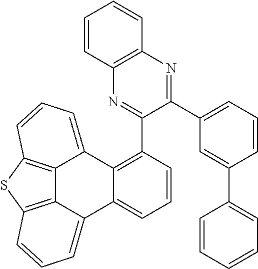 [SMILES: c1ccc(-c2cccc(-c3nc4ccccc4nc3-c3cccc4c5cccc6sc7cccc(c34)c7c65)c2)cc1]

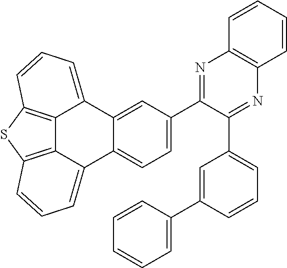 [SMILES: c1ccc(-c2cccc(-c3nc4ccccc4nc3-c3ccc4c(c3)c3cccc5sc6cccc4c6c53)c2)cc1]

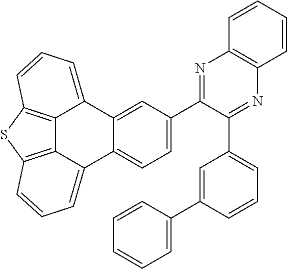 [SMILES: c1ccc(-c2cccc(-c3nc4ccccc4nc3-c3ccc4c(c3)c3cccc5sc6cccc4c6c53)c2)cc1]

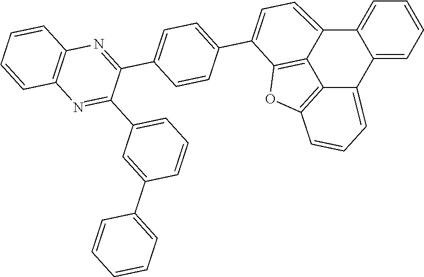 [SMILES: c1ccc(-c2cccc(-c3nc4ccccc4nc3-c3ccc(-c4ccc5c6ccccc6c6cccc7oc4c5c76)cc3)c2)cc1]

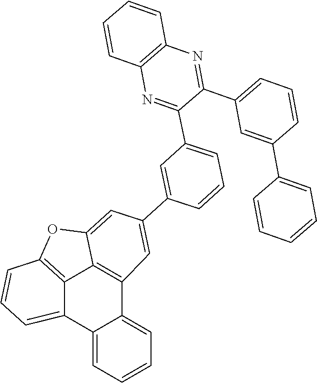 [SMILES: c1ccc(-c2cccc(-c3nc4ccccc4nc3-c3cccc(-c4cc5oc6cccc7c8ccccc8c(c4)c5c67)c3)c2)cc1]

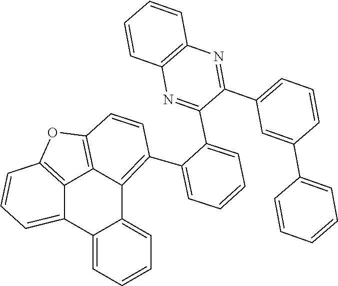 [SMILES: c1ccc(-c2cccc(-c3nc4ccccc4nc3-c3ccccc3-c3ccc4oc5cccc6c7ccccc7c3c4c56)c2)cc1]

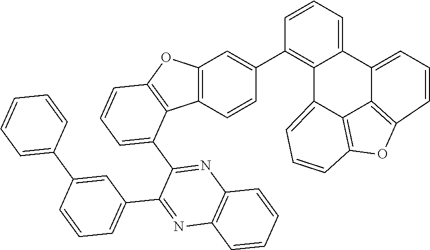 [SMILES: c1ccc(-c2cccc(-c3nc4ccccc4nc3-c3cccc4oc5cc(-c6cccc7c8cccc9oc%10cccc(c67)c%10c98)ccc5c34)c2)cc1]

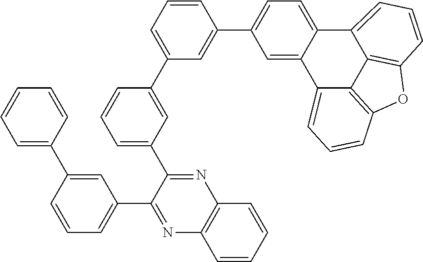 [SMILES: c1ccc(-c2cccc(-c3nc4ccccc4nc3-c3cccc(-c4cccc(-c5ccc6c(c5)c5cccc7oc8cccc6c8c75)c4)c3)c2)cc1]

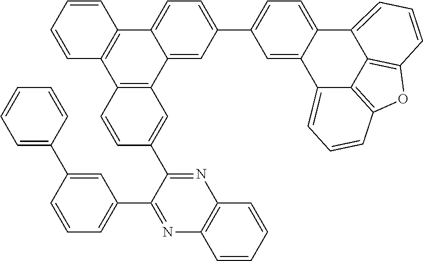 [SMILES: c1ccc(-c2cccc(-c3nc4ccccc4nc3-c3ccc4c5ccccc5c5ccc(-c6ccc7c(c6)c6cccc8oc9cccc7c9c86)cc5c4c3)c2)cc1]

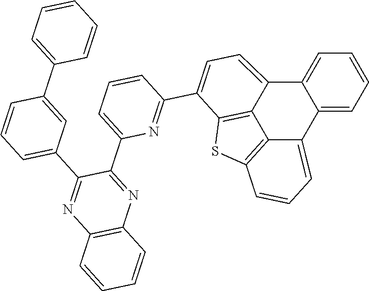 [SMILES: c1ccc(-c2cccc(-c3nc4ccccc4nc3-c3cccc(-c4ccc5c6ccccc6c6cccc7sc4c5c76)n3)c2)cc1]

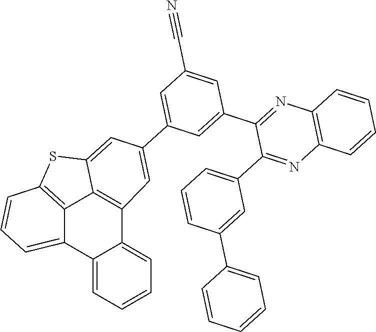 [SMILES: N#Cc1cc(-c2cc3sc4cccc5c6ccccc6c(c2)c3c45)cc(-c2nc3ccccc3nc2-c2cccc(-c3ccccc3)c2)c1]

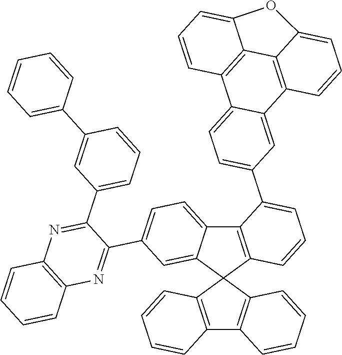 [SMILES: c1ccc(-c2cccc(-c3nc4ccccc4nc3-c3ccc4c(c3)C3(c5ccccc5-c5ccccc53)c3cccc(-c5ccc6c(c5)c5cccc7oc8cccc6c8c75)c3-4)c2)cc1]

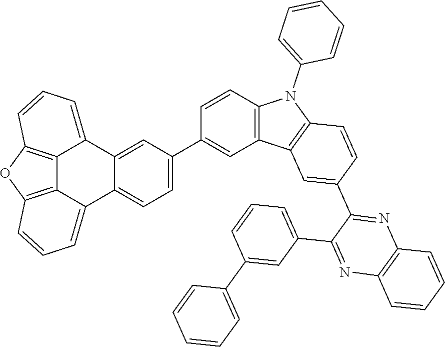 [SMILES: c1ccc(-c2cccc(-c3nc4ccccc4nc3-c3ccc4c(c3)c3cc(-c5ccc6c(c5)c5cccc7oc8cccc6c8c75)ccc3n4-c3ccccc3)c2)cc1]

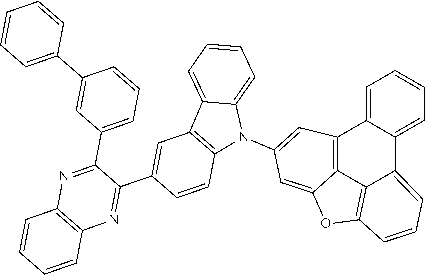 [SMILES: c1ccc(-c2cccc(-c3nc4ccccc4nc3-c3ccc4c(c3)c3ccccc3n4-c3cc4oc5cccc6c7ccccc7c(c3)c4c56)c2)cc1]

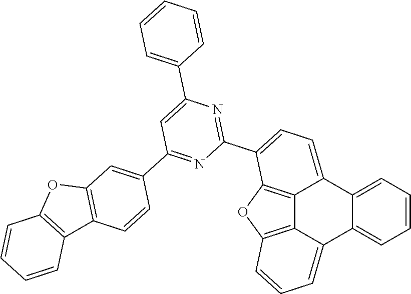 [SMILES: c1ccc(-c2cc(-c3ccc4c(c3)oc3ccccc34)nc(-c3ccc4c5ccccc5c5cccc6oc3c4c65)n2)cc1]

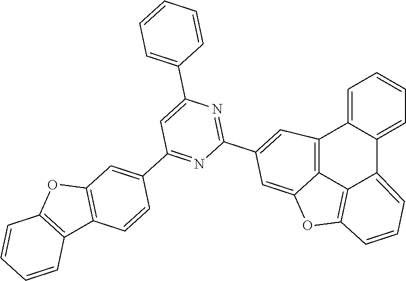 [SMILES: c1ccc(-c2cc(-c3ccc4c(c3)oc3ccccc34)nc(-c3cc4oc5cccc6c7ccccc7c(c3)c4c56)n2)cc1]

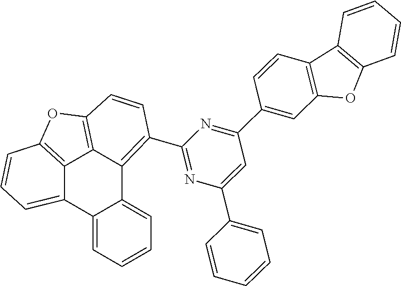 [SMILES: c1ccc(-c2cc(-c3ccc4c(c3)oc3ccccc34)nc(-c3ccc4oc5cccc6c7ccccc7c3c4c56)n2)cc1]

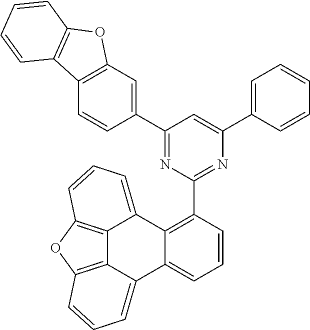 [SMILES: c1ccc(-c2cc(-c3ccc4c(c3)oc3ccccc34)nc(-c3cccc4c5cccc6oc7cccc(c34)c7c65)n2)cc1]

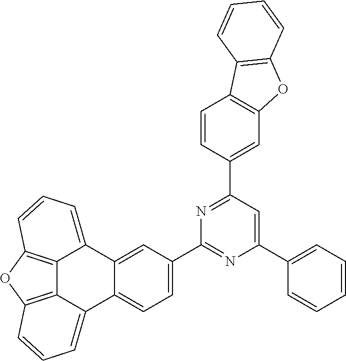 [SMILES: c1ccc(-c2cc(-c3ccc4c(c3)oc3ccccc34)nc(-c3ccc4c(c3)c3cccc5oc6cccc4c6c53)n2)cc1]

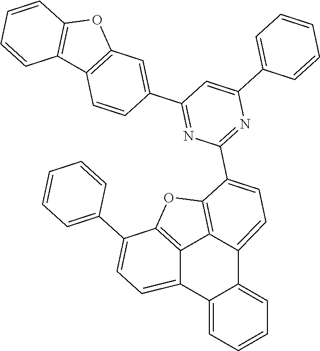 [SMILES: c1ccc(-c2cc(-c3ccc4c(c3)oc3ccccc34)nc(-c3ccc4c5ccccc5c5ccc(-c6ccccc6)c6oc3c4c65)n2)cc1]

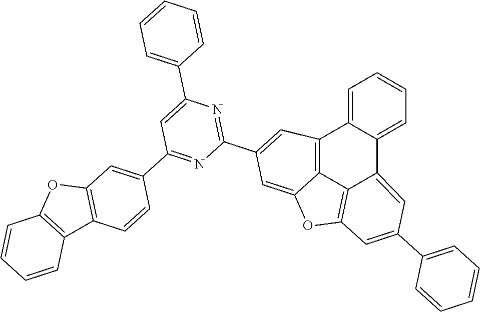 [SMILES: c1ccc(-c2cc3oc4cc(-c5nc(-c6ccccc6)cc(-c6ccc7c(c6)oc6ccccc67)n5)cc5c6ccccc6c(c2)c3c45)cc1]

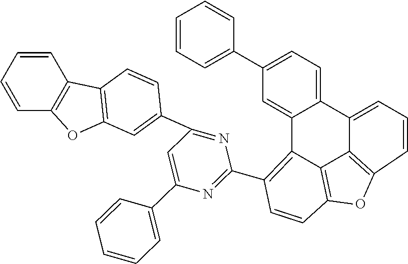 [SMILES: c1ccc(-c2ccc3c(c2)c2c(-c4nc(-c5ccccc5)cc(-c5ccc6c(c5)oc5ccccc56)n4)ccc4oc5cccc3c5c42)cc1]

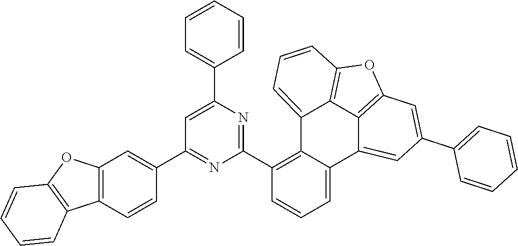 [SMILES: c1ccc(-c2cc3oc4cccc5c6c(-c7nc(-c8ccccc8)cc(-c8ccc9c(c8)oc8ccccc89)n7)cccc6c(c2)c3c45)cc1]

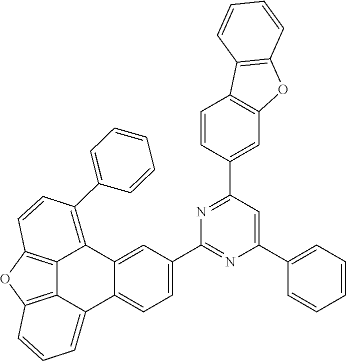 [SMILES: c1ccc(-c2cc(-c3ccc4c(c3)oc3ccccc34)nc(-c3ccc4c(c3)c3c(-c5ccccc5)ccc5oc6cccc4c6c53)n2)cc1]

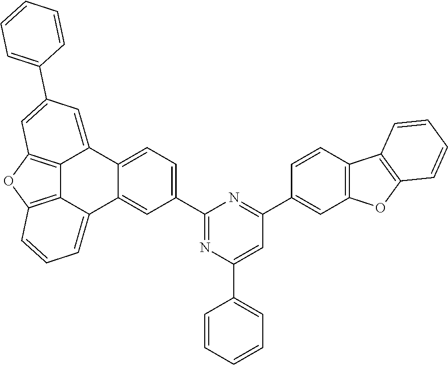 [SMILES: c1ccc(-c2cc3oc4cccc5c6cc(-c7nc(-c8ccccc8)cc(-c8ccc9c(c8)oc8ccccc89)n7)ccc6c(c2)c3c45)cc1]

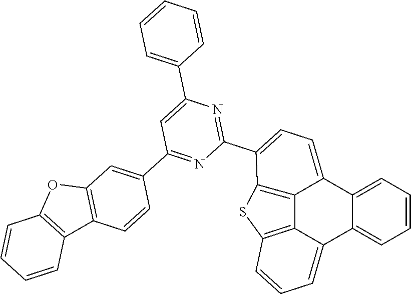 [SMILES: c1ccc(-c2cc(-c3ccc4c(c3)oc3ccccc34)nc(-c3ccc4c5ccccc5c5cccc6sc3c4c65)n2)cc1]

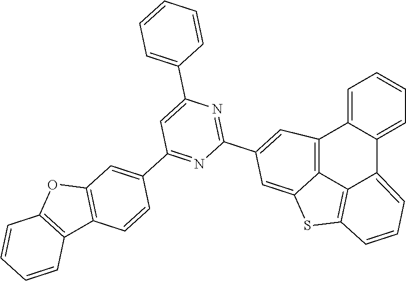 [SMILES: c1ccc(-c2cc(-c3ccc4c(c3)oc3ccccc34)nc(-c3cc4sc5cccc6c7ccccc7c(c3)c4c56)n2)cc1]

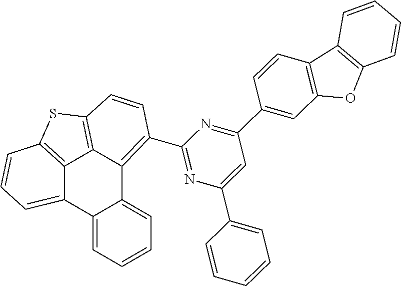 [SMILES: c1ccc(-c2cc(-c3ccc4c(c3)oc3ccccc34)nc(-c3ccc4sc5cccc6c7ccccc7c3c4c56)n2)cc1]

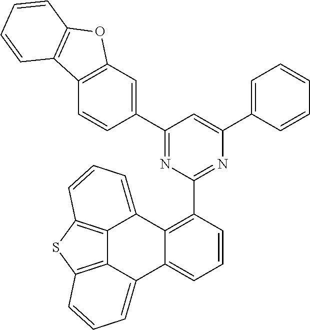 [SMILES: c1ccc(-c2cc(-c3ccc4c(c3)oc3ccccc34)nc(-c3cccc4c5cccc6sc7cccc(c34)c7c65)n2)cc1]

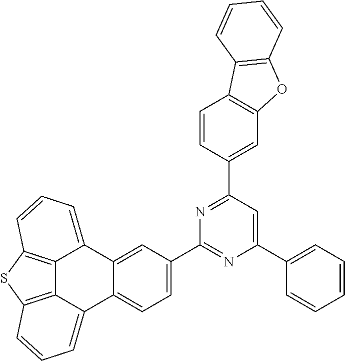 [SMILES: c1ccc(-c2cc(-c3ccc4c(c3)oc3ccccc34)nc(-c3ccc4c(c3)c3cccc5sc6cccc4c6c53)n2)cc1]

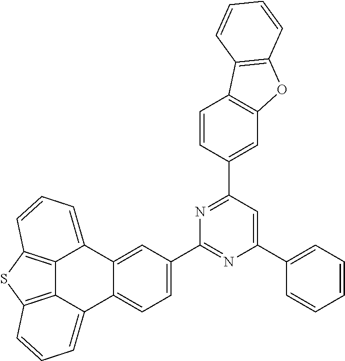 [SMILES: c1ccc(-c2cc(-c3ccc4c(c3)oc3ccccc34)nc(-c3ccc4c(c3)c3cccc5sc6cccc4c6c53)n2)cc1]

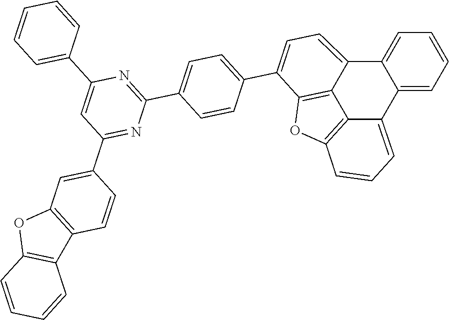 [SMILES: c1ccc(-c2cc(-c3ccc4c(c3)oc3ccccc34)nc(-c3ccc(-c4ccc5c6ccccc6c6cccc7oc4c5c76)cc3)n2)cc1]

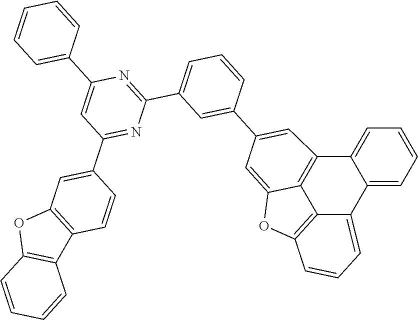 [SMILES: c1ccc(-c2cc(-c3ccc4c(c3)oc3ccccc34)nc(-c3cccc(-c4cc5oc6cccc7c8ccccc8c(c4)c5c67)c3)n2)cc1]

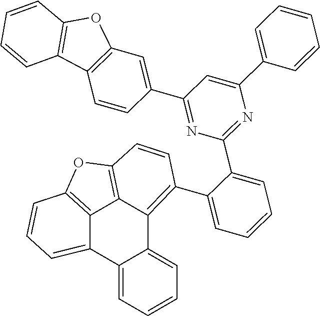 [SMILES: c1ccc(-c2cc(-c3ccc4c(c3)oc3ccccc34)nc(-c3ccccc3-c3ccc4oc5cccc6c7ccccc7c3c4c56)n2)cc1]

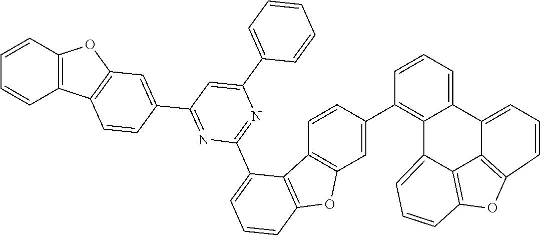 [SMILES: c1ccc(-c2cc(-c3ccc4c(c3)oc3ccccc34)nc(-c3cccc4oc5cc(-c6cccc7c8cccc9oc%10cccc(c67)c%10c98)ccc5c34)n2)cc1]

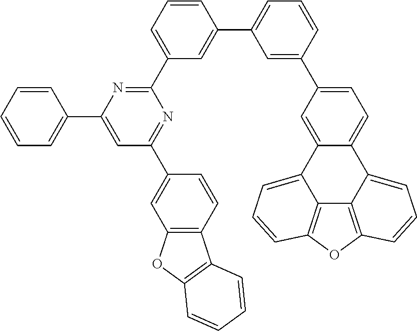 [SMILES: c1ccc(-c2cc(-c3ccc4c(c3)oc3ccccc34)nc(-c3cccc(-c4cccc(-c5ccc6c(c5)c5cccc7oc8cccc6c8c75)c4)c3)n2)cc1]

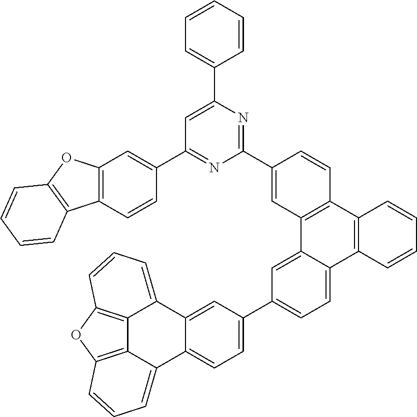 [SMILES: c1ccc(-c2cc(-c3ccc4c(c3)oc3ccccc34)nc(-c3ccc4c5ccccc5c5ccc(-c6ccc7c(c6)c6cccc8oc9cccc7c9c86)cc5c4c3)n2)cc1]

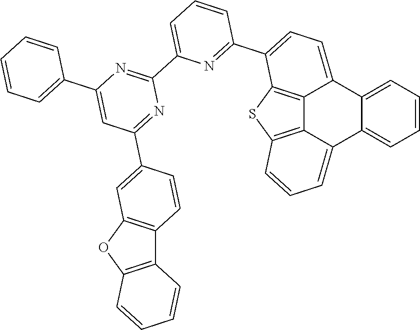 [SMILES: c1ccc(-c2cc(-c3ccc4c(c3)oc3ccccc34)nc(-c3cccc(-c4ccc5c6ccccc6c6cccc7sc4c5c76)n3)n2)cc1]

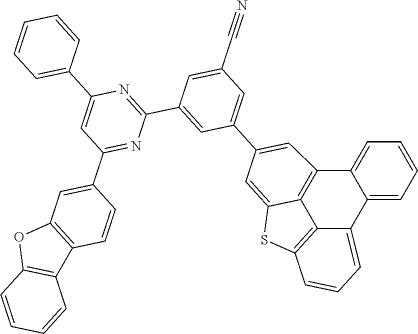 [SMILES: N#Cc1cc(-c2cc3sc4cccc5c6ccccc6c(c2)c3c45)cc(-c2nc(-c3ccccc3)cc(-c3ccc4c(c3)oc3ccccc34)n2)c1]

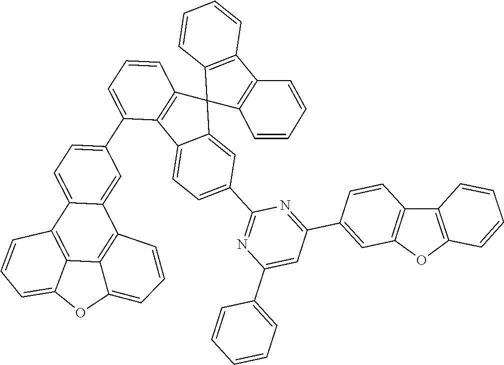 [SMILES: c1ccc(-c2cc(-c3ccc4c(c3)oc3ccccc34)nc(-c3ccc4c(c3)C3(c5ccccc5-c5ccccc53)c3cccc(-c5ccc6c(c5)c5cccc7oc8cccc6c8c75)c3-4)n2)cc1]

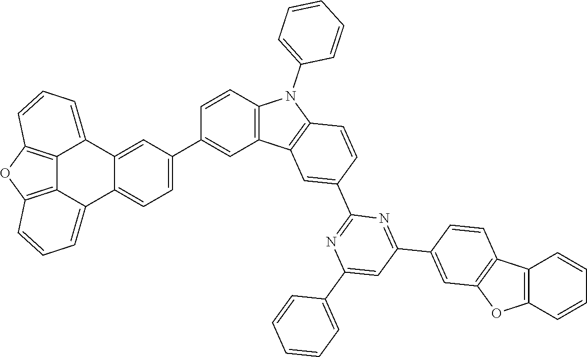 [SMILES: c1ccc(-c2cc(-c3ccc4c(c3)oc3ccccc34)nc(-c3ccc4c(c3)c3cc(-c5ccc6c(c5)c5cccc7oc8cccc6c8c75)ccc3n4-c3ccccc3)n2)cc1]

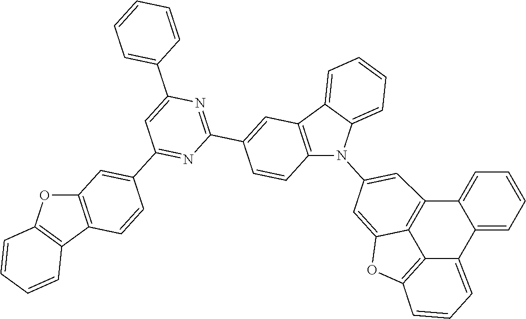 [SMILES: c1ccc(-c2cc(-c3ccc4c(c3)oc3ccccc34)nc(-c3ccc4c(c3)c3ccccc3n4-c3cc4oc5cccc6c7ccccc7c(c3)c4c56)n2)cc1]

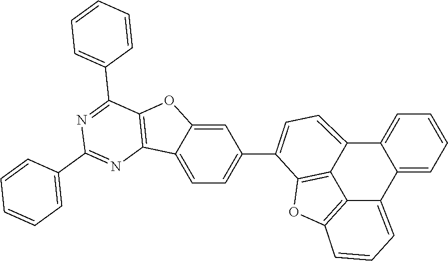 [SMILES: c1ccc(-c2nc(-c3ccccc3)c3oc4cc(-c5ccc6c7ccccc7c7cccc8oc5c6c87)ccc4c3n2)cc1]

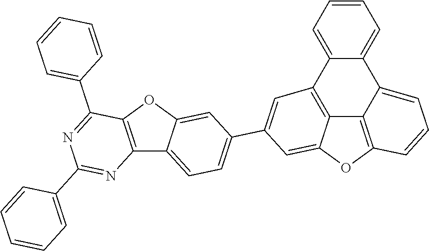 [SMILES: c1ccc(-c2nc(-c3ccccc3)c3oc4cc(-c5cc6oc7cccc8c9ccccc9c(c5)c6c78)ccc4c3n2)cc1]

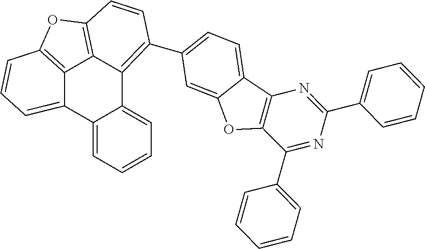 [SMILES: c1ccc(-c2nc(-c3ccccc3)c3oc4cc(-c5ccc6oc7cccc8c9ccccc9c5c6c78)ccc4c3n2)cc1]

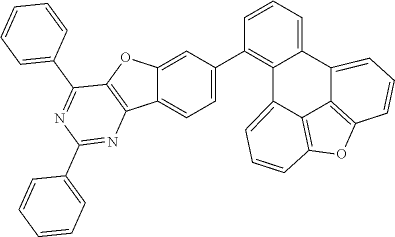 [SMILES: c1ccc(-c2nc(-c3ccccc3)c3oc4cc(-c5cccc6c7cccc8oc9cccc(c56)c9c87)ccc4c3n2)cc1]

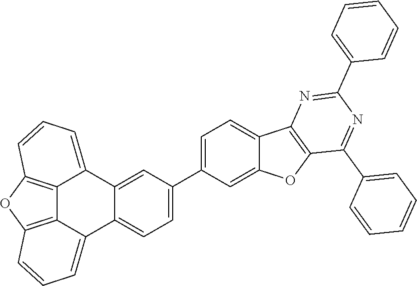 [SMILES: c1ccc(-c2nc(-c3ccccc3)c3oc4cc(-c5ccc6c(c5)c5cccc7oc8cccc6c8c75)ccc4c3n2)cc1]

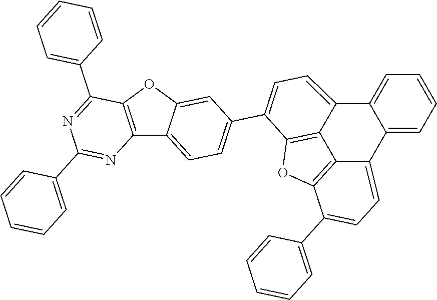 [SMILES: c1ccc(-c2nc(-c3ccccc3)c3oc4cc(-c5ccc6c7ccccc7c7ccc(-c8ccccc8)c8oc5c6c87)ccc4c3n2)cc1]

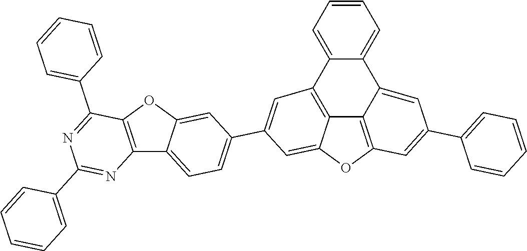 [SMILES: c1ccc(-c2cc3oc4cc(-c5ccc6c(c5)oc5c(-c7ccccc7)nc(-c7ccccc7)nc56)cc5c6ccccc6c(c2)c3c45)cc1]

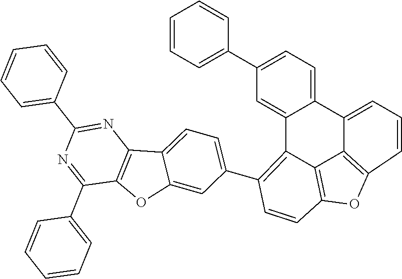 [SMILES: c1ccc(-c2ccc3c(c2)c2c(-c4ccc5c(c4)oc4c(-c6ccccc6)nc(-c6ccccc6)nc45)ccc4oc5cccc3c5c42)cc1]

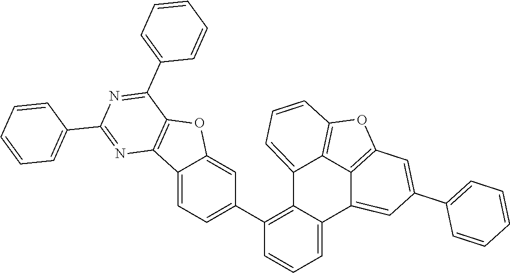 [SMILES: c1ccc(-c2cc3oc4cccc5c6c(-c7ccc8c(c7)oc7c(-c9ccccc9)nc(-c9ccccc9)nc78)cccc6c(c2)c3c45)cc1]

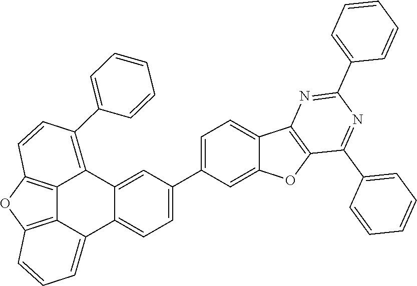 [SMILES: c1ccc(-c2nc(-c3ccccc3)c3oc4cc(-c5ccc6c(c5)c5c(-c7ccccc7)ccc7oc8cccc6c8c75)ccc4c3n2)cc1]

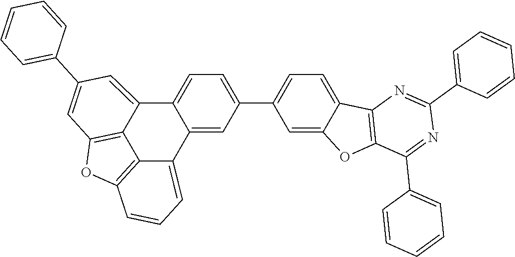 [SMILES: c1ccc(-c2cc3oc4cccc5c6cc(-c7ccc8c(c7)oc7c(-c9ccccc9)nc(-c9ccccc9)nc78)ccc6c(c2)c3c45)cc1]

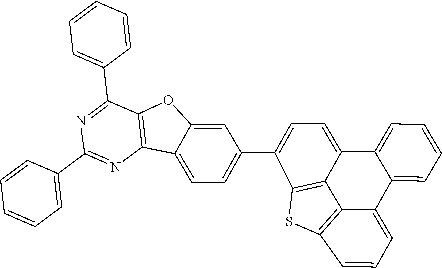 [SMILES: c1ccc(-c2nc(-c3ccccc3)c3oc4cc(-c5ccc6c7ccccc7c7cccc8sc5c6c87)ccc4c3n2)cc1]

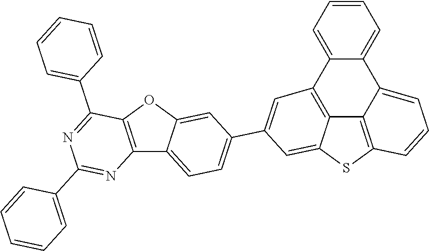 [SMILES: c1ccc(-c2nc(-c3ccccc3)c3oc4cc(-c5cc6sc7cccc8c9ccccc9c(c5)c6c78)ccc4c3n2)cc1]

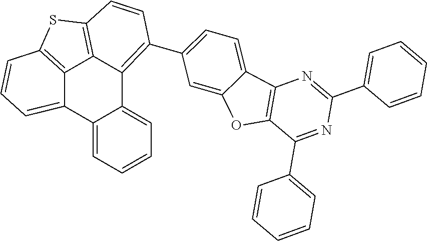 [SMILES: c1ccc(-c2nc(-c3ccccc3)c3oc4cc(-c5ccc6sc7cccc8c9ccccc9c5c6c78)ccc4c3n2)cc1]

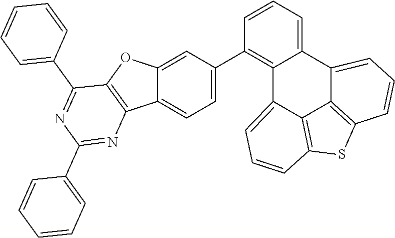 [SMILES: c1ccc(-c2nc(-c3ccccc3)c3oc4cc(-c5cccc6c7cccc8sc9cccc(c56)c9c87)ccc4c3n2)cc1]

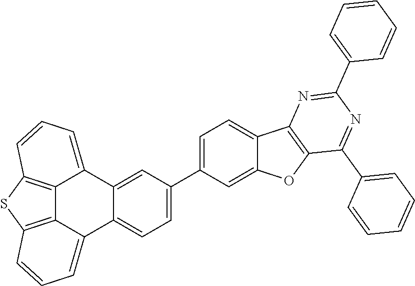 [SMILES: c1ccc(-c2nc(-c3ccccc3)c3oc4cc(-c5ccc6c(c5)c5cccc7sc8cccc6c8c75)ccc4c3n2)cc1]

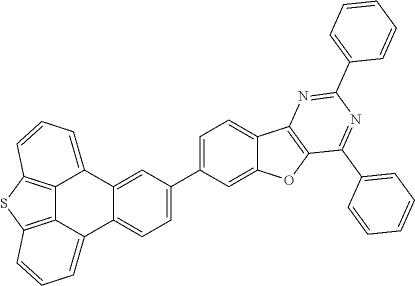 [SMILES: c1ccc(-c2nc(-c3ccccc3)c3oc4cc(-c5ccc6c(c5)c5cccc7sc8cccc6c8c75)ccc4c3n2)cc1]

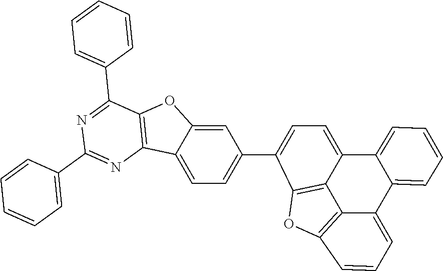 [SMILES: c1ccc(-c2nc(-c3ccccc3)c3oc4cc(-c5ccc6c7ccccc7c7cccc8oc5c6c87)ccc4c3n2)cc1]

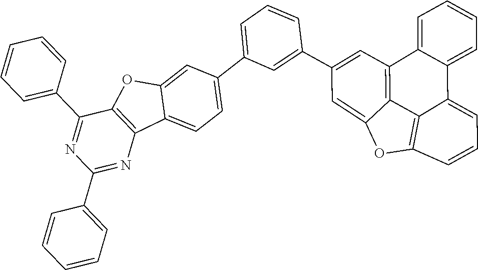 [SMILES: c1ccc(-c2nc(-c3ccccc3)c3oc4cc(-c5cccc(-c6cc7oc8cccc9c%10ccccc%10c(c6)c7c89)c5)ccc4c3n2)cc1]

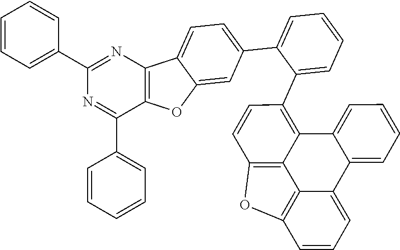 [SMILES: c1ccc(-c2nc(-c3ccccc3)c3oc4cc(-c5ccccc5-c5ccc6oc7cccc8c9ccccc9c5c6c78)ccc4c3n2)cc1]

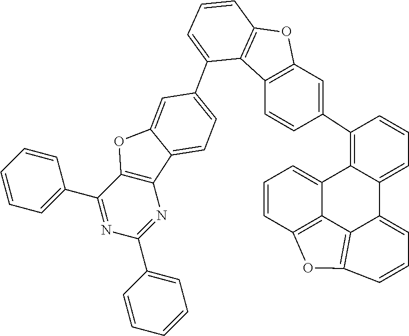 [SMILES: c1ccc(-c2nc(-c3ccccc3)c3oc4cc(-c5cccc6oc7cc(-c8cccc9c%10cccc%11oc%12cccc(c89)c%12c%11%10)ccc7c56)ccc4c3n2)cc1]

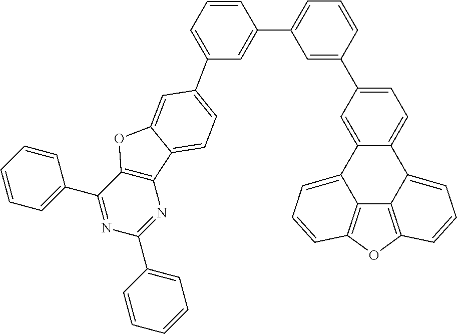 [SMILES: c1ccc(-c2nc(-c3ccccc3)c3oc4cc(-c5cccc(-c6cccc(-c7ccc8c(c7)c7cccc9oc%10cccc8c%10c97)c6)c5)ccc4c3n2)cc1]

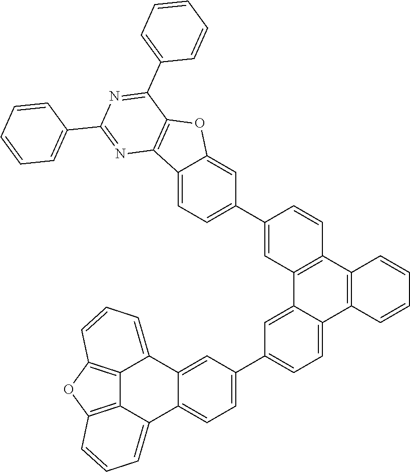 [SMILES: c1ccc(-c2nc(-c3ccccc3)c3oc4cc(-c5ccc6c7ccccc7c7ccc(-c8ccc9c(c8)c8cccc%10oc%11cccc9c%11c%108)cc7c6c5)ccc4c3n2)cc1]

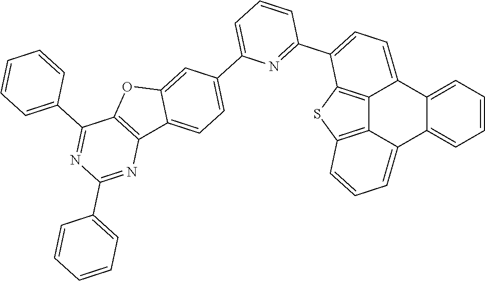 [SMILES: c1ccc(-c2nc(-c3ccccc3)c3oc4cc(-c5cccc(-c6ccc7c8ccccc8c8cccc9sc6c7c98)n5)ccc4c3n2)cc1]

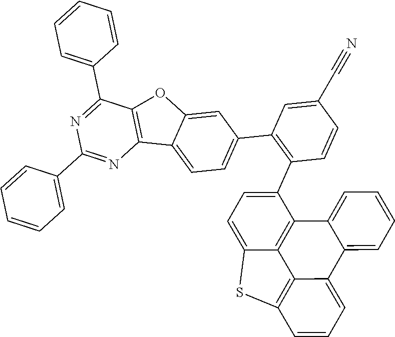 [SMILES: N#Cc1ccc(-c2ccc3sc4cccc5c6ccccc6c2c3c45)c(-c2ccc3c(c2)oc2c(-c4ccccc4)nc(-c4ccccc4)nc23)c1]

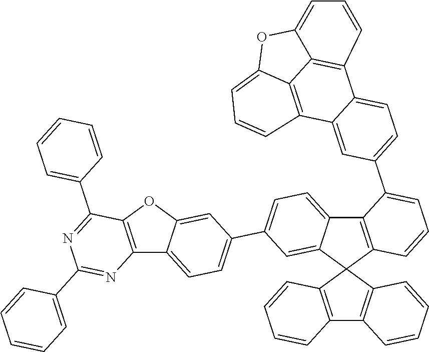 [SMILES: c1ccc(-c2nc(-c3ccccc3)c3oc4cc(-c5ccc6c(c5)C5(c7ccccc7-c7ccccc75)c5cccc(-c7ccc8c(c7)c7cccc9oc%10cccc8c%10c97)c5-6)ccc4c3n2)cc1]

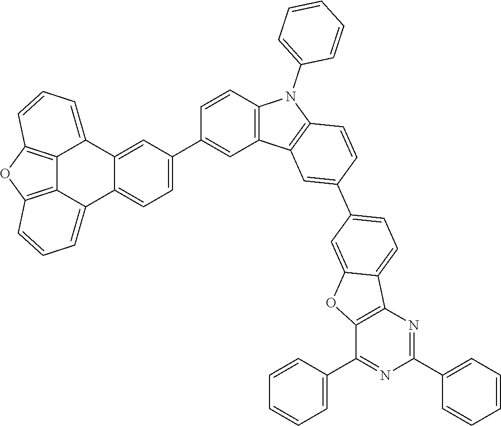 [SMILES: c1ccc(-c2nc(-c3ccccc3)c3oc4cc(-c5ccc6c(c5)c5cc(-c7ccc8c(c7)c7cccc9oc%10cccc8c%10c97)ccc5n6-c5ccccc5)ccc4c3n2)cc1]

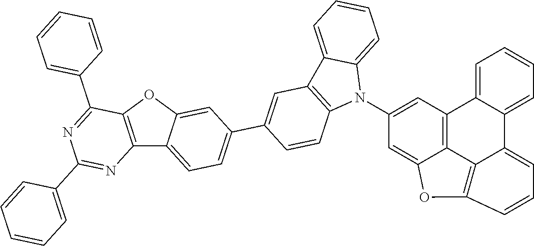 [SMILES: c1ccc(-c2nc(-c3ccccc3)c3oc4cc(-c5ccc6c(c5)c5ccccc5n6-c5cc6oc7cccc8c9ccccc9c(c5)c6c78)ccc4c3n2)cc1]

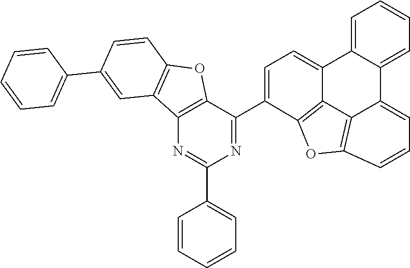 [SMILES: c1ccc(-c2ccc3oc4c(-c5ccc6c7ccccc7c7cccc8oc5c6c87)nc(-c5ccccc5)nc4c3c2)cc1]

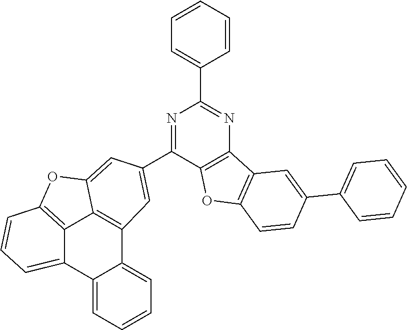 [SMILES: c1ccc(-c2ccc3oc4c(-c5cc6oc7cccc8c9ccccc9c(c5)c6c78)nc(-c5ccccc5)nc4c3c2)cc1]

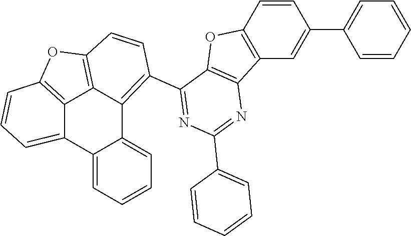 [SMILES: c1ccc(-c2ccc3oc4c(-c5ccc6oc7cccc8c9ccccc9c5c6c78)nc(-c5ccccc5)nc4c3c2)cc1]

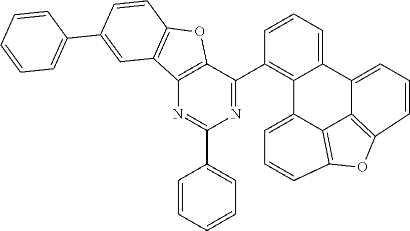 [SMILES: c1ccc(-c2ccc3oc4c(-c5cccc6c7cccc8oc9cccc(c56)c9c87)nc(-c5ccccc5)nc4c3c2)cc1]

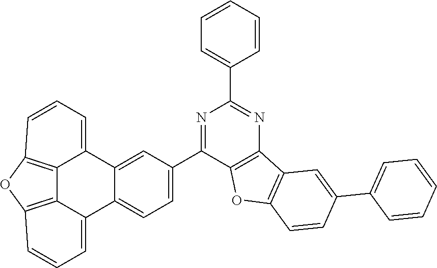 [SMILES: c1ccc(-c2ccc3oc4c(-c5ccc6c(c5)c5cccc7oc8cccc6c8c75)nc(-c5ccccc5)nc4c3c2)cc1]

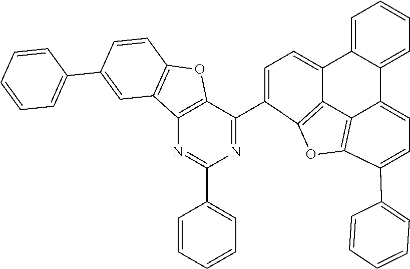 [SMILES: c1ccc(-c2ccc3oc4c(-c5ccc6c7ccccc7c7ccc(-c8ccccc8)c8oc5c6c87)nc(-c5ccccc5)nc4c3c2)cc1]

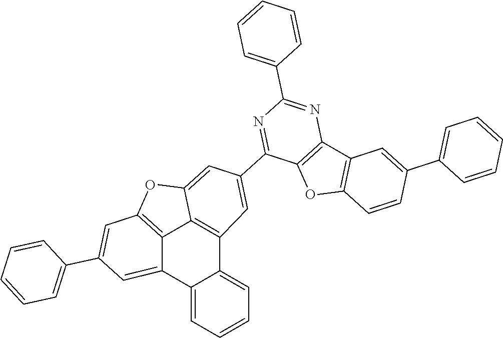 [SMILES: c1ccc(-c2ccc3oc4c(-c5cc6oc7cc(-c8ccccc8)cc8c9ccccc9c(c5)c6c78)nc(-c5ccccc5)nc4c3c2)cc1]

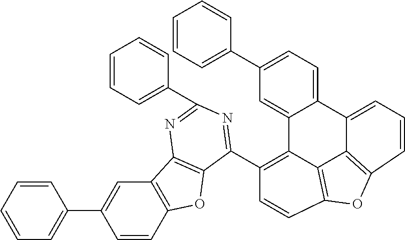 [SMILES: c1ccc(-c2ccc3oc4c(-c5ccc6oc7cccc8c9ccc(-c%10ccccc%10)cc9c5c6c78)nc(-c5ccccc5)nc4c3c2)cc1]

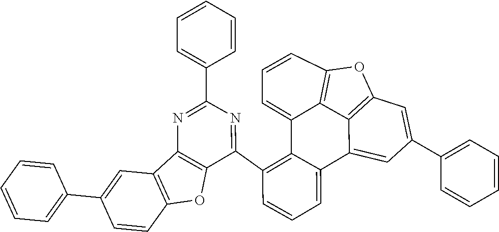 [SMILES: c1ccc(-c2ccc3oc4c(-c5cccc6c7cc(-c8ccccc8)cc8oc9cccc(c56)c9c87)nc(-c5ccccc5)nc4c3c2)cc1]

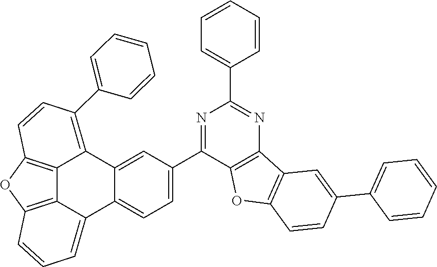 [SMILES: c1ccc(-c2ccc3oc4c(-c5ccc6c(c5)c5c(-c7ccccc7)ccc7oc8cccc6c8c75)nc(-c5ccccc5)nc4c3c2)cc1]

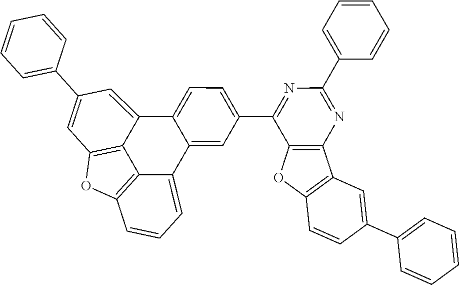 [SMILES: c1ccc(-c2ccc3oc4c(-c5ccc6c(c5)c5cccc7oc8cc(-c9ccccc9)cc6c8c75)nc(-c5ccccc5)nc4c3c2)cc1]

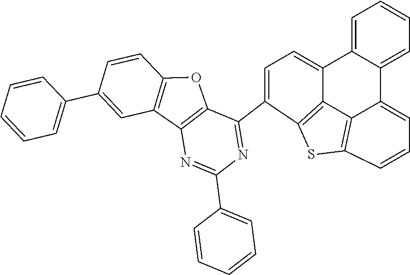 [SMILES: c1ccc(-c2ccc3oc4c(-c5ccc6c7ccccc7c7cccc8sc5c6c87)nc(-c5ccccc5)nc4c3c2)cc1]

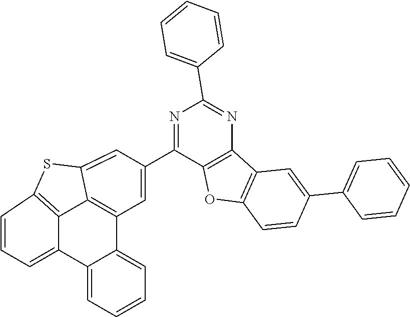 [SMILES: c1ccc(-c2ccc3oc4c(-c5cc6sc7cccc8c9ccccc9c(c5)c6c78)nc(-c5ccccc5)nc4c3c2)cc1]

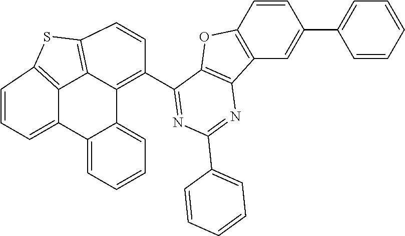 [SMILES: c1ccc(-c2ccc3oc4c(-c5ccc6sc7cccc8c9ccccc9c5c6c78)nc(-c5ccccc5)nc4c3c2)cc1]

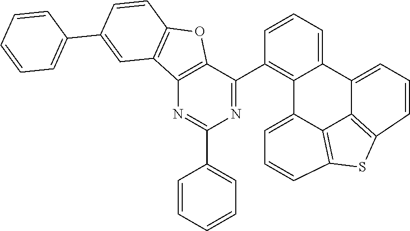 [SMILES: c1ccc(-c2ccc3oc4c(-c5cccc6c7cccc8sc9cccc(c56)c9c87)nc(-c5ccccc5)nc4c3c2)cc1]

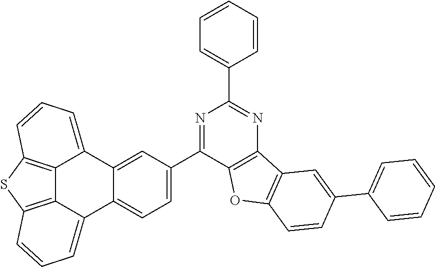 [SMILES: c1ccc(-c2ccc3oc4c(-c5ccc6c(c5)c5cccc7sc8cccc6c8c75)nc(-c5ccccc5)nc4c3c2)cc1]

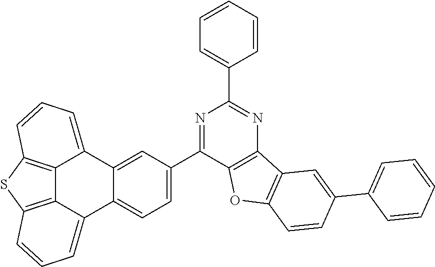 [SMILES: c1ccc(-c2ccc3oc4c(-c5ccc6c(c5)c5cccc7sc8cccc6c8c75)nc(-c5ccccc5)nc4c3c2)cc1]

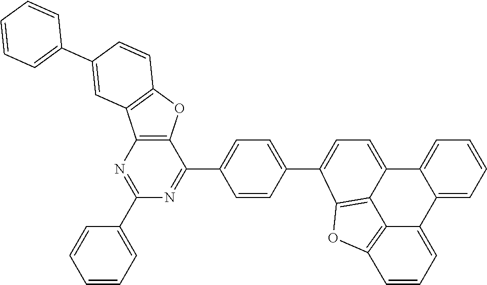 [SMILES: c1ccc(-c2ccc3oc4c(-c5ccc(-c6ccc7c8ccccc8c8cccc9oc6c7c98)cc5)nc(-c5ccccc5)nc4c3c2)cc1]

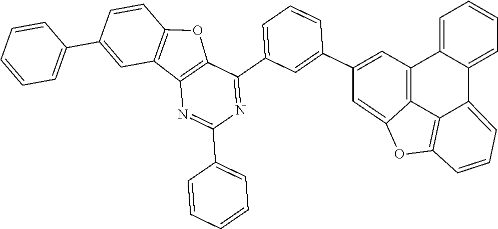 [SMILES: c1ccc(-c2ccc3oc4c(-c5cccc(-c6cc7oc8cccc9c%10ccccc%10c(c6)c7c89)c5)nc(-c5ccccc5)nc4c3c2)cc1]

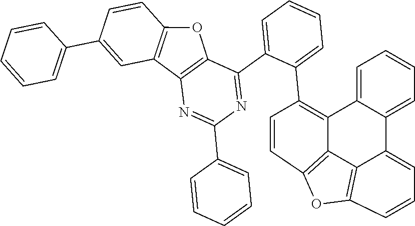 [SMILES: c1ccc(-c2ccc3oc4c(-c5ccccc5-c5ccc6oc7cccc8c9ccccc9c5c6c78)nc(-c5ccccc5)nc4c3c2)cc1]

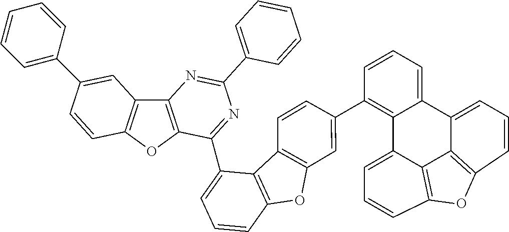 [SMILES: c1ccc(-c2ccc3oc4c(-c5cccc6oc7cc(-c8cccc9c%10cccc%11oc%12cccc(c89)c%12c%11%10)ccc7c56)nc(-c5ccccc5)nc4c3c2)cc1]

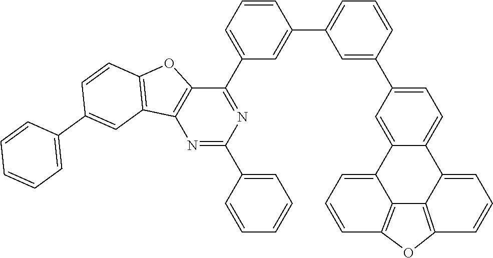 [SMILES: c1ccc(-c2ccc3oc4c(-c5cccc(-c6cccc(-c7ccc8c(c7)c7cccc9oc%10cccc8c%10c97)c6)c5)nc(-c5ccccc5)nc4c3c2)cc1]

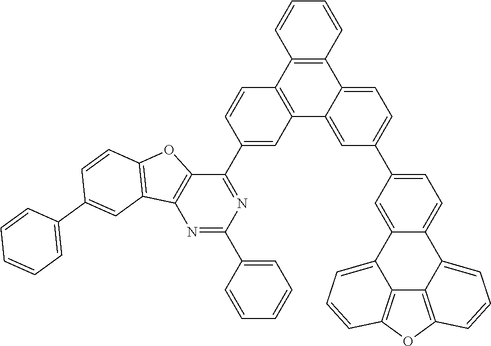 [SMILES: c1ccc(-c2ccc3oc4c(-c5ccc6c7ccccc7c7ccc(-c8ccc9c(c8)c8cccc%10oc%11cccc9c%11c%108)cc7c6c5)nc(-c5ccccc5)nc4c3c2)cc1]

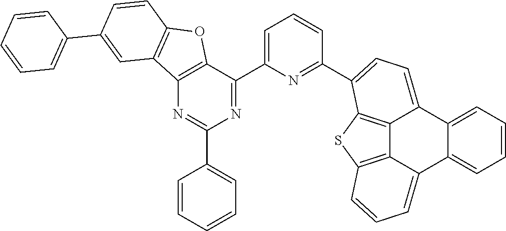 [SMILES: c1ccc(-c2ccc3oc4c(-c5cccc(-c6ccc7c8ccccc8c8cccc9sc6c7c98)n5)nc(-c5ccccc5)nc4c3c2)cc1]

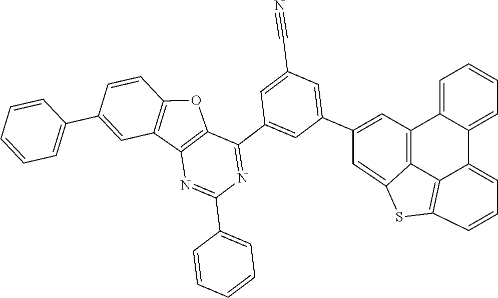 [SMILES: N#Cc1cc(-c2cc3sc4cccc5c6ccccc6c(c2)c3c45)cc(-c2nc(-c3ccccc3)nc3c2oc2ccc(-c4ccccc4)cc23)c1]

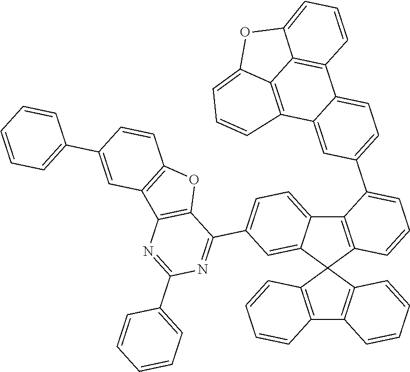 [SMILES: c1ccc(-c2ccc3oc4c(-c5ccc6c(c5)C5(c7ccccc7-c7ccccc75)c5cccc(-c7ccc8c(c7)c7cccc9oc%10cccc8c%10c97)c5-6)nc(-c5ccccc5)nc4c3c2)cc1]

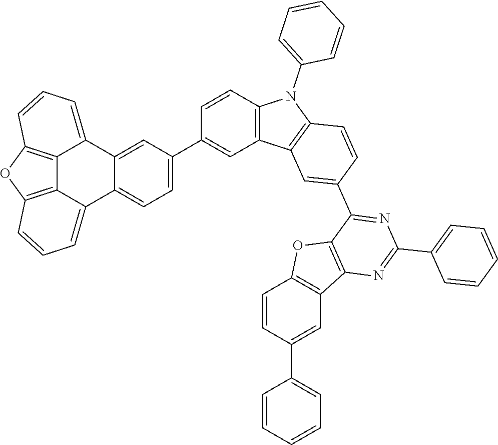 [SMILES: c1ccc(-c2ccc3oc4c(-c5ccc6c(c5)c5cc(-c7ccc8c(c7)c7cccc9oc%10cccc8c%10c97)ccc5n6-c5ccccc5)nc(-c5ccccc5)nc4c3c2)cc1]

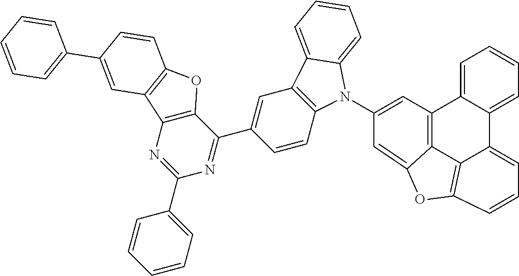 [SMILES: c1ccc(-c2ccc3oc4c(-c5ccc6c(c5)c5ccccc5n6-c5cc6oc7cccc8c9ccccc9c(c5)c6c78)nc(-c5ccccc5)nc4c3c2)cc1]

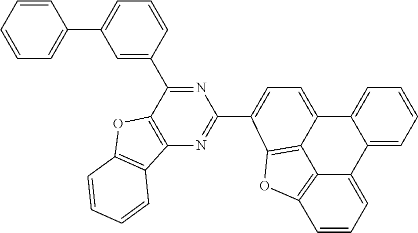 [SMILES: c1ccc(-c2cccc(-c3nc(-c4ccc5c6ccccc6c6cccc7oc4c5c76)nc4c3oc3ccccc34)c2)cc1]

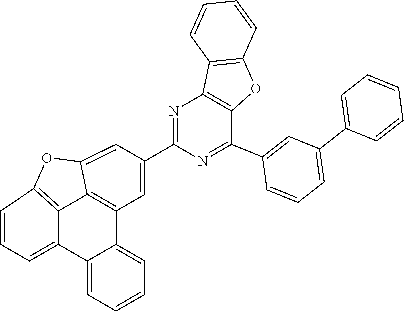 [SMILES: c1ccc(-c2cccc(-c3nc(-c4cc5oc6cccc7c8ccccc8c(c4)c5c67)nc4c3oc3ccccc34)c2)cc1]

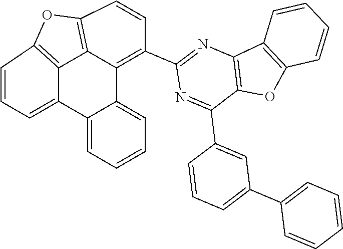 [SMILES: c1ccc(-c2cccc(-c3nc(-c4ccc5oc6cccc7c8ccccc8c4c5c67)nc4c3oc3ccccc34)c2)cc1]

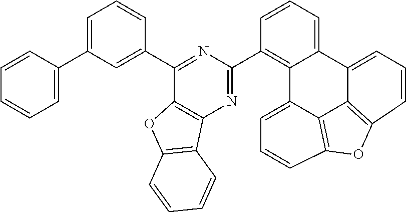 [SMILES: c1ccc(-c2cccc(-c3nc(-c4cccc5c6cccc7oc8cccc(c45)c8c76)nc4c3oc3ccccc34)c2)cc1]

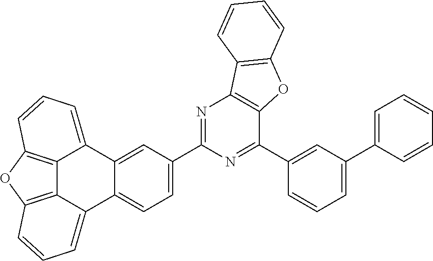 [SMILES: c1ccc(-c2cccc(-c3nc(-c4ccc5c(c4)c4cccc6oc7cccc5c7c64)nc4c3oc3ccccc34)c2)cc1]

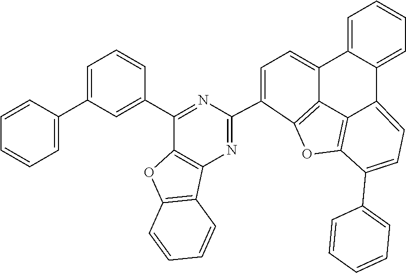 [SMILES: c1ccc(-c2cccc(-c3nc(-c4ccc5c6ccccc6c6ccc(-c7ccccc7)c7oc4c5c76)nc4c3oc3ccccc34)c2)cc1]

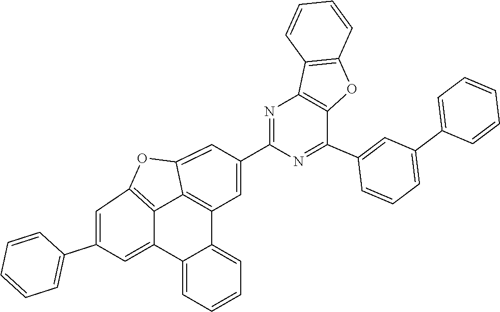 [SMILES: c1ccc(-c2cccc(-c3nc(-c4cc5oc6cc(-c7ccccc7)cc7c8ccccc8c(c4)c5c67)nc4c3oc3ccccc34)c2)cc1]

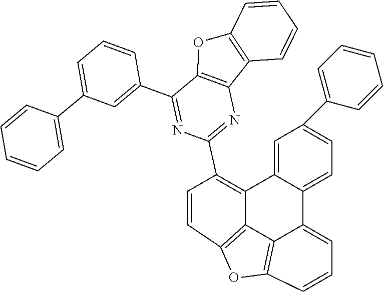 [SMILES: c1ccc(-c2cccc(-c3nc(-c4ccc5oc6cccc7c8ccc(-c9ccccc9)cc8c4c5c67)nc4c3oc3ccccc34)c2)cc1]

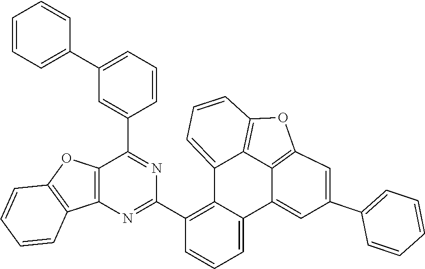 [SMILES: c1ccc(-c2cccc(-c3nc(-c4cccc5c6cc(-c7ccccc7)cc7oc8cccc(c45)c8c76)nc4c3oc3ccccc34)c2)cc1]

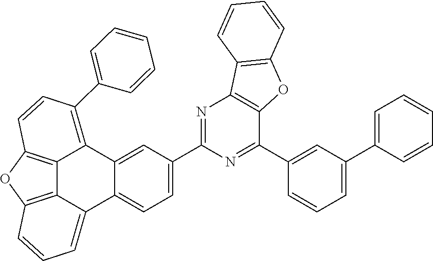 [SMILES: c1ccc(-c2cccc(-c3nc(-c4ccc5c(c4)c4c(-c6ccccc6)ccc6oc7cccc5c7c64)nc4c3oc3ccccc34)c2)cc1]

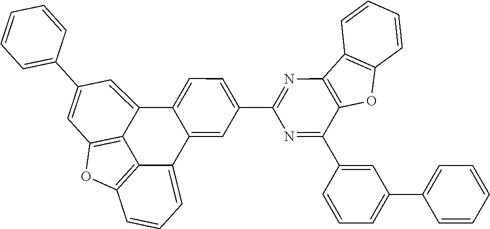 [SMILES: c1ccc(-c2cccc(-c3nc(-c4ccc5c(c4)c4cccc6oc7cc(-c8ccccc8)cc5c7c64)nc4c3oc3ccccc34)c2)cc1]

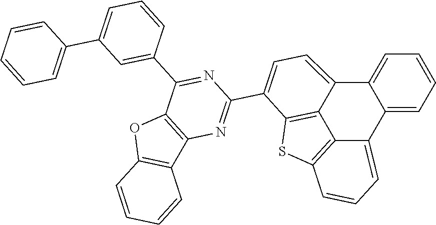 [SMILES: c1ccc(-c2cccc(-c3nc(-c4ccc5c6ccccc6c6cccc7sc4c5c76)nc4c3oc3ccccc34)c2)cc1]

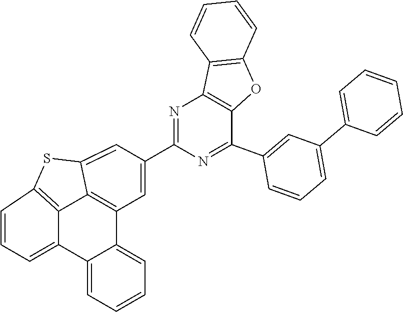 [SMILES: c1ccc(-c2cccc(-c3nc(-c4cc5sc6cccc7c8ccccc8c(c4)c5c67)nc4c3oc3ccccc34)c2)cc1]

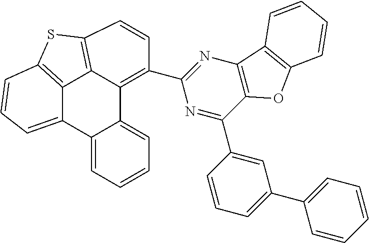 [SMILES: c1ccc(-c2cccc(-c3nc(-c4ccc5sc6cccc7c8ccccc8c4c5c67)nc4c3oc3ccccc34)c2)cc1]

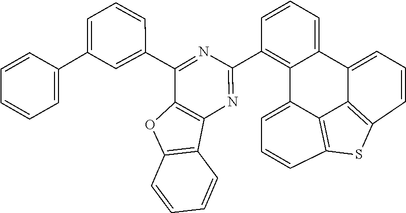 [SMILES: c1ccc(-c2cccc(-c3nc(-c4cccc5c6cccc7sc8cccc(c45)c8c76)nc4c3oc3ccccc34)c2)cc1]

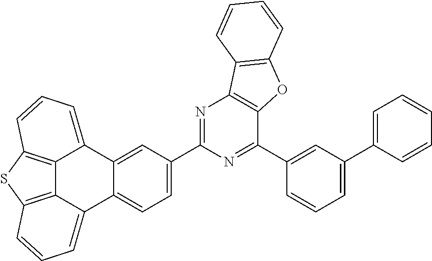 [SMILES: c1ccc(-c2cccc(-c3nc(-c4ccc5c(c4)c4cccc6sc7cccc5c7c64)nc4c3oc3ccccc34)c2)cc1]

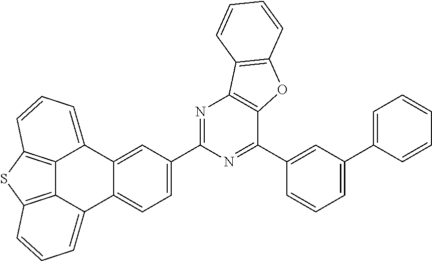 [SMILES: c1ccc(-c2cccc(-c3nc(-c4ccc5c(c4)c4cccc6sc7cccc5c7c64)nc4c3oc3ccccc34)c2)cc1]

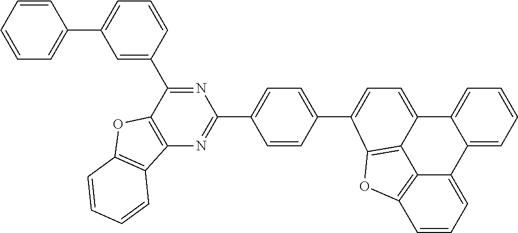 [SMILES: c1ccc(-c2cccc(-c3nc(-c4ccc(-c5ccc6c7ccccc7c7cccc8oc5c6c87)cc4)nc4c3oc3ccccc34)c2)cc1]

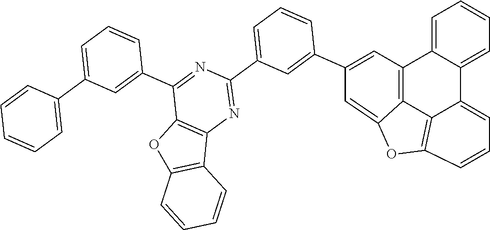 [SMILES: c1ccc(-c2cccc(-c3nc(-c4cccc(-c5cc6oc7cccc8c9ccccc9c(c5)c6c78)c4)nc4c3oc3ccccc34)c2)cc1]

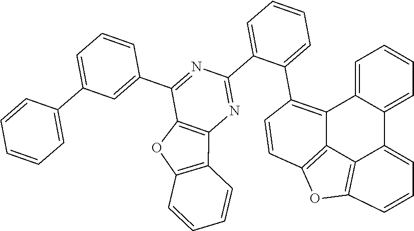 [SMILES: c1ccc(-c2cccc(-c3nc(-c4ccccc4-c4ccc5oc6cccc7c8ccccc8c4c5c67)nc4c3oc3ccccc34)c2)cc1]

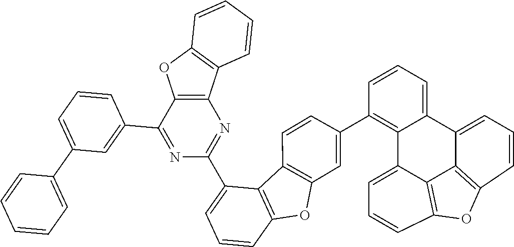 [SMILES: c1ccc(-c2cccc(-c3nc(-c4cccc5oc6cc(-c7cccc8c9cccc%10oc%11cccc(c78)c%11c%109)ccc6c45)nc4c3oc3ccccc34)c2)cc1]

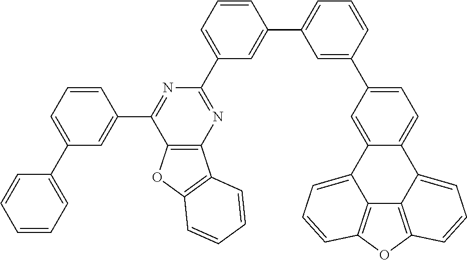 [SMILES: c1ccc(-c2cccc(-c3nc(-c4cccc(-c5cccc(-c6ccc7c(c6)c6cccc8oc9cccc7c9c86)c5)c4)nc4c3oc3ccccc34)c2)cc1]

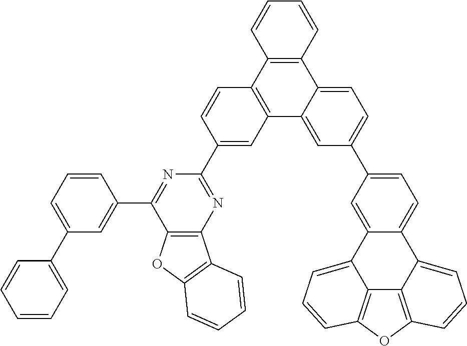 [SMILES: c1ccc(-c2cccc(-c3nc(-c4ccc5c6ccccc6c6ccc(-c7ccc8c(c7)c7cccc9oc%10cccc8c%10c97)cc6c5c4)nc4c3oc3ccccc34)c2)cc1]

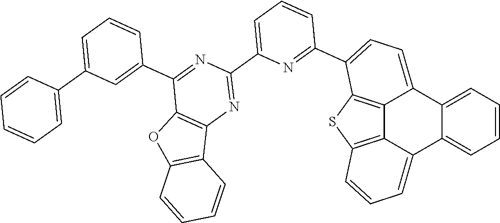 [SMILES: c1ccc(-c2cccc(-c3nc(-c4cccc(-c5ccc6c7ccccc7c7cccc8sc5c6c87)n4)nc4c3oc3ccccc34)c2)cc1]

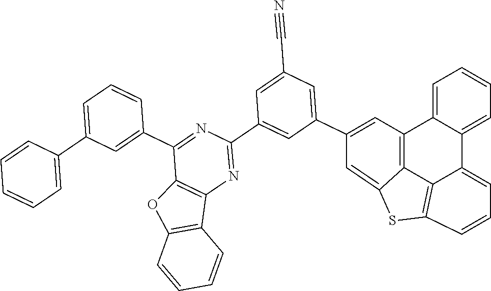 [SMILES: N#Cc1cc(-c2cc3sc4cccc5c6ccccc6c(c2)c3c45)cc(-c2nc(-c3cccc(-c4ccccc4)c3)c3oc4ccccc4c3n2)c1]

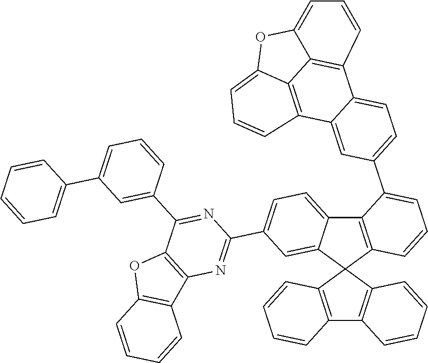 [SMILES: c1ccc(-c2cccc(-c3nc(-c4ccc5c(c4)C4(c6ccccc6-c6ccccc64)c4cccc(-c6ccc7c(c6)c6cccc8oc9cccc7c9c86)c4-5)nc4c3oc3ccccc34)c2)cc1]

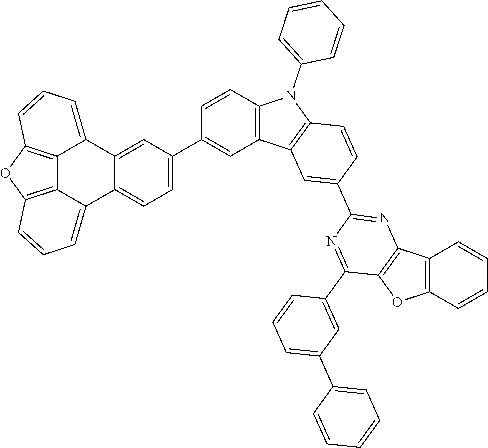 [SMILES: c1ccc(-c2cccc(-c3nc(-c4ccc5c(c4)c4cc(-c6ccc7c(c6)c6cccc8oc9cccc7c9c86)ccc4n5-c4ccccc4)nc4c3oc3ccccc34)c2)cc1]

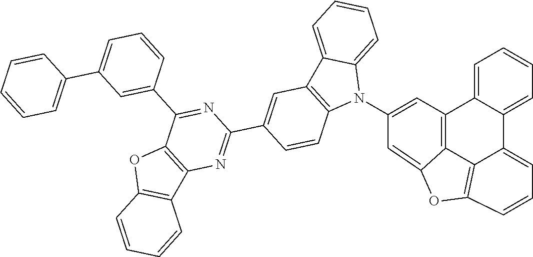 [SMILES: c1ccc(-c2cccc(-c3nc(-c4ccc5c(c4)c4ccccc4n5-c4cc5oc6cccc7c8ccccc8c(c4)c5c67)nc4c3oc3ccccc34)c2)cc1]

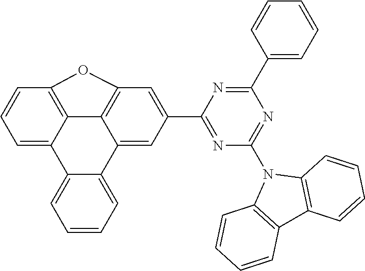 [SMILES: c1ccc(-c2nc(-c3cc4oc5cccc6c7ccccc7c(c3)c4c56)nc(-n3c4ccccc4c4ccccc43)n2)cc1]

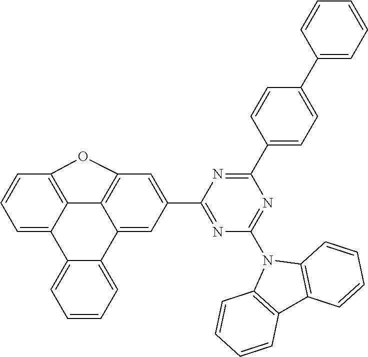 [SMILES: c1ccc(-c2ccc(-c3nc(-c4cc5oc6cccc7c8ccccc8c(c4)c5c67)nc(-n4c5ccccc5c5ccccc54)n3)cc2)cc1]

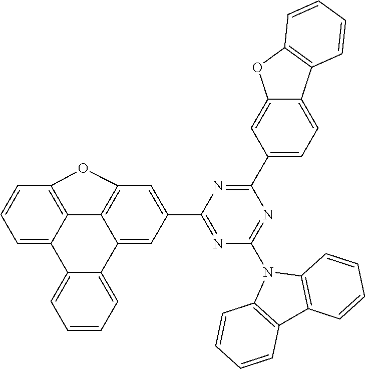 [SMILES: c1ccc2c(c1)oc1cc(-c3nc(-c4cc5oc6cccc7c8ccccc8c(c4)c5c67)nc(-n4c5ccccc5c5ccccc54)n3)ccc12]

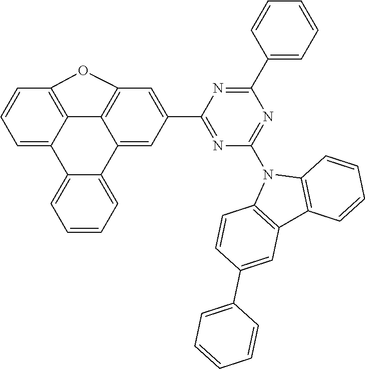 [SMILES: c1ccc(-c2ccc3c(c2)c2ccccc2n3-c2nc(-c3ccccc3)nc(-c3cc4oc5cccc6c7ccccc7c(c3)c4c56)n2)cc1]

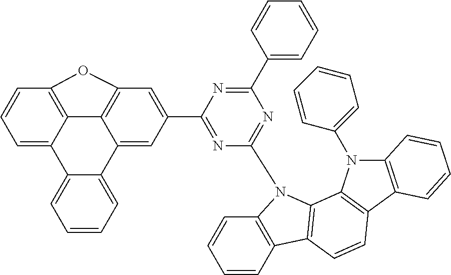 [SMILES: c1ccc(-c2nc(-c3cc4oc5cccc6c7ccccc7c(c3)c4c56)nc(-n3c4ccccc4c4ccc5c6ccccc6n(-c6ccccc6)c5c43)n2)cc1]

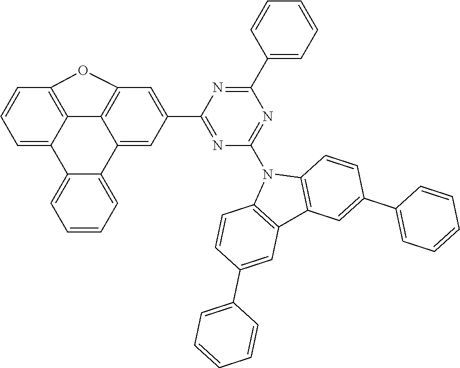 [SMILES: c1ccc(-c2ccc3c(c2)c2cc(-c4ccccc4)ccc2n3-c2nc(-c3ccccc3)nc(-c3cc4oc5cccc6c7ccccc7c(c3)c4c56)n2)cc1]

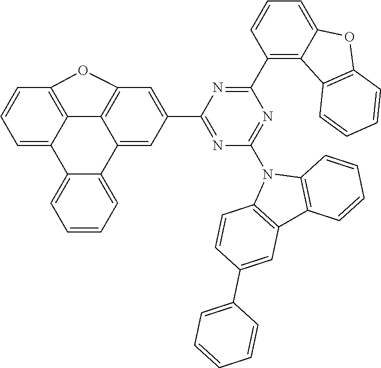 [SMILES: c1ccc(-c2ccc3c(c2)c2ccccc2n3-c2nc(-c3cc4oc5cccc6c7ccccc7c(c3)c4c56)nc(-c3cccc4oc5ccccc5c34)n2)cc1]

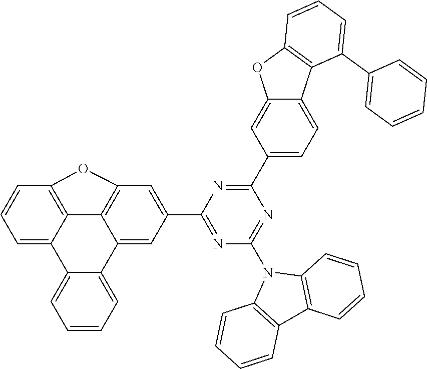 [SMILES: c1ccc(-c2cccc3oc4cc(-c5nc(-c6cc7oc8cccc9c%10ccccc%10c(c6)c7c89)nc(-n6c7ccccc7c7ccccc76)n5)ccc4c23)cc1]

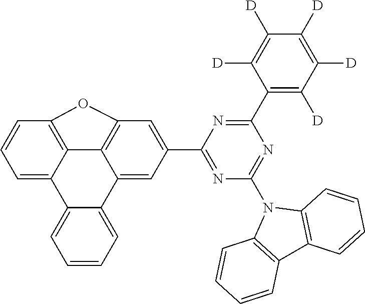 [SMILES: [2H]c1c([2H])c([2H])c(-c2nc(-c3cc4oc5cccc6c7ccccc7c(c3)c4c56)nc(-n3c4ccccc4c4ccccc43)n2)c([2H])c1[2H]]

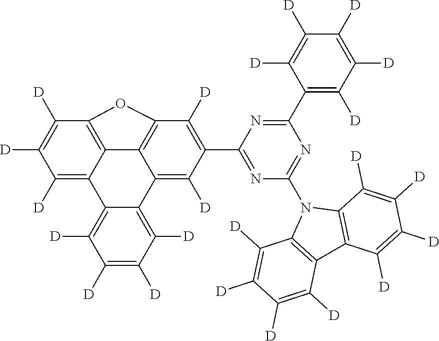 [SMILES: [2H]c1c([2H])c([2H])c(-c2nc(-c3c([2H])c4oc5c([2H])c([2H])c([2H])c6c7c([2H])c([2H])c([2H])c([2H])c7c(c3[2H])c4c56)nc(-n3c4c([2H])c([2H])c([2H])c([2H])c4c4c([2H])c([2H])c([2H])c([2H])c43)n2)c([2H])c1[2H]]

The base structure of the compounds of the invention is known in the literature. These can be prepared and functionalized by the routes outlined in schemes 1 and 2 that follow.

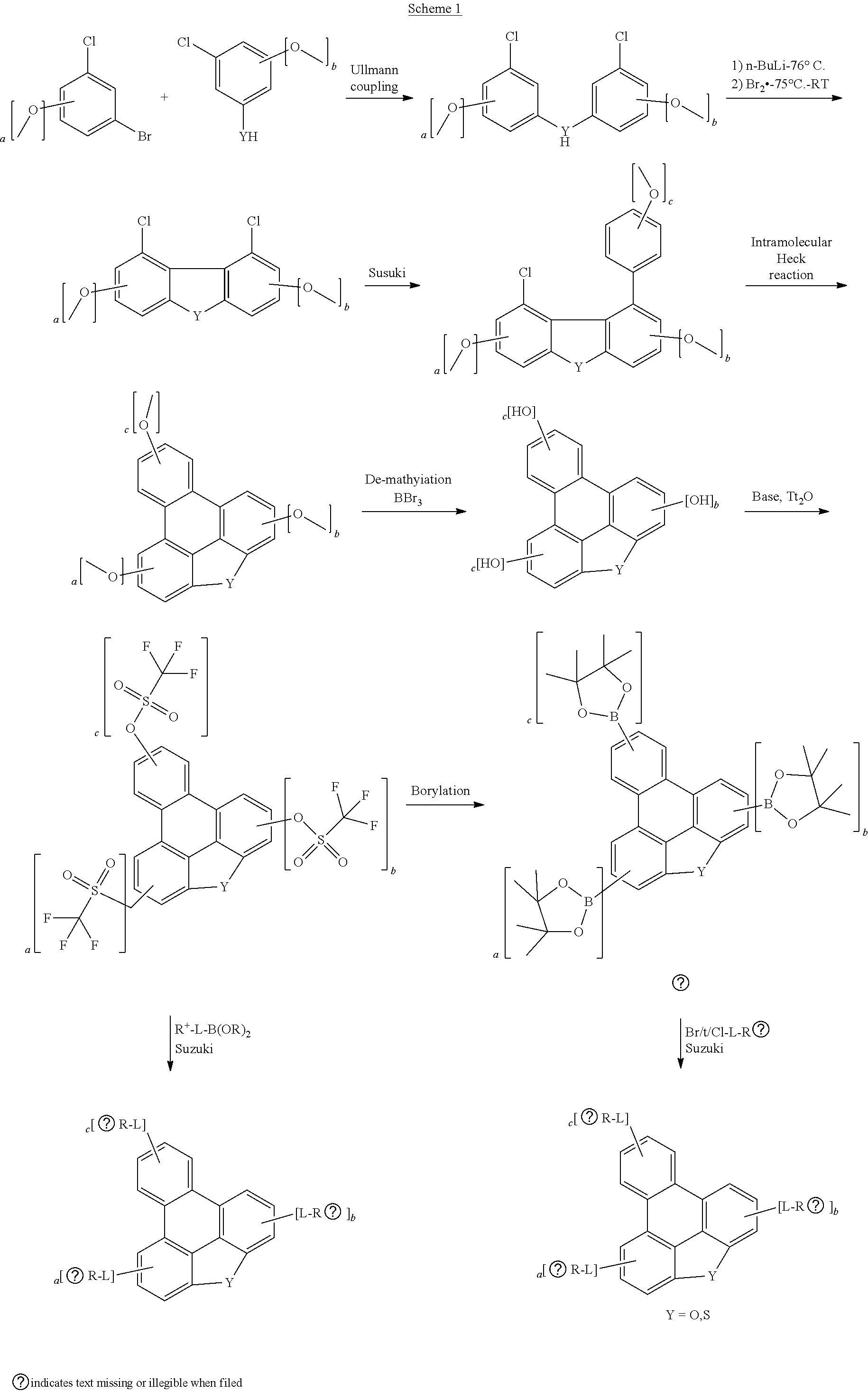

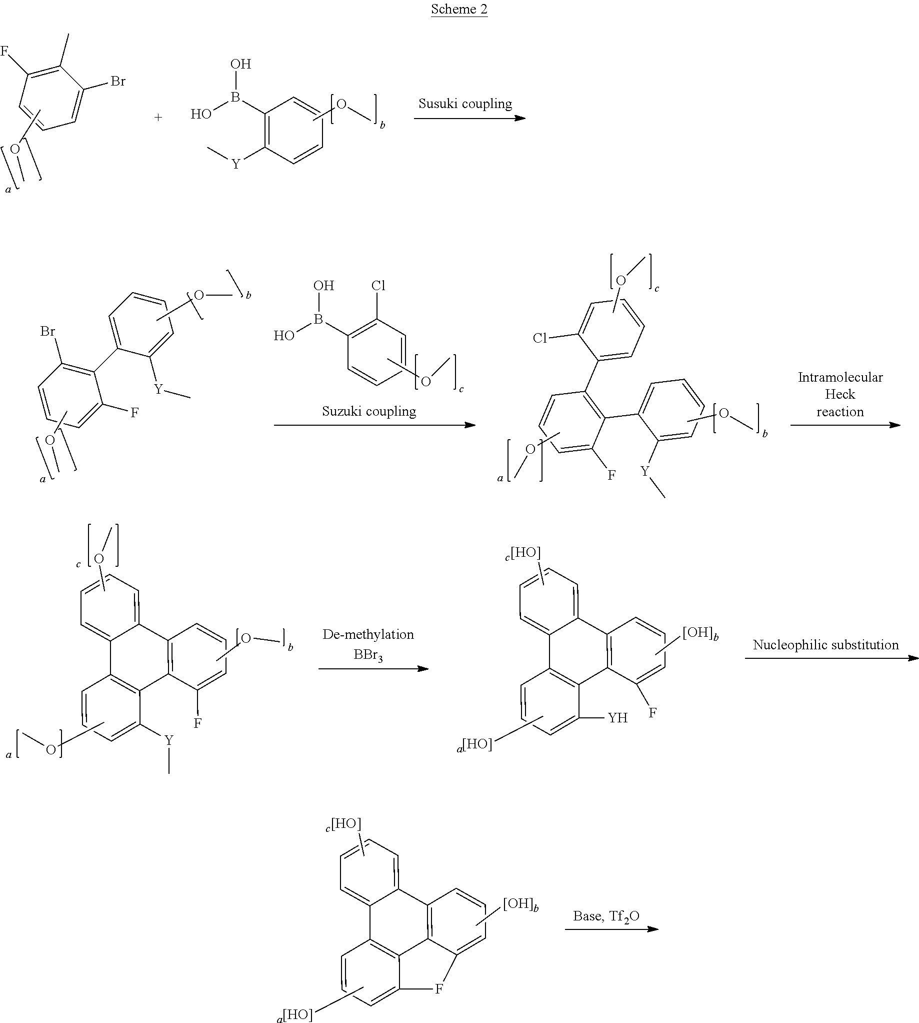

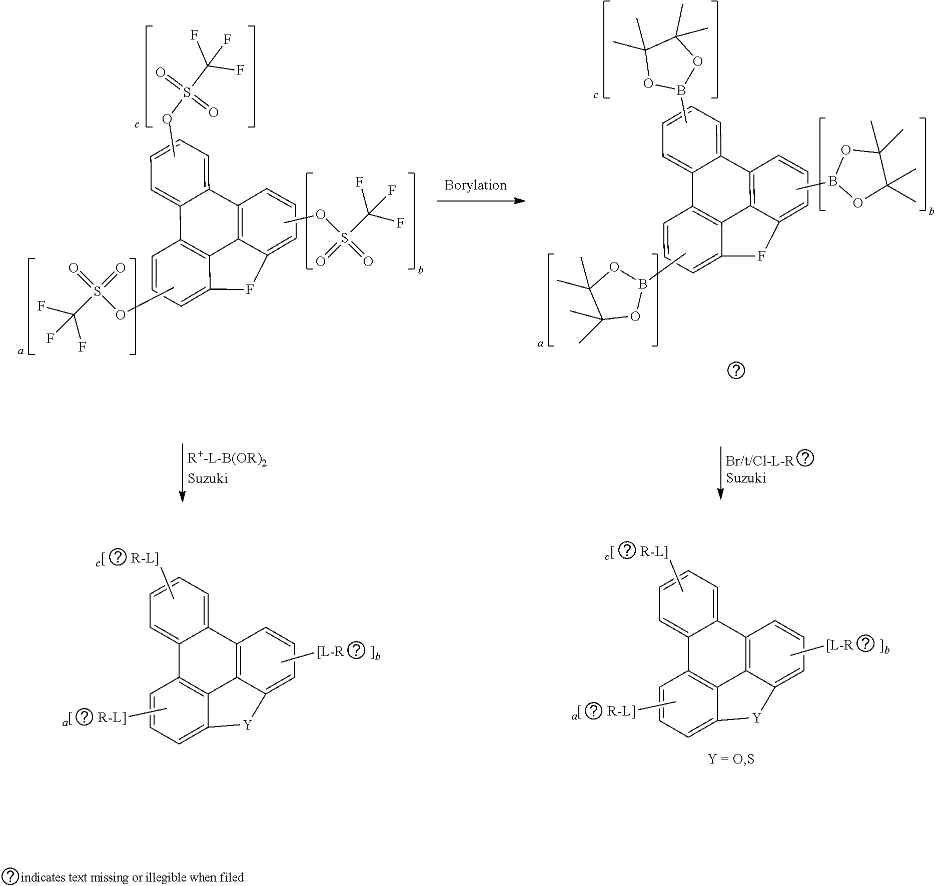

The present invention therefore further provides a process for preparing the compounds of the invention, characterized by the following steps:
(a) synthesizing the base skeleton that bears, in place of the substituent R*, a reactive leaving group, especially Cl, Br, I, triflate or a boronic acid derivative, and
(b) introducing the substituent R* by a coupling reaction.

For the processing of the compounds of formula (1) or the preferred embodiments from the liquid phase, for example by spin-coating or by printing methods, formulations of the compounds of the invention are required. The present invention therefore further provides formulations containing at least one compound of formula (1) or the preferred embodiments and at least one solvent. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The compounds of the formula (1) or the above-detailed preferred embodiments are used in accordance with the invention in an electronic device, especially in an organic electroluminescent device. The present invention therefore further provides for the use of the compounds of formula (1) or the preferred embodiments in an electronic device, especially in an OLED.

The present invention still further provides an electronic device, especially in organic electroluminescent device, comprising at least one compound of the invention. An electronic device in the context of the present invention is a device comprising at least one layer comprising at least one organic compound. This component may also comprise inorganic materials or else layers formed entirely from inorganic materials.

The electronic device is preferably selected from the group consisting of organic electroluminescent devices (OLEDs), organic integrated circuits (O-ICs), organic field-effect transistors (O-FETs), organic thin-film transistors (O-TFTs), organic light-emitting transistors (O-LETs), organic solar cells (O-SCs), dye-sensitized organic solar cells (DSSCs), organic optical detectors, organic photoreceptors, organic field-quench devices (O-FQDs), light-emitting electrochemical cells (LECs), organic laser diodes (O-lasers) and organic plasmon emitting devices, but preferably organic electroluminescent devices (OLEDs), more preferably phosphorescent OLEDs.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem OLED, especially for white-emitting OLEDs.

The compound according to the above-detailed embodiments may be used in different layers, according to the exact structure. Preference is given to an organic electroluminescent device comprising a compound of formula (1) or the above-recited preferred embodiments in an emitting layer as matrix material for phosphorescent or fluorescent emitters or for emitters that exhibit TADF (thermally activated delayed fluorescence), especially as matrix material for phosphorescent emitters. In this case, the organic electroluminescent device may contain an emitting layer, or it may contain a plurality of emitting layers, where at least one emitting layer contains at least one compound of the invention as matrix material. In addition, the compound of the invention may also be used in an electron transport layer and/or in a hole blocker layer.

When the compound is used as matrix material for a phosphorescent compound in an emitting layer, it is preferably used in combination with one or more phosphorescent materials (triplet emitters). Phosphorescence in the context of this invention is understood to mean luminescence from an excited state having higher spin multiplicity, i.e. a spin state>1, especially from an excited triplet state. In the context of this application, all luminescent complexes with transition metals or lanthanides, especially all iridium, platinum and copper complexes, shall be regarded as phosphorescent compounds.

The mixture of the compound of the formula (1) or of the preferred embodiments and the emitting compound contains between 99% and 1% by volume, preferably between 98% and 10% by volume, more preferably between 97% and 60% by volume and especially between 95% and 80% by volume of the compound of the formula (1) or of the preferred embodiments, based on the overall mixture of emitter and matrix material. Correspondingly, the mixture contains between 1% and 99% by volume, preferably between 2% and 90% by volume, more preferably between 3% and 40% by volume and especially between 5% and 20% by volume of the emitter, based on the overall mixture of emitter and matrix material.

A further preferred embodiment of the present invention is the use of the compound of the formula (1) or of the preferred embodiments as matrix material for a phosphorescent emitter in combination with a further matrix material. Suitable matrix materials which can be used in combination with the inventive compounds are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or the carbazole derivatives disclosed in WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, or dibenzofuran derivatives, for example according to WO 2015/169412, WO 2016/015810, WO 2016/023608, WO 2017/148564 or WO 2017/148565. It is likewise possible for a further phosphorescent emitter having shorter-wavelength emission than the actual emitter to be present as co-host in the mixture, or a compound not involved in charge transport to a significant extent, if at all, as described, for example, in WO 2010/108579.

In a preferred embodiment of the invention, the materials are used in combination with a further matrix material. The compounds of the formula (1) or the preferred embodiments are electron-deficient compounds. Preferred co-matrix materials are therefore hole-transporting compounds that are preferably selected from the group of the arylamine or carbazole derivatives.

Depicted below are examples of compounds that are suitable as co-matrix materials together with the compounds of the invention.

Preferred biscarbazoles are the structures of the following formulae (19) to (25):

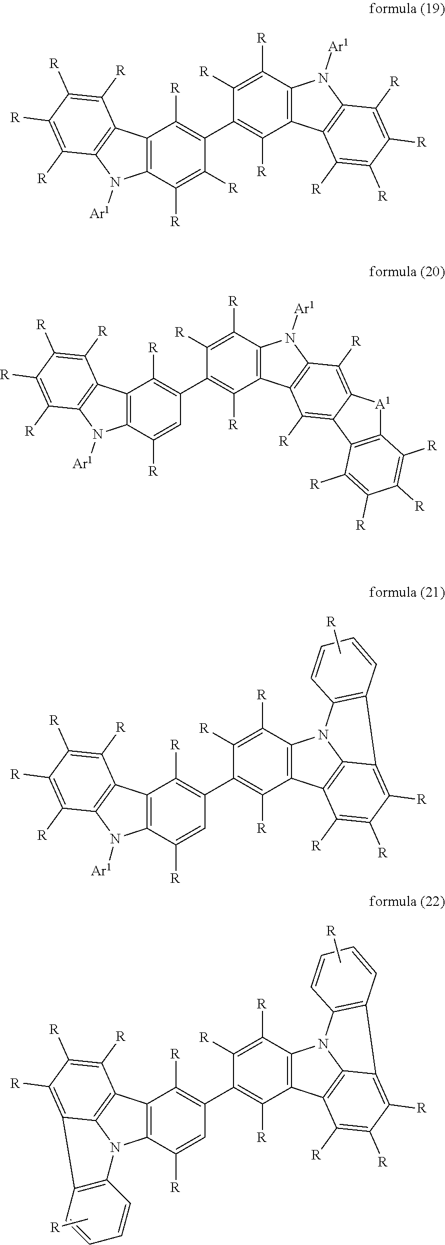

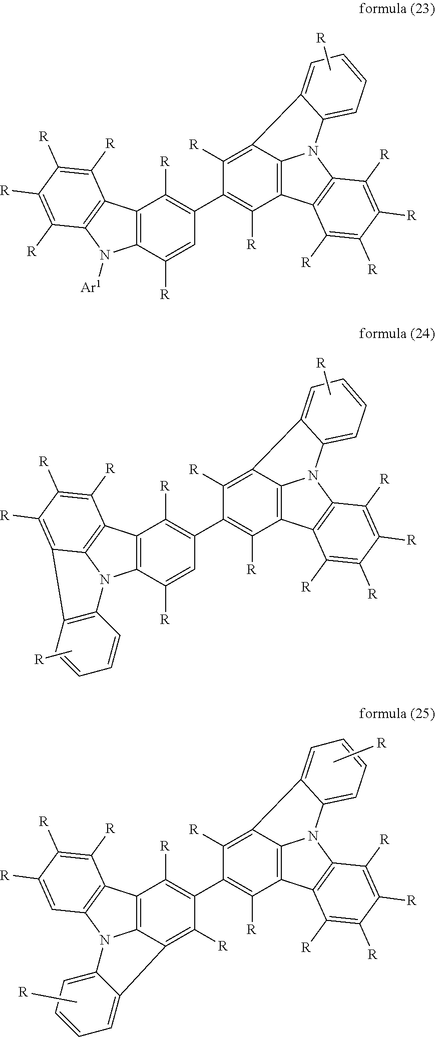

where A¹ has the definitions given above and Ar¹ if the same or different at each instance and is selected from an aromatic heteroaromatic ring system which has 5 to 40 aromatic ring atoms and may be substituted by one or more R¹ radicals. In a preferred embodiment of the invention, A¹ is CR₂. Preferred embodiments of Ar¹ are the preferred structures listed above for aromatic or heteroaromatic R radicals, especially the groups (R-1) to (R-83).

Preferred embodiments of the compounds of the formulae (19) to (25) are the compounds of the following formulae (19a) to (25a):

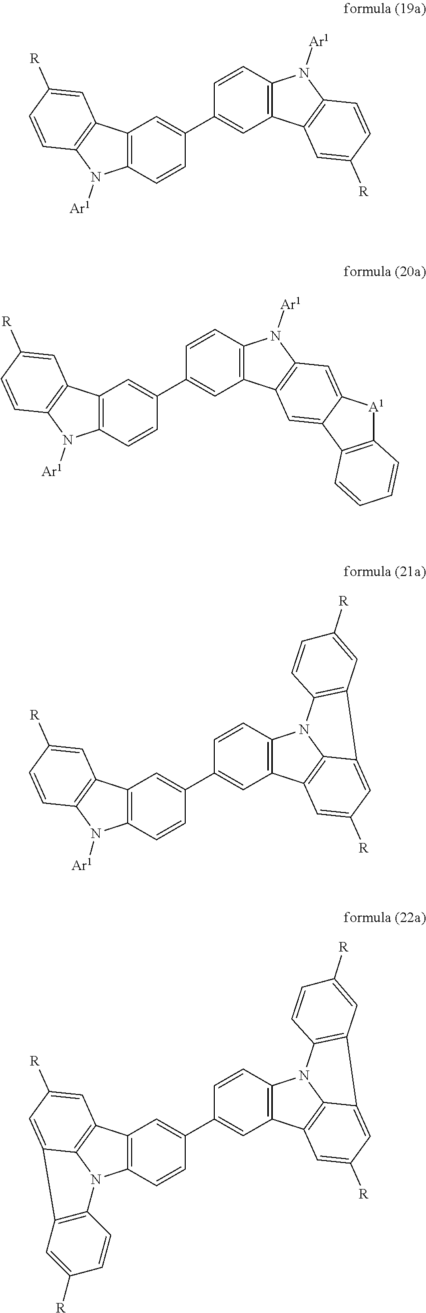

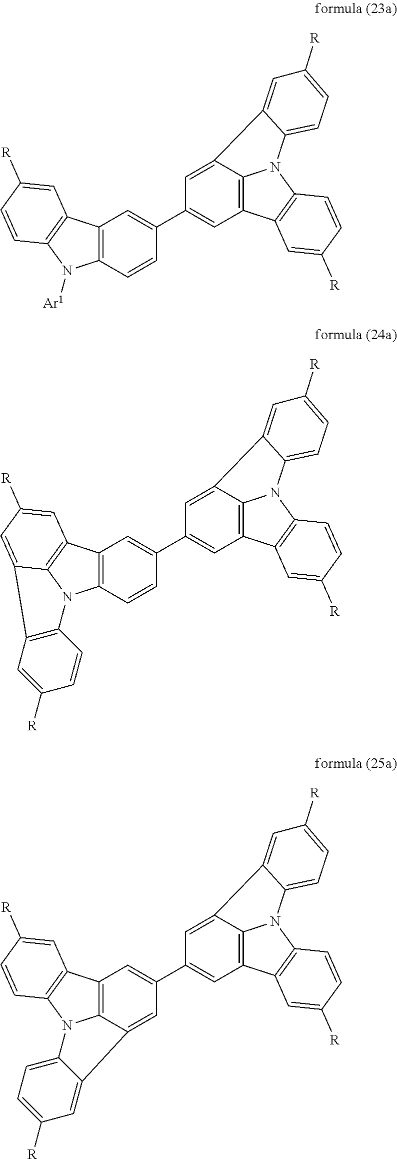

where the symbols used have the definitions given above.

Examples of suitable compounds of formulae (19) to (25) are the compounds depicted below:

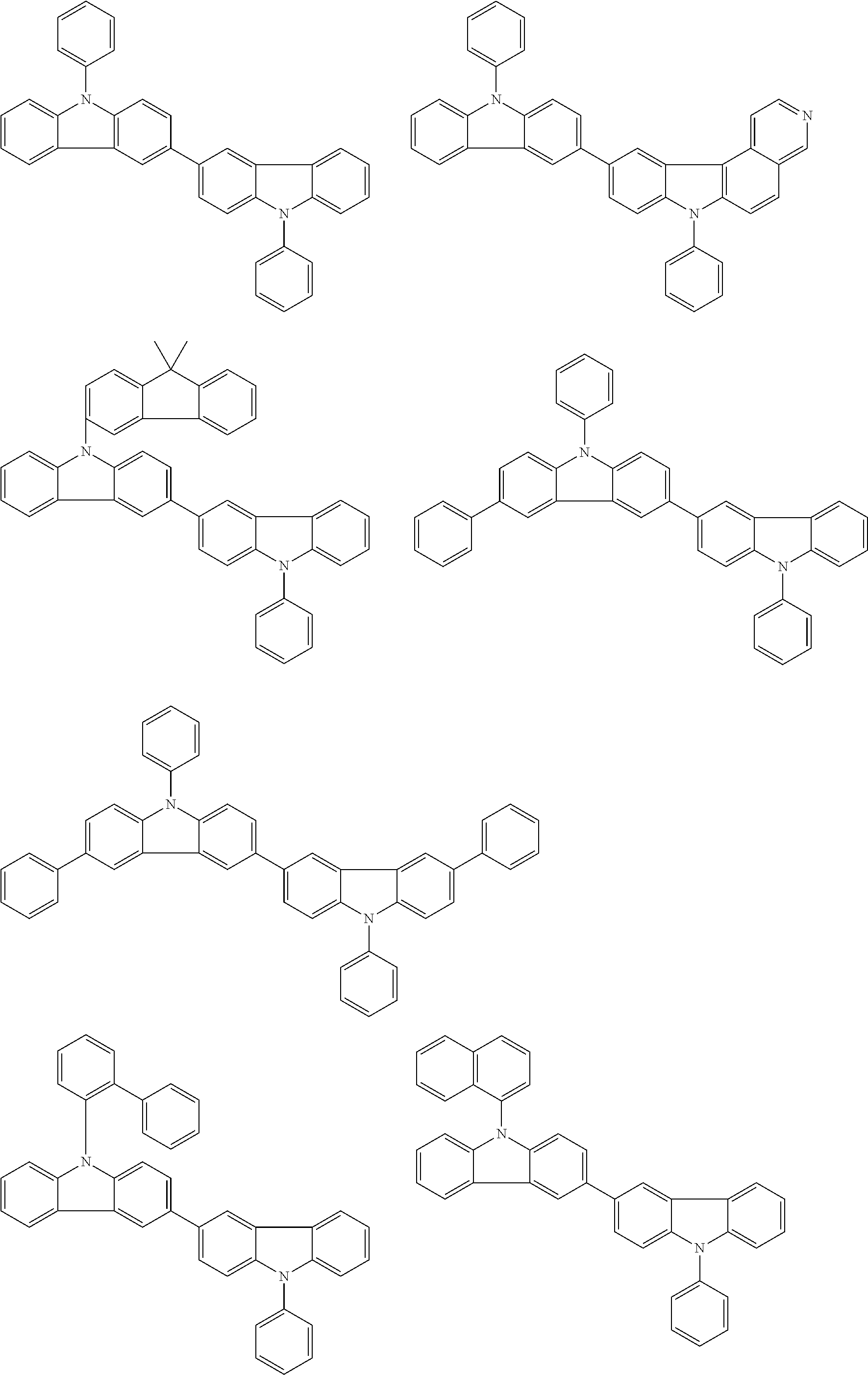

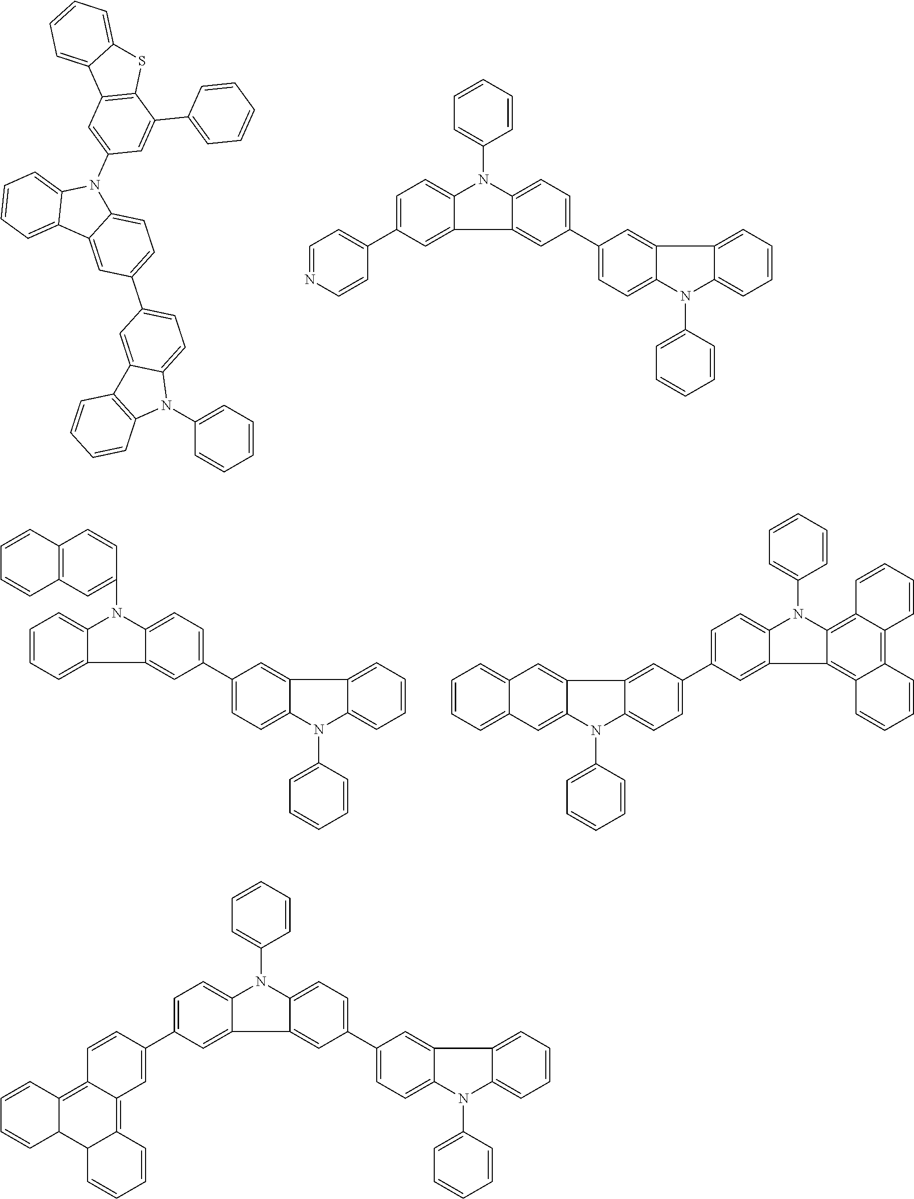

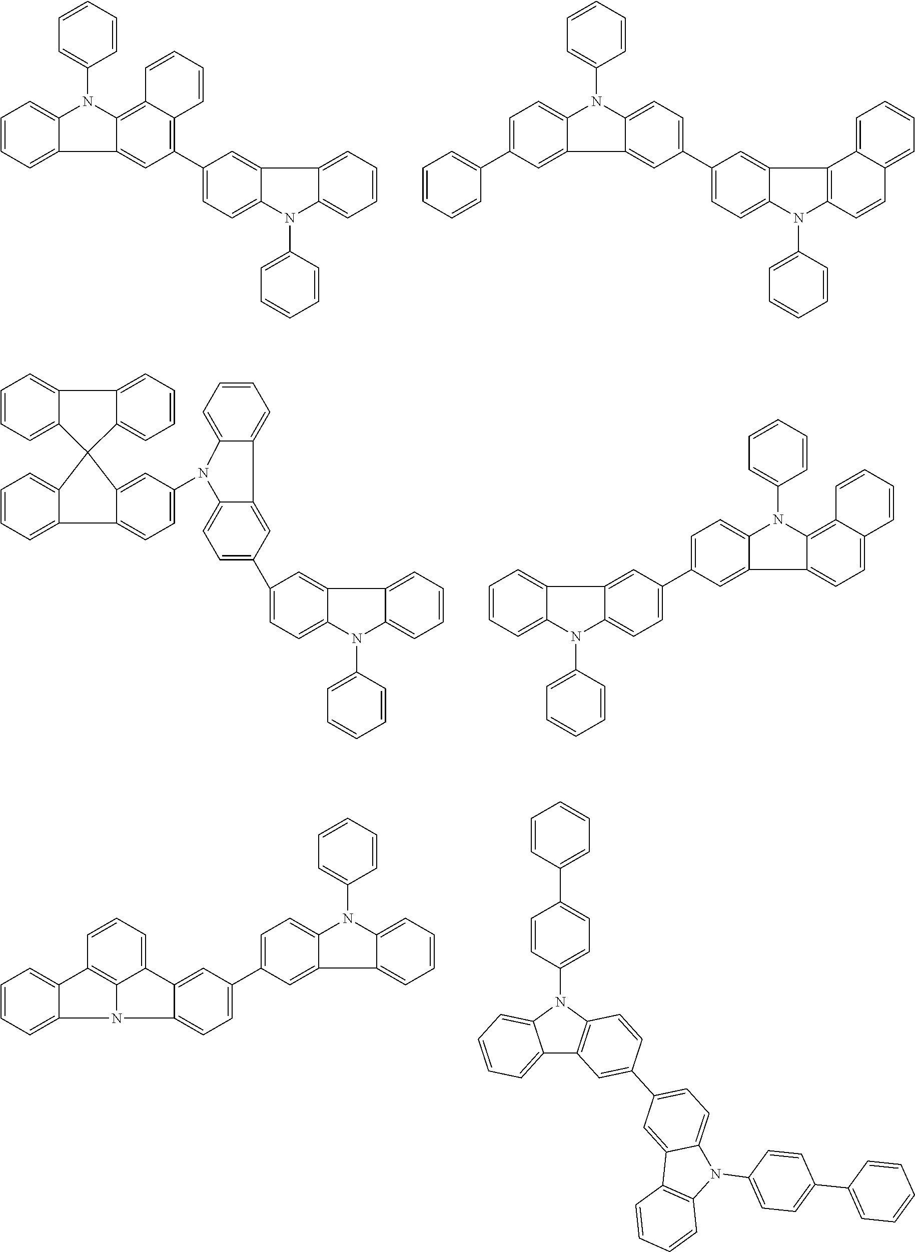

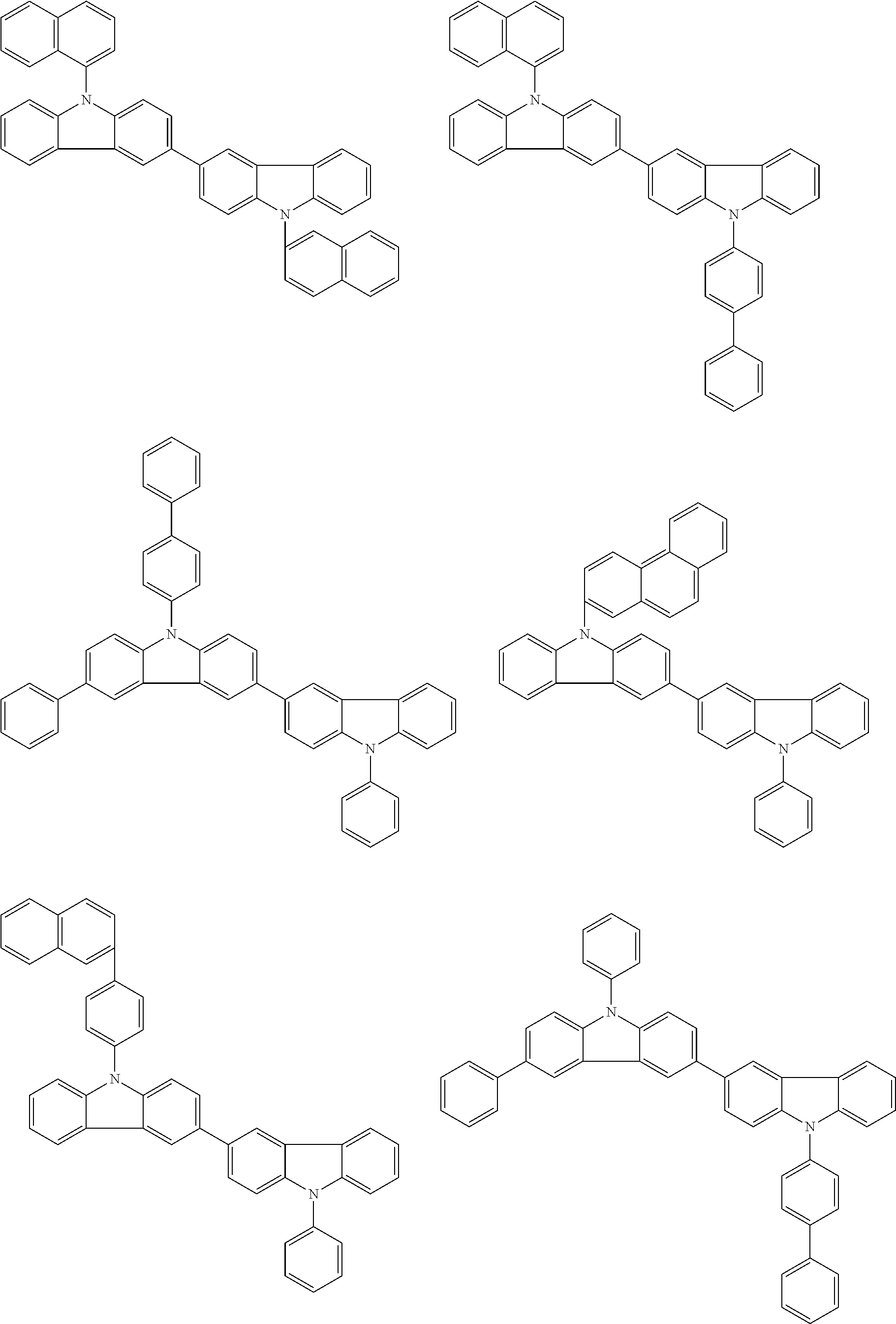 [SMILES: c1ccc2cc(-n3c4ccccc4c4cc(-c5ccc6c(c5)c5ccccc5n6-c5cccc6ccccc56)ccc43)ccc2c1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc5c(ccc6ccccc65)c4)ccc32)cc1 ; c1ccc(-n2c3ccccc3c3cc(-c4ccc5c(c4)c4ccccc4n5-c4ccc(-c5ccc6ccccc6c5)cc4)ccc32)cc1 ; c1ccc(-c2ccc(-n3c4ccccc4c4cc(-c5ccc6c(c5)c5cc(-c7ccccc7)ccc5n6-c5ccccc5)ccc43)cc2)cc1]

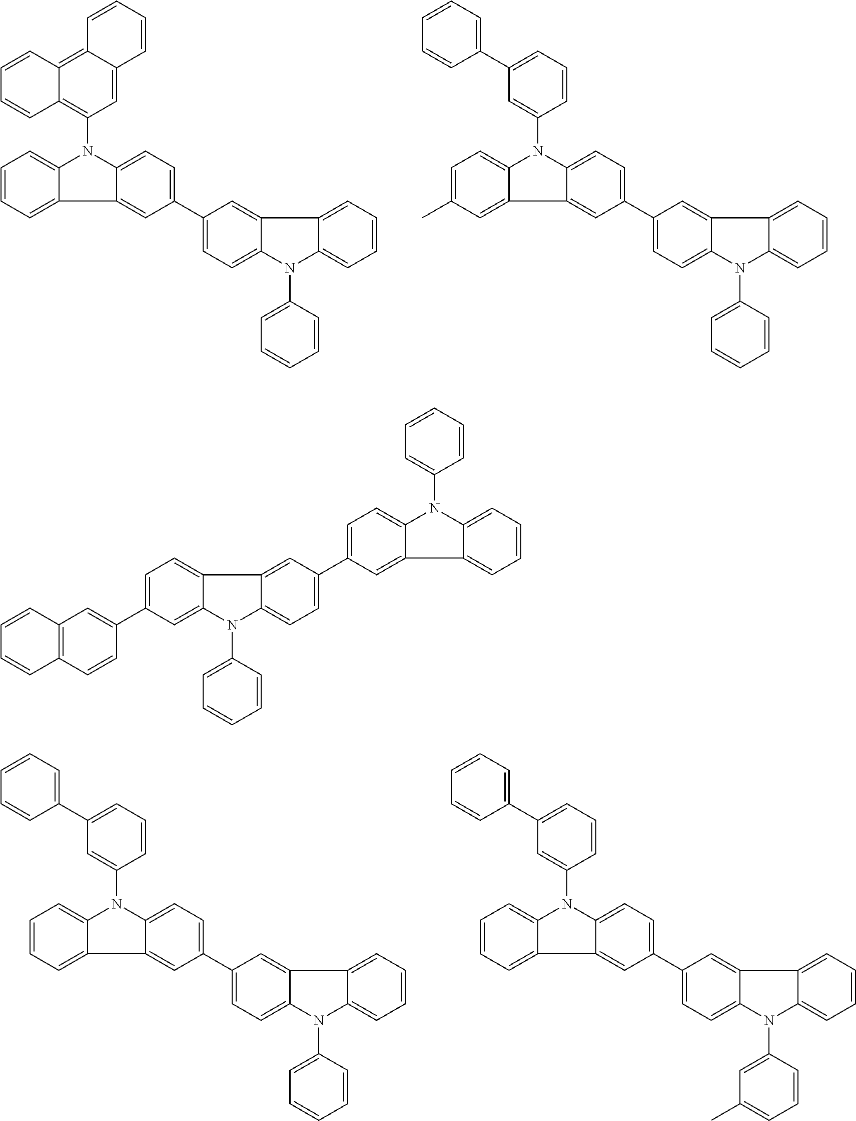

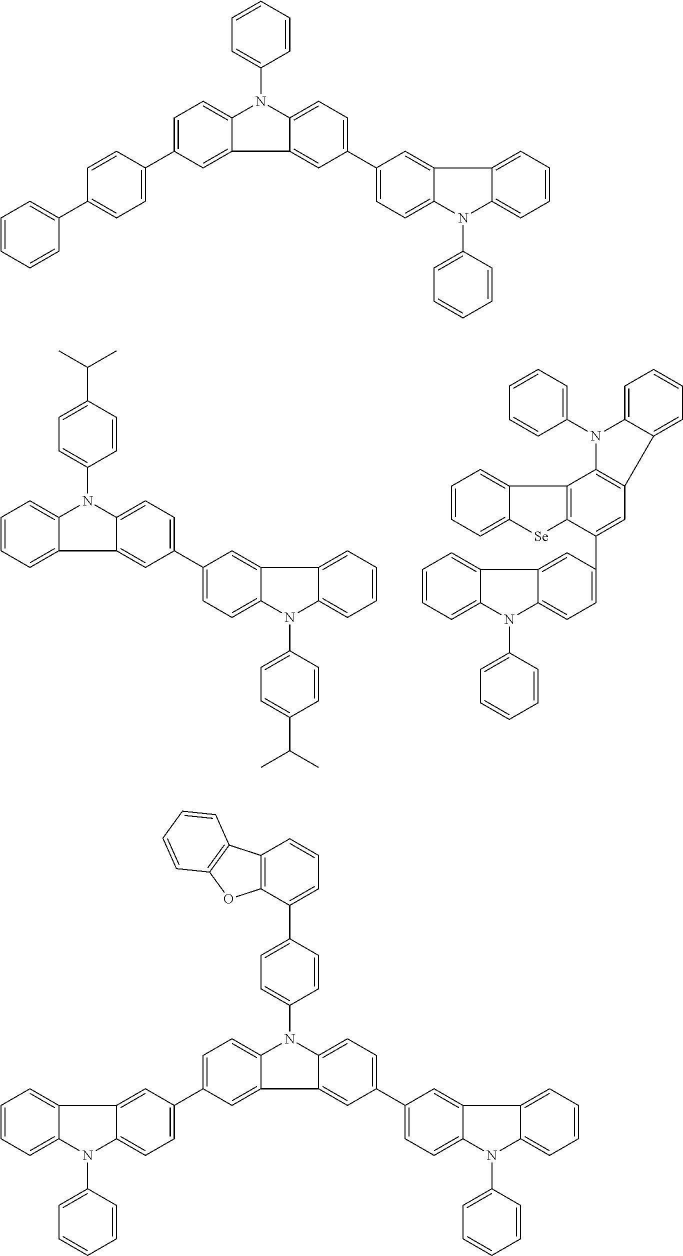

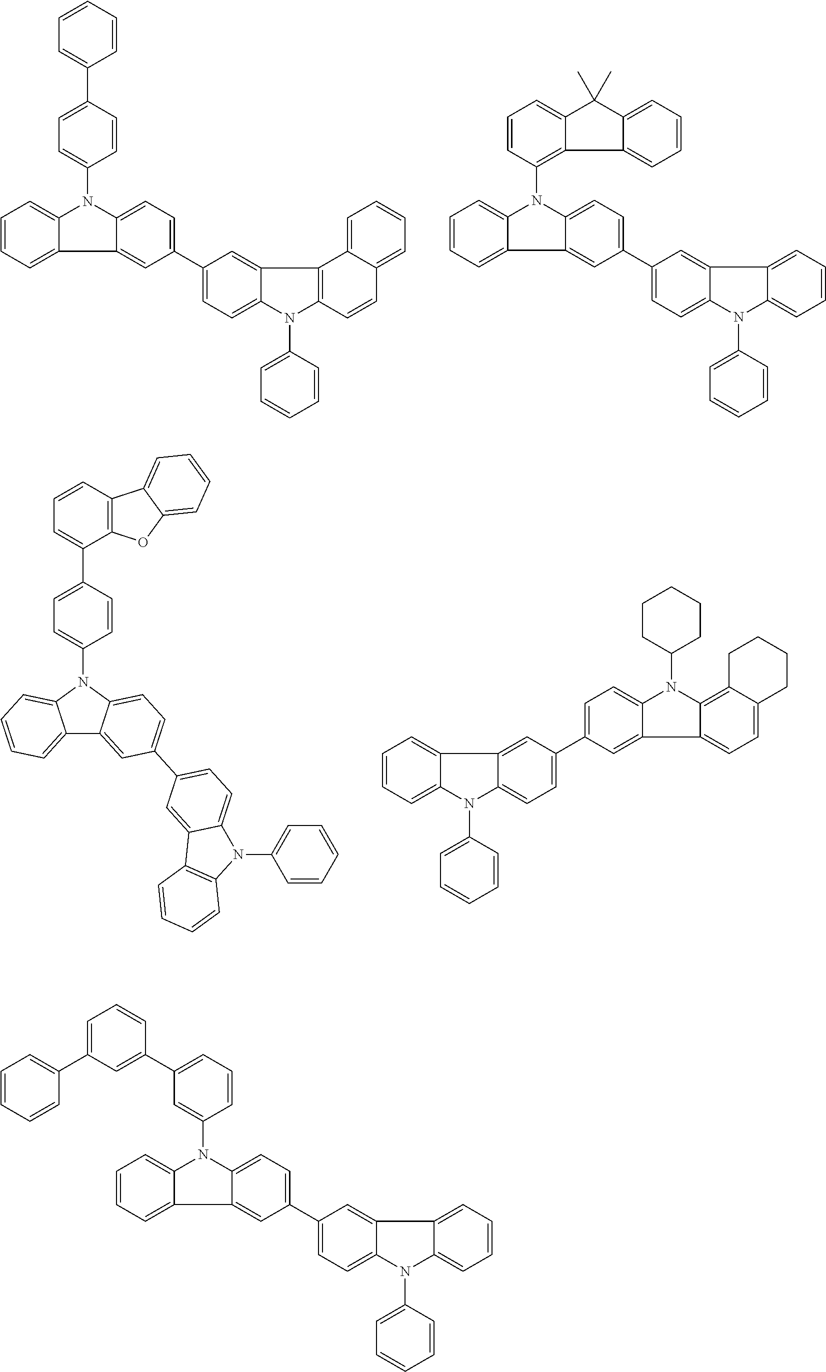

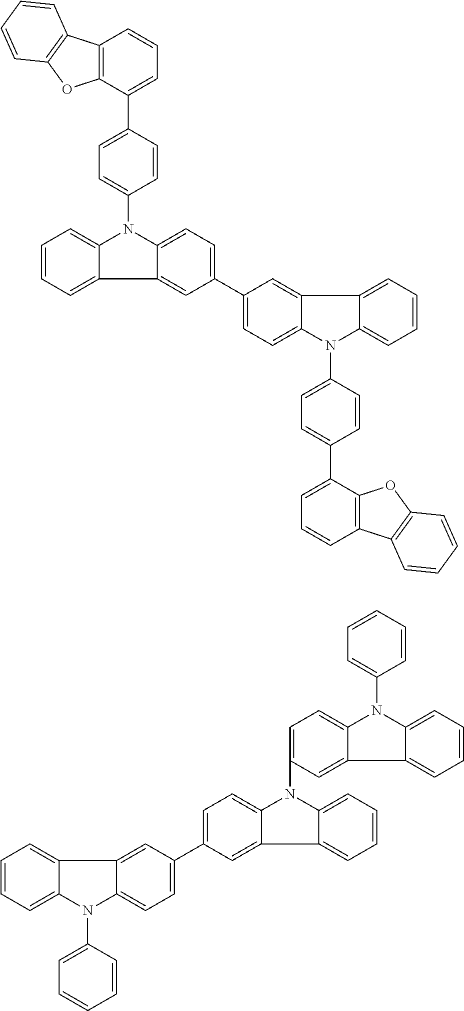 [SMILES: c1ccc2c(c1)oc1c(-c3ccc(-n4c5ccccc5c5cc(-c6ccc7c(c6)c6ccccc6n7-c6ccc(-c7cccc8c7oc7ccccc78)cc6)ccc54)cc3)cccc12 ; c1ccc(-n2c3ccccc3c3cc(-c4ccc5c(c4)c4ccccc4n5-c4ccc5c(c4)c4ccccc4n5-c4ccccc4)ccc32)cc1]

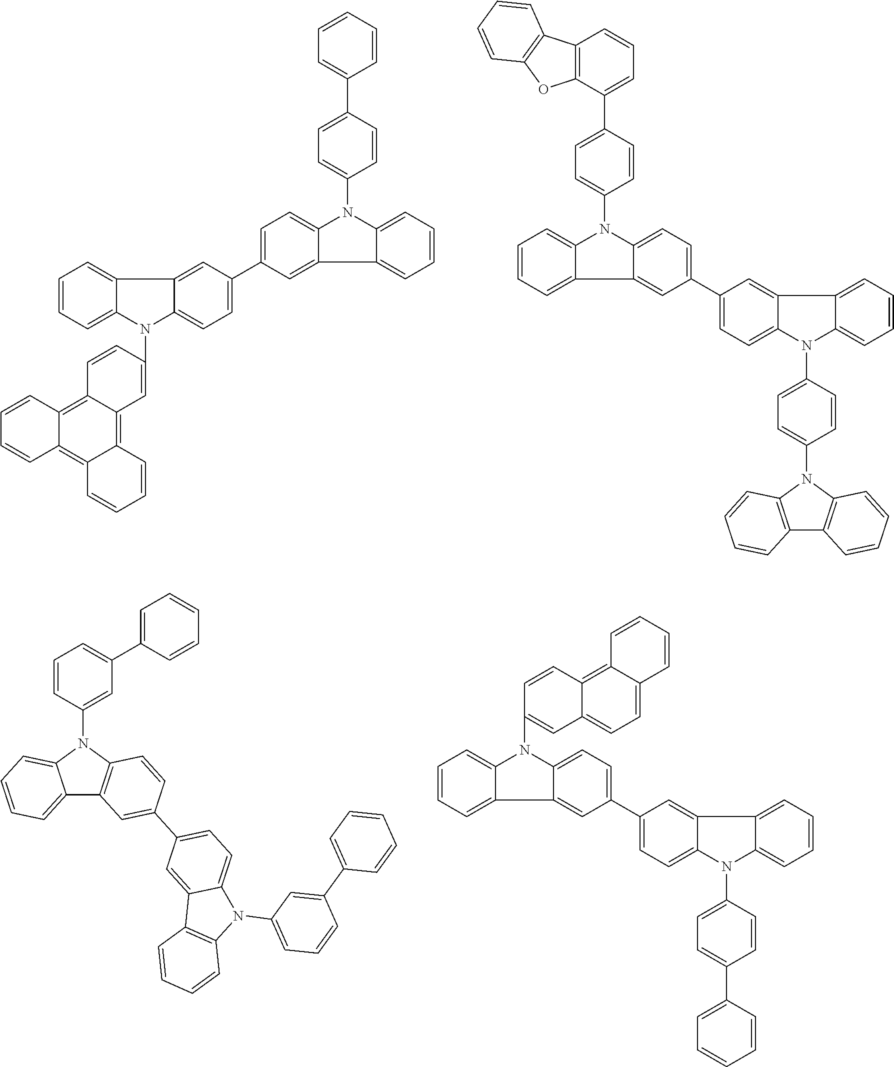 [SMILES: c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; c1ccc2c(c1)oc1c(-c3ccc(-n4c5ccccc5c5cc(-c6ccc7c(c6)c6ccccc6n7-c6ccc(-n7c8ccccc8c8ccccc87)cc6)ccc54)cc3)cccc12 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4cc(-c5ccc6c(c5)c5ccccc5n6-c5ccc6c(ccc7ccccc76)c5)ccc43)cc2)cc1]

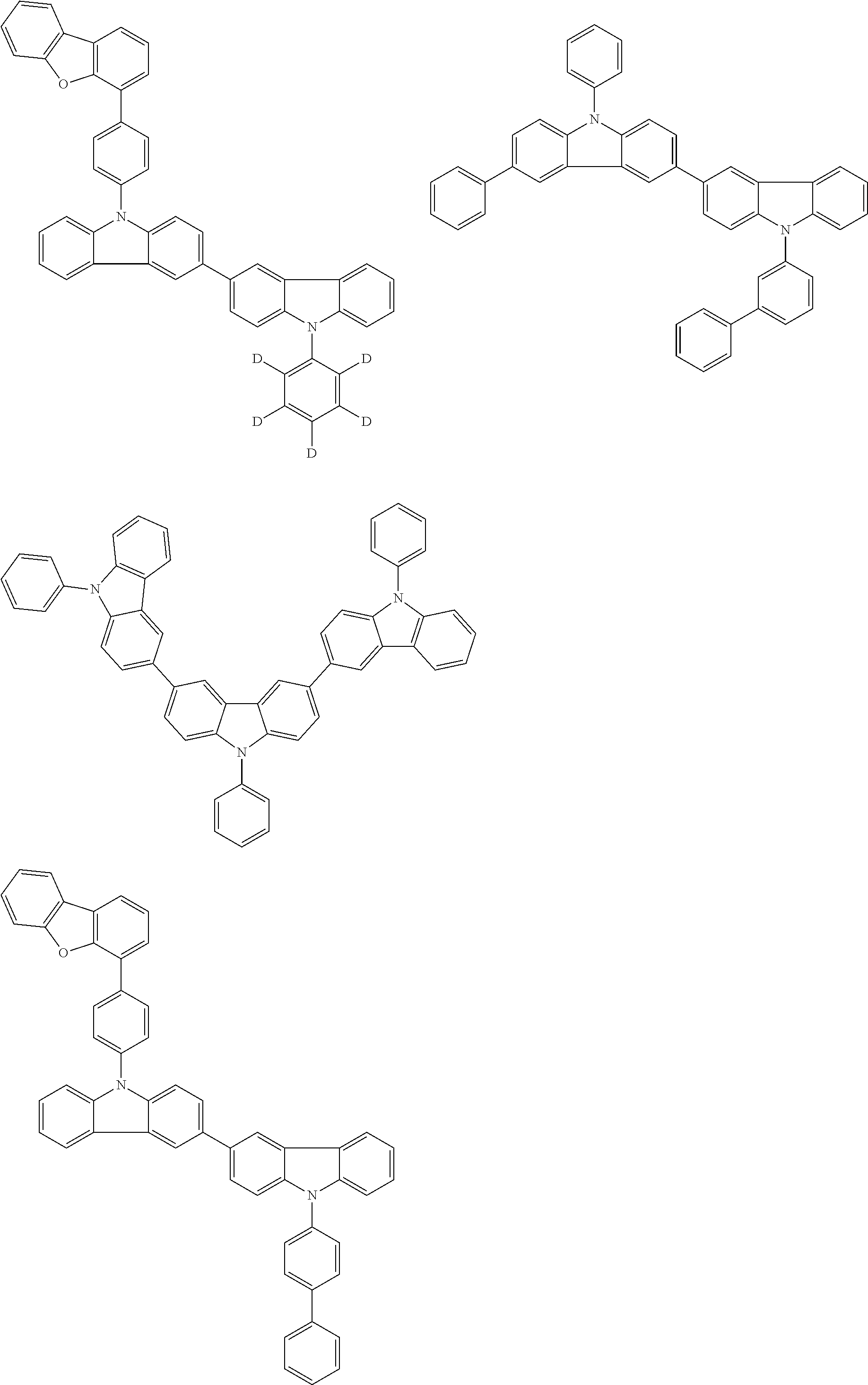 [SMILES: [2H]c1c([2H])c([2H])c(-n2c3ccccc3c3cc(-c4ccc5c(c4)c4ccccc4n5-c4ccc(-c5cccc6c5oc5ccccc56)cc4)ccc32)c([2H])c1[2H] ; c1ccc(-c2cccc(-n3c4ccccc4c4cc(-c5ccc6c(c5)c5cc(-c7ccccc7)ccc5n6-c5ccccc5)ccc43)c2)cc1 ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-c2ccc(-n3c4ccccc4c4cc(-c5ccc6c(c5)c5ccccc5n6-c5ccc(-c6cccc7c6oc6ccccc67)cc5)ccc43)cc2)cc1]

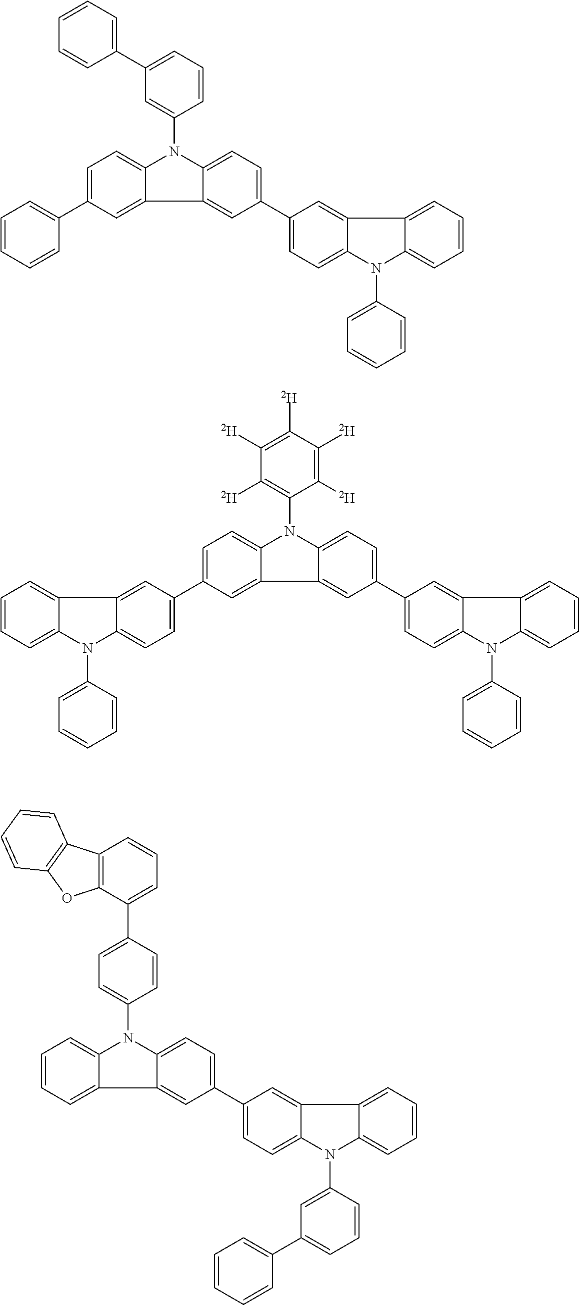 [SMILES: c1ccc(-c2cccc(-n3c4ccc(-c5ccccc5)cc4c4cc(-c5ccc6c(c5)c5ccccc5n6-c5ccccc5)ccc43)c2)cc1 ; [2H]c1c([2H])c([2H])c(-n2c3ccc(-c4ccc5c(c4)c4ccccc4n5-c4ccccc4)cc3c3cc(-c4ccc5c(c4)c4ccccc4n5-c4ccccc4)ccc32)c([2H])c1[2H] ; c1ccc(-c2cccc(-n3c4ccccc4c4cc(-c5ccc6c(c5)c5ccccc5n6-c5ccc(-c6cccc7c6oc6ccccc67)cc5)ccc43)c2)cc1]

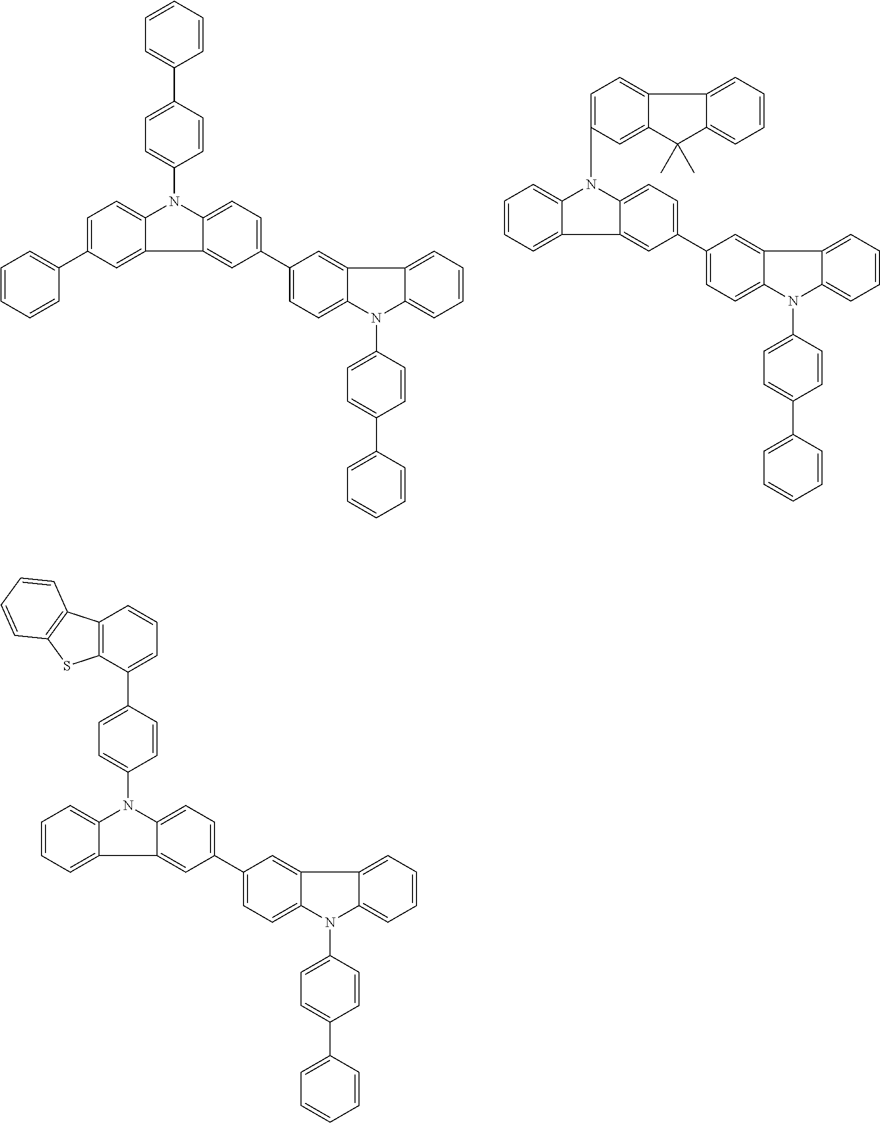 [SMILES: c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1]

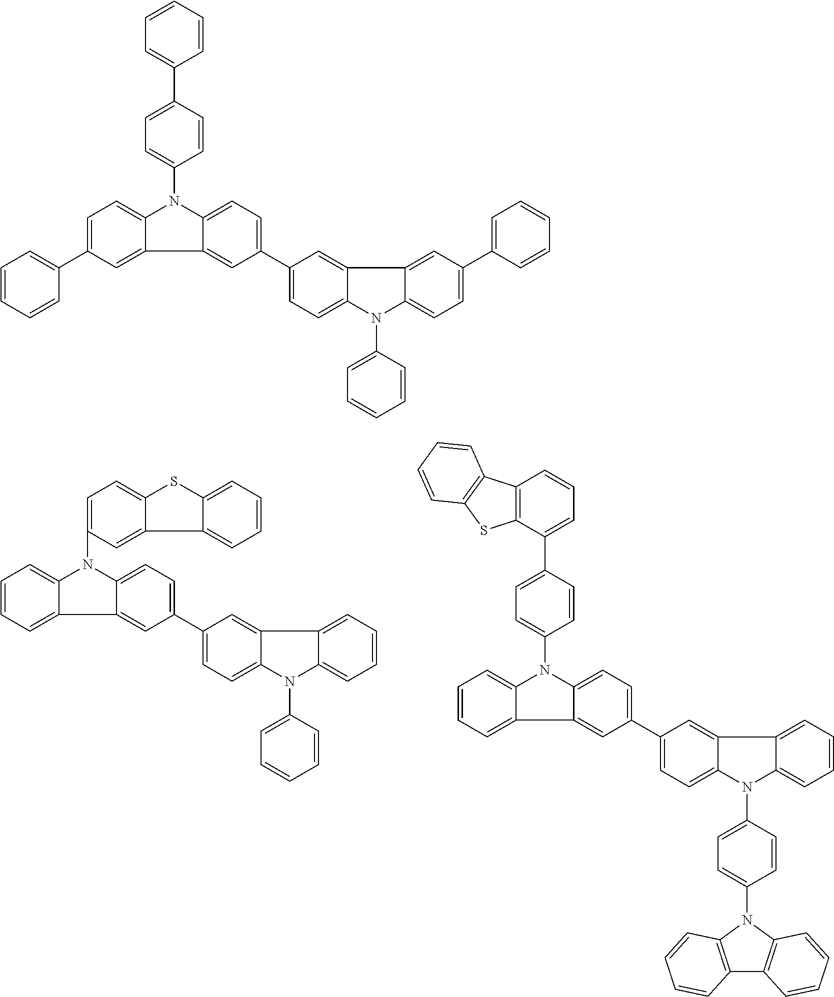 [SMILES: c1ccc(-c2ccc(-n3c4ccc(-c5ccccc5)cc4c4cc(-c5ccc6c(c5)c5cc(-c7ccccc7)ccc5n6-c5ccccc5)ccc43)cc2)cc1 ; c1ccc(-n2c3ccccc3c3cc(-c4ccc5c(c4)c4ccccc4n5-c4ccc5sc6ccccc6c5c4)ccc32)cc1 ; c1ccc2c(c1)sc1c(-c3ccc(-n4c5ccccc5c5cc(-c6ccc7c(c6)c6ccccc6n7-c6ccc(-n7c8ccccc8c8ccccc87)cc6)ccc54)cc3)cccc12]

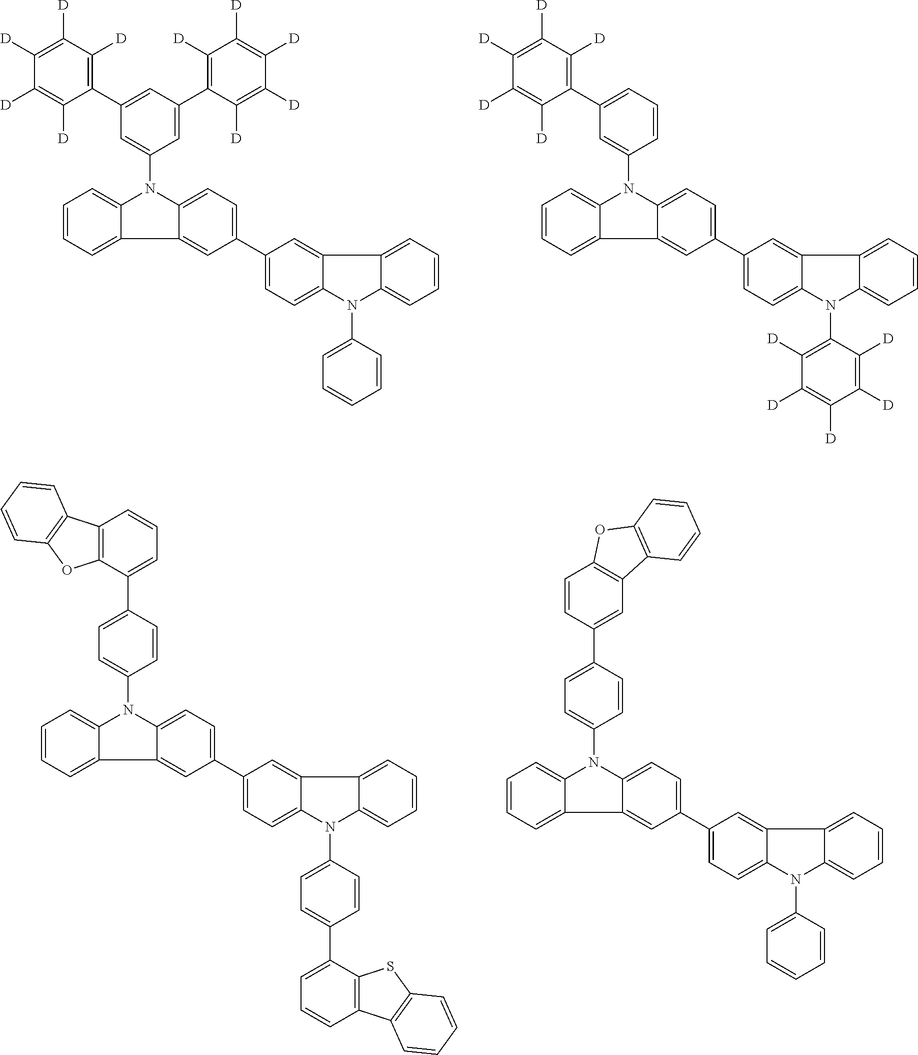 [SMILES: [2H]c1c([2H])c([2H])c(-c2cc(-c3c([2H])c([2H])c([2H])c([2H])c3[2H])cc(-n3c4ccccc4c4cc(-c5ccc6c(c5)c5ccccc5n6-c5ccccc5)ccc43)c2)c([2H])c1[2H] ; [2H]c1c([2H])c([2H])c(-c2cccc(-n3c4ccccc4c4cc(-c5ccc6c(c5)c5ccccc5n6-c5c([2H])c([2H])c([2H])c([2H])c5[2H])ccc43)c2)c([2H])c1[2H] ; c1ccc2c(c1)oc1c(-c3ccc(-n4c5ccccc5c5cc(-c6ccc7c(c6)c6ccccc6n7-c6ccc(-c7cccc8c7sc7ccccc78)cc6)ccc54)cc3)cccc12 ; c1ccc(-n2c3ccccc3c3cc(-c4ccc5c(c4)c4ccccc4n5-c4ccc(-c5ccc6oc7ccccc7c6c5)cc4)ccc32)cc1]

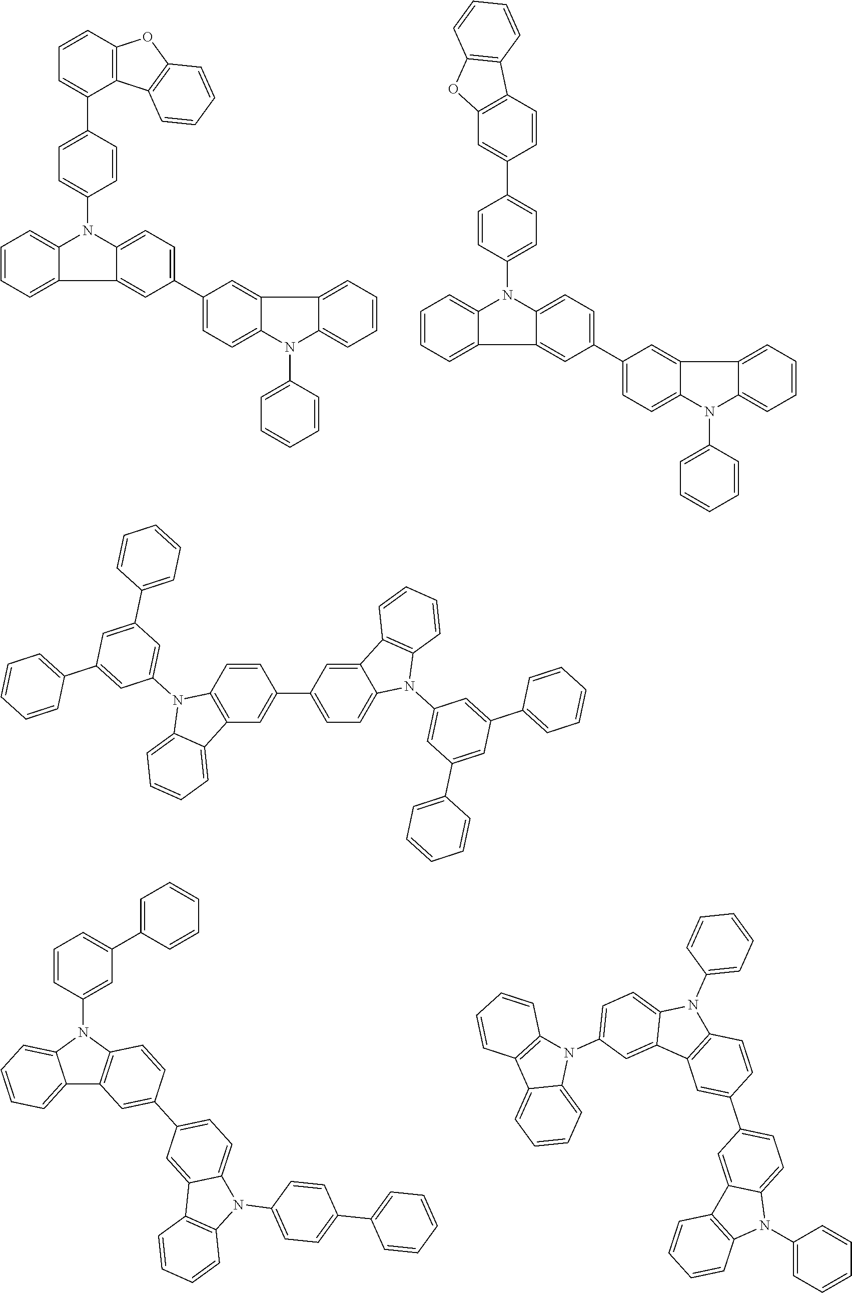

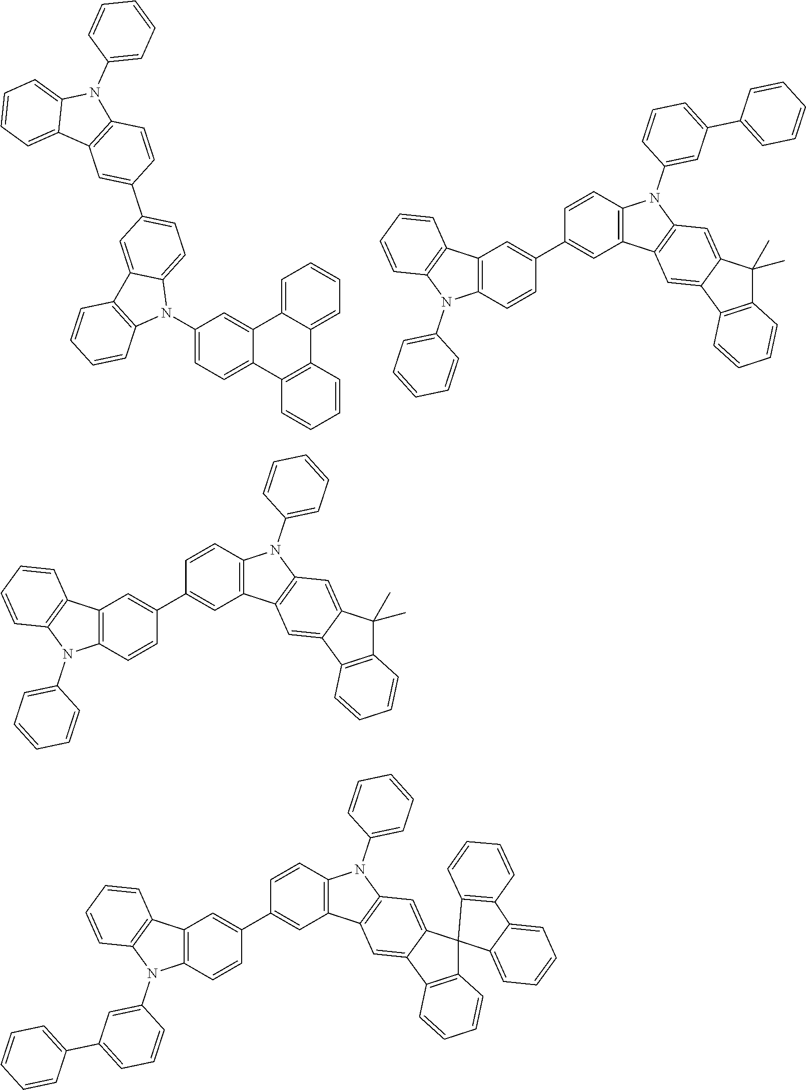

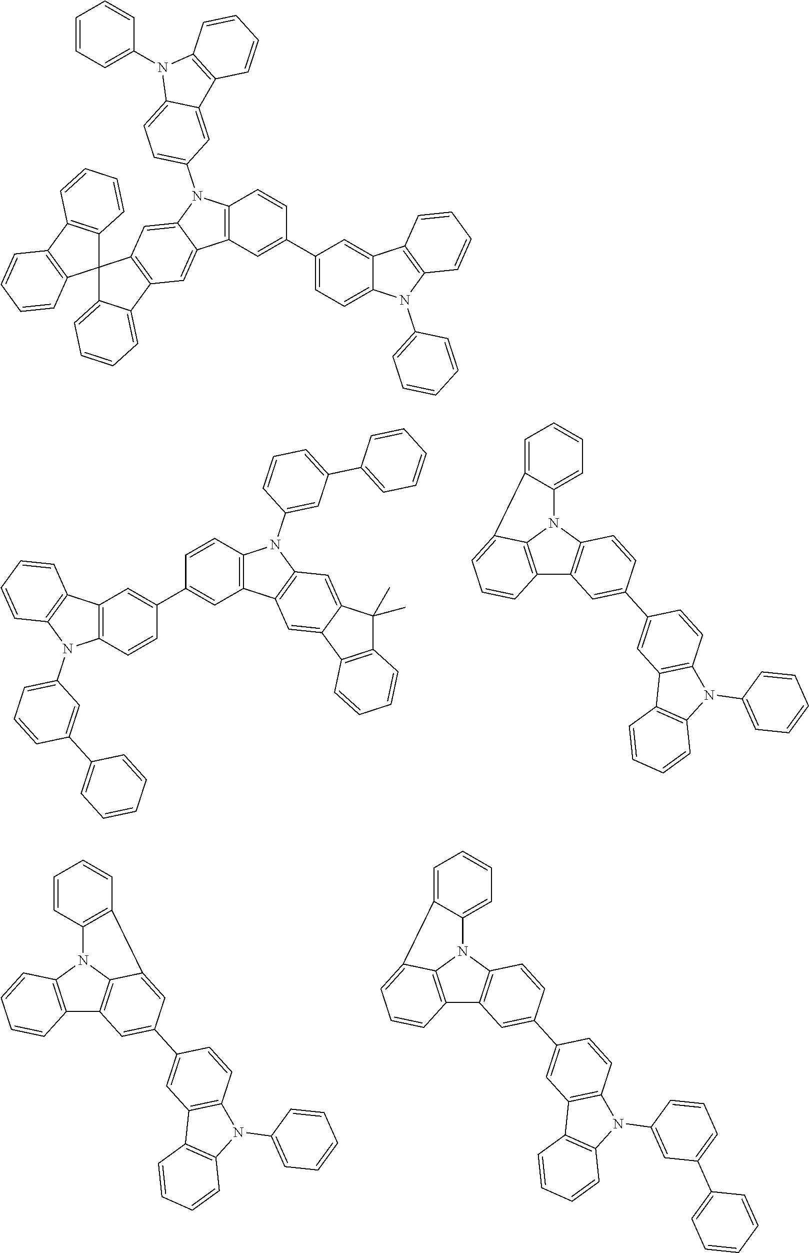

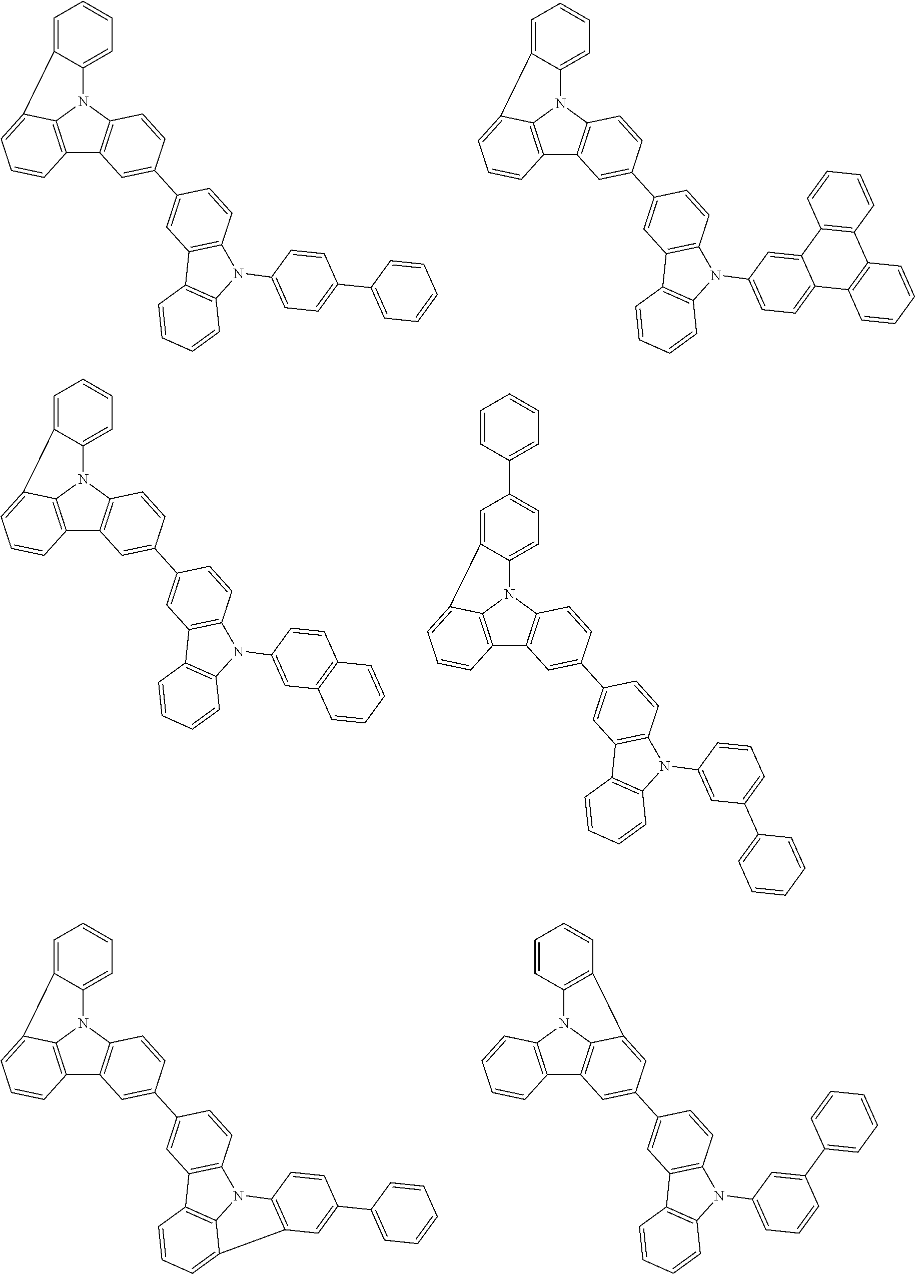 [SMILES: c1ccc(-c2ccc(-n3c4ccccc4c4cc(-c5ccc6c(c5)c5cccc7c8ccccc8n6c75)ccc43)cc2)cc1 ; c1ccc2c(c1)c1ccccc1c1cc(-n3c4ccccc4c4cc(-c5ccc6c(c5)c5cccc7c8ccccc8n6c75)ccc43)ccc21 ; c1ccc2cc(-n3c4ccccc4c4cc(-c5ccc6c(c5)c5cccc7c8ccccc8n6c75)ccc43)ccc2c1 ; c1ccc(-c2cccc(-n3c4ccccc4c4cc(-c5ccc6c(c5)c5cccc7c8cc(-c9ccccc9)ccc8n6c75)ccc43)c2)cc1 ; c1ccc(-c2ccc3c(c2)c2cccc4c5cc(-c6ccc7c(c6)c6cccc8c9ccccc9n7c86)ccc5n3c24)cc1 ; c1ccc(-c2cccc(-n3c4ccccc4c4cc(-c5cc6c7ccccc7n7c8ccccc8c(c5)c67)ccc43)c2)cc1]

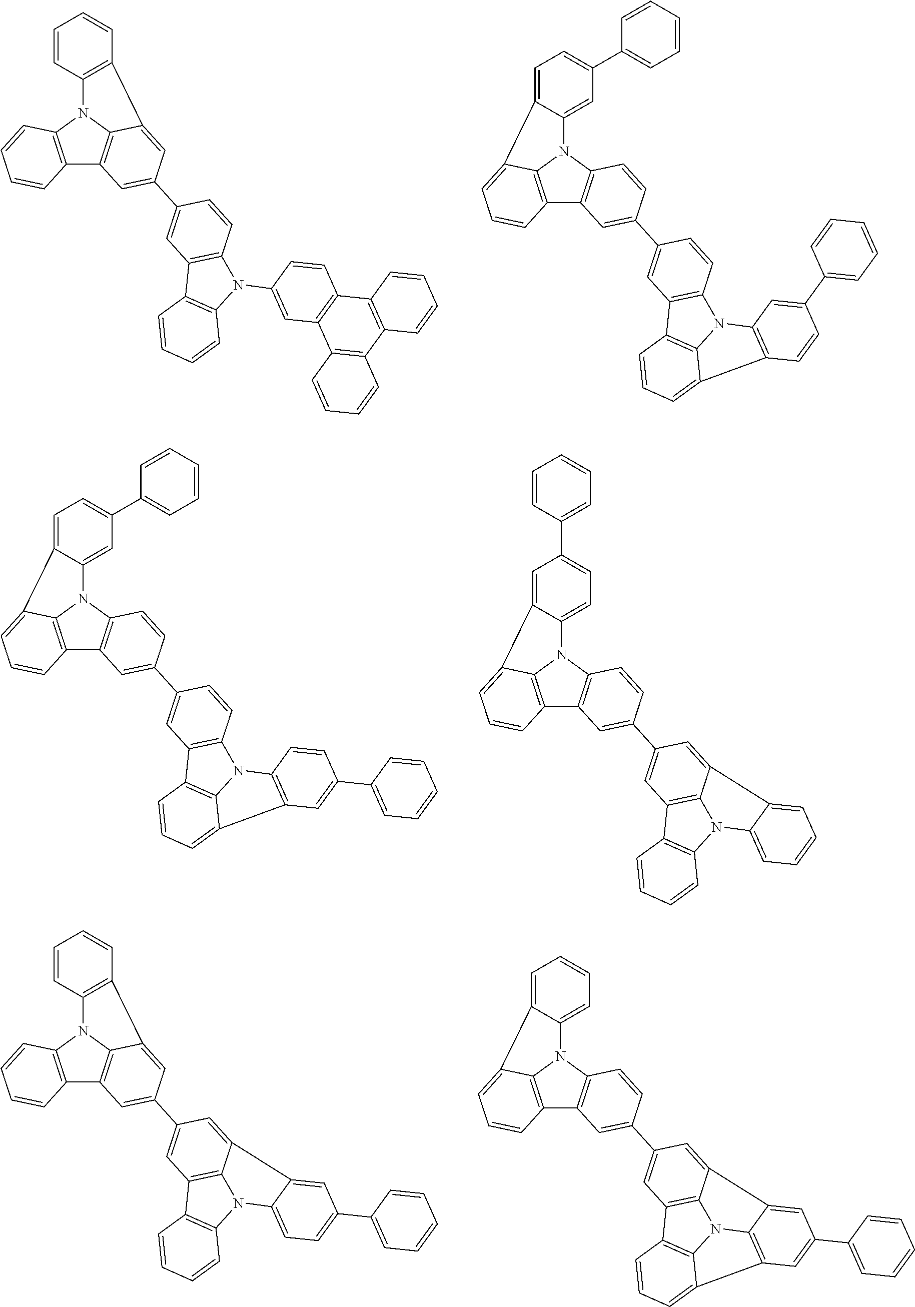

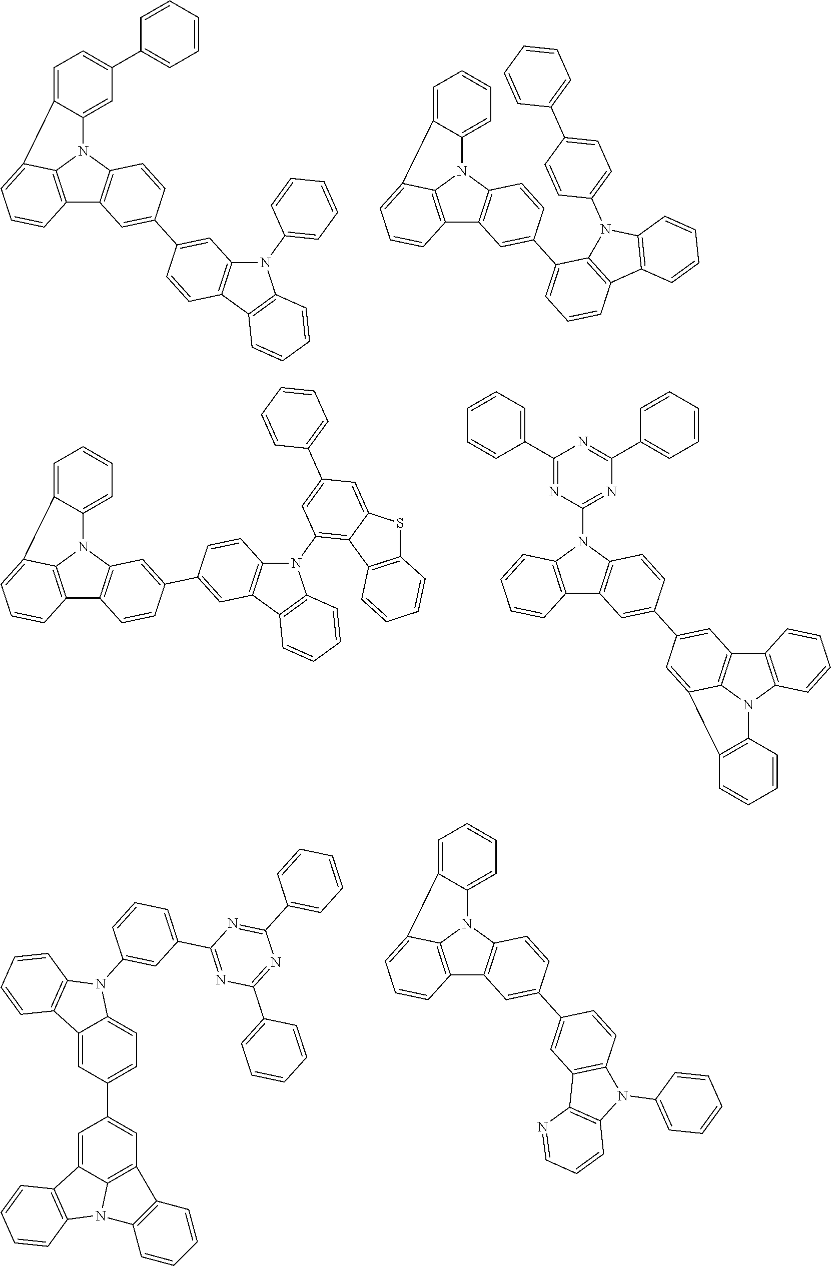

Preferred bridged carbazoles are the structures of the following formula (26):

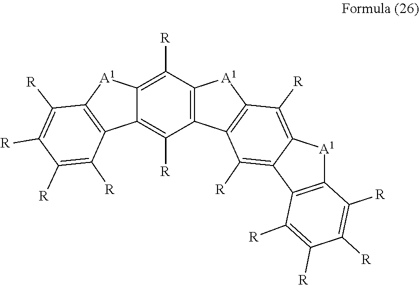

where A¹ and R have the definitions given above and A¹ is preferably the same or different at each instance and is selected from the group consisting of NR¹ where R¹ is an aromatic heteroaromatic ring system which has 5 to 24 aromatic ring atoms and may be substituted by one or more R² radicals, and C(R¹)₂.

Preferred dibenzofuran derivatives are the compounds of the following formula (27):

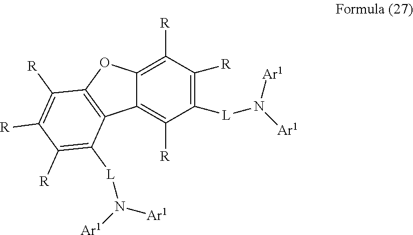

where the oxygen may also be replaced by sulfur, so as to form a dibenzothiophene, and L, R and Ar¹ have the definitions given above. It is also possible here for the two Ar¹ groups that bind to the same nitrogen atom, or for one Ar¹ group and one L group that bind to the same nitrogen atom, to be bonded to one another, for example to give a carbazole.

Examples of suitable dibenzofuran derivatives are the compounds depicted below.

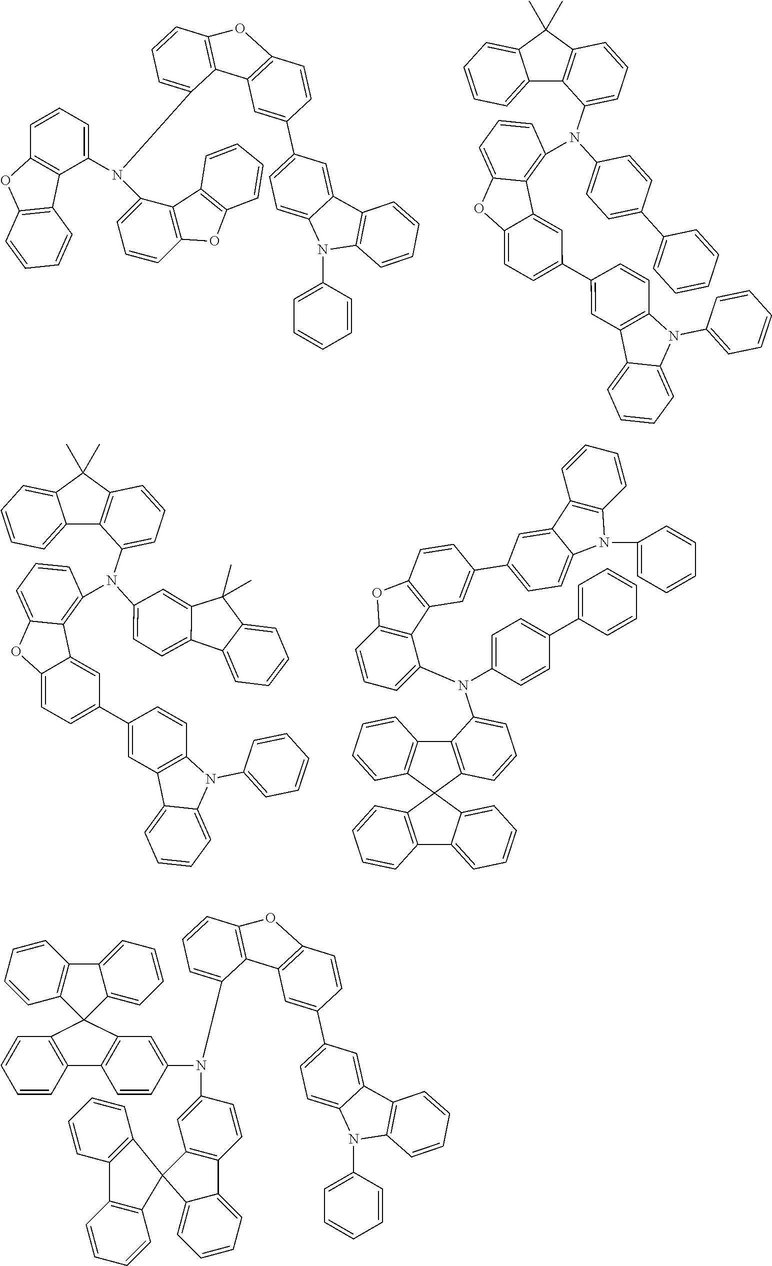

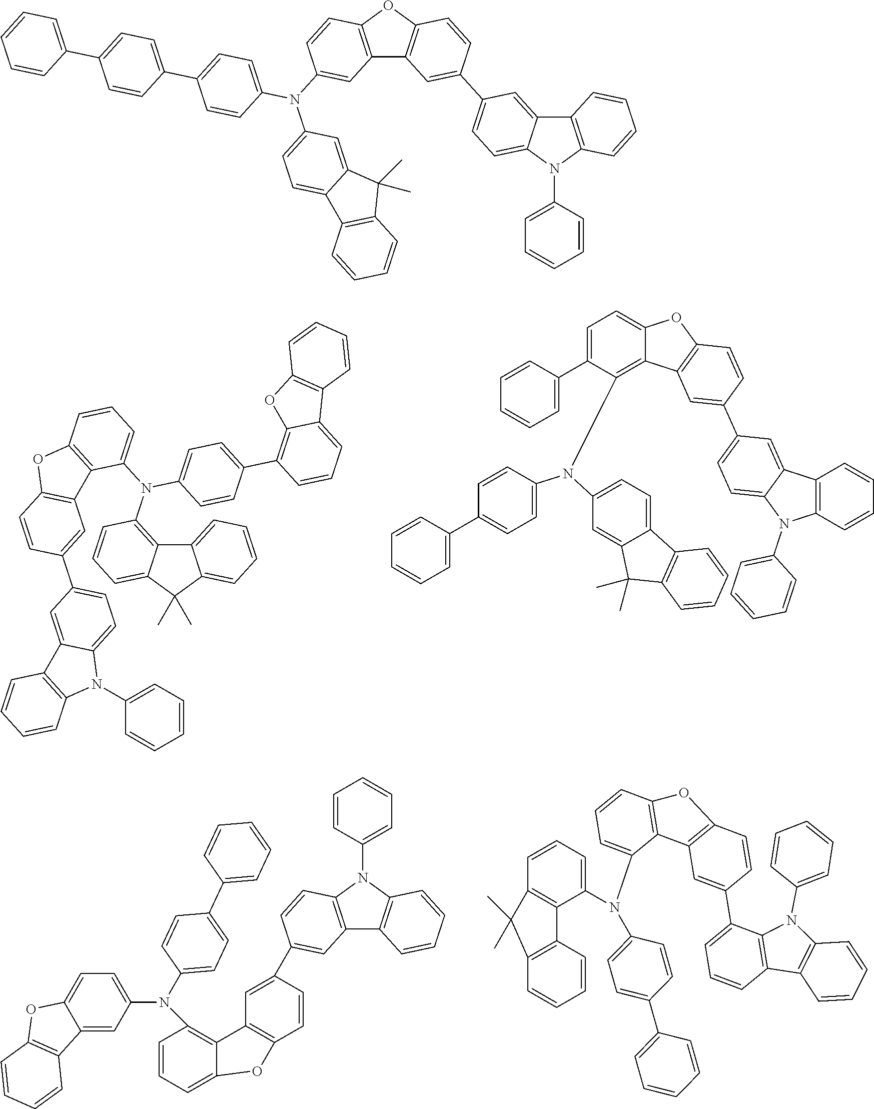

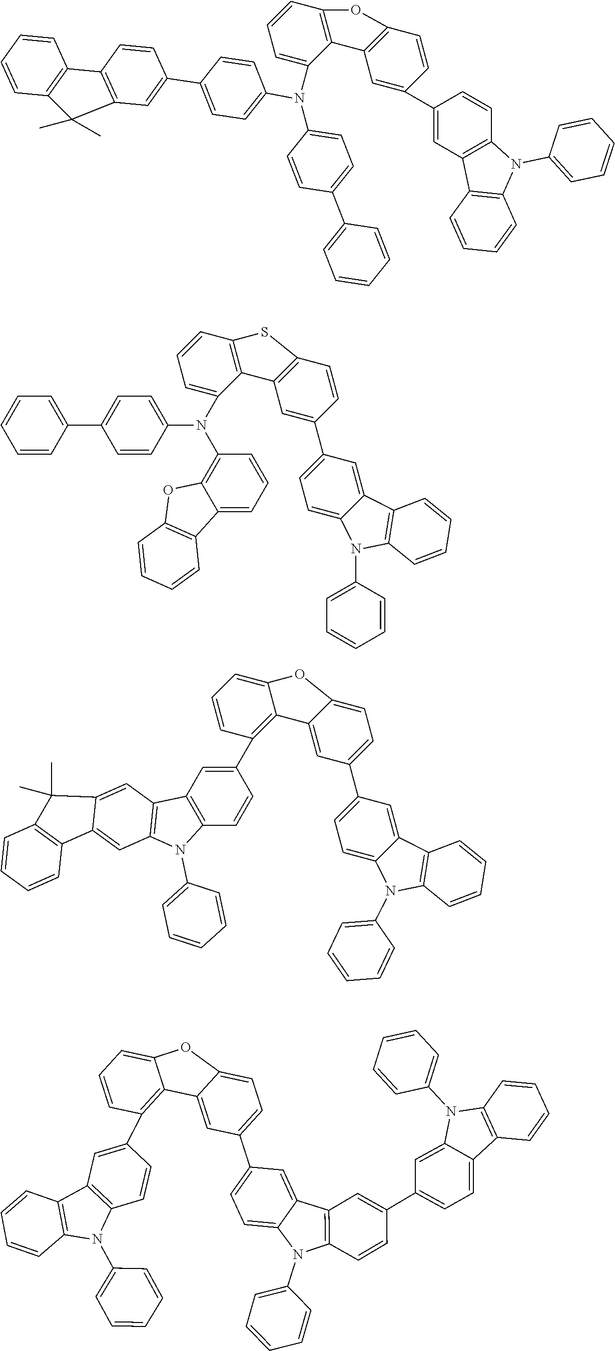 [SMILES: CC1(C)c2ccccc2-c2ccc(-c3ccc(N(c4ccc(-c5ccccc5)cc4)c4cccc5oc6ccc(-c7ccc8c(c7)c7ccccc7n8-c7ccccc7)cc6c45)cc3)cc21 ; c1ccc(-c2ccc(N(c3cccc4c3oc3ccccc34)c3cccc4sc5ccc(-c6ccc7c(c6)c6ccccc6n7-c6ccccc6)cc5c34)cc2)cc1 ; CC1(C)c2ccccc2-c2cc3c(cc21)c1cc(-c2cccc4oc5ccc(-c6ccc7c(c6)c6ccccc6n7-c6ccccc6)cc5c24)ccc1n3-c1ccccc1 ; c1ccc(-n2c3ccc(-c4ccc5oc6cccc(-c7ccc8c(c7)c7ccccc7n8-c7ccccc7)c6c5c4)cc3c3cc(-c4ccc5c6ccccc6n(-c6ccccc6)c5c4)ccc32)cc1]

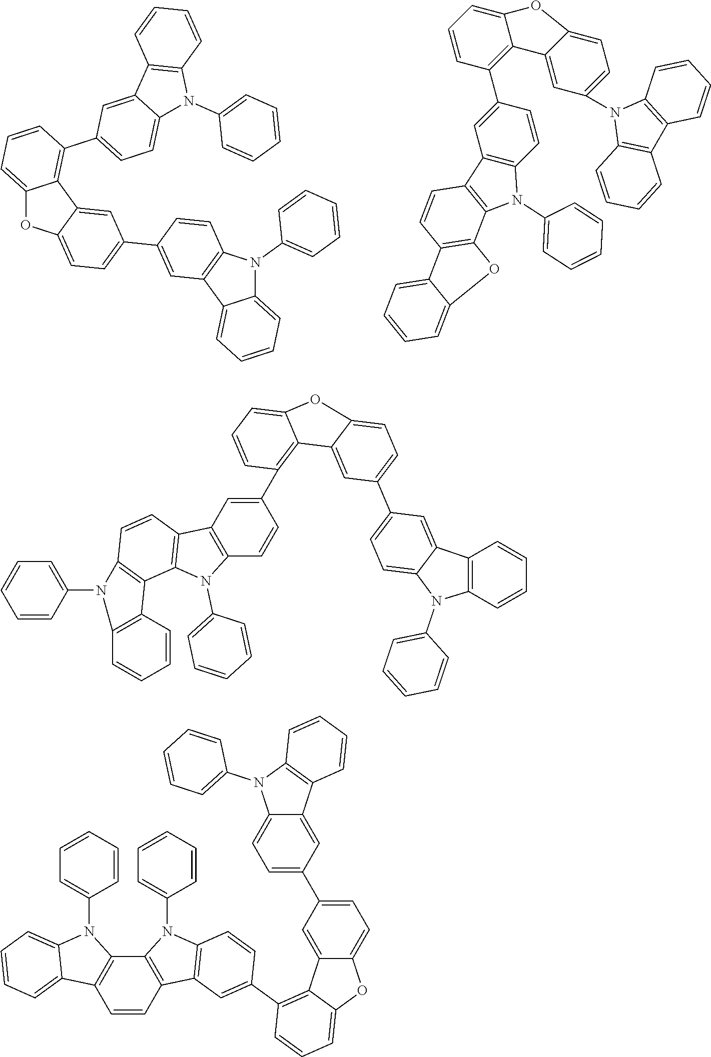 [SMILES: c1ccc(-n2c3ccccc3c3cc(-c4ccc5oc6cccc(-c7ccc8c(c7)c7ccccc7n8-c7ccccc7)c6c5c4)ccc32)cc1 ; c1ccc(-n2c3ccc(-c4cccc5oc6ccc(-n7c8ccccc8c8ccccc87)cc6c45)cc3c3ccc4c5ccccc5oc4c32)cc1 ; c1ccc(-n2c3ccccc3c3cc(-c4ccc5oc6cccc(-c7ccc8c(c7)c7ccc9c(c%10ccccc%10n9-c9ccccc9)c7n8-c7ccccc7)c6c5c4)ccc32)cc1 ; c1ccc(-n2c3ccccc3c3cc(-c4ccc5oc6cccc(-c7ccc8c(c7)c7ccc9c%10ccccc%10n(-c%10ccccc%10)c9c7n8-c7ccccc7)c6c5c4)ccc32)cc1]

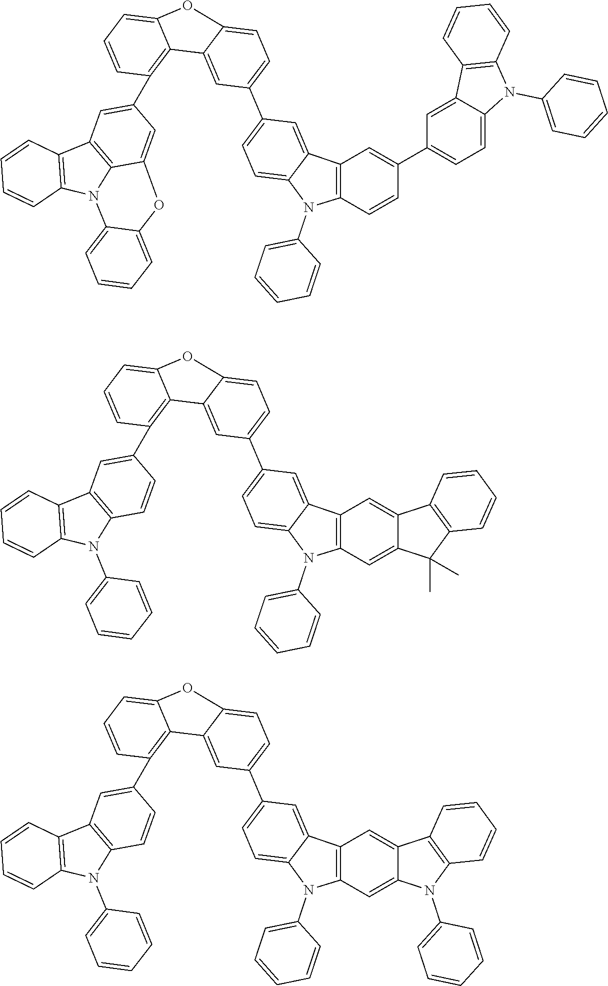 [SMILES: c1ccc(-n2c3ccccc3c3cc(-c4ccc5c(c4)c4cc(-c6ccc7oc8cccc(-c9cc%10c%11c(c9)c9ccccc9n%11-c9ccccc9O%10)c8c7c6)ccc4n5-c4ccccc4)ccc32)cc1 ; CC1(C)c2ccccc2-c2cc3c4cc(-c5ccc6oc7cccc(-c8ccc9c(c8)c8ccccc8n9-c8ccccc8)c7c6c5)ccc4n(-c4ccccc4)c3cc21 ; c1ccc(-n2c3ccccc3c3cc(-c4cccc5oc6ccc(-c7ccc8c(c7)c7cc9c%10ccccc%10n(-c%10ccccc%10)c9cc7n8-c7ccccc7)cc6c45)ccc32)cc1]

Preferred carbazoleamines are the structures of the following formulae (28), (29) and (30):

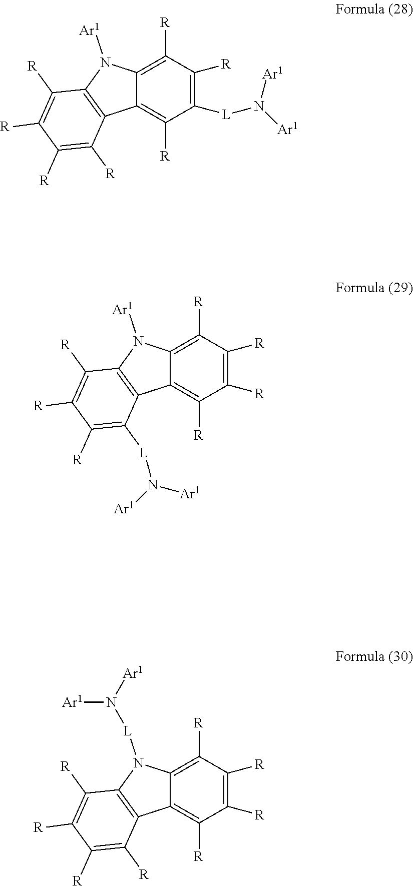

where L, R and Ar¹ have the definitions given above.

Examples of suitable carbazoleamine derivatives are the compounds depicted below.

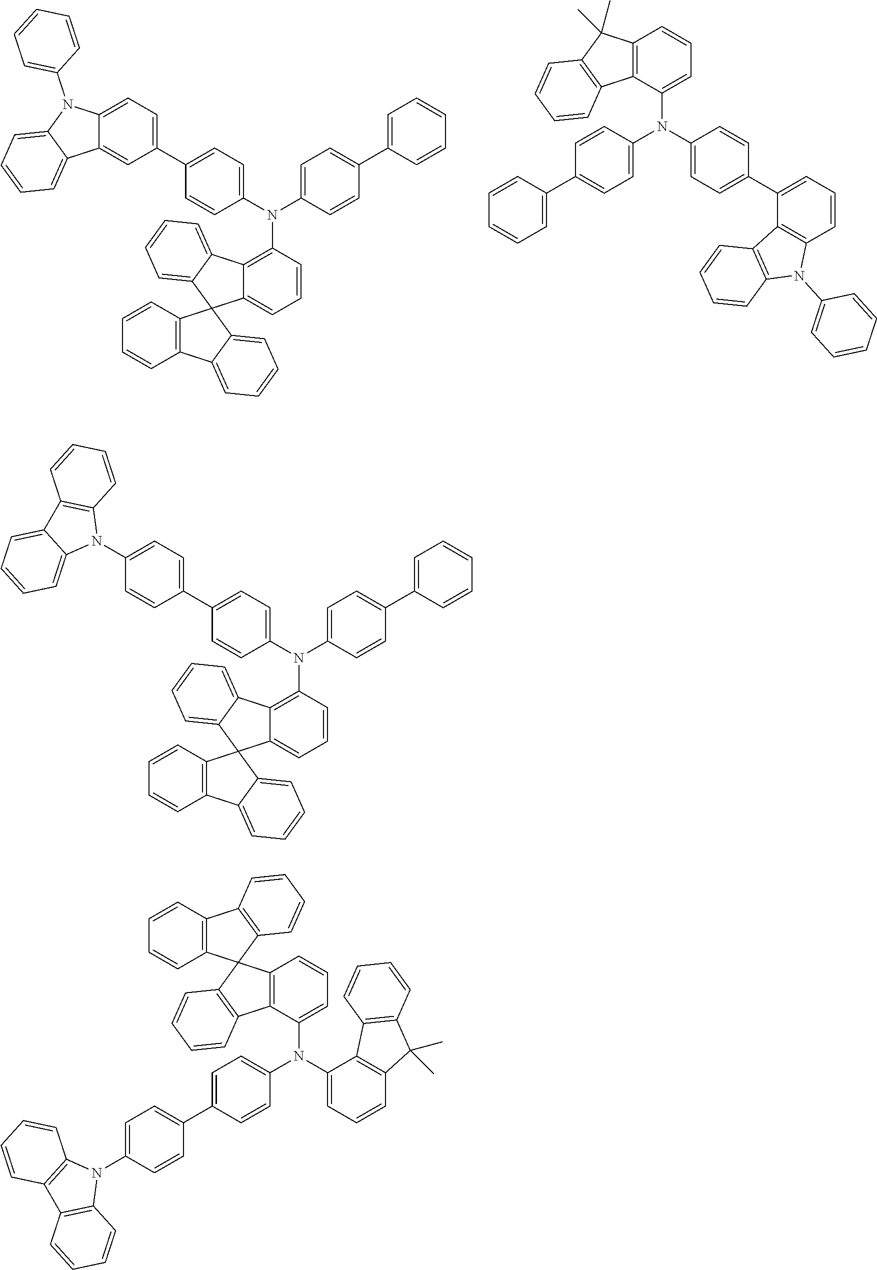

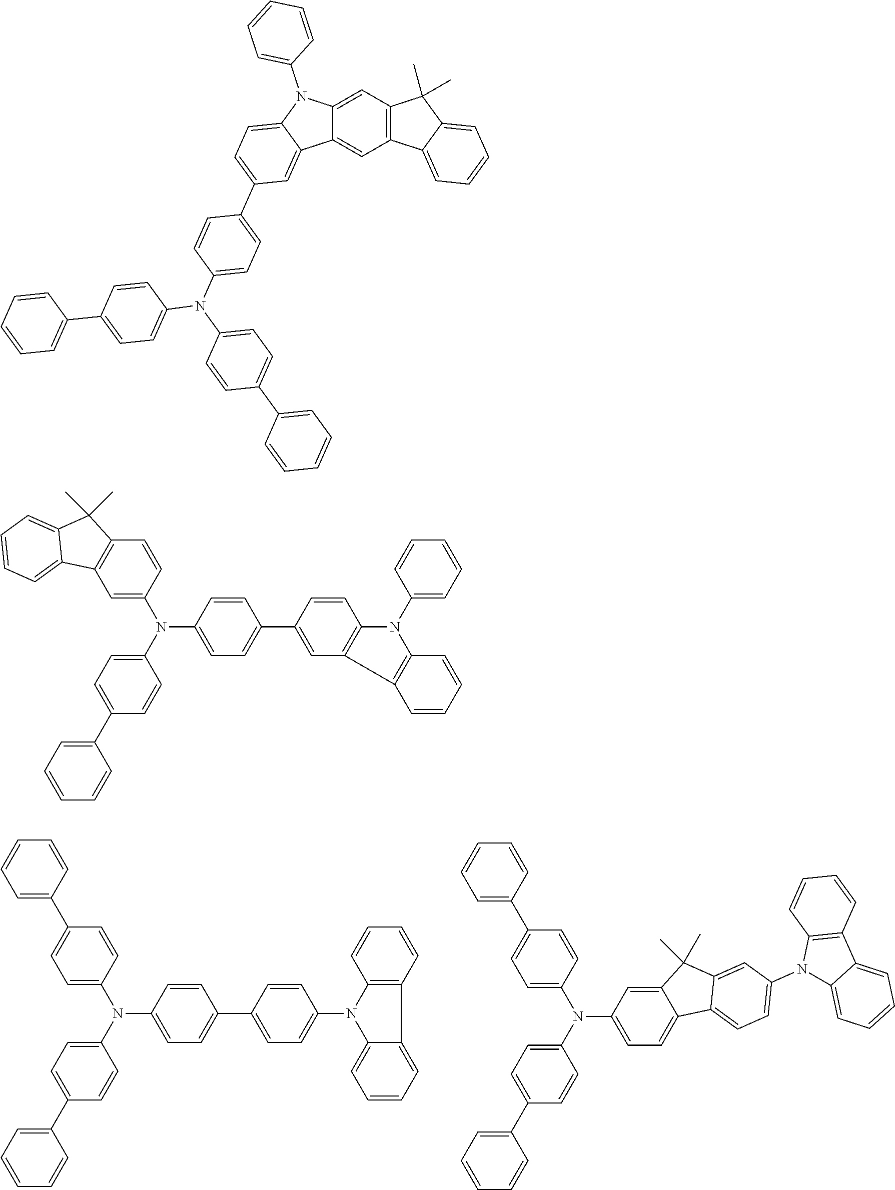

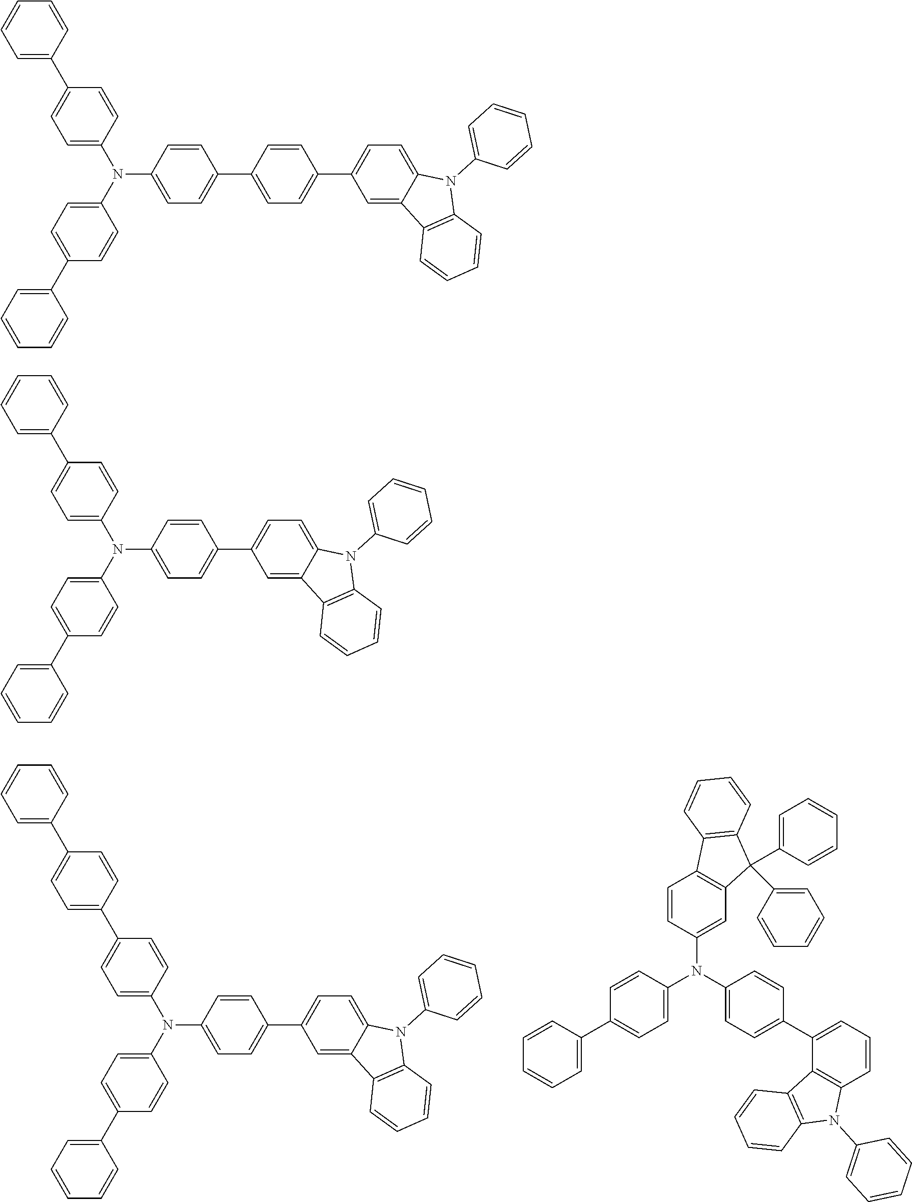

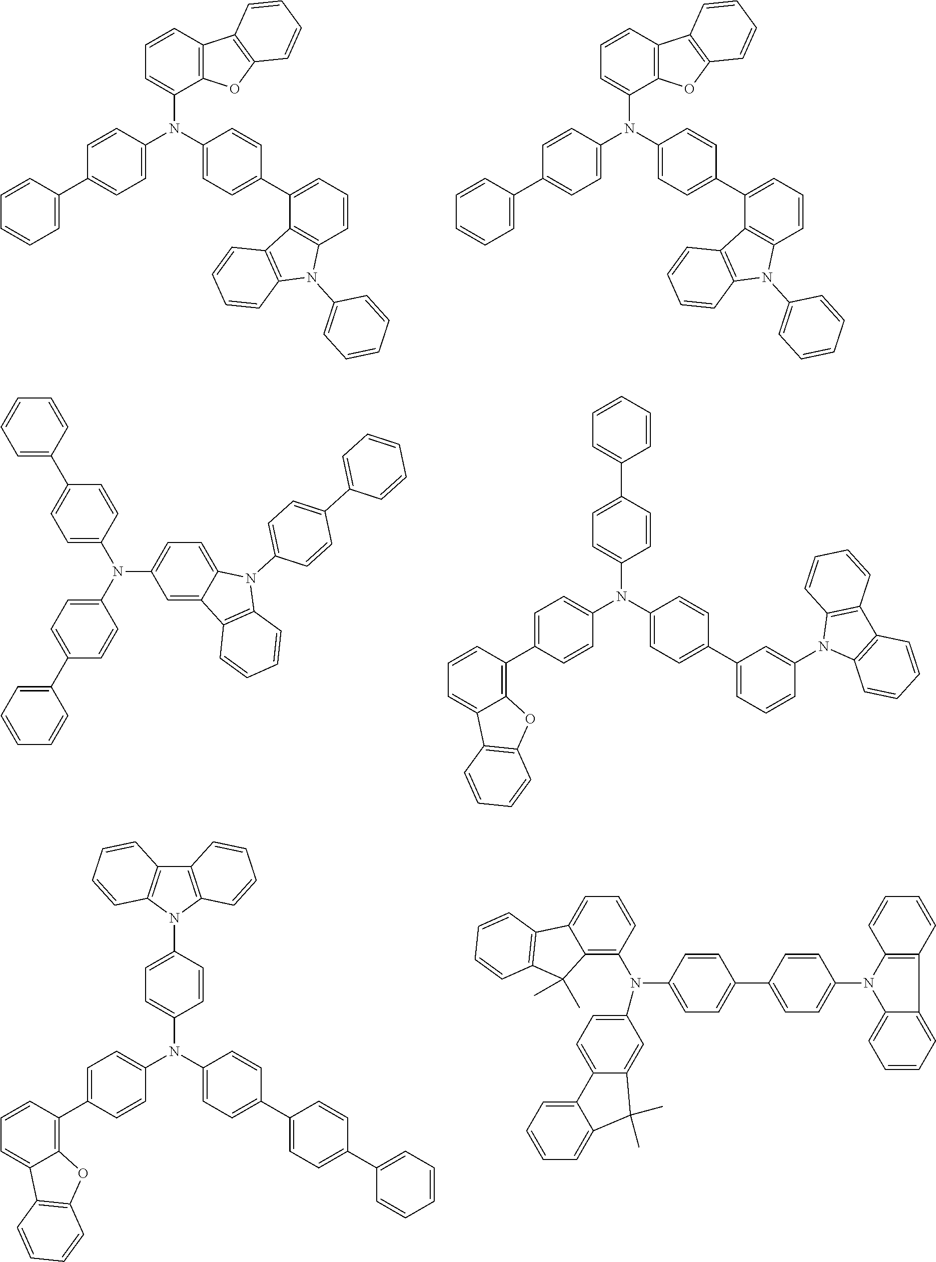

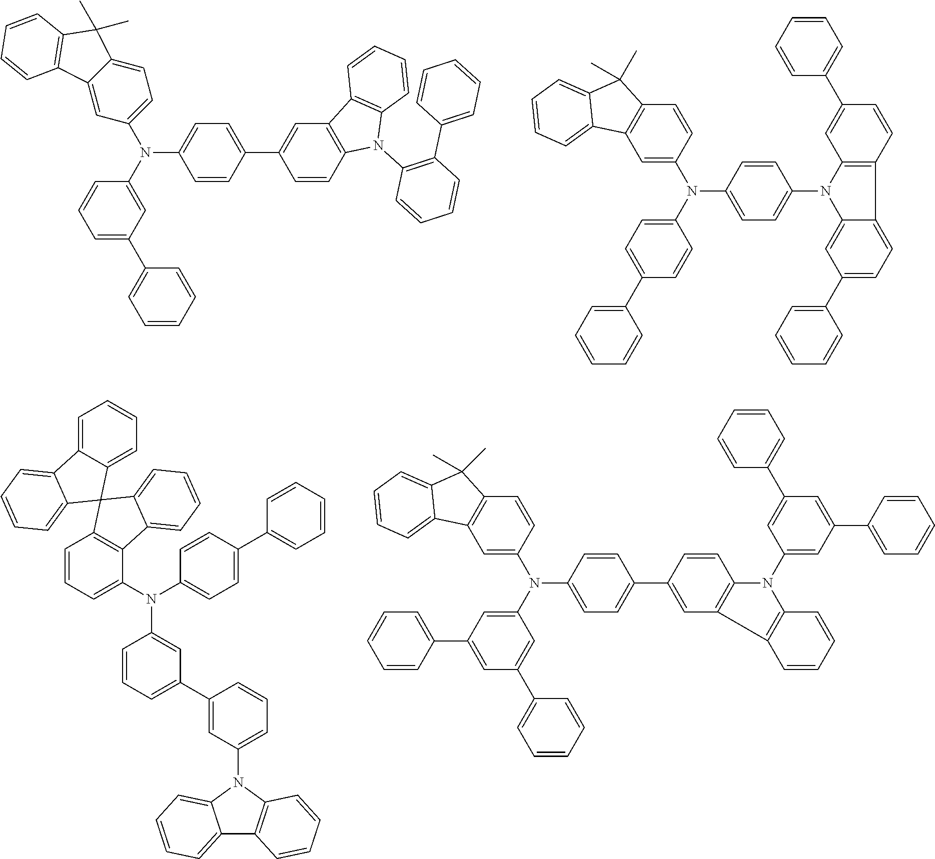 [SMILES: CC1(C)c2ccccc2-c2cc(N(c3ccc(-c4ccc5c(c4)c4ccccc4n5-c4ccccc4-c4ccccc4)cc3)c3cccc(-c4ccccc4)c3)ccc21 ; CC1(C)c2ccccc2-c2cc(N(c3ccc(-c4ccccc4)cc3)c3ccc(-n4c5cc(-c6ccccc6)ccc5c5ccc(-c6ccccc6)cc54)cc3)ccc21 ; c1ccc(-c2ccc(N(c3cccc(-c4cccc(-n5c6ccccc6c6ccccc65)c4)c3)c3cccc4c3-c3ccccc3C43c4ccccc4-c4ccccc43)cc2)cc1 ; CC1(C)c2ccccc2-c2cc(N(c3ccc(-c4ccc5c(c4)c4ccccc4n5-c4cc(-c5ccccc5)cc(-c5ccccc5)c4)cc3)c3cc(-c4ccccc4)cc(-c4ccccc4)c3)ccc21]

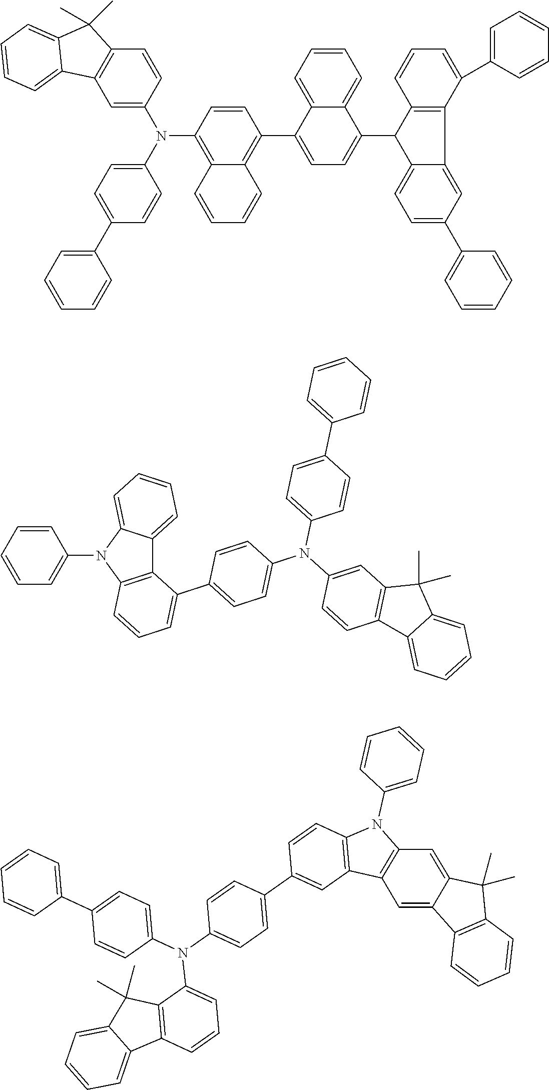 [SMILES: CC1(C)c2ccccc2-c2cc(N(c3ccc(-c4ccccc4)cc3)c3ccc(-c4ccc(C5c6ccc(-c7ccccc7)cc6-c6c(-c7ccccc7)cccc65)c5ccccc45)c4ccccc34)ccc21 ; CC1(C)c2ccccc2-c2ccc(N(c3ccc(-c4ccccc4)cc3)c3ccc(-c4cccc5c4c4ccccc4n5-c4ccccc4)cc3)cc21 ; CC1(C)c2ccccc2-c2cc3c4cc(-c5ccc(N(c6ccc(-c7ccccc7)cc6)c6cccc7c6C(C)(C)c6ccccc6-7)cc5)ccc4n(-c4ccccc4)c3cc21]

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the emitters described above can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439, WO 2018/011186 and WO 2018/041769, WO 2019/020538, WO 2018/178001, WO 2019/115423 and WO 2019/158453. In general, all phosphorescent complexes as used for phosphorescent OLEDs according to the prior art and as known to those skilled in the art in the field of organic electroluminescence are suitable, and the person skilled in the art will be able to use further phosphorescent complexes without exercising inventive skill.

In the further layers of the organic electroluminescent device of the invention, it is possible to use any materials as typically used according to the prior art. The person skilled in the art will therefore be able, without exercising inventive skill, to use any materials known for organic electroluminescent devices in combination with the compounds of formula (1) or the above-recited preferred embodiments.

Additionally preferred is an organic electroluminescent device, characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

In addition, hybrid methods are possible, in which, for example, one or more layers are applied from solution and one or more further layers are applied by vapor deposition.

These methods are known in general terms to those skilled in the art and can be applied by those skilled in the art without exercising inventive skill to organic electroluminescent devices comprising the compounds of formula (1).

The materials of the invention and the organic electroluminescent devices of the invention are notable for one or more of the following surprising advantages over the prior art:
1. OLEDs containing the compounds of formula (1) as matrix material for phosphorescent emitters lead to long lifetimes. This is especially true when the compounds are used as matrix material for a phosphorescent emitter. More particularly, the OLEDs show an improved lifetime compared to OLEDs with matrix materials that do have the same bridged triphenylene base structure but have a different substitution pattern and no substituent R*.
2. OLEDs containing the compounds of formula (1) lead to high efficiencies. This is especially true when the compounds are used as matrix material for a phosphorescent emitter.
3. OLEDs containing the compounds of formula (1) lead to low operating voltages. This is especially true when the compounds are used as matrix material for a phosphorescent emitter.
4. The compounds of the invention may also be used with very good properties in an electron transport layer, including in combination with a fluorescent emission layer, or in a hole blocker layer. Especially when the compounds are used in an electron transport layer in combination with a blue-fluorescent emitter layer, improved efficiency is obtained in combination with simultaneously lower operating voltage.

The invention is illustrated in detail by the examples which follow, without any intention of restricting it thereby. The person skilled in the art will be able to use the information given to execute the invention over the entire scope disclosed and produce further inventive electronic devices without exercising inventive skill.

### EXAMPLES

The syntheses which follow, unless stated otherwise, are conducted under a protective gas atmosphere in dried solvents. The solvents and reagents can be purchased, for example, from Sigma-ALDRICH or ABCR. For the compounds known from the literature, the corresponding CAS numbers are also reported in each case.

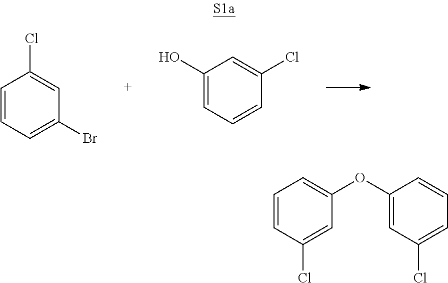 [SMILES: Clc1cccc(Br)c1 ; Oc1cccc(Cl)c1 ; Clc1cccc(Oc2cccc(Cl)c2)c1]

An initial charge under an inert atmosphere is formed by DMSO (50 ml), K₃PO₄ (53.08 g, 250 mmol), pyridine-2-carboxylic acid (1.53 g, 12.44 mmol) and CuI (1.19 g, 6.22 mmol). Subsequently, 3-chlorophenol (19.20 g, 150 mmol) [108-43-0] and 3-bromo-1-chlorobenzene (23.93 g, 125 mmol) [108-37-2] are gradually added successively, and the reaction mixture is heated at 85° C. for 16 h. After cooling, the reaction mixture is worked up by extraction with aqueous ammonia solution and methyl tert-butyl ether. The organic phase is washed five times with water and twice with saturated NaCl solution, the combined organic phases are dried over Na₂SO₄, and the solvent is drawn off on a rotary evaporator. The crude product is purified further via fractional distillation. Yield: 26.88 g (106 mmol), 85%; purity; 96% by ¹H NMR

The following compounds can be prepared analogously: Purification can be effected not only by distillation but also using column chromatography, or recrystallization can be effected using other standard solvents such as ethanol, butanol, acetone, ethyl acetate, acetonitrile, toluene, xylene, dichloromethane, methanol, tetrahydrofuran, n-butyl acetate, 1,4-dioxane, dimethyl sulfoxide, N,N-dimethylformamide, N,N-dimethylacetamide, N-methylpyrrolidone etc.

| Reactant 1 | Reactant 2 | Product | Yield |
|---|---|---|---|
| 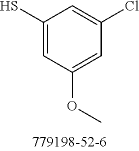 | 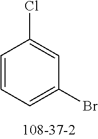 | 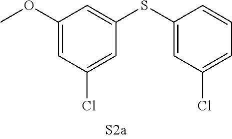 [SMILES: COc1cc(Cl)cc(Sc2cccc(Cl)c2)c1] | 80% |
| 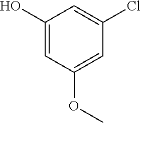 | 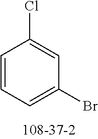 | 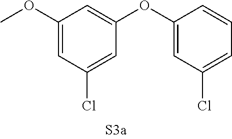 [SMILES: COc1cc(Cl)cc(Oc2cccc(Cl)c2)c1] | 86% |
| 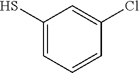 | 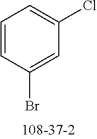 | 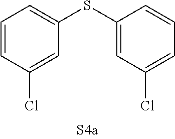 [SMILES: Clc1cccc(Sc2cccc(Cl)c2)c1] | 74% |
| 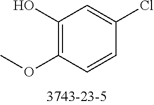 | 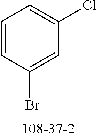 | 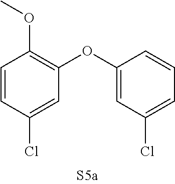 [SMILES: COc1ccc(Cl)cc1Oc1cccc(Cl)c1] | 75% |
| 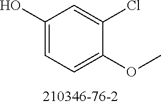 | 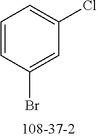 | 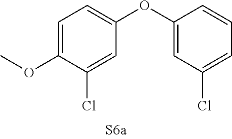 [SMILES: COc1ccc(Oc2cccc(Cl)c2)cc1Cl] | 68% |

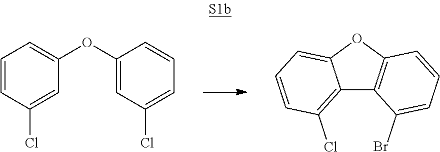 [SMILES: Clc1cccc(Oc2cccc(Cl)c2)c1 ; Clc1cccc2oc3cccc(Br)c3c12]

An initial charge of S1a (23.90 g, 100 mmol) in THF (150 ml) under an inert atmosphere is cooled down to −75° C. Subsequently, n-butyllithium (2.5 mol/l in hexane, 80 ml, 200 mmol) is added dropwise in such a way that the internal temperature does not exceed −65° C. The mixture is left to stir −75° C. for a further 4 h, and then bromine (5.6 ml, 109.3 mmol) is added in such a way that the internal temperature does not exceed −65° C. After the addition has ended, the mixture is stirred at −75° C. for 1 h, then allowed to warm up gradually to 10° C. within 1 h and stirred at 10° C. for 1 h. This is followed by cooling to 0° C. and cautious quenching of the mixture with saturated Na₂SO₃ solution (50 ml). The mixture is worked up by extraction with toluene and water, the combined organic phases are washed three times with water and once with saturated NaCl solution and dried over Na₂SO₄, and the solvent is removed on a rotary evaporator. The crude product is twice extracted by stirring with 2-propanol under reflux. Yield: 24.21 g (86 mmol, 86%), purity: 97% by ¹H NMR.

The following compounds can be prepared analogously: Purification can be effected not only by extractive stirring but also by distillation, or recrystallization can be effected using other standard solvents such as ethanol, butanol, acetone, ethyl acetate, acetonitrile, toluene, xylene, dichloromethane, methanol, tetrahydrofuran, n-butyl acetate, 1,4-dioxane, dimethyl sulfoxide, N,N-dimethylformamide, N,N-dimethylacetamide, N-methylpyrrolidone etc.

| Reactant | Product | Yield |
|---|---|---|
| 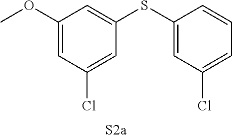 [SMILES: COc1cc(Cl)cc(Sc2cccc(Cl)c2)c1] | 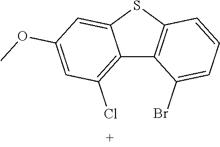 [SMILES: COc1cc(Cl)c2c(c1)sc1cccc(Br)c12] 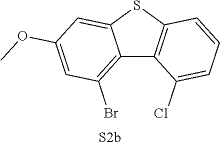 [SMILES: COc1cc(Br)c2c(c1)sc1cccc(Cl)c12] | 70% |
| 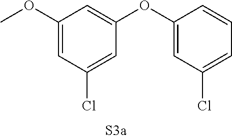 [SMILES: COc1cc(Cl)cc(Oc2cccc(Cl)c2)c1] | 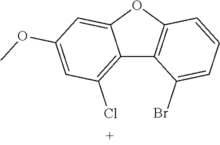 [SMILES: COc1cc(Cl)c2c(c1)oc1cccc(Br)c12] | 79% |
| | 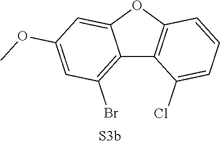 [SMILES: COc1cc(Br)c2c(c1)oc1cccc(Cl)c12] | |
| 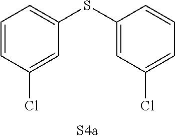 [SMILES: Clc1cccc(Sc2cccc(Cl)c2)c1] | 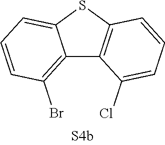 [SMILES: Clc1cccc2sc3cccc(Br)c3c12] | 79% |
| 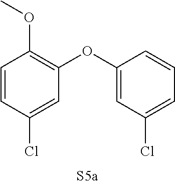 [SMILES: COc1ccc(Cl)cc1Oc1cccc(Cl)c1] | 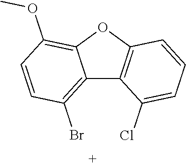 [SMILES: COc1ccc(Br)c2c1oc1cccc(Cl)c12] 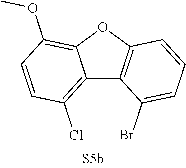 [SMILES: COc1ccc(Cl)c2c1oc1cccc(Br)c12] | 74% |
| 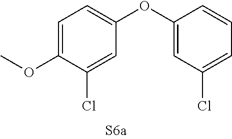 [SMILES: COc1ccc(Oc2cccc(Cl)c2)cc1Cl] | 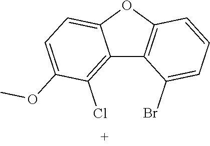 [SMILES: COc1ccc2oc3cccc(Br)c3c2c1Cl] | 53% |
| | 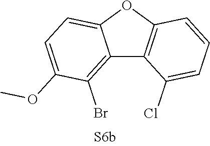 [SMILES: COc1ccc2oc3cccc(Cl)c3c2c1Br] | |

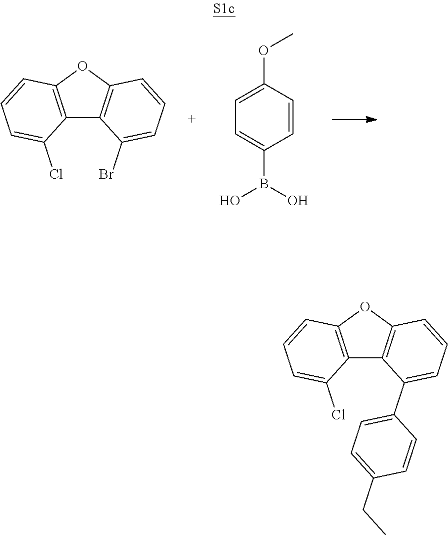 [SMILES: Clc1cccc2oc3cccc(Br)c3c12 ; COc1ccc(B(O)O)cc1 ; CCc1ccc(-c2cccc3oc4cccc(Cl)c4c23)cc1]

An initial charge of S1b (39.19 g, 140.0 mmol), 4-methoxyphenylboronic acid (22.79 g, 150.0 mmol) [5720-07-0] and K₂CO₃ (38.70 g, 280.0 mmol) in THF (70 ml) and water (170 ml) is inertized for 30 min. Subsequently, tetrakis(triphenylphosphine)palladium [14221-01-3] (1.78 mg, 1.54 mmol) is added and the reaction mixture is stirred under reflux for 20 h. The mixture is worked up by extraction with toluene and water, the combined organic phases are washed with water and saturated NaCl solution and dried over Na₂SO₄, and the solvent is drawn off on a rotary evaporator. The crude product is recrystallized from ethanol. Yield: 33.7 g (109 mmol, 78%), 96% by ¹H NMR.

The following compounds can be prepared analogously: Purification can be effected by column chromatography, or recrystallization can be effected using other standard solvents such as ethanol, butanol, acetone, ethyl acetate, acetonitrile, toluene, xylene, dichloromethane, methanol, tetrahydrofuran, n-butyl acetate, 1,4-dioxane, dimethyl sulfoxide, N,N-dimethylformamide, N,N-dimethylacetamide, N-methyl pyrrolidone etc.

| Reactant 1 | Reactant 2 | Product | Yield |
|---|---|---|---|
| 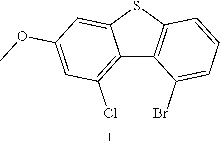 [SMILES: COc1cc(Cl)c2c(c1)sc1cccc(Br)c12] 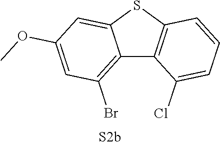 [SMILES: COc1cc(Br)c2c(c1)sc1cccc(Cl)c12] | 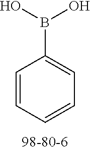 | 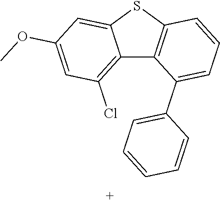 [SMILES: COc1cc(Cl)c2c(c1)sc1cccc(-c3ccccc3)c12] 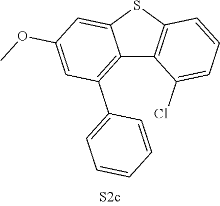 [SMILES: COc1cc(-c2ccccc2)c2c(c1)sc1cccc(Cl)c12] | 64% |
| 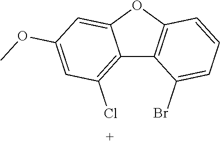 [SMILES: COc1cc(Cl)c2c(c1)oc1cccc(Br)c12] 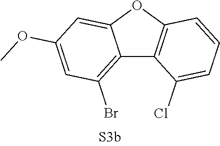 [SMILES: COc1cc(Br)c2c(c1)oc1cccc(Cl)c12] | 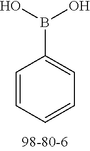 | 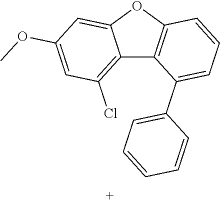 [SMILES: COc1cc(Cl)c2c(c1)oc1cccc(-c3ccccc3)c12] 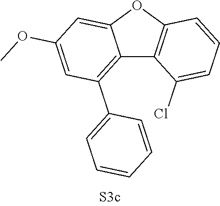 [SMILES: COc1cc(-c2ccccc2)c2c(c1)oc1cccc(Cl)c12] | 69% |
| 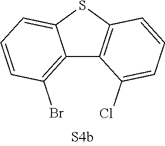 [SMILES: Clc1cccc2sc3cccc(Br)c3c12] | 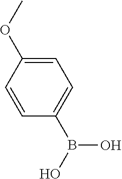 | 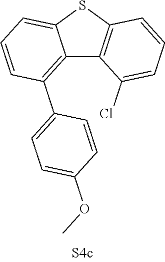 [SMILES: COc1ccc(-c2cccc3sc4cccc(Cl)c4c23)cc1] | 62% |
| 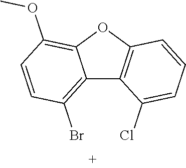 [SMILES: COc1ccc(Br)c2c1oc1cccc(Cl)c12] 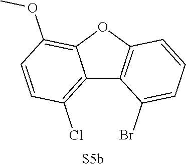 [SMILES: COc1ccc(Cl)c2c1oc1cccc(Br)c12] | 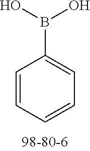 | 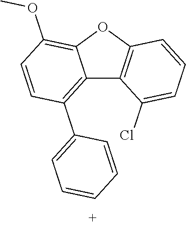 [SMILES: COc1ccc(-c2ccccc2)c2c1oc1cccc(Cl)c12] 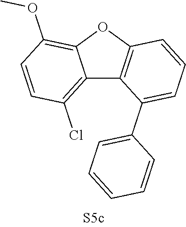 [SMILES: COc1ccc(Cl)c2c1oc1cccc(-c3ccccc3)c12] | 59% |
| 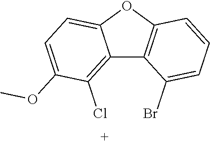 [SMILES: COc1ccc2oc3cccc(Br)c3c2c1Cl] 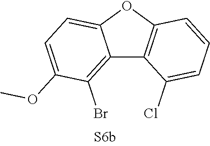 [SMILES: COc1ccc2oc3cccc(Cl)c3c2c1Br] | 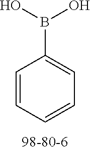 | 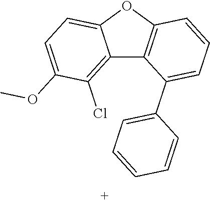 [SMILES: COc1ccc2oc3cccc(-c4ccccc4)c3c2c1Cl] 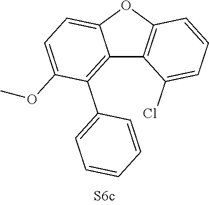 [SMILES: COc1ccc2oc3cccc(Cl)c3c2c1-c1ccccc1] | 51% |
| 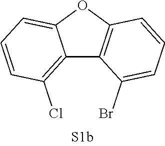 [SMILES: Clc1cccc2oc3cccc(Br)c3c12] | 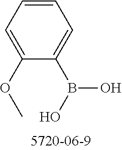 | 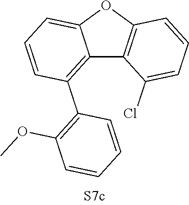 [SMILES: COc1ccccc1-c1cccc2oc3cccc(Cl)c3c12] | 56% |
| 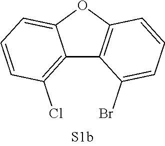 [SMILES: Clc1cccc2oc3cccc(Br)c3c12] | 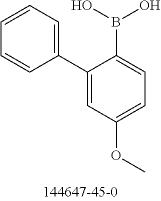 [SMILES: COc1ccc(B(O)O)c(-c2ccccc2)c1] | 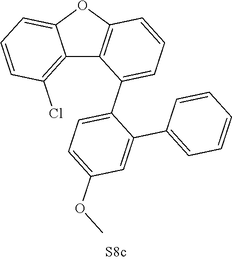 [SMILES: COc1ccc(-c2cccc3oc4cccc(Cl)c4c23)c(-c2ccccc2)c1] | 36% |
| 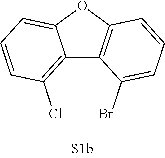 [SMILES: Clc1cccc2oc3cccc(Br)c3c12] | 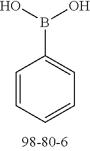 | 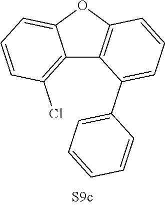 [SMILES: Clc1cccc2oc3cccc(-c4ccccc4)c3c12] | 75% |

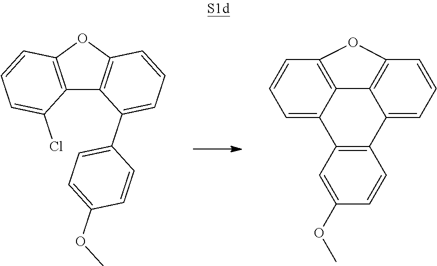 [SMILES: COc1ccc(-c2cccc3oc4cccc(Cl)c4c23)cc1 ; COc1ccc2c(c1)c1cccc3oc4cccc2c4c31]

To an initial charge of S1c (30.88 g, 100 mmol) and K₂CO₃ (41.46 g, 300 mmol) under an inert atmosphere is added DMAc (450 ml), and the mixture is inertized for 30 min. Subsequently, Pd(OAc)₂ (447 mg, 1.99 mmol) and 1,3-bis(2,6-diisopropylphenyl)-3H-imidazol-1-ium chloride (1.69 g, 3.98 mmol) are added, and the reaction mixture is stirred at 150° C. for 16 h. After cooling, the mixture is poured into ethanol/water (1:1, 600 ml) and stirred for a further 30 min. The precipitated solids are filtered off with suction and washed five times with water and three times with ethanol. The crude product is extracted by stirring under reflux with 2-propanol, and the solids are filtered off with suction after cooling. Yield: 22.9 g (84 mmol, 84%), 98% by 1H NMR.

The following compounds can be prepared analogously: It is possible here to use not only 1,3-bis(2,6-diisopropylphenyl)-3H-imidazol-1-ium chloride but also tri-tert-butylphosphine or tricyclohexylphosphine, or as Pd source to use not only Pd(OAc)₂ but also Pd₂(dba)₋₃. Purification can be effected by column chromatography, or recrystallization can be effected using other standard solvents such as ethanol, butanol, acetone, ethyl acetate, acetonitrile, toluene, xylene, dichloromethane, methanol, tetrahydrofuran, n-butyl acetate, 1,4-dioxane, dimethyl sulfoxide, N,N-dimethylformamide, N,N-dimethylacetamide, N-methylpyrrolidone etc.

| Reactant | Product | Yield |
|---|---|---|
| 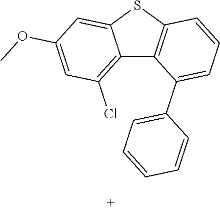 [SMILES: COc1cc(Cl)c2c(c1)sc1cccc(-c3ccccc3)c12] | 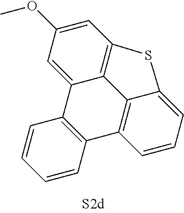 [SMILES: COc1cc2sc3cccc4c5ccccc5c(c1)c2c34] | 75% |
| 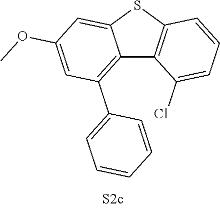 [SMILES: COc1cc(-c2ccccc2)c2c(c1)sc1cccc(Cl)c12] | | |
| 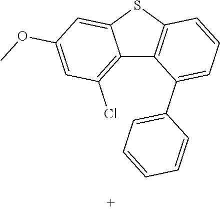 [SMILES: COc1cc(Cl)c2c(c1)sc1cccc(-c3ccccc3)c12] | 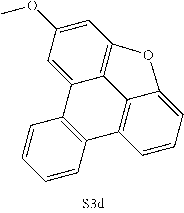 [SMILES: COc1cc2oc3cccc4c5ccccc5c(c1)c2c34] | 73% |
| 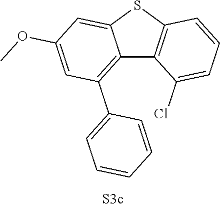 [SMILES: COc1cc(-c2ccccc2)c2c(c1)sc1cccc(Cl)c12] | | |
| 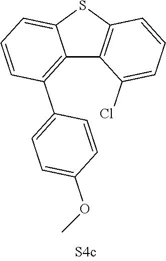 [SMILES: COc1ccc(-c2cccc3sc4cccc(Cl)c4c23)cc1] | 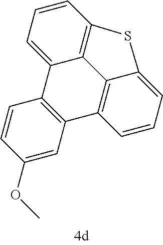 [SMILES: COc1ccc2c(c1)c1cccc3sc4cccc2c4c31] | 78% |
| 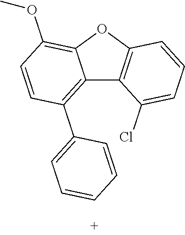 [SMILES: COc1ccc(-c2ccccc2)c2c1oc1cccc(Cl)c12] 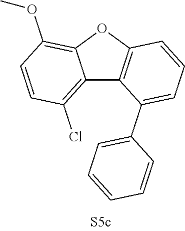 [SMILES: COc1ccc(Cl)c2c1oc1cccc(-c3ccccc3)c12] | 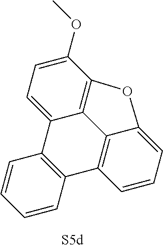 [SMILES: COc1ccc2c3ccccc3c3cccc4oc1c2c43] | 80% |
| 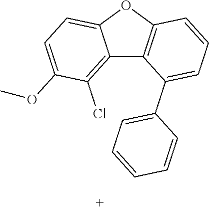 [SMILES: COc1ccc2oc3cccc(-c4ccccc4)c3c2c1Cl] | 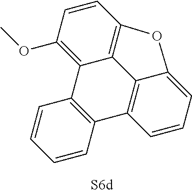 [SMILES: COc1ccc2oc3cccc4c5ccccc5c1c2c34] | 63% |
| 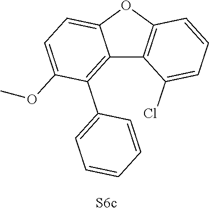 [SMILES: COc1ccc2oc3cccc(Cl)c3c2c1-c1ccccc1] | | |
| 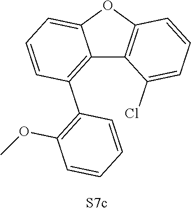 [SMILES: COc1ccccc1-c1cccc2oc3cccc(Cl)c3c12] | 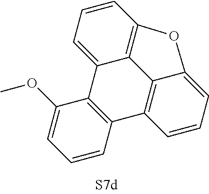 [SMILES: COc1cccc2c3cccc4oc5cccc(c12)c5c43] | 46% |
| 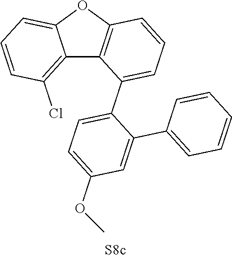 [SMILES: COc1ccc(-c2cccc3oc4cccc(Cl)c4c23)c(-c2ccccc2)c1] | 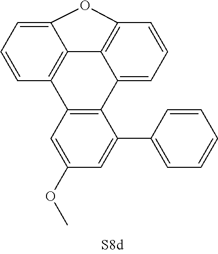 [SMILES: COc1cc(-c2ccccc2)c2c(c1)c1cccc3oc4cccc2c4c31] | 25% |
| 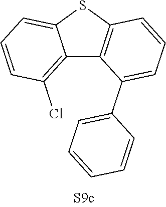 [SMILES: Clc1cccc2sc3cccc(-c4ccccc4)c3c12] | 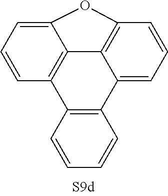 [SMILES: c1ccc2c(c1)c1cccc3oc4cccc2c4c31] | 81% |

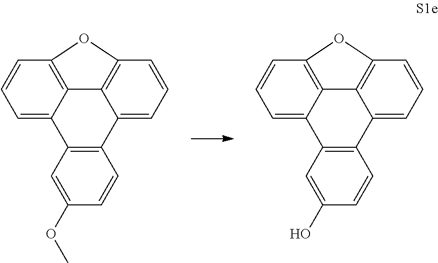 [SMILES: COc1ccc2c(c1)c1cccc3oc4cccc2c4c31 ; Oc1ccc2c(c1)c1cccc3oc4cccc2c4c31]

An initial charge of S1d (27.23 g, 100 mmol) in dichloromethane (620 ml) is cooled in an ice bath to 0° C. Subsequently, BBr₃ (6.0 ml, 63.2 mmol) is cautiously added dropwise. After the addition has ended, the mixture is allowed to warm up to room temperature. On completion of conversion, the mixture is cooled again to 0° C. and quenched cautiously with MeOH (150 ml). The solvent is drawn off on a rotary evaporator. Subsequently, each of three additions of 300 ml of MeOH to the mixture is followed by removal thereof on a rotary evaporator. Another 200 ml of MeOH is added, and the solids are filtered off with suction. The crude product is dried and used in the next stage without further purification. Yield: 17.05 g (66 mmol, 66%).

The following compounds can be prepared analogously: Purification can be effected by column chromatography, or recrystallization can be effected using other standard solvents such as ethanol, butanol, acetone, ethyl acetate, acetonitrile, toluene, xylene, dichloromethane, methanol, tetrahydrofuran, n-butyl acetate, 1,4-dioxane, dimethyl sulfoxide, N,N-dimethylformamide, N,N-dimethylacetamide, N-methylpyrrolidone etc.

| Reactant | Product | Yield |
|---|---|---|
| 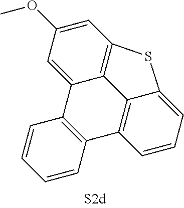 [SMILES: COc1cc2sc3cccc4c5ccccc5c(c1)c2c34] | 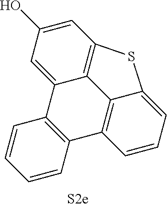 [SMILES: Oc1cc2sc3cccc4c5ccccc5c(c1)c2c34] | 64% |
| 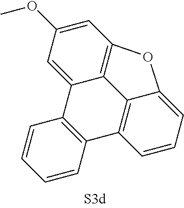 [SMILES: COc1cc2oc3cccc4c5ccccc5c(c1)c2c34] | 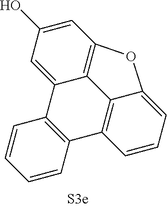 [SMILES: Oc1cc2oc3cccc4c5ccccc5c(c1)c2c34] | 71% |
| 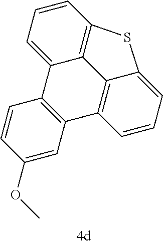 [SMILES: COc1ccc2c(c1)c1cccc3sc4cccc2c4c31] | 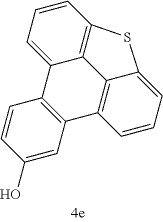 [SMILES: Oc1ccc2c(c1)c1cccc3sc4cccc2c4c31] | 72% |
| 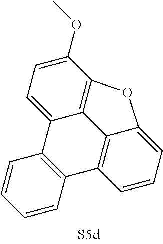 [SMILES: COc1ccc2c3ccccc3c3cccc4oc1c2c43] | 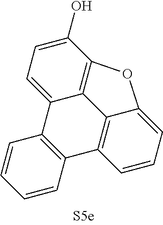 [SMILES: Oc1ccc2c3ccccc3c3cccc4oc1c2c43] | 63% |
| 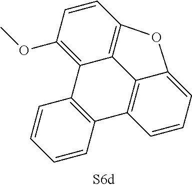 [SMILES: COc1ccc2oc3cccc4c5ccccc5c1c2c34] | 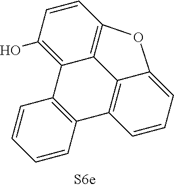 [SMILES: Oc1ccc2oc3cccc4c5ccccc5c1c2c34] | 49% |
| 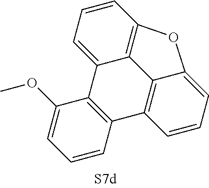 [SMILES: COc1cccc2c3cccc4oc5cccc(c12)c5c43] | 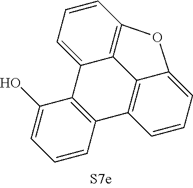 [SMILES: Oc1cccc2c3cccc4oc5cccc(c12)c5c43] | 57% |
| 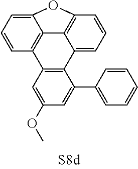 [SMILES: COc1cc(-c2ccccc2)c2c(c1)c1cccc3oc4cccc2c4c31] | 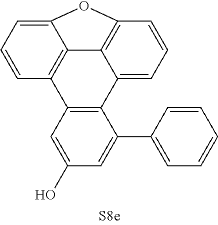 [SMILES: Oc1cc(-c2ccccc2)c2c(c1)c1cccc3oc4cccc2c4c31] | 55% |

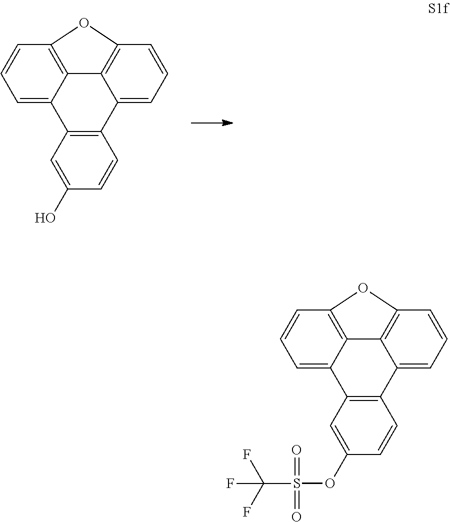 [SMILES: Oc1ccc2c(c1)c1cccc3oc4cccc2c4c31 ; O=S(=O)(Oc1ccc2c(c1)c1cccc3oc4cccc2c4c31)C(F)(F)F]

An initial charge of S1e (12.91 g, 50.0 mmol) and triethylamine (20.8 ml, 150 mmol) in dichloromethane (700 ml) is cooled to 0° C. in an ice bath. Subsequently, trifluoromethanesulfonic anhydride (10.9 ml, 65.0 mmol) is slowly added dropwise. After the addition has ended, the mixture is allowed to warm up to room temperature. On completion of conversion, the mixture is subjected to extractive workup with dichloromethane and water, the combined organic phases are dried over Na₂SO₄, and the solvent is removed on a rotary evaporator. The residue is taken up in 300 ml of cyclohexane, and the mixture is stirred at room temperature for 30 min. The solids are filtered off with suction and dried in a vacuum drying cabinet. Yield 13.74 g (35.2 mmol, 70%).

The following compounds can be prepared analogously: Purification can be effected by column chromatography, or recrystallization can be effected using other standard solvents such as ethanol, butanol, acetone, ethyl acetate, acetonitrile, toluene, xylene, dichloromethane, methanol, tetrahydrofuran, n-butyl acetate, 1,4-dioxane, dimethyl sulfoxide, N,N-dimethylformamide, N,N-dimethylacetamide, N-methylpyrrolidone etc.

| Reactant | Product | Yield |
|---|---|---|
| 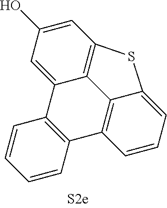 [SMILES: Oc1cc2sc3cccc4c5ccccc5c(c1)c2c34] | 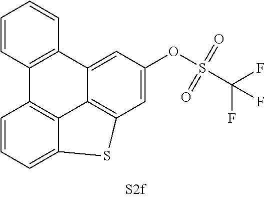 [SMILES: O=S(=O)(Oc1cc2sc3cccc4c5ccccc5c(c1)c2c34)C(F)(F)F] | 76% |
| 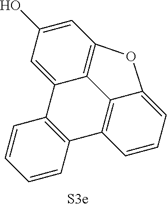 [SMILES: Oc1cc2oc3cccc4c5ccccc5c(c1)c2c34] | 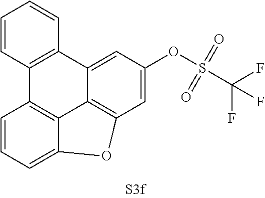 [SMILES: O=S(=O)(Oc1cc2oc3cccc4c5ccccc5c(c1)c2c34)C(F)(F)F] | 79% |
| 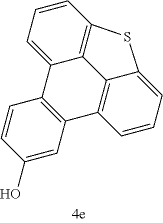 [SMILES: Oc1ccc2c(c1)c1cccc3sc4cccc2c4c31] | 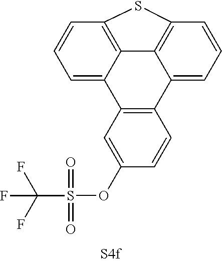 [SMILES: O=S(=O)(Oc1ccc2c(c1)c1cccc3sc4cccc2c4c31)C(F)(F)F] | 67% |
| 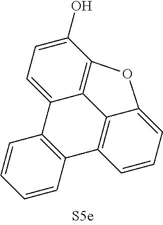 [SMILES: Oc1ccc2c3ccccc3c3cccc4oc1c2c43] | 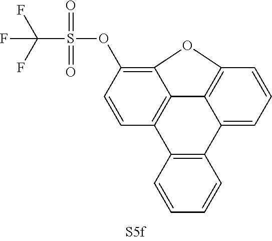 [SMILES: O=S(=O)(Oc1ccc2c3ccccc3c3cccc4oc1c2c43)C(F)(F)F] | 64% |
| 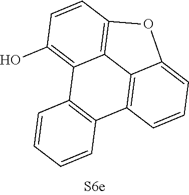 [SMILES: Oc1ccc2oc3cccc4c5ccccc5c1c2c34] | 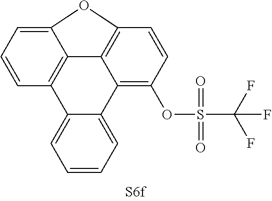 [SMILES: O=S(=O)(Oc1ccc2oc3cccc4c5ccccc5c1c2c34)C(F)(F)F] | 50% |
| 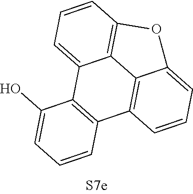 [SMILES: Oc1cccc2c3cccc4oc5cccc(c12)c5c43] | 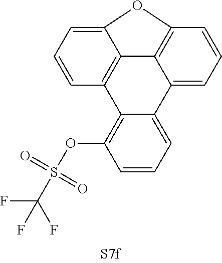 [SMILES: O=S(=O)(Oc1cccc2c3cccc4oc5cccc(c12)c5c43)C(F)(F)F] | 56% |
| 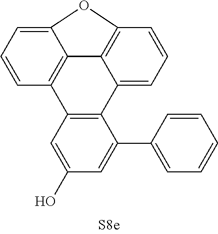 [SMILES: Oc1cc(-c2ccccc2)c2c(c1)c1cccc3oc4cccc2c4c31] | 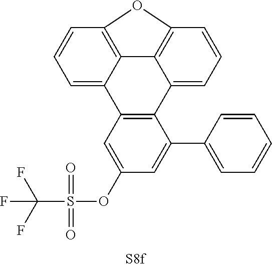 [SMILES: O=S(=O)(Oc1cc(-c2ccccc2)c2c(c1)c1cccc3oc4cccc2c4c31)C(F)(F)F] | 47% |

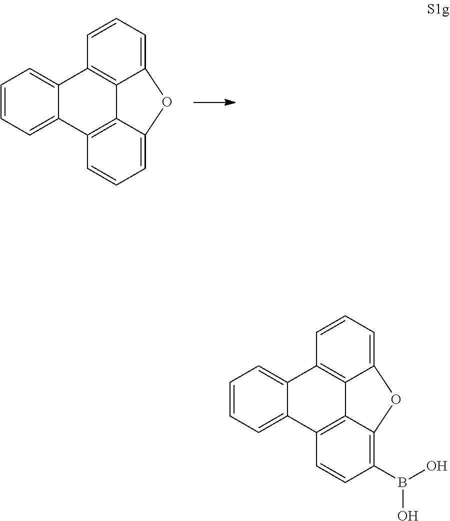 [SMILES: c1ccc2c(c1)c1cccc3oc4cccc2c4c31 ; OB(O)c1ccc2c3ccccc3c3cccc4oc1c2c43]

An initial charge of S9c (24.23 g, 100 mmol) in 300 ml of THF is cooled to −75° C. Subsequently, hexyllithium (44.0 ml, c=2.5 mol/l, 110 mmol) is added in such a way that the temperature does not rise above −65° C. After the addition has ended, the mixture is stirred at −75° C. for 1 h. Subsequently, the reaction mixture is allowed to warm up gradually to room temperature and stirred at room temperature for 1 h. Subsequently, the reaction mixture is cooled back down to −75° C., and trimethyl borate (15.59 g, 150.0 mmol) is added dropwise in such a way that the temperature does not rise above −65° C. The mixture is allowed to come to room temperature overnight and quenched cautiously the next day with HCl (c=5 mol/l, 50 ml). The mixture is worked up by extraction with water, and the organic phase is washed three times with water. The THF is removed by rotary evaporation down to 50 ml, then 150 ml of n-heptane is added, and the precipitated solids are filtered off with suction and washed with n-heptane. Yield: 24.03 g (84.2 mmol, 84%), 96% by 1H NMR.

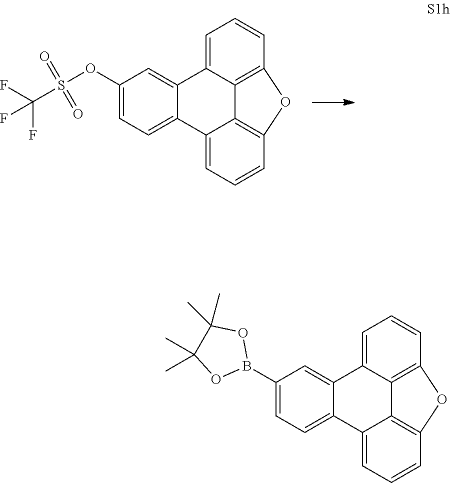 [SMILES: O=S(=O)(Oc1ccc2c(c1)c1cccc3oc4cccc2c4c31)C(F)(F)F ; CC1(C)OB(c2ccc3c(c2)c2cccc4oc5cccc3c5c42)OC1(C)C]

An initial charge of S1f (11.71 g, 30.0 mmol), bis(pinacolato)diboron (9.40 g, 36.3 mmol) and KOAc (8.90 g, 90.68 mmol) in 1,4-dioxane (200 ml) is inertized with argon for 30 min. Subsequently, Pd(dppf)Cl₂ (740 mg, 0.91 mmol) is added, and the mixture is stirred under reflux for 20 h. After cooling, the solvent is removed on a rotary evaporator, and the residue is worked up by extraction with dichloromethane and water. The combined organic phases are dried over Na₂SO₄, ethanol (150 ml) is added, and the dichloromethane is drawn off on a rotary evaporator. The precipitated solids are filtered off with suction and dried in a vacuum drying cabinet. The crude product is used in the next stage without further purification. Yield: 9.06 g (24.6 mmol, 82%), purity 95% by 1H NMR.

The following compounds can be prepared analogously: As an alternative, the catalyst system used may also be Pd(PCy₃)₂Cl₂ or Pd₂(dba)₃ with S-Phos (1:3). Purification can be effected not only by column chromatography but also by hot extraction, or recrystallization or hot extraction using other standard solvents such as ethanol, butanol, acetone, ethyl acetate, acetonitrile, toluene, xylene, dichloromethane, methanol, tetrahydrofuran, n-butyl acetate, 1,4-dioxane, or recrystallization using high boilers such as dimethyl sulfoxide, N,N-dimethylformamide, N,N-dimethylacetamide, N-methyl pyrrolidone, etc.

| Reactant | Product | Yield |
|---|---|---|
| 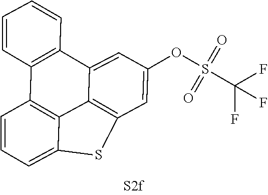 [SMILES: O=S(=O)(Oc1cc2sc3cccc4c5ccccc5c(c1)c2c34)C(F)(F)F] | 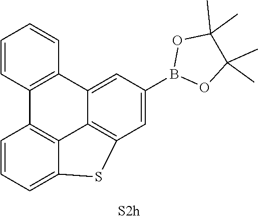 [SMILES: CC1(C)OB(c2cc3sc4cccc5c6ccccc6c(c2)c3c45)OC1(C)C] | 80% |
| 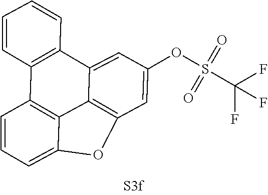 [SMILES: O=S(=O)(Oc1cc2oc3cccc4c5ccccc5c(c1)c2c34)C(F)(F)F] | 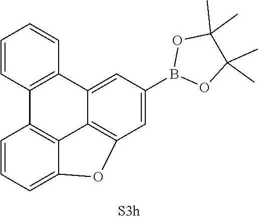 [SMILES: CC1(C)OB(c2cc3oc4cccc5c6ccccc6c(c2)c3c45)OC1(C)C] | 85% |
| 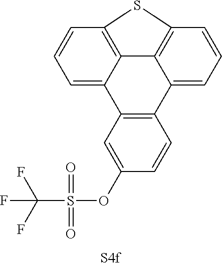 [SMILES: O=S(=O)(Oc1ccc2c(c1)c1cccc3sc4cccc2c4c31)C(F)(F)F] | 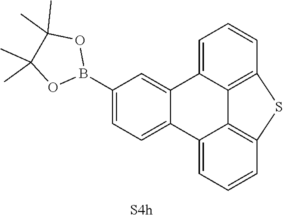 [SMILES: CC1(C)OB(c2ccc3c(c2)c2cccc4sc5cccc3c5c42)OC1(C)C] | 74% |
| 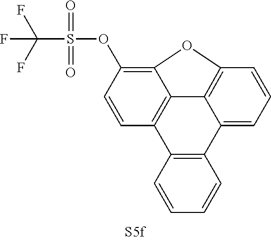 [SMILES: O=S(=O)(Oc1ccc2c3ccccc3c3cccc4oc1c2c43)C(F)(F)F] | 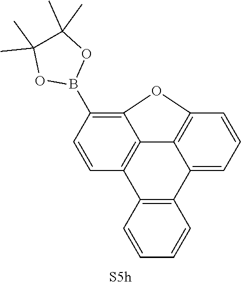 [SMILES: CC1(C)OB(c2ccc3c4ccccc4c4cccc5oc2c3c54)OC1(C)C] | 70% |
| 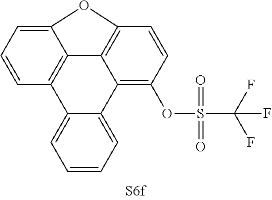 [SMILES: O=S(=O)(Oc1ccc2oc3cccc4c5ccccc5c1c2c34)C(F)(F)F] | 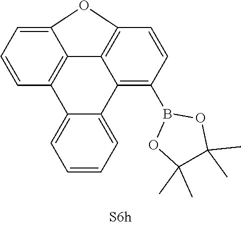 [SMILES: CC1(C)OB(c2ccc3oc4cccc5c6ccccc6c2c3c45)OC1(C)C] | 37% |
| 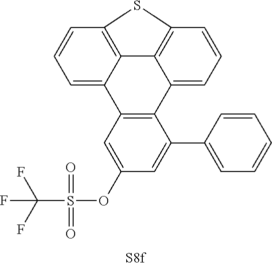 [SMILES: O=S(=O)(Oc1cc(-c2ccccc2)c2c(c1)c1cccc3sc4cccc2c4c31)C(F)(F)F] | 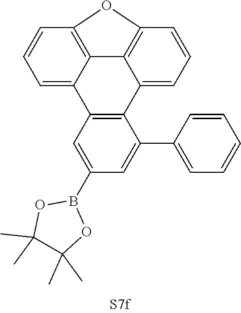 [SMILES: CC1(C)OB(c2cc(-c3ccccc3)c3c(c2)c2cccc4oc5cccc3c5c42)OC1(C)C] | 65% |

### Preparation of the Compounds of the Invention

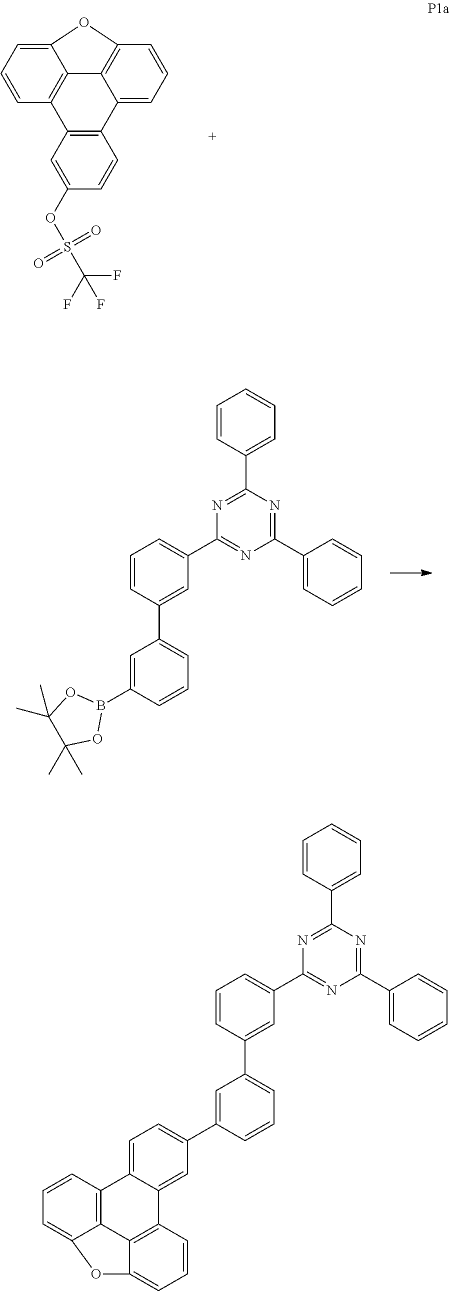 [SMILES: O=S(=O)(Oc1ccc2c(c1)c1cccc3oc4cccc2c4c31)C(F)(F)F ; CC1(C)OB(c2cccc(-c3cccc(-c4nc(-c5ccccc5)nc(-c5ccccc5)n4)c3)c2)OC1(C)C ; c1ccc(-c2nc(-c3ccccc3)nc(-c3cccc(-c4cccc(-c5ccc6c(c5)c5cccc7oc8cccc6c8c75)c4)c3)n2)cc1]

An initial charge of S1f (13.00 g, 33.30 mmol), 2,4-diphenyl-6-[3′-(4,4,5,5-tetramethyl-1,3,2-dioxaborolan-2-yl)[1,1′-biphenyl]-3-yl]-1,3,5-triazine (17.88 g, 34.97 mmol) [1802232-96-7] and K₃PO₄ (14.14 g, 66.61 mmol) in toluene (200 ml), dioxane (200 ml) and water (100 ml) is inertized with argon for 30 min. Subsequently, Pd₂(dba)₃ (305 mg, 0.33 mmol) and triphenylphosphine (175 mg, 0.67 mmol) are added successively, and the reaction mixture is heated to reflux for 18 h. After cooling, the precipitated solids are filtered off with suction and washed with water and ethanol. The crude product is subjected to hot extraction once with toluene and three times with o-xylene, and finally sublimed under high vacuum. Yield: 12.92 g (20.7 mmol; 62%).

The following compounds can be prepared analogously: The catalyst system used may not only be Pd₂(dba)₃ with triphenylphosphine but also S-Phos or X-Phos with Pd(OAc)₂ or Pd₂(dba)₃. Purification can be effected using column chromatography, hot extraction or recrystallization. Recrystallization or hot extraction can be effected using standard solvents such as ethanol, butanol, acetone, ethyl acetate, acetonitrile, toluene, xylene, dichloromethane, methanol, tetrahydrofuran, n-butyl acetate, 1,4-dioxane, or recrystallization using high boilers such as dimethyl sulfoxide, N,N-dimethylformamide, N,N-dimethylacetamide, N-methylpyrrolidone, etc.

| Reactant 1 | Reactant 2 | Product | Yield |
|---|---|---|---|
| 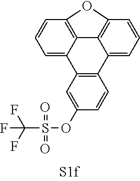 [SMILES: O=S(=O)(Oc1ccc2c(c1)c1cccc3oc4cccc2c4c31)C(F)(F)F] | 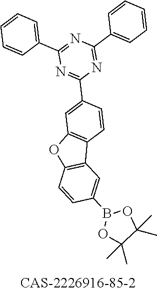 [SMILES: CC1(C)OB(c2ccc3oc4cc(-c5nc(-c6ccccc6)nc(-c6ccccc6)n5)ccc4c3c2)OC1(C)C] | 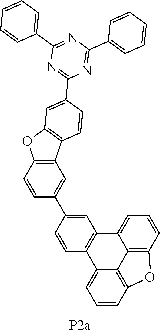 [SMILES: c1ccc(-c2nc(-c3ccccc3)nc(-c3ccc4c(c3)oc3ccc(-c5ccc6c(c5)c5cccc7oc8cccc6c8c75)cc34)n2)cc1] | 58% |
| 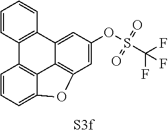 [SMILES: O=S(=O)(Oc1cc2oc3cccc4c5ccccc5c(c1)c2c34)C(F)(F)F] | 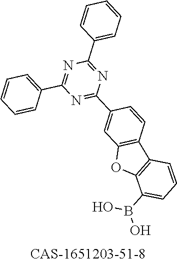 [SMILES: OB(O)c1cccc2c1oc1cc(-c3nc(-c4ccccc4)nc(-c4ccccc4)n3)ccc12] | 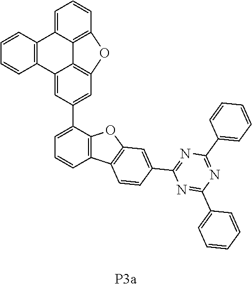 [SMILES: c1ccc(-c2nc(-c3ccccc3)nc(-c3ccc4c(c3)oc3c(-c5cc6oc7cccc8c9ccccc9c(c5)c6c78)cccc34)n2)cc1] | 54% |
| 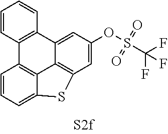 [SMILES: O=S(=O)(Oc1cc2sc3cccc4c5ccccc5c(c1)c2c34)C(F)(F)F] | 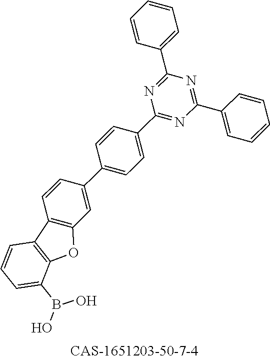 [SMILES: OB(O)c1cccc2c1oc1cc(-c3ccc(-c4nc(-c5ccccc5)nc(-c5ccccc5)n4)cc3)ccc12] | 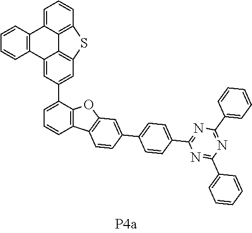 [SMILES: c1ccc(-c2nc(-c3ccccc3)nc(-c3ccc(-c4ccc5c(c4)oc4c(-c6cc7sc8cccc9c%10ccccc%10c(c6)c7c89)cccc45)cc3)n2)cc1] | 45% |
| 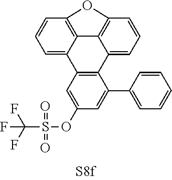 [SMILES: O=S(=O)(Oc1cc(-c2ccccc2)c2c(c1)c1cccc3oc4cccc2c4c31)C(F)(F)F] | 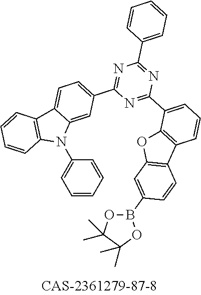 [SMILES: CC1(C)OB(c2ccc3c(c2)oc2c(-c4nc(-c5ccccc5)nc(-c5ccc6c7ccccc7n(-c7ccccc7)c6c5)n4)cccc23)OC1(C)C] | 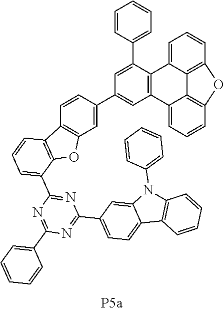 [SMILES: c1ccc(-c2nc(-c3ccc4c5ccccc5n(-c5ccccc5)c4c3)nc(-c3cccc4c3oc3cc(-c5cc(-c6ccccc6)c6c(c5)c5cccc7oc8cccc6c8c75)ccc34)n2)cc1] | 38% |
| 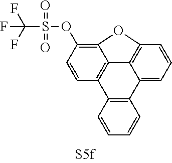 [SMILES: O=S(=O)(Oc1ccc2c3ccccc3c3cccc4oc1c2c43)C(F)(F)F] | 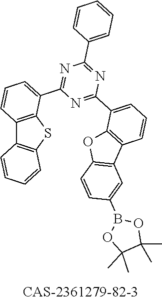 [SMILES: CC1(C)OB(c2ccc3oc4c(-c5nc(-c6ccccc6)nc(-c6cccc7c6sc6ccccc67)n5)cccc4c3c2)OC1(C)C] | 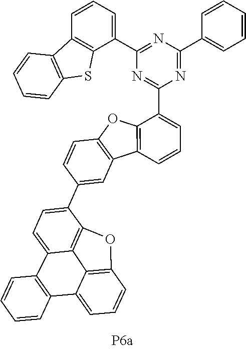 [SMILES: c1ccc(-c2nc(-c3cccc4c3oc3ccc(-c5ccc6c7ccccc7c7cccc8oc5c6c87)cc34)nc(-c3cccc4c3sc3ccccc34)n2)cc1] | 41% |
| 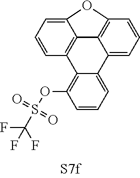 [SMILES: O=S(=O)(Oc1cccc2c3cccc4oc5cccc(c12)c5c43)C(F)(F)F] | 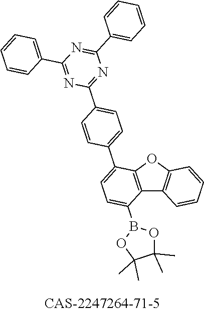 [SMILES: CC1(C)OB(c2ccc(-c3ccc(-c4nc(-c5ccccc5)nc(-c5ccccc5)n4)cc3)c3oc4ccccc4c23)OC1(C)C] | 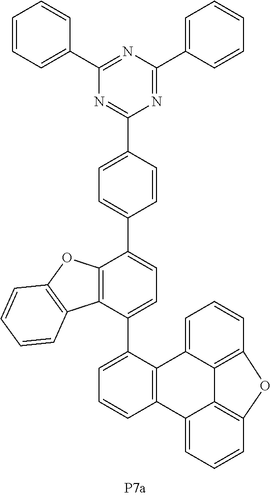 [SMILES: c1ccc(-c2nc(-c3ccccc3)nc(-c3ccc(-c4ccc(-c5cccc6c7cccc8oc9cccc(c56)c9c87)c5c4oc4ccccc45)cc3)n2)cc1] | 17% |
| 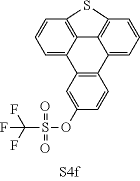 [SMILES: O=S(=O)(Oc1ccc2c(c1)c1cccc3sc4cccc2c4c31)C(F)(F)F] | 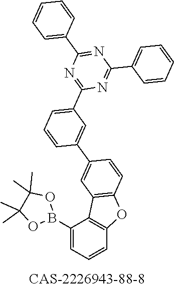 [SMILES: CC1(C)OB(c2cccc3oc4ccc(-c5cccc(-c6nc(-c7ccccc7)nc(-c7ccccc7)n6)c5)cc4c23)OC1(C)C] | 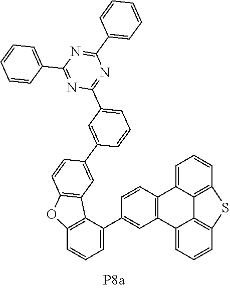 [SMILES: c1ccc(-c2nc(-c3ccccc3)nc(-c3cccc(-c4ccc5oc6cccc(-c7ccc8c(c7)c7cccc9sc%10cccc8c%10c97)c6c5c4)c3)n2)cc1] | 40% |
| 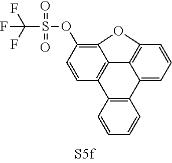 [SMILES: O=S(=O)(Oc1ccc2c3ccccc3c3cccc4oc1c2c43)C(F)(F)F] | 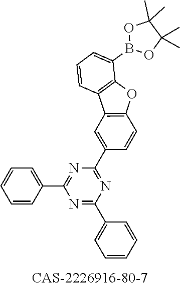 [SMILES: CC1(C)OB(c2cccc3c2oc2ccc(-c4nc(-c5ccccc5)nc(-c5ccccc5)n4)cc23)OC1(C)C] | 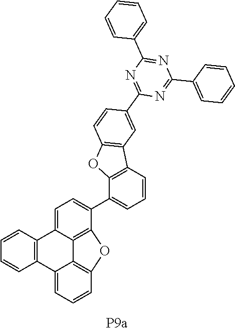 [SMILES: c1ccc(-c2nc(-c3ccccc3)nc(-c3ccc4oc5c(-c6ccc7c8ccccc8c8cccc9oc6c7c98)cccc5c4c3)n2)cc1] | 33% |
| 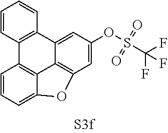 [SMILES: O=S(=O)(Oc1cc2oc3cccc4c5ccccc5c(c1)c2c34)C(F)(F)F] | 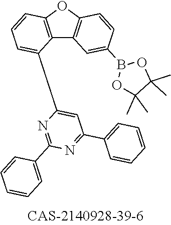 [SMILES: CC1(C)OB(c2ccc3oc4cccc(-c5cc(-c6ccccc6)nc(-c6ccccc6)n5)c4c3c2)OC1(C)C] | 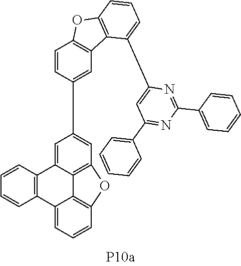 [SMILES: c1ccc(-c2cc(-c3cccc4oc5ccc(-c6cc7oc8cccc9c%10ccccc%10c(c6)c7c89)cc5c34)nc(-c3ccccc3)n2)cc1] | 55% |
| 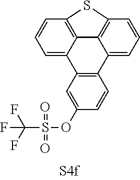 [SMILES: O=S(=O)(Oc1ccc2c(c1)c1cccc3sc4cccc2c4c31)C(F)(F)F] | 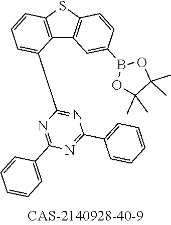 [SMILES: CC1(C)OB(c2ccc3sc4cccc(-c5nc(-c6ccccc6)nc(-c6ccccc6)n5)c4c3c2)OC1(C)C] | 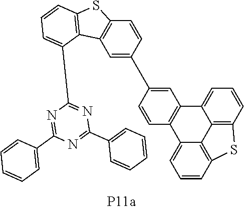 [SMILES: c1ccc(-c2nc(-c3ccccc3)nc(-c3cccc4sc5ccc(-c6ccc7c(c6)c6cccc8sc9cccc7c9c86)cc5c34)n2)cc1] | 61% |
| 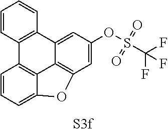 [SMILES: O=S(=O)(Oc1cc2oc3cccc4c5ccccc5c(c1)c2c34)C(F)(F)F] | 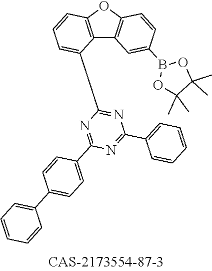 [SMILES: CC1(C)OB(c2ccc3oc4cccc(-c5nc(-c6ccccc6)nc(-c6ccc(-c7ccccc7)cc6)n5)c4c3c2)OC1(C)C] | 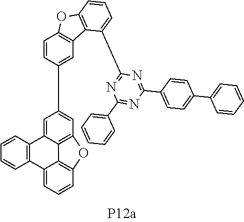 [SMILES: c1ccc(-c2ccc(-c3nc(-c4ccccc4)nc(-c4cccc5oc6ccc(-c7cc8oc9cccc%10c%11ccccc%11c(c7)c8c9%10)cc6c45)n3)cc2)cc1] | 62% |
| 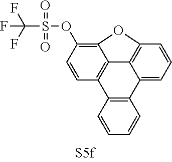 [SMILES: O=S(=O)(Oc1ccc2c3ccccc3c3cccc4oc1c2c43)C(F)(F)F] | 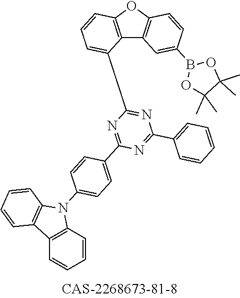 [SMILES: CC1(C)OB(c2ccc3oc4cccc(-c5nc(-c6ccccc6)nc(-c6ccc(-n7c8ccccc8c8ccccc87)cc6)n5)c4c3c2)OC1(C)C] | 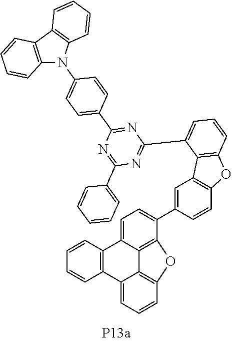 [SMILES: c1ccc(-c2nc(-c3ccc(-n4c5ccccc5c5ccccc54)cc3)nc(-c3cccc4oc5ccc(-c6ccc7c8ccccc8c8cccc9oc6c7c98)cc5c34)n2)cc1] | 37% |
| 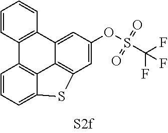 [SMILES: O=S(=O)(Oc1cc2sc3cccc4c5ccccc5c(c1)c2c34)C(F)(F)F] | 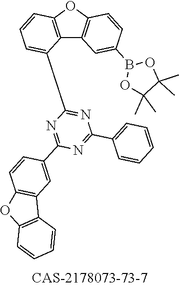 [SMILES: CC1(C)OB(c2ccc3oc4cccc(-c5nc(-c6ccccc6)nc(-c6ccc7oc8ccccc8c7c6)n5)c4c3c2)OC1(C)C] | 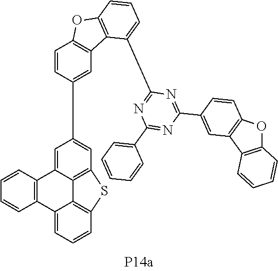 [SMILES: c1ccc(-c2nc(-c3ccc4oc5ccccc5c4c3)nc(-c3cccc4oc5ccc(-c6cc7sc8cccc9c%10ccccc%10c(c6)c7c89)cc5c34)n2)cc1] | 44% |

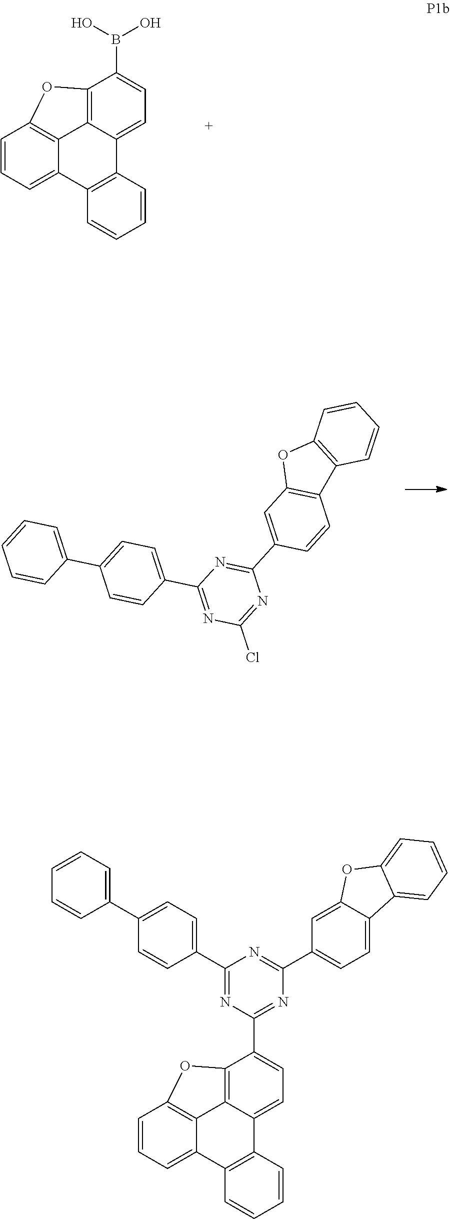 [SMILES: OB(O)c1ccc2c3ccccc3c3cccc4oc1c2c43 ; Clc1nc(-c2ccc(-c3ccccc3)cc2)nc(-c2ccc3c(c2)oc2ccccc23)n1 ; c1ccc(-c2ccc(-c3nc(-c4ccc5c(c4)oc4ccccc45)nc(-c4ccc5c6ccccc6c6cccc7oc4c5c76)n3)cc2)cc1]

An initial charge of S1g (28.61 g, 100 mmol), 2-[1,1′-biphenyl]-4-yl-4-chloro-6-(3-dibenzofuranyl)triazine (43.49 g, 100 mmol) [2170887-83-7] and K₃PO₄ (63.79 g, 300 mmol) in THF (1200 ml) and water (300 ml) is inertized with argon for 30 min. Subsequently, Pd(OAc)₂ (224 mg, 1.00 mmol) and X-Phos (1.00 g, 2.00 mmol) are added successively, and the mixture is stirred under reflux for 9 h. After cooling, the precipitated solids are filtered off with suction and washed with water and ethanol. The crude product is subjected to basic hot extraction twice with toluene and twice with n-butyl acetate over aluminum oxide, and finally sublimed under high vacuum. Yield: 32.7 g, (51.2 mmol, 51%), purity>99.9% by HPLC

The following compounds can be prepared analogously: The catalyst system used may not only be X-Phos but also S-Phos with not only Pd(OAc)₂ but also Pd₂(dba)₃, or Pd(PPh₃)₂Cl₂ or Pd(PPh₃)₄. Purification can be effected using column chromatography, hot extraction or recrystallization. Recrystallization or hot extraction can be effected using standard solvents such as ethanol, butanol, acetone, ethyl acetate, acetonitrile, toluene, xylene, dichloromethane, methanol, tetrahydrofuran, n-butyl acetate, 1,4-dioxane, or recrystallization using high boilers such as dimethyl sulfoxide, N,N-dimethylformamide, N,N-dimethylacetamide, N-methylpyrrolidone, etc.

| Reactant 1 | Reactant 2 | Product | Yield |
|---|---|---|---|
| 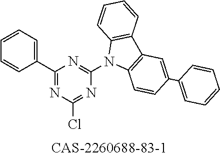 [SMILES: Clc1nc(-c2ccccc2)nc(-n2c3ccccc3c3cc(-c4ccccc4)ccc32)n1] | 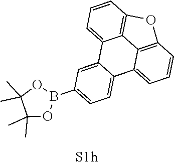 [SMILES: CC1(C)OB(c2ccc3c(c2)c2cccc4oc5cccc3c5c42)OC1(C)C] | 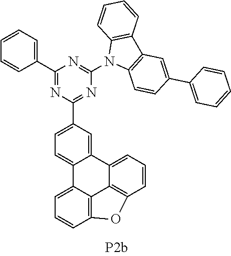 [SMILES: c1ccc(-c2ccc3c(c2)c2ccccc2n3-c2nc(-c3ccccc3)nc(-c3ccc4c(c3)c3cccc5oc6cccc4c6c53)n2)cc1] | 45% |
| 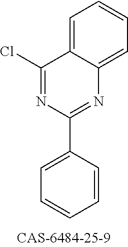 [SMILES: Clc1nc(-c2ccccc2)nc2ccccc12] | 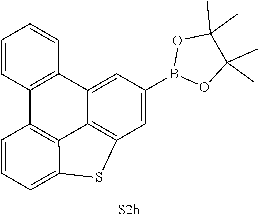 [SMILES: CC1(C)OB(c2cc3sc4cccc5c6ccccc6c(c2)c3c45)OC1(C)C] | 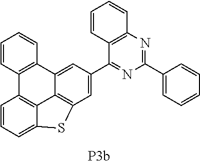 [SMILES: c1ccc(-c2nc(-c3cc4sc5cccc6c7ccccc7c(c3)c4c56)c3ccccc3n2)cc1] | 33% |
| 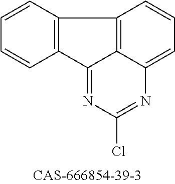 [SMILES: Clc1nc2c3c(cccc3n1)-c1ccccc1-2] | 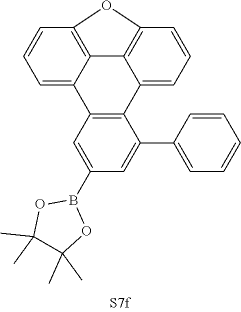 | 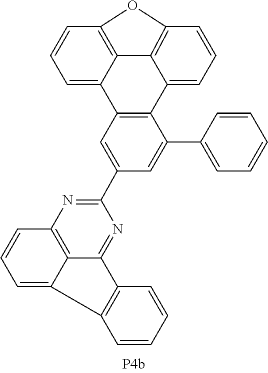 [SMILES: c1ccc(-c2cc(-c3nc4c5c(cccc5n3)-c3ccccc3-4)cc3c4cccc5oc6cccc(c23)c6c54)cc1] | 38% |
| 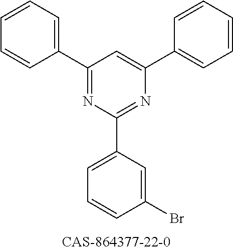 [SMILES: Brc1cccc(-c2nc(-c3ccccc3)cc(-c3ccccc3)n2)c1] | 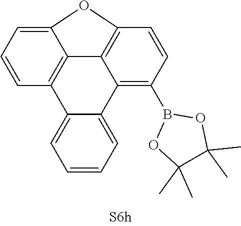 [SMILES: CC1(C)OB(c2ccc3oc4cccc5c6ccccc6c2c3c45)OC1(C)C] | 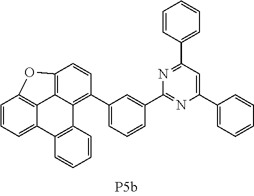 [SMILES: c1ccc(-c2cc(-c3ccccc3)nc(-c3cccc(-c4ccc5oc6cccc7c8ccccc8c4c5c67)c3)n2)cc1] | 28% |
| 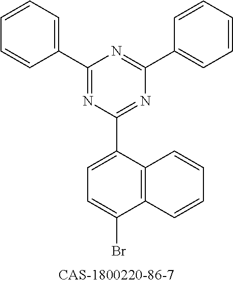 [SMILES: Brc1ccc(-c2nc(-c3ccccc3)nc(-c3ccccc3)n2)c2ccccc12] | 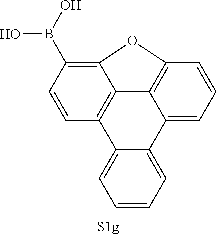 [SMILES: OB(O)c1ccc2c3ccccc3c3cccc4oc1c2c43] | 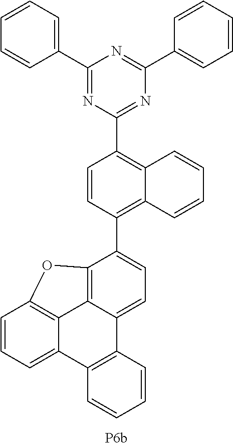 [SMILES: c1ccc(-c2nc(-c3ccccc3)nc(-c3ccc(-c4ccc5c6ccccc6c6cccc7oc4c5c76)c4ccccc34)n2)cc1] | 32% |
| 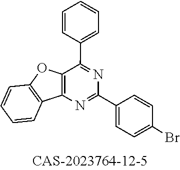 [SMILES: Brc1ccc(-c2nc(-c3ccccc3)c3oc4ccccc4c3n2)cc1] | 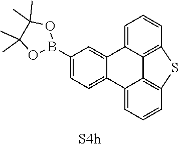 [SMILES: CC1(C)OB(c2ccc3c(c2)c2cccc4sc5cccc3c5c42)OC1(C)C] | 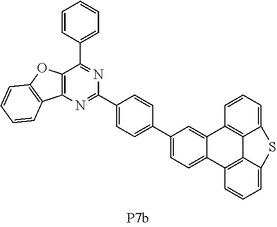 [SMILES: c1ccc(-c2nc(-c3ccc(-c4ccc5c(c4)c4cccc6sc7cccc5c7c64)cc3)nc3c2oc2ccccc23)cc1] | 54% |
| 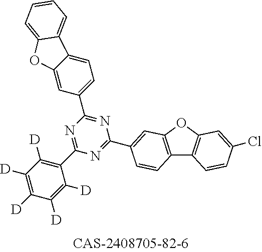 [SMILES: [2H]c1c([2H])c([2H])c(-c2nc(-c3ccc4c(c3)oc3ccccc34)nc(-c3ccc4c(c3)oc3cc(Cl)ccc34)n2)c([2H])c1[2H]] | 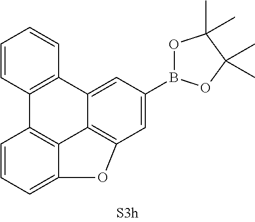 [SMILES: CC1(C)OB(c2cc3oc4cccc5c6ccccc6c(c2)c3c45)OC1(C)C] | 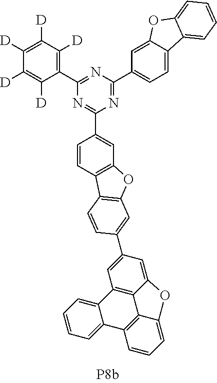 [SMILES: [2H]c1c([2H])c([2H])c(-c2nc(-c3ccc4c(c3)oc3ccccc34)nc(-c3ccc4c(c3)oc3cc(-c5cc6oc7cccc8c9ccccc9c(c5)c6c78)ccc34)n2)c([2H])c1[2H]] | 44% |
| 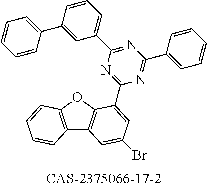 [SMILES: Brc1cc(-c2nc(-c3ccccc3)nc(-c3cccc(-c4ccccc4)c3)n2)c2oc3ccccc3c2c1] | 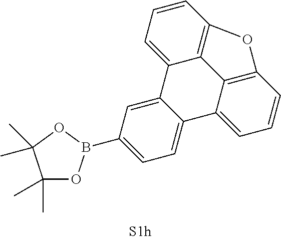 [SMILES: CC1(C)OB(c2ccc3c(c2)c2cccc4oc5cccc3c5c42)OC1(C)C] | 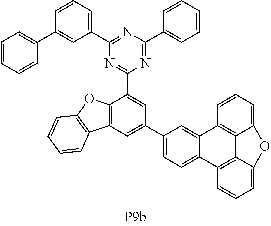 [SMILES: c1ccc(-c2cccc(-c3nc(-c4ccccc4)nc(-c4cc(-c5ccc6c(c5)c5cccc7oc8cccc6c8c75)cc5c4oc4ccccc45)n3)c2)cc1] | 44% |
| 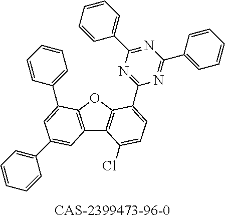 [SMILES: Clc1ccc(-c2nc(-c3ccccc3)nc(-c3ccccc3)n2)c2oc3c(-c4ccccc4)cc(-c4ccccc4)cc3c12] | 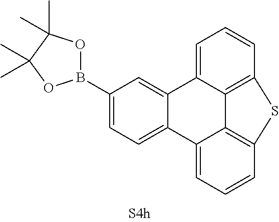 [SMILES: CC1(C)OB(c2ccc3c(c2)c2cccc4sc5cccc3c5c42)OC1(C)C] | 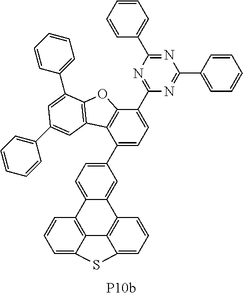 [SMILES: c1ccc(-c2cc(-c3ccccc3)c3oc4c(-c5nc(-c6ccccc6)nc(-c6ccccc6)n5)ccc(-c5ccc6c(c5)c5cccc7sc8cccc6c8c75)c4c3c2)cc1] | 34% |
| 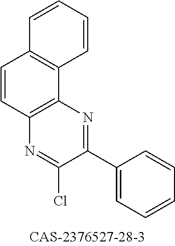 [SMILES: Clc1nc2ccc3ccccc3c2nc1-c1ccccc1] | 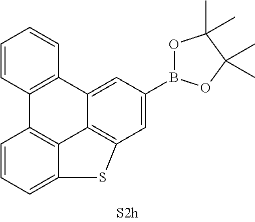 [SMILES: CC1(C)OB(c2cc3sc4cccc5c6ccccc6c(c2)c3c45)OC1(C)C] | 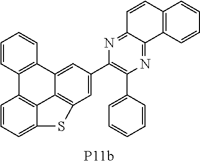 [SMILES: c1ccc(-c2nc3c(ccc4ccccc43)nc2-c2cc3sc4cccc5c6ccccc6c(c2)c3c45)cc1] | 17% |
| 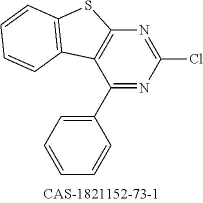 [SMILES: Clc1nc(-c2ccccc2)c2c(n1)sc1ccccc12] | 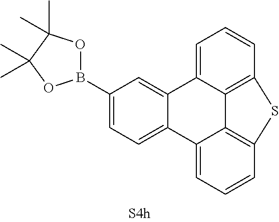 [SMILES: CC1(C)OB(c2ccc3c(c2)c2cccc4sc5cccc3c5c42)OC1(C)C] | 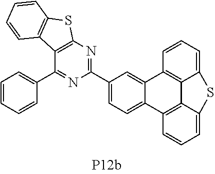 [SMILES: c1ccc(-c2nc(-c3ccc4c(c3)c3cccc5sc6cccc4c6c53)nc3sc4ccccc4c23)cc1] | 42% |
| 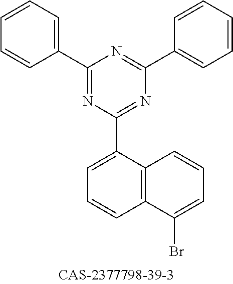 [SMILES: Brc1cccc2c(-c3nc(-c4ccccc4)nc(-c4ccccc4)n3)cccc12] | 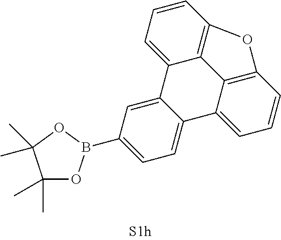 [SMILES: CC1(C)OB(c2ccc3c(c2)c2cccc4oc5cccc3c5c42)OC1(C)C] | 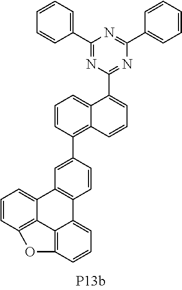 [SMILES: c1ccc(-c2nc(-c3ccccc3)nc(-c3cccc4c(-c5ccc6c(c5)c5cccc7oc8cccc6c8c75)cccc34)n2)cc1] | 33% |
| 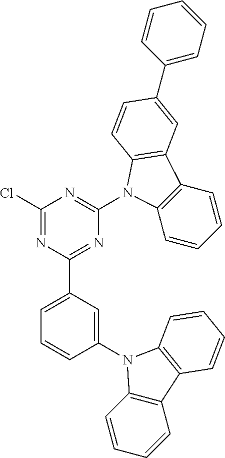 [SMILES: Clc1nc(-c2cccc(-n3c4ccccc4c4ccccc43)c2)nc(-n2c3ccccc3c3cc(-c4ccccc4)ccc32)n1] | 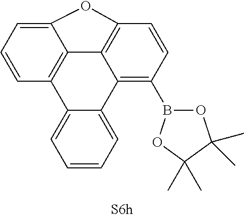 [SMILES: CC1(C)OB(c2ccc3oc4cccc5c6ccccc6c2c3c45)OC1(C)C] | 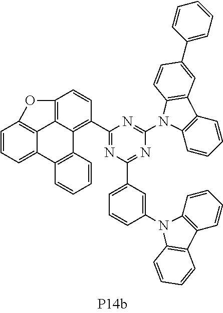 [SMILES: c1ccc(-c2ccc3c(c2)c2ccccc2n3-c2nc(-c3cccc(-n4c5ccccc5c5ccccc54)c3)nc(-c3ccc4oc5cccc6c7ccccc7c3c4c56)n2)cc1] | 15% |
| 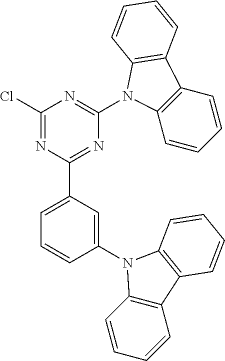 [SMILES: Clc1nc(-c2cccc(-n3c4ccccc4c4ccccc43)c2)nc(-n2c3ccccc3c3ccccc32)n1] | 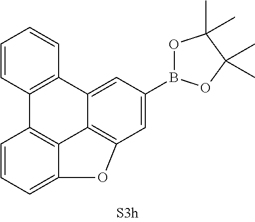 [SMILES: CC1(C)OB(c2cc3oc4cccc5c6ccccc6c(c2)c3c45)OC1(C)C] | 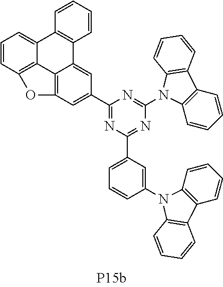 [SMILES: c1cc(-c2nc(-c3cc4oc5cccc6c7ccccc7c(c3)c4c56)nc(-n3c4ccccc4c4ccccc43)n2)cc(-n2c3ccccc3c3ccccc32)c1] | 57% |
| 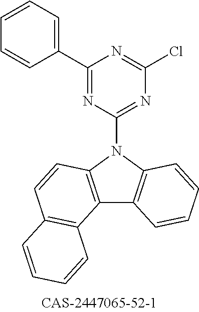 [SMILES: Clc1nc(-c2ccccc2)nc(-n2c3ccccc3c3c4ccccc4ccc32)n1] | 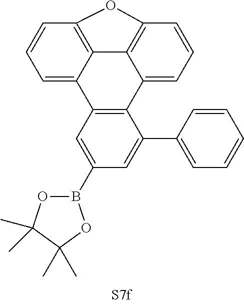 [SMILES: CC1(C)OB(c2cc(-c3ccccc3)c3c(c2)c2cccc4oc5cccc3c5c42)OC1(C)C] | 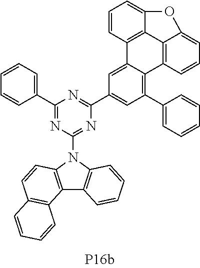 [SMILES: c1ccc(-c2nc(-c3cc(-c4ccccc4)c4c(c3)c3cccc5oc6cccc4c6c53)nc(-n3c4ccccc4c4c5ccccc5ccc43)n2)cc1] | 39% |
| 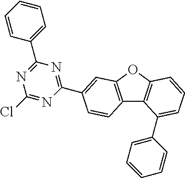 [SMILES: Clc1nc(-c2ccccc2)nc(-c2ccc3c(c2)oc2cccc(-c4ccccc4)c23)n1] | 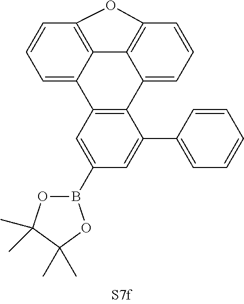 [SMILES: CC1(C)OB(c2cc(-c3ccccc3)c3c(c2)c2cccc4oc5cccc3c5c42)OC1(C)C] | 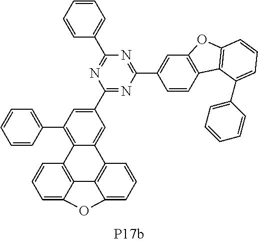 [SMILES: c1ccc(-c2nc(-c3ccc4c(c3)oc3cccc(-c5ccccc5)c34)nc(-c3cc(-c4ccccc4)c4c(c3)c3cccc5oc6cccc4c6c53)n2)cc1] | 33% |
| 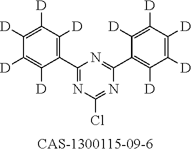 [SMILES: [2H]c1c([2H])c([2H])c(-c2nc(Cl)nc(-c3c([2H])c([2H])c([2H])c([2H])c3[2H])n2)c([2H])c1[2H]] | 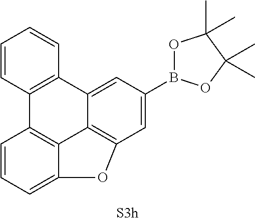 [SMILES: CC1(C)OB(c2cc3oc4cccc5c6ccccc6c(c2)c3c45)OC1(C)C] | 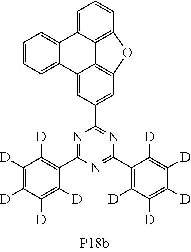 [SMILES: [2H]c1c([2H])c([2H])c(-c2nc(-c3cc4oc5cccc6c7ccccc7c(c3)c4c56)nc(-c3c([2H])c([2H])c([2H])c([2H])c3[2H])n2)c([2H])c1[2H]] | 53% |
| 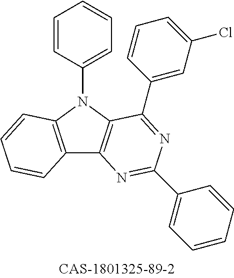 [SMILES: Clc1cccc(-c2nc(-c3ccccc3)nc3c4ccccc4n(-c4ccccc4)c23)c1] | 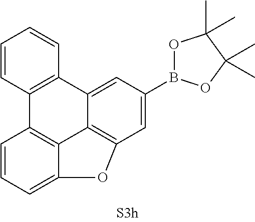 [SMILES: CC1(C)OB(c2cc3oc4cccc5c6ccccc6c(c2)c3c45)OC1(C)C] | 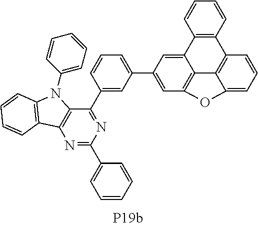 [SMILES: c1ccc(-c2nc(-c3cccc(-c4cc5oc6cccc7c8ccccc8c(c4)c5c67)c3)c3c(n2)c2ccccc2n3-c2ccccc2)cc1] | 34% |
| 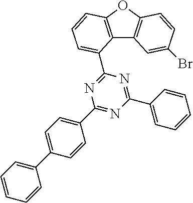 [SMILES: Brc1ccc2oc3cccc(-c4nc(-c5ccccc5)nc(-c5ccc(-c6ccccc6)cc5)n4)c3c2c1] | 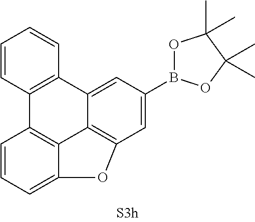 [SMILES: CC1(C)OB(c2cc3oc4cccc5c6ccccc6c(c2)c3c45)OC1(C)C] | 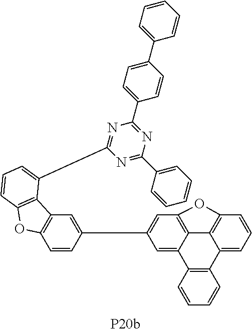 [SMILES: c1ccc(-c2ccc(-c3nc(-c4ccccc4)nc(-c4cccc5oc6ccc(-c7cc8oc9cccc%10c%11ccccc%11c(c7)c8c9%10)cc6c45)n3)cc2)cc1] | 42% |
| 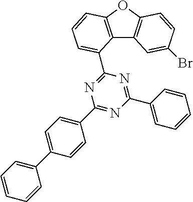 [SMILES: Brc1ccc2oc3cccc(-c4nc(-c5ccccc5)nc(-c5ccc(-c6ccccc6)cc5)n4)c3c2c1] | 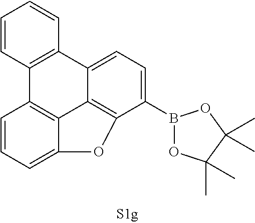 [SMILES: CC1(C)OB(c2ccc3c4ccccc4c4cccc5oc2c3c54)OC1(C)C] | 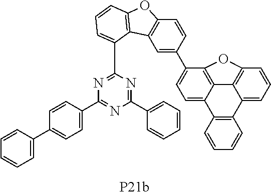 [SMILES: c1ccc(-c2ccc(-c3nc(-c4ccccc4)nc(-c4cccc5oc6ccc(-c7ccc8c9ccccc9c9cccc%10oc7c8c%109)cc6c45)n3)cc2)cc1] | 48% |
| 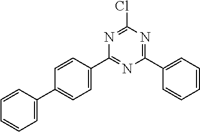 [SMILES: Clc1nc(-c2ccccc2)nc(-c2ccc(-c3ccccc3)cc2)n1] | 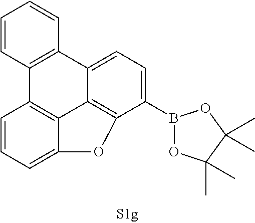 [SMILES: CC1(C)OB(c2ccc3c4ccccc4c4cccc5oc2c3c54)OC1(C)C] | 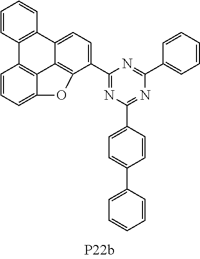 [SMILES: c1ccc(-c2ccc(-c3nc(-c4ccccc4)nc(-c4ccc5c6ccccc6c6cccc7oc4c5c76)n3)cc2)cc1] | 55% |
| 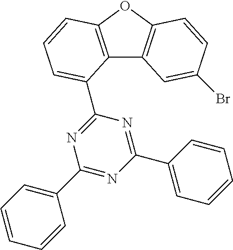 [SMILES: Brc1ccc2oc3cccc(-c4nc(-c5ccccc5)nc(-c5ccccc5)n4)c3c2c1] | 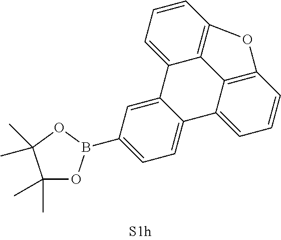 [SMILES: CC1(C)OB(c2ccc3c(c2)c2cccc4oc5cccc3c5c42)OC1(C)C] | 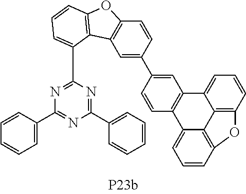 [SMILES: c1ccc(-c2nc(-c3ccccc3)nc(-c3cccc4oc5ccc(-c6ccc7c(c6)c6cccc8oc9cccc7c9c86)cc5c34)n2)cc1] | 36% |
| 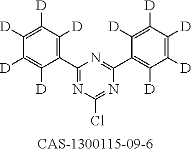 [SMILES: [2H]c1c([2H])c([2H])c(-c2nc(Cl)nc(-c3c([2H])c([2H])c([2H])c([2H])c3[2H])n2)c([2H])c1[2H]] | 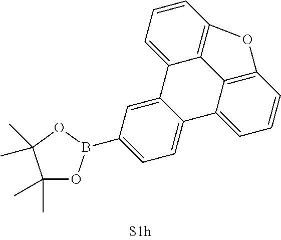 [SMILES: CC1(C)OB(c2ccc3c(c2)c2cccc4oc5cccc3c5c42)OC1(C)C] | 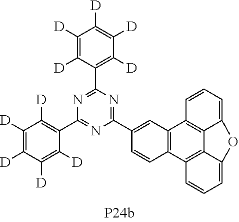 [SMILES: [2H]c1c([2H])c([2H])c(-c2nc(-c3ccc4c(c3)c3cccc5oc6cccc4c6c53)nc(-c3c([2H])c([2H])c([2H])c([2H])c3[2H])n2)c([2H])c1[2H]] | 31% |
| 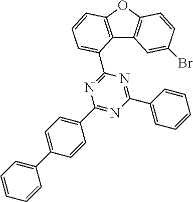 [SMILES: Brc1ccc2oc3cccc(-c4nc(-c5ccccc5)nc(-c5ccc(-c6ccccc6)cc5)n4)c3c2c1] | 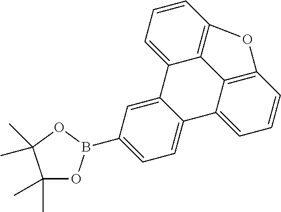 [SMILES: CC1(C)OB(c2ccc3c(c2)c2cccc4oc5cccc3c5c42)OC1(C)C] | 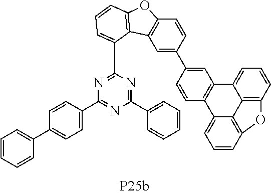 [SMILES: c1ccc(-c2ccc(-c3nc(-c4ccccc4)nc(-c4cccc5oc6ccc(-c7ccc8c(c7)c7cccc9oc%10cccc8c%10c97)cc6c45)n3)cc2)cc1] | 40% |
| 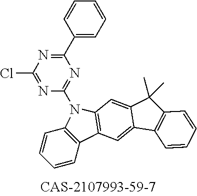 [SMILES: CC1(C)c2ccccc2-c2cc3c4ccccc4n(-c4nc(Cl)nc(-c5ccccc5)n4)c3cc21] | 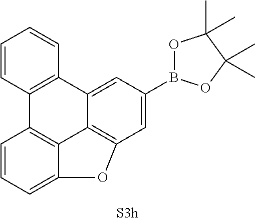 [SMILES: CC1(C)OB(c2cc3oc4cccc5c6ccccc6c(c2)c3c45)OC1(C)C] | 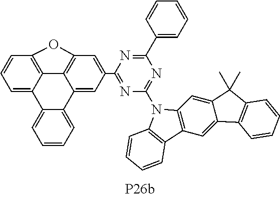 [SMILES: CC1(C)c2ccccc2-c2cc3c4ccccc4n(-c4nc(-c5ccccc5)nc(-c5cc6oc7cccc8c9ccccc9c(c5)c6c78)n4)c3cc21] | 44% |

Production of the OLEDs

The examples which follow (see tables 1 to 7) present the use of the compounds of the invention in OLEDs by comparison with materials from the prior art.

Pretreatment for Examples V1 to V7 and E1a to E7b:

Glass plates coated with structured ITO (indium tin oxide) of thickness 50 nm are treated prior to coating, first with an oxygen plasma, followed by an argon plasma. These plasma-treated glass plates form the substrates to which the OLEDs are applied.

The OLEDs basically have the following layer structure: substrate/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminum layer of thickness 100 nm. The exact structure of the OLEDs can be found in tables 1, 3 and 5. The materials required for production of the OLEDs, if they have not already been described before, are shown in table 7. The device data of the OLEDs are listed in tables 2, 4 and 6. Examples V1 to V7 are comparative examples. Examples E1a-g, E2a-g, E3a-f, E4a-f E5a-e, E6a-c, E7a, E7b show data of inventive OLEDs.

All materials are applied by thermal vapor deposition in a vacuum chamber. In this case, the emission layer always consists of at least two matrix materials and an emitting dopant (emitter) which is added to the matrix material(s) in a particular proportion by volume by co-evaporation. Details given in such a form as P1a:H1:TE2 (32%:60%:8%) mean here that the material P1a is present in the layer in a proportion by volume of 32%, H1 in a proportion by volume of 60% and TE2 in a proportion by volume of 8%. Analogously, the electron transport layer may also consist of a mixture of two materials.

Electroluminescence spectra are determined at a luminance of 1000 cd/m², and these are used to calculate the CIE 1931 x and y color coordinates. The parameter U10 in tables 2 and 6 refers to the voltage which is required for a current density of 10 mA/cm². EQE10 denotes the external quantum efficiency which is attained at 10 mA/cm². The lifetime LD is defined as the time after which luminance, measured in cd/m² in forward direction, drops from the starting luminance to a certain proportion L1 in the course of operation with constant current density j₀. A figure of L1=80% in table 2 means that the lifetime reported in the LD column corresponds to the time after which luminance in cd/m² falls to 80% of its starting value.

The parameter U1000 in table 4 refers to the voltage which is required for a luminance of 1000 cd/m². EQE1000 denotes the external quantum efficiency which is attained at 1000 cd/m². The lifetime LT is defined as the time after which the luminance drops from the starting luminance to a certain proportion L1 in the course of operation with constant current density j⁰. A figure of L1=95% in table 4 means that the lifetime reported in the LT column corresponds to the time after which the luminance falls to 95% of its starting value.

Use of Compounds of the Invention in OLEDs

The inventive materials are used in examples E1a-g, E2a-g, E3a-f, E4a-f E5a-e as matrix materials in the emission layer of green- or red-phosphorescent OLEDs, in examples E6a-c as hole blocker material in the hole blocker layer of blue-fluorescent OLEDs, and in examples E7a and E7b as electron transport materials in the electron transport layer of blue-fluorescent OLEDs. As a comparison from the prior art, materials SdT1, SdT2, SdT3 and SdT4 are used in combination with the host materials H1, H2 and H3 in comparative examples V1 to V5. In the comparison of the inventive examples with the corresponding comparative examples, it is clearly apparent that the inventive examples each show a distinct benefit in the lifetime of the OLED and, for the blue-fluorescent OLEDs in examples 6a-c, 7a und b by comparison with V6 and V7, a benefit in operating voltage and efficiency with otherwise comparable performance data of the OLED.

TABLE 1
Structure of the OLEDs for green emission

| | HIL | HTL | EBL | EML | HBL | ETL | EIL |
|---|---|---|---|---|---|---|---|
| Ex. | thickness | thickness | thickness | thickness | thickness | thickness | thickness |
| V1 | SpMA1:PD1 | SpMA1 | SpMA2 | SdT1:H1:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1a | SpMA1:PD1 | SpMA1 | SpMA2 | P1a:H1:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1b | SpMA1:PD1 | SpMA1 | SpMA2 | P1a:H2:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1c | SpMA1:PD1 | SpMA1 | SpMA2 | P4a:H1:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1d | SpMA1:PD1 | SpMA1 | SpMA2 | P10a:H1:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1e | SpMA1:PD1 | SpMA1 | SpMA2 | P12a:H1TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1f | SpMA1:PD1 | SpMA1 | SpMA2 | P5b:H1:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1g | SpMA1:PD1 | SpMA1 | SpMA2 | P7b:H1:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| V2 | SpMA1:PD1 | SpMA1 | SpMA2 | SdT2:H1:TE2 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E2a | SpMA1:PD1 | SpMA1 | SpMA2 | P1a:H1:TE2 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E2b | SpMA1:PD1 | SpMA1 | SpMA2 | P1a:H2:TE2 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E2c | SpMA1:PD1 | SpMA1 | SpMA2 | P7a:H2:TE2 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E2d | SpMA1:PD1 | SpMA1 | SpMA2 | P1b:H2:TE2 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E2e | SpMA1:PD1 | SpMA1 | SpMA2 | P8b:H1:TE2 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E2f | SpMA1:PD1 | SpMA1 | SpMA2 | P9b:H1:TE2 | ST2 | ST2:LiQ | LiQ |
| | (95%:5%) | 200 nm | 20 nm | (32%:50%:12%) | 5 nm | (50%:50%) | 1 nm |
| | 20 nm | | | 40 nm | | 30 nm | |
| E2g | SpMA1:PD1 | SpMA1 | SpMA2 | P19b:H1:TE2 | ST2 | ST2:LiQ | LiQ |
| | (95%:5%) | 200 nm | 20 nm | (32%:50%:12%) | 5 nm | (50%:50%) | 1 nm |
| | 20 nm | | | 40 nm | | 30 nm | |
| V3 | SpMA1:PD1 | SpMA1 | SpMA2 | SdT3:H1:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E3a | SpMA1:PD1 | SpMA1 | SpMA2 | P1a:H1:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E3b | SpMA1:PD1 | SpMA1 | SpMA2 | P2a:H1:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E3c | SpMA1:PD1 | SpMA1 | SpMA2 | P2b:H2:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E3d | SpMA1:PD1 | SpMA1 | SpMA2 | P14b:H1:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E3e | SpMA1:PD1 | SpMA1 | SpMA2 | P3ab:H2:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E3f | SpMA1:PD1 | SpMA1 | SpMA2 | P12b:H2:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| V4 | SpMA1:PD1 | SpMA1 | SpMA2 | SdT4:H1:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E4a | SpMA1:PD1 | SpMA1 | SpMA2 | P11a:H2:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E4b | SpMA1:PD1 | SpMA1 | SpMA2 | P15b:H1:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E4c | SpMA1:PD1 | SpMA1 | SpMA2 | P17b:H2:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E4d | SpMA1:PD1 | SpMA1 | SpMA2 | P18b:H2:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E4e | SpMA1:PD1 | SpMA1 | SpMA2 | P25b:H2:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E4f | SpMA1:PD1 | SpMA1 | SpMA2 | P26b:H2:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |

TABLE 2
Data of the OLEDs for green emission

| | U10 | EQE10 | CIE x/y at | j₀ | L1 | LD |
|---|---|---|---|---|---|---|
| Ex. | (V) | (%) | 1000 cd/m² | (mA/cm²) | (%) | (h) |
| V1 | 4.4 | 20.5 | 0.34/0.63 | 40 | 80 | 200 |
| E1a | 4.2 | 23.0 | 0.34/0.63 | 40 | 80 | 650 |
| E1b | 4.4 | 22.5 | 0.34/0.63 | 40 | 80 | 770 |
| E1c | 4.2 | 22.0 | 0.34/0.63 | 40 | 80 | 810 |
| E1d | 4.2 | 22.3 | 0.34/0.63 | 40 | 80 | 500 |
| E1e | 4.3 | 23.2 | 0.35/0.63 | 40 | 80 | 880 |
| E1f | 4.1 | 23.5 | 0.34/0.63 | 40 | 80 | 630 |
| E1g | 4.3 | 22.2 | 0.34/0.63 | 40 | 80 | 720 |
| V2 | 5.0 | 16.8 | 0.34/0.63 | 40 | 80 | 180 |
| E2a | 4.1 | 21.9 | 0.35/0.63 | 40 | 80 | 845 |
| E2b | 4.2 | 21.5 | 0.35/0.63 | 40 | 80 | 970 |
| E2c | 4.3 | 22.0 | 0.35/0.63 | 40 | 80 | 745 |
| E2d | 4.2 | 21.7 | 0.35/0.63 | 40 | 80 | 770 |
| E2e | 4.1 | 21.9 | 0.35/0.63 | 40 | 80 | 1220 |
| E2f | 4.3 | 20.9 | 0.35/0.63 | 40 | 80 | 1060 |
| E2g | 4.3 | 21.1 | 0.34/0.63 | 40 | 80 | 715 |
| V3 | 6.0 | 13.0 | 0.34/0.62 | 40 | 80 | 140 |
| E3a | 4.9 | 19.2 | 0.34/0.62 | 40 | 80 | 490 |
| E3b | 4.7 | 18.8 | 0.33/0.62 | 40 | 80 | 690 |
| E3c | 4.9 | 19.0 | 0.33/0.62 | 40 | 80 | 975 |
| E3d | 4.6 | 18.8 | 0.33/0.62 | 40 | 80 | 785 |
| E3e | 4.6 | 18.2 | 0.33/0.62 | 40 | 80 | 955 |
| E3f | 4.5 | 18.6 | 0.33/0.62 | 40 | 80 | 810 |
| V4 | 4.7 | 21.2 | 0.34/0.63 | 40 | 80 | 310 |
| E4a | 4.4 | 21.7 | 0.35/0.63 | 40 | 80 | 1020 |
| E4b | 4.2 | 21.9 | 0.34/0.63 | 40 | 80 | 1090 |
| E4c | 4.1 | 23.5 | 0.34/0.63 | 40 | 80 | 675 |
| E4d | 4.4 | 23.0 | 0.34/0.63 | 40 | 80 | 520 |
| E4e | 4.2 | 22.5 | 0.35/0.63 | 40 | 80 | 1170 |
| E4f | 4.3 | 23.3 | 0.35/0.63 | 40 | 80 | 965 |

TABLE 3
Structure of the OLEDs for red emission

| | HIL | HTL | EBL | EML | HBL | ETL | EIL |
|---|---|---|---|---|---|---|---|
| Ex. | thickness | thickness | thickness | thickness | thickness | thickness | thickness |
| V5 | SpMA1:PD1 | SpMA1 | SpMA2 | SdT1:H3:TER1 | ST2 | ST2:LiQ | LiQ |
| | (95%:5%) | 110 nm | 10 nm | (57%:40%:3%) | 10 nm | (50%:50%) | 1 nm |
| | 20 nm | | | 35 nm | | 30 nm | |
| E5a | SpMA1:PD1 | SpMA1 | SpMA2 | P4b:H3:TER1 | ST2 | ST2:LIQ | LiQ |
| | (95%:5%) | 110 nm | 10 nm | (57%:40%:3%) | 10 nm | (50%:50%) | 1 nm |
| | 20 nm | | | 35 nm | | 30 nm | |
| E5b | SpMA1:PD1 | SpMA1 | SpMA2 | P6b:H1:TER1 | ST2 | ST2:LiQ | LiQ |
| | (95%:5%) | 110 nm | 10 nm | (57%:40%:3%) | 10 nm | (50%:50%) | 1 nm |
| | 20 nm | | | 35 nm | | 30 nm | |
| E5c | SpMA1:PD1 | SpMA1 | SpMA2 | P11b:H2:TER1 | ST2 | ST2:LiQ | LiQ |
| | (95%:5%) | 110 nm | 10 nm | (57%:40%:3%) | 10 nm | (50%:50%) | 1 nm |
| | 20 nm | | | 35 nm | | 30 nm | |
| E5d | SpMA1:PD1 | SpMA1 | SpMA2 | P13b:H2:TER1 | ST2 | ST2:LiQ | LiQ |
| | (95%:5%) | 110 nm | 10 nm | (57%:40%:3%) | 10 nm | (50%:50%) | 1 nm |
| | 20 nm | | | 35 nm | | 30 nm | |
| E5e | SpMA1:PD1 | SpMA1 | SpMA2 | P16b:H2:TER1 | ST2 | ST2:LiQ | LiQ |
| | (95%:5%) | 110 nm | 10 nm | (57%:40%:3%) | 10 nm | (50%:50%) | 1 nm |
| | 20 nm | | | 35 nm | | 30 nm | |

TABLE 4
Data of the OLEDs for red emission

| | U1000 | EQE 1000 | CIE x/y at | j₀ | L1 | LD |
|---|---|---|---|---|---|---|
| Ex. | (V) | (%) | 1000 cd/m² | (mA/cm²) | (%) | (h) |
| V5 | 3.7 | 24.1 | 0.66/0.33 | 60 | 95 | 12 |
| E5a | 3.4 | 25.5 | 0.66/0.33 | 60 | 95 | 110 |
| E5b | 3.3 | 26.0 | 0.67/0.33 | 60 | 95 | 70 |
| E5c | 3.4 | 26.1 | 0.67/0.33 | 60 | 95 | 65 |
| E5d | 3.4 | 25.2 | 0.66/0.33 | 60 | 95 | 57 |
| E5e | 3.2 | 26.1 | 0.66/0.33 | 60 | 95 | 49 |

TABLE 5
Structure of the OLEDs for blue emission

| | HIL | HTL | EBL | EML | HBL | ETL | EIL |
|---|---|---|---|---|---|---|---|
| | Thickness/ | Thickness/ | Thickness/ | Thickness/ | Thickness/ | Thickness/ | Thickness/ |
| Ex. | nm | nm | nm | nm | nm | nm | nm |
| V6 | SpMA1:PD1(5%) | SpMA1 | SpMA2 | H5:SEB(5%) | SdT1 | ST2:LiQ(50%) | LiQ |
| | 20 nm | 180 nm | 10 nm | 20 nm | 10 nm | 20 nm | 1 nm |
| E6a | SpMA1:PD1(5%) | SpMA1 | SpMA2 | H5:SEB(5%) | P1a | ST2:LiQ(50%) | LiQ |
| | 20 nm | 180 nm | 10 nm | 20 nm | 10 nm | 20 nm | 1 nm |
| E6b | SpMA1:PD1(5%) | SpMA1 | SpMA2 | H5:SEB(5%) | P8b | ST2:LiQ(50%) | LiQ |
| | 20 nm | 180 nm | 10 nm | 20 nm | 10 nm | 20 nm | 1 nm |
| E6c | SpMA1:PD1(5%) | SpMA1 | SpMA2 | H5:SEB(5%) | ST2 | P11b:LiQ(50%) | LiQ |
| | 20 nm | 180 nm | 10 nm | 20 nm | 10 nm | 20 nm | 1 nm |
| V7 | SpMA1:PD1(5%) | SpMA1 | SpMA2 | H5:SEB(5%) | SdT1 | SdT1:LiQ(50%) | LiQ |
| | 20 nm | 180 nm | 10 nm | 20 nm | 10 nm | 20 nm | 1 nm |
| E7a | SpMA1:PD1(5%) | SpMA1 | SpMA2 | H5:SEB(5%) | P1a | P1a:LiQ(50%) | LiQ |
| | 20 nm | 180 nm | 10 nm | 20 nm | 10 nm | 20 nm | 1 nm |
| E7b | SpMA1:PD1(5%) | SpMA1 | SpMA2 | H5:SEB(5%) | P8a | P8a:LiQ(50%) | LiQ |
| | 20 nm | 180 nm | 10 nm | 20 nm | 10 nm | 20 nm | 1 nm |

TABLE 6
Data of the OLEDs for blue emission

| Ex. | U10 [V] | EQE10 [%] |
|---|---|---|
| V6 | 5.2 | 4.5 |
| E6a | 4.0 | 7.0 |
| E6b | 4.4 | 6.6 |
| E6c | 4.4 | 6.3 |
| V7 | 6.2 | 4.3 |
| E7a | 5.0 | 6.7 |
| E7b | 5.4 | 6.0 |

TABLE 7
Structural formulae of the materials of the OLEDs used, if not
already described before:

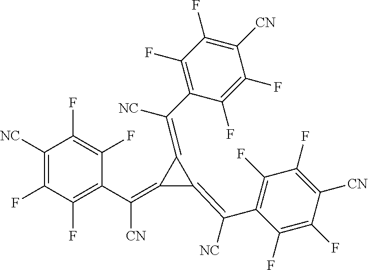 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

PD1 (CAS Reg. No. 1224447-88-4)

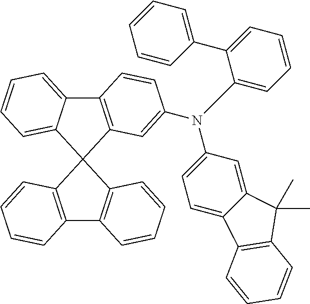 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

SpMA1

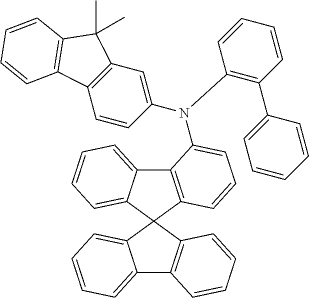 [SMILES: CC1(C)c2ccccc2-c2ccc(N(c3ccccc3-c3ccccc3)c3cccc4c3-c3ccccc3C43c4ccccc4-c4ccccc43)cc21]

SpMA2

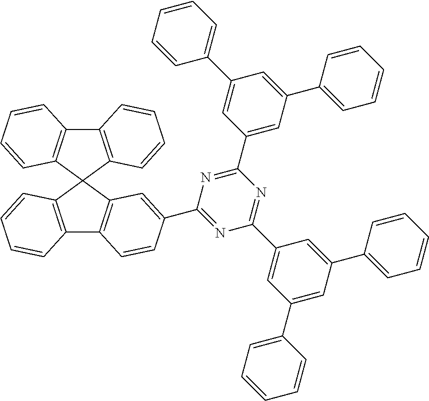 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

ST2

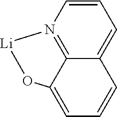

LiQ

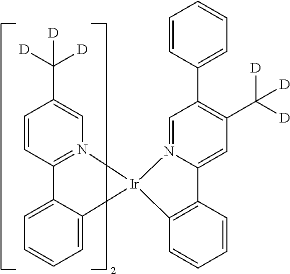

TE1

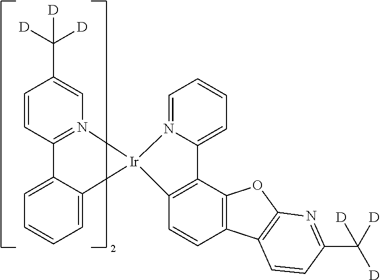

TE2

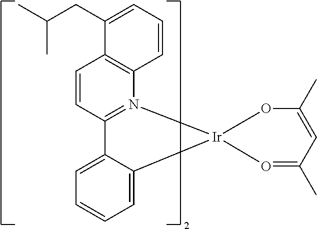

TER1

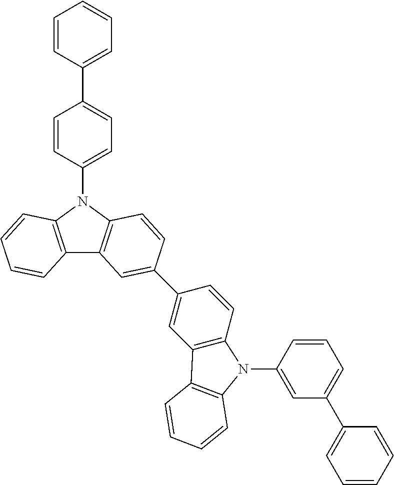 [SMILES: c1ccc(-c2ccc(-n3c4ccccc4c4cc(-c5ccc6c(c5)c5ccccc5n6-c5cccc(-c6ccccc6)c5)ccc43)cc2)cc1]

H1

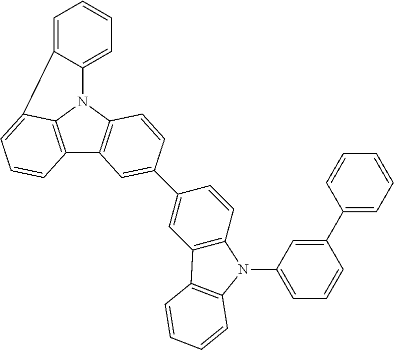 [SMILES: c1ccc(-c2cccc(-n3c4ccccc4c4cc(-c5ccc6c(c5)c5cccc7c8ccccc8n6c75)ccc43)c2)cc1]

H2

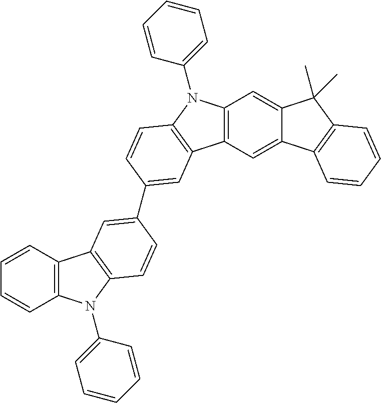 [SMILES: CC1(C)c2ccccc2-c2cc3c4cc(-c5ccc6c(c5)c5ccccc5n6-c5ccccc5)ccc4n(-c4ccccc4)c3cc21]

H3

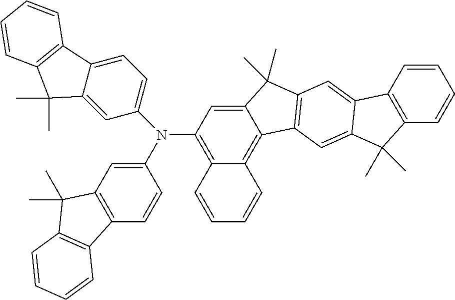 [SMILES: CC1(C)c2ccccc2-c2ccc(N(c3ccc4c(c3)C(C)(C)c3ccccc3-4)c3cc4c(c5ccccc35)-c3cc5c(cc3C4(C)C)-c3ccccc3C5(C)C)cc21]

SEB

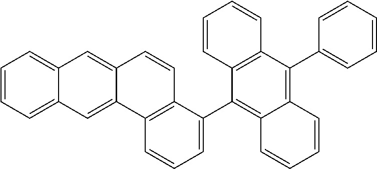 [SMILES: c1ccc(-c2c3ccccc3c(-c3cccc4c3ccc3cc5ccccc5cc34)c3ccccc23)cc1]

H5

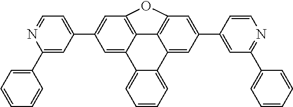 [SMILES: c1ccc(-c2cc(-c3cc4oc5cc(-c6ccnc(-c7ccccc7)c6)cc6c7ccccc7c(c3)c4c56)ccn2)cc1]

SdT1 (WO 2012/048781)

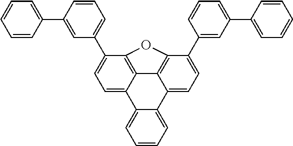 [SMILES: c1ccc(-c2cccc(-c3ccc4c5ccccc5c5ccc(-c6cccc(-c7ccccc7)c6)c6oc3c4c65)c2)cc1]

SdT2 (WO 2012/048781)

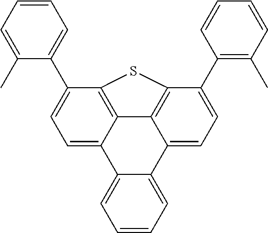 [SMILES: Cc1ccccc1-c1ccc2c3ccccc3c3ccc(-c4ccccc4C)c4sc1c2c43]

SdT3 (WO 2012/048781)

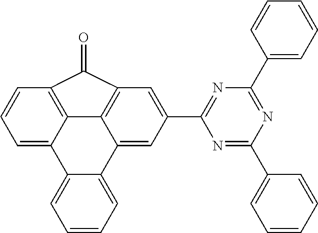 [SMILES: O=c1c2cccc3c4ccccc4c4cc(-c5nc(-c6ccccc6)nc(-c6ccccc6)n5)cc1c4c23]

SdT4 (WO 2012/048781)

1.-14. (canceled)

15. A compound of formula (1) 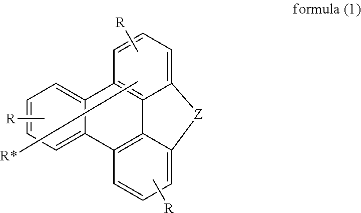
where the R radicals may also occur more than once and the symbols used are:
Z is O or S;
R* is a group of the formula (2), where the dotted bond represents the bond to the base skeleton in the formula (1), 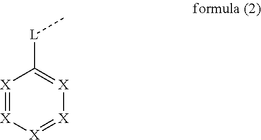
X is the same or different at each instance and is CR or N; or two adjacent X groups are a group of the following formula (3), (4) or (5), with the proviso that at least two and at most three X groups are N, 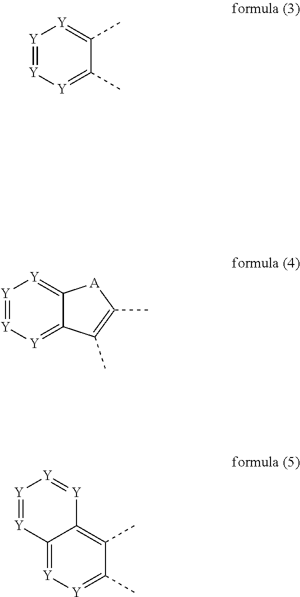
where the dotted bonds indicate the linkage of this group in the formula (2);
Y is the same or different at each instance and is CR or N;
A is NR, O, S or CR₂;
L is a single bond or an aromatic or heteroaromatic ring system which has 5 to 24 aromatic ring atoms and may be substituted by one or more R radicals;
R is the same or different at each instance and is H, D, F, Cl, Br, I, N(R¹)₂, CN, NO₂, OR¹, SR¹, COOR¹, C(═O)N(R¹)₂, Si(R¹)₃, B(OR¹)₂, C(═O)R¹, P(═O)(R¹)₂, S(═O)R¹, S(═O)₂R¹, OSO₂R¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals, where one or more nonadjacent CH₂ groups may be replaced by Si(R¹)₂, C═O, NR¹, O, S or CONR¹, or an aromatic or heteroaromatic ring system which has 5 to 60 aromatic ring atoms, and may be substituted in each case by one or more R¹ radicals; at the same time, two R radicals together may also form an aliphatic or heteroaliphatic ring system;
R¹ is the same or different at each instance and is H, D, F, Cl, Br, I, CN, NO₂, OR², SR², Si(R²)₃, B(OR²)₂, C(═O)R², P(═O)(R²)₂, S(═O)R², S(═O)₂R², OSO₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may each be substituted by one or more R² radicals, where one or more nonadjacent CH₂ groups may be replaced by Si(R²)₂, C═O, NR², O, S or CONR² and where one or more hydrogen atoms in the alkyl, alkenyl or alkynyl group may be replaced by D, F, Cl, Br, I or CN, or an aromatic or heteroaromatic ring system which has 5 to 40 aromatic ring atoms and may be substituted in each case by one or more R² radicals; at the same time, two or more R¹ radicals together may form an aliphatic ring system; and
R² is the same or different at each instance and is H, D, F, CN or an aliphatic, aromatic or heteroaromatic organic radical having 1 to 20 carbon atoms, in which one or more hydrogen atoms may also be replaced by F.

16. The compound as claimed in claim 15, wherein the compound is selected from the compounds of the formulae (6a), (6b), (7a) and (7b) 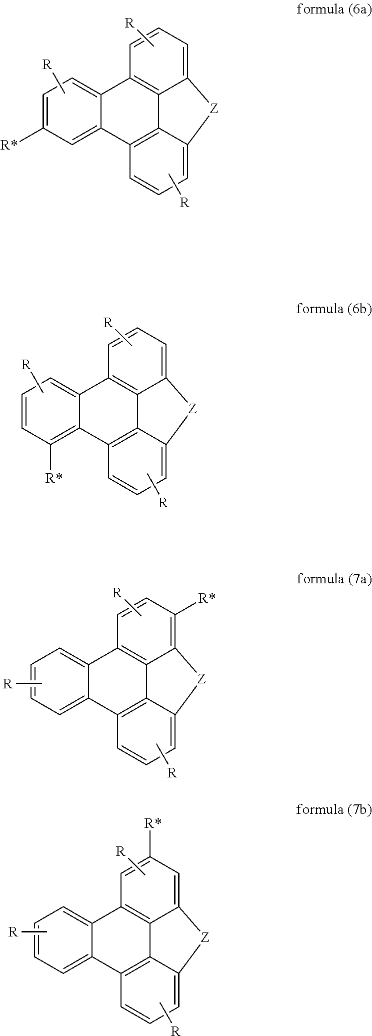
where the R radicals may also occur more than once, and the symbols have the definitions given in claim 15.

17. The compound as claimed in claim 15, characterized in that Z is O.

18. The compound as claimed in claim 15, characterized in that not more than two substituents R are a group other than H or D.

19. The compound as claimed in claim 15, wherein the compound is selected from the formulae (6a-1) to (7b-4) 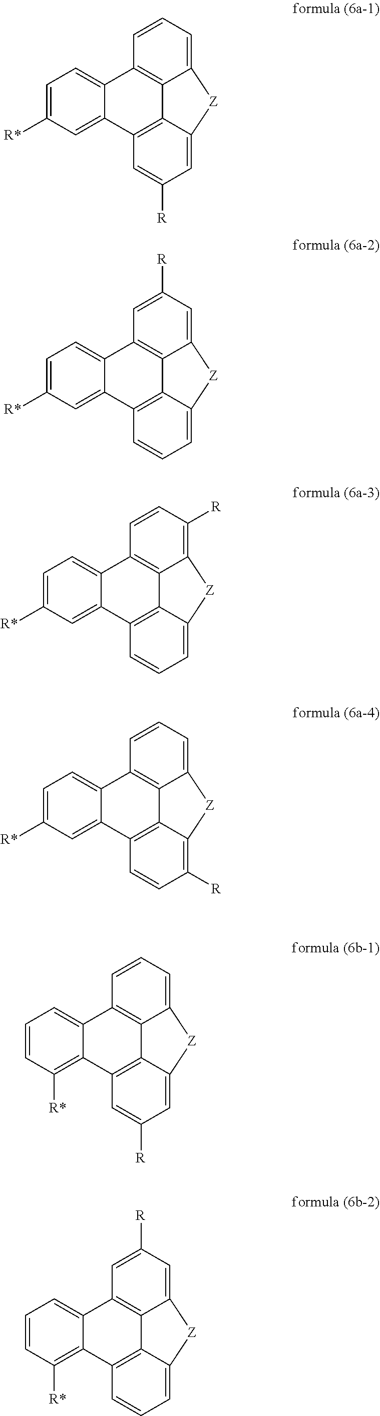 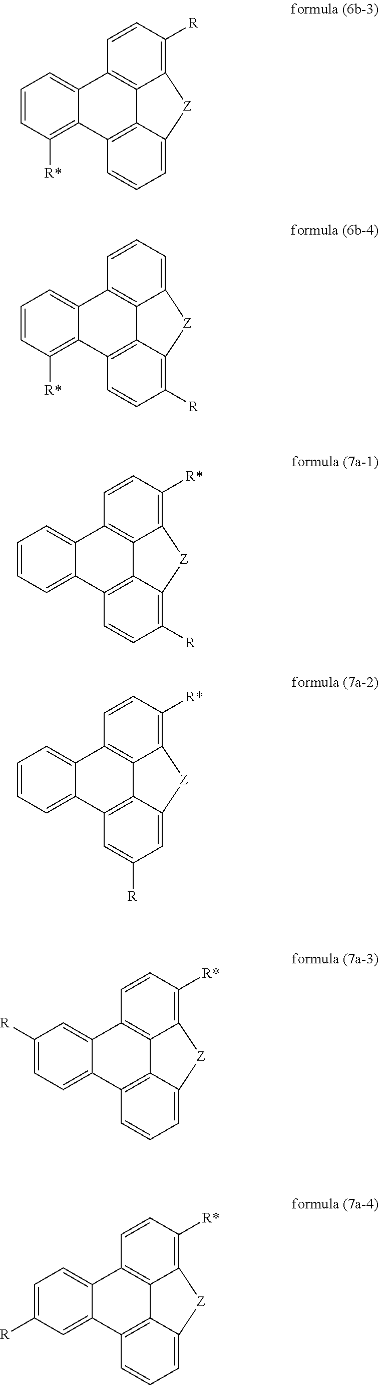 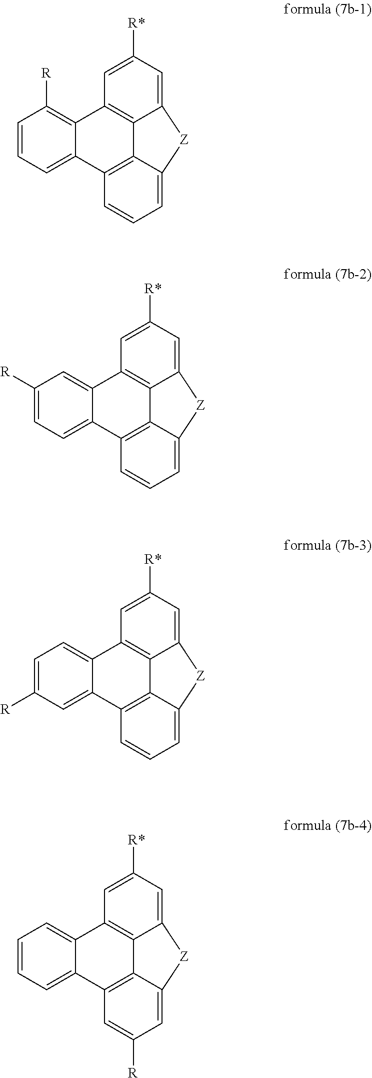
where the symbols have the definitions given in claim 15.

20. The compound as claimed in claim 15, characterized in that L is a single bond or a bivalent aromatic ring system which has 6 to 12 aromatic ring atoms and may be substituted by one or more R radicals, or a dibenzofuran or dibenzothiophene group that may be substituted by one or more R radicals.

21. The compound as claimed in claim 15, characterized in that L, when L is an aromatic or heteroaromatic ring system, is selected from the structures of the formulae (L-1) to (L-26) 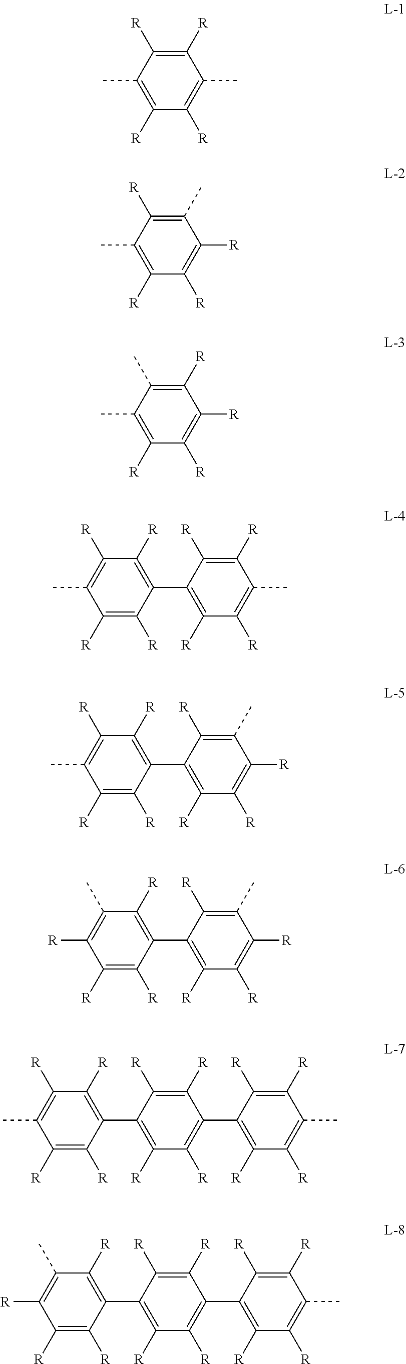 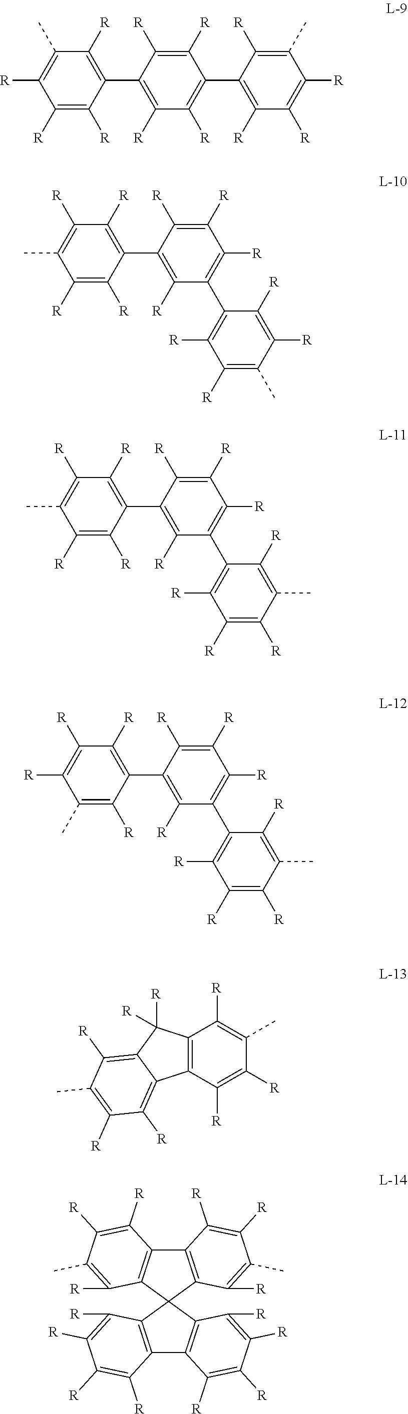 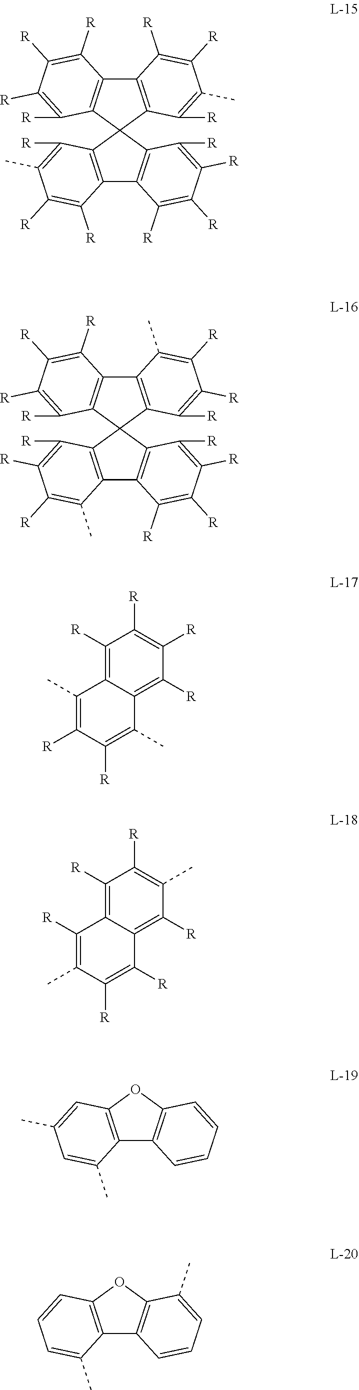 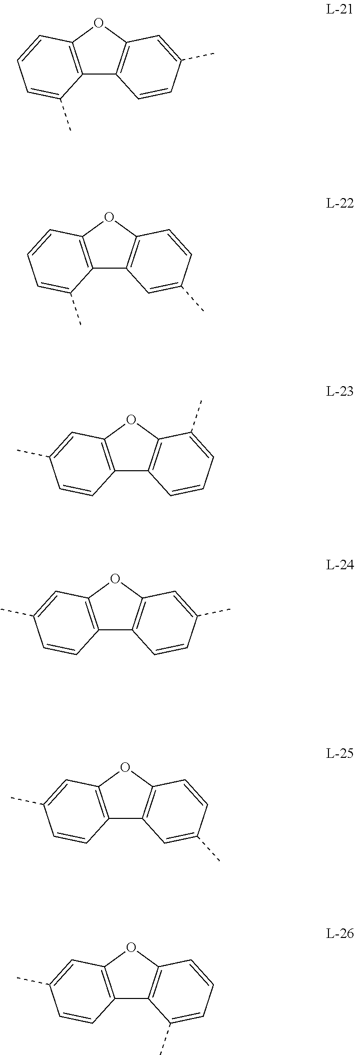
where the symbols have the meanings given in claim 15 and the dotted bonds represent the bonds to the heteroaryl group in the group of the formula (2) and to the base skeleton of the compound of the formula (1).

22. The compound as claimed in claim 15, characterized in that the group of the formula (2) is selected from the structures of the formulae (8) to (18): 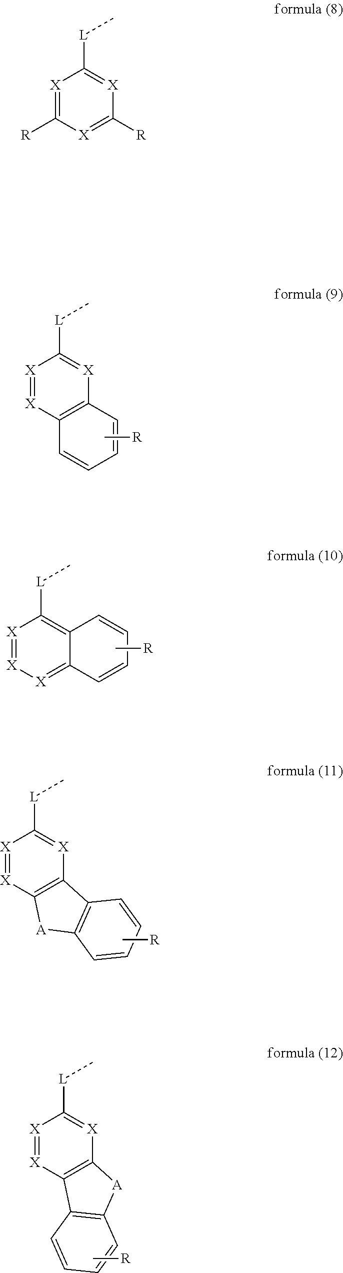 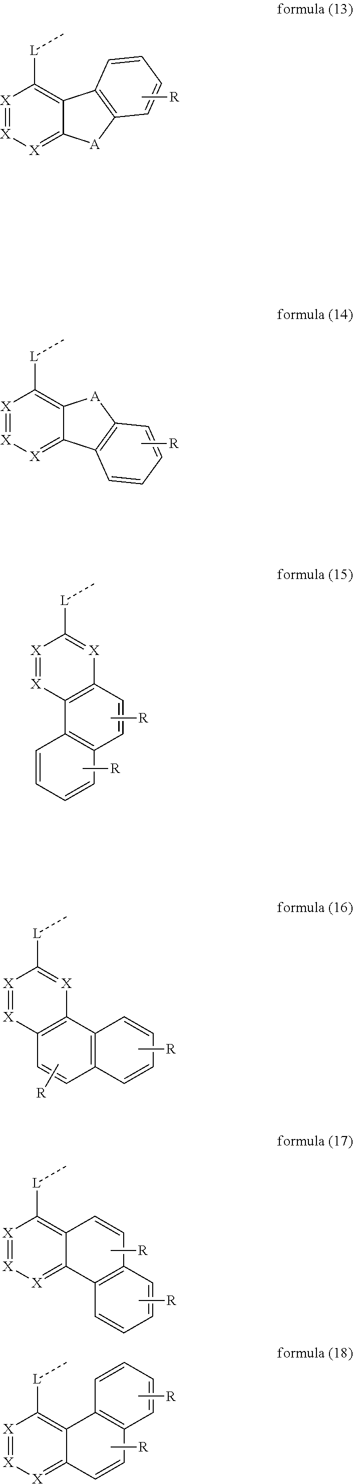
where the symbols used have the definitions given in claim 15 and, in formula (8), two or three X are N and R is the same or different at each instance and is an aromatic heteroaromatic ring system which has 5 to 40 aromatic ring atoms and may be substituted by one or more R¹ radicals, and where, in the formulae (9) to (18), exactly two X are N and R may also occur repeatedly.

23. The compound as claimed in claim 15, characterized in that the group of the formula (2) is selected from the groups of the formulae (8a) to (18a): 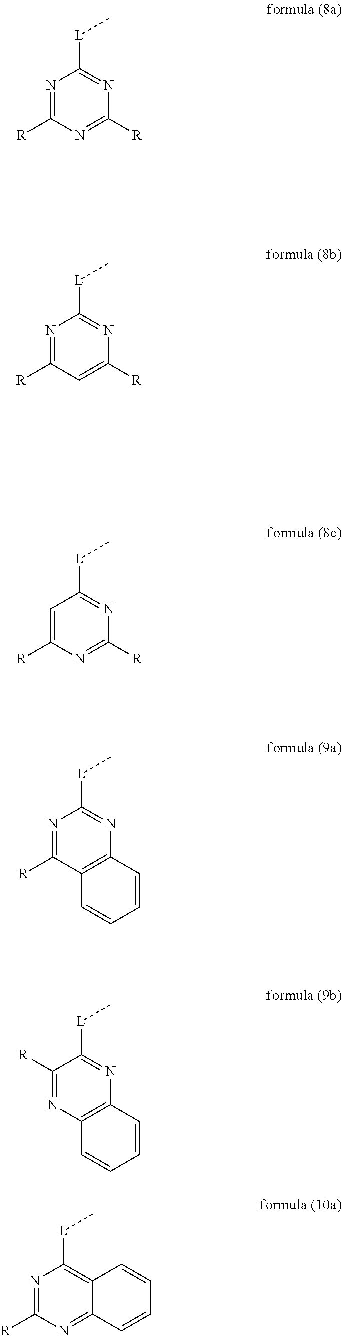 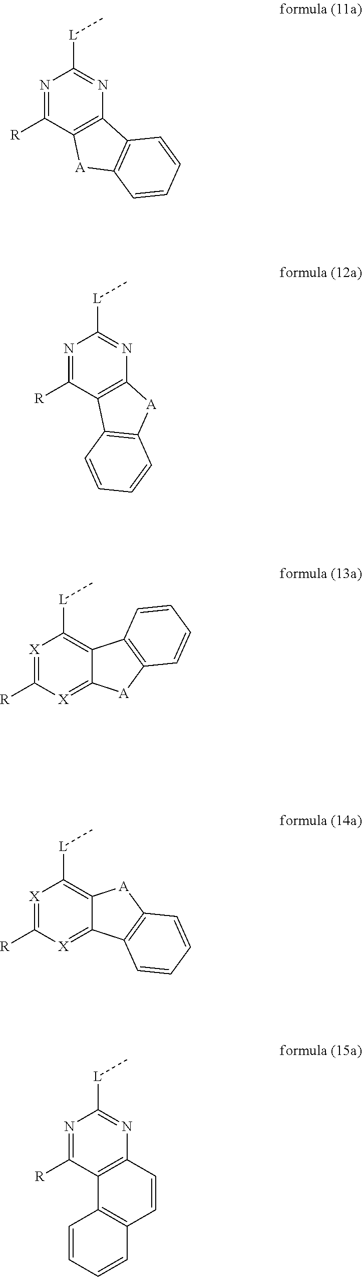 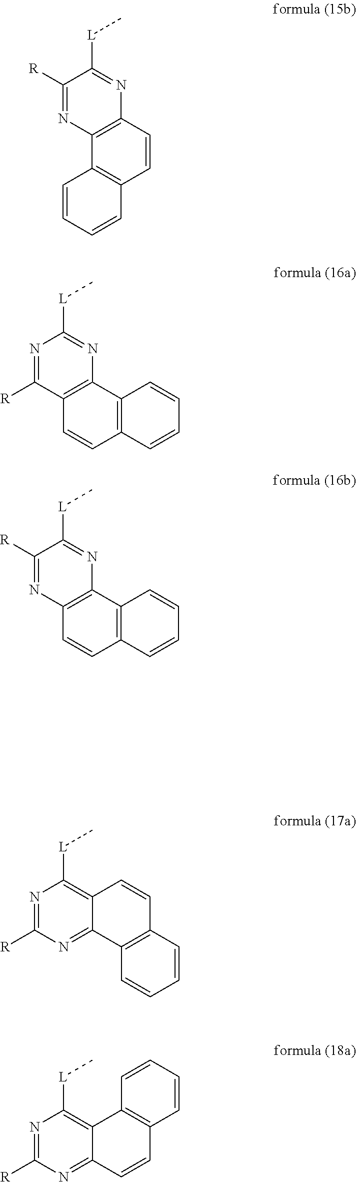
where the symbols have the definitions given in claim 15 and R is the same or different at each instance and is an aromatic or heteroaromatic ring system which has 5 to 40 aromatic ring atoms and may be substituted by one or more R¹ radicals.

24. A process for preparing the compound as claimed in claim 15, characterized by the steps of:
a) synthesizing the base skeleton that bears a reactive leaving group rather than the R* group; and
b) introducing the substituent R* by a coupling reaction.

25. A formulation comprising at least one compound as claimed in claim 15 and at least one further compound and/or solvent.

26. An electronic device comprising at least one compound as claimed in claim 15.

27. An organic electroluminescent device comprising the compound as claimed in claim 15 wherein the compound is used in an emitting layer as matrix material for phosphorescent or fluorescent emitters or for emitters that exhibit TADF (thermally activated delayed fluorescence), and/or in an electron transport layer and/or in a hole blocker layer.
